(19) United States
(12) Patent Application Publication
(10) Pub. No.: US 2023/0102999 A1
(43) Pub. Date: Mar. 30, 2023

(54) DEUTERATED NICLOSAMIDE

(71) Applicant: First Wave Bio, Inc., Ann Arbor, MI (US)

(72) Inventor: Gary D. Glick, Ann Arbor, MI (US)

(21) Appl. No.: 17/791,666

(22) PCT Filed: Jan. 8, 2021
(86) PCT No.: PCT/US2021/012673

§ 371 Date: Jul. 8, 2022

Related U.S. Application Data
(60) Provisional application No. 62/959,410, filed on Jan. 10, 2020.

(51) Int. Cl. C07B 59/00; A61P 31/04

(52) U.S. Cl. CPC C07B 59/001; A61P 31/04

(57) ABSTRACT

This disclosure features chemical entities (e.g., a compound or a pharmaceutically acceptable salt, and/or hydrate, and/or cocrystal, and/or drug combination of the compound) that exhibit activity as mitochondrial uncoupling agents. Said chemical entities are useful, e.g., for treating one or more symptoms of a pathology characterized by an abnormal inflammatory response (e.g., inflammatory bowel diseases) in a subject (e.g., a human). This disclosure also features compositions containing the same as well as methods of using and making the same.

### CROSS REFERENCE TO RELATED APPLICATIONS

This application claims the benefit of U.S. Provisional Application Ser. No. 62/959,410, filed on Jan. 10, 2020, the disclosure of which is incorporated herein by reference in its entirety.

### TECHNICAL FIELD

This disclosure features chemical entities (e.g., a compound or a pharmaceutically acceptable salt, and/or hydrate, and/or cocrystal, and/or drug combination of the compound) that exhibit activity as mitochondrial uncoupling agents. Said chemical entities are useful, e.g., for treating one or more symptoms of a pathology characterized by an abnormal inflammatory response (e.g., inflammatory bowel diseases) in a subject (e.g., a human). This disclosure also features compositions containing the same as well as methods of using and making the same.

### BACKGROUND

Ulcerative colitis (UC) and Crohn's disease (CD) are the predominant chronic, inflammatory bowel diseases (IBD) in humans. These disorders are autoimmune in nature and occur in the absence of infection. IBD effects up to 2,000,000 Americans (increasing ˜15% annually) and it is associated with unacceptably high rates of morbidity and mortality. IBD is also a significant burden on the U.S. health care system as the most effective treatments are biological drugs that are quite costly.

IBD occurs as the result of inappropriate immune responses in genetically susceptible individuals mediated by complex interactions between environmental stimuli, microbial factors, and the intestinal immune system. The hallmark of IBD is represented by excessive immune responses that mediate gastrointestinal tissue damage, either directly or through the release of soluble, pro-inflammatory mediators.

T cells are a type of immune cell that infiltrate the intestinal mucosa and are key drivers of gastrointestinal tissue damage in IBD. These cells persist and accumulate in the intestinal mucosa because normal physiologic mechanisms designed to censor or eliminate activated T cells are inoperative in the context of IBD. While the exact basis for T cell accumulation in IBD is not fully elucidated, chronic activation by microbial stimuli along with the cytokine milieu at the sites of inflammation within gastrointestinal tissue are thought to be important. Regardless of how these cells persist, enhancing T cell death in the intestinal mucosa is linked with resolution of IBD and drugs that are most effective in managing IBD function (in part), by killing pathogenic T cells resident in the gut.

Although different forms of IBD show pathophysiological and clinical differences, the therapeutic approach to managing IBD shares many common elements. Medical management of IBD is largely empirical, employing anti-inflammatory or immunosuppressive drugs. Salicylazosulfapyridine and 5-aminosalicylic acid are used to treat mild IBD and as maintenance therapy if disease remission can be achieved. Corticosteroids are used in patients with moderate to severe disease. However, clinical remission can only be obtained in ˜60% of patients, and just about half of these stay in remission after treatment is discontinued. This last point is significant because long-term use of corticosteroids carries a significant risk of serious side effects.

Immunosuppressive drugs can also be used to treat moderate to severe cases of IBD, often as a replacement for steroid therapy. However, immunosuppressive drugs (e.g., azathioprine) usually cannot ensure control of symptoms, and treatment is accompanied by numerous contraindications and severe side effects.

Drugs that often show the best efficacy in treating IBD are systemically administered (via injection or infusion) monoclonal antibodies that block TNF-alpha, a pro-inflammatory cytokine overproduced during all forms of IBD (e.g., UC, CD, graft-versus-host disease, celiac disease, iatrogenic colitis such as that induced by checkpoint inhibitors, etc.). Reducing levels of TNF-alpha in the context of IBD has two consequences. First, as an inflammatory cytokine, TNF-alpha mediates tissue damage. Second, high levels of TNF-alpha help disease causing T cells to survive and blocking TNF-alpha activity eventually leads to T cell death. Indeed, the induction of cell death by anti-TNF-alpha drugs like infliximab can predict clinical improvement in patients.

Although effective, use of anti-TNF-alpha drugs is associated with severe, systemic side effects including, re-activation of latent pathogens, hypersensitivity phenomena, cancer, and the formation of autoantibodies. Some patients are inherently resistant to anti-TNF-alpha drugs and over time, almost half of all patients that do show a response, develop resistance.

From the foregoing it is clear that there is need for new drugs to treat IBD that are more effective, less toxic, less expensive, and more convenient to administer versus standard of care.

Niclosamide (5-chloro-N-(2-chloro-4-nitrophenyl)-2-hydrobenzamide) is a halogenated salicylanilide that belongs to a group of medicines known as anthelmintics. Anthelmintics are medicines used in the treatment of worm infections. Niclosamide, which has low systemic bioavailability and an excellent safety profile, is used to treat broad or fish tapeworm, dwarf tapeworm, and beef tapeworm infections. It is believed that Niclosamide inhibits oxidative phosphorylation and stimulates adenosine triphosphatase activity in the mitochondria of cestodes (e.g., tapeworm), killing the scolex and proximal segments of the tapeworm both in vitro and in vivo (see, Li, Y., et al., Cancer Lett. 2014 349, 8-14.).

Deuterium (D or ²H) is a stable and non-radioactive isotope of hydrogen. Carbon-hydrogen bonds contain a naturally occurring distribution of hydrogen isotopes, namely ¹H or protium (about 99.9844%), D or deuterium (about 0.0156%), and T or tritium (in the range between about 0.5 and 67 tritium atoms per 10¹⁸ protium atoms). Attempts to use deuteration of pharmaceuticals to improve pharmacokinetic (PK) profiles have been described previously.

The effects of deuterium substitution on the rate of metabolism have been reported to be unpredictable. For some compounds deuteration caused an increase in metabolic stability. For some compounds there was no change in metabolic stability. In some cases a decrease in metabolic stability was observed. See, e.g., Blake, M I et al, J Pharm Sci, 1975, 64:367-91; Foster, A B, Adv Drug Res 1985, 14:1-40; Kushner, D J et al, Can J Physiol Pharmacol 1999, 79-88; Fisher, M B et al, Curr Opin Drug Discov Devel, 2006, 9:101-09. The magnitude and nature of the effect deuterium benefit cannot be predicted a priori. See Concert Pharmaceuticals et al., “Precision Deuterium Chemistry Backgrounder,” Concert Pharmaceuticals Inc., 2007, 1-6.

### SUMMARY

This disclosure features chemical entities (e.g., a compound or a pharmaceutically acceptable salt, and/or hydrate, and/or cocrystal, and/or drug combination of the compound) that exhibit activity as mitochondrial uncoupling agents. Said chemical entities are useful, e.g., for treating one or more symptoms of a pathology characterized by an abnormal inflammatory response (e.g., inflammatory bowel diseases) in a subject (e.g., a human). This disclosure also features compositions containing the same as well as methods of using and making the same.

In one aspect, compounds of Formula (I), or a pharmaceutically acceptable salt thereof, are featured:

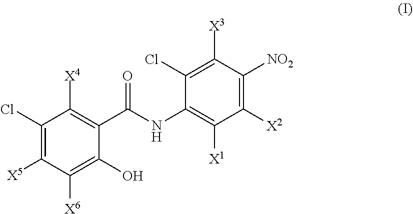

in which X¹, X², X³, X⁴, X⁵, and X⁶ can be as defined anywhere herein.

The recitations “compound of Formula (I)” and “deuterated niclosamide” are used interchangeably herein.

In one aspect, pharmaceutical compositions are featured that include a chemical entity described herein (e.g., a compound described generically or specifically herein or a pharmaceutically acceptable salt thereof or compositions containing the same) and one or more pharmaceutically acceptable excipients.

In one aspect, methods for inducing cell death of one or more T cells (e.g., in the digestive and/or gastrointestinal tract (GI)), of a subject are provided. The methods include contacting the one or more T cells with an effective amount of a compound of Formula (I) or a pharmaceutically acceptable salt and/or cocrystal thereof.

In another aspect, methods for treating a subject having a condition associated with unregulated (abnormal, elevated) recruitment and/or retention of one or more T cells (e.g., at the digestive and/or gastrointestinal tract (GI)) of the subject) are provided. The methods include administering to the subject in need thereof a compound of Formula (I) or a pharmaceutically acceptable salt and/or cocrystal thereof. In some embodiments, the methods include orally administering the compound of Formula (I). In some embodiments, the methods include rectally (e.g., via enema) administering the compound of Formula (I).

In some embodiments, at least some of said T cells are located in the small bowel (e.g., in the ileum portion of the small bowel) of the subject. As such, the methods described herein can be used, e.g., in the treatment of an inflammatory bowel disease, such as Crohn's disease.

In some embodiments, at least some of said T cells are located in the colon of the subject. As such, the methods described herein can be used, e.g., in the treatment of in the treatment of an inflammatory bowel disease, such as ulcerative colitis.

In a further aspect, methods for treating a condition (or one or more symptoms thereof) characterized by an abnormal inflammatory response in a subject in need thereof are provided (e.g., an autoimmune disorder, e.g., colitis, e.g., autoimmune colitis, e.g, an inflammatory bowel disease, e.g., Crohn's disease, ulcerative colitis). The methods include administering to the subject an effective amount of a compound of Formula (I) or a pharmaceutically acceptable salt and/or cocrystal thereof. In some embodiments, the methods include orally administering the compound of Formula (I). In some embodiments, the methods include rectally (e.g., via enema) administering the compound of Formula (I).

In a further aspect, methods for treating colitis (or one or more symptoms thereof) in a subject are provided. The methods include administering to the subject an effective amount of a compound of Formula (I) or a pharmaceutically acceptable salt and/or cocrystal thereof. In some embodiments, the methods include orally administering the compound of Formula (I). In some embodiments, the methods include rectally (e.g., via enema) administering the compound of Formula (I).

In another aspect, methods for treating an autoimmune disease, cancer, a metabolic disorder, a cardiovascular disease, a condition associated with microbial (e.g., viral or bacterial) infection, an allergic disease, a condition associated with NF-κB activation and/or inflammatory cytokine production, a condition mediated by an aquaporin, or a neurodenegerative or psychiatric disease are provided. The methods include administering to the subject an effective amount of a compound of Formula (I) or a pharmaceutically acceptable salt and/or cocrystal thereof. In some embodiments, the methods include orally administering the compound of Formula (I). In some embodiments, the methods include rectally (e.g., via enema) administering the compound of Formula (I).

In another aspect, provided herein is a method for treating a condition in a subject by administering an effective amount of a compound of Formula (I), wherein the condition is a condition as described in U.S. patent application Ser. No. 62/990,414, filed on Mar. 16, 2020; U.S. patent application Ser. No. 62/993,688, filed on Mar. 23, 2020; and U.S. patent application Ser. No. 63/002,324, filed on Mar. 30, 2020, each of which is incorporated herein by reference in its entirety.

In another aspect, this disclosure features methods useful for preventing the progression of COVID-19 in a subject (e.g., methods for reducing the likelihood of a subject's developing COVID-19 as well as methods for reducing or slowing the progression of COVID-19 in a subject, e.g., reducing the likelihood that a subject will experience one or more severe or life-threating COVID-19 symptoms), wherein the method includes administering to the subject an effective amount of a compound of Formula (I) or a pharmaceutically acceptable salt and/or cocrystal thereof.

In a further aspect, provided herein is a method for treating a condition associated with viral infection (e.g., COVID-19-related viral infection, a COVID-19 viral infection, a SARS-CoV-2 infection). The method includes administering to the subject an effective amount of a compound of Formula (I) or a pharmaceutically acceptable salt and/or cocrystal thereof. In some embodiments, the method includes orally administering the compound of Formula (I) so as to treat the COVID-19-related viral infection.

In another aspect, a method of reducing the risk of developing COVID-19 in a subject at risk thereof is provided. The method includes administering an effective amount (e.g., a prophylactically effective amount) of the compound of Formula (I), or a pharmaceutically acceptable salt and/or cocrystal thereof, to the subject.

In another aspect, a method for treating COVID-19 in a subject in need thereof is provided. The method includes administering an effective amount of the compound of Formula (I), or a pharmaceutically acceptable salt and/or cocrystal thereof, to the subject, such as to the GI tract of the subject. In another aspect, a method for treating one or more digestive symptoms in a subject having COVID-19 is provided. The method includes administering the compound of Formula (I), or a pharmaceutically acceptable salt and/or cocrystal thereof, to the subject.

In another aspect, a method for treating mild COVID-19 in a subject in need thereof is provided. The method includes administering an effective amount of the compound of Formula (I), or a pharmaceutically acceptable salt and/or cocrystal thereof, to the subject. In another aspect, a method for treating severe COVID-19 in a subject in need thereof is provided. The method includes administering an effective amount of the compound of Formula (I), or a pharmaceutically acceptable salt and/or cocrystal thereof, to the subject.

In a further aspect, provided herein is a method for reducing SARS-CoV-2 transmission from a subject. The method includes administering the compound of Formula (I), or a pharmaceutically acceptable salt and/or cocrystal thereof, to a subject having COVID-19. In another aspect, a method for reducing the viral load of SARS-CoV-2 in a subject is provided. The method includes administering the compound of Formula (I), or a pharmaceutically acceptable salt and/or cocrystal thereof, to a subject having COVID-19.

In another aspect, this disclosure features a method of treating a subject having COVID-19, the method comprising: identifying a subject that has: (i) a respiratory rate of <30 breaths per min; (ii) an oxygen saturation at rest of >93%; and (iii) a ratio of partial pressure of arterial oxygen to fractional concentration of oxygen inspired air of >300 mm Hg; and administering to the nasal cavity of the identified subject a treatment that includes a compound of Formula (I), or a pharmaceutically acceptable salt and/or cocrystal thereof. In certain embodiments, a compound of Formula (I), or a pharmaceutically acceptable salt and/or cocrystal thereof, is formulated for delivery by inhalation.

In another aspect, this disclosure features a method of treating a subject having COVID-19, the method comprising: identifying a subject that has at least one of (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication; and administering to the lungs of the identified subject a treatment that includes a a compound of Formula (I), or a pharmaceutically acceptable salt and/or cocrystal thereof. In certain embodiments, the compound of Formula (I), or a pharmaceutically acceptable salt and/or cocrystal thereof, is formulated for delivery by inhalation.

In another aspect, this disclosure features a method for treating severe COVID-19 in a subject in need thereof, the method comprising administering an effective amount of a a compound of Formula (I), or a pharmaceutically acceptable salt and/or cocrystal thereof, to the GI tract of the subject (e.g., by oral delivery).

In another aspect, this disclosure features a method of treating a subject having COVID-19, the method comprising: (a) identifying a subject having (i) a respiratory rate of <30 breaths per min; (ii) an oxygen saturation at rest of >93%; and (iii) a ratio of partial pressure of arterial oxygen to fractional concentration of oxygen inspired air of >300 mm Hg; (b) administering one or more doses of a a compound of Formula (I), or a pharmaceutically acceptable salt and/or cocrystal thereof, to the nasal cavity of the subject; (c) after (a) and (b), identifying whether the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; and (d) administering one or more doses of a a compound of Formula (I), or a pharmaceutically acceptable salt and/or cocrystal thereof, to the lungs of the subject in which the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication; or (e) administering additional doses of the a compound of Formula (I), or a pharmaceutically acceptable salt and/or cocrystal thereof, to the nasal cavity of the subject in which (i) a respiratory rate of <30 breaths per min; (ii) an oxygen saturation at rest of >93%; and (iii) a ratio of partial pressure of arterial oxygen to fractional concentration of oxygen inspired air of >300 mm Hg.

In another aspect, this disclosure features a method of treating a subject having COVID-19, the method comprising: (a) identifying a subject having (i) a respiratory rate of <30 breaths per min; (ii) an oxygen saturation at rest of >93%; and (iii) a ratio of partial pressure of arterial oxygen to fractional concentration of oxygen inspired air of >300 mm Hg; (b) administering one or more doses of a a compound of Formula (I), or a pharmaceutically acceptable salt and/or cocrystal thereof, to the nasal cavity of the subject; (c) after (a) and (b), identifying whether the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication; and (d) administering one or more doses of a a compound of Formula (I), or a pharmaceutically acceptable salt and/or cocrystal thereof, to the GI tract of the subject in which the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; or (e) administering additional doses of the compound of Formula (I), or a pharmaceutically acceptable salt thereof, to the nasal cavity of the subject in which (i) a respiratory rate of <30 breaths per min; (ii) an oxygen saturation at rest of >93%; and (iii) a ratio of partial pressure of arterial oxygen to fractional concentration of oxygen inspired air of >300 mm Hg.

In another aspect, this disclosure features a method of treating a subject having COVID-19, the method comprising: (a) identifying a subject having (i) a respiratory rate of <30 breaths per min; (ii) an oxygen saturation at rest of >93%; and (iii) a ratio of partial pressure of arterial oxygen to fractional concentration of oxygen inspired air of >300 mm Hg; (b) administering one or more doses of a a compound of Formula (I), or a pharmaceutically acceptable salt and/or cocrystal thereof, to the nasal cavity of the subject; (c) after (a) and (b), identifying whether the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication; and (d) administering one or more doses of a a compound of Formula (I), or a pharmaceutically acceptable salt and/or cocrystal thereof, to the GI tract of the subject and administering one or more doses of a a compound of Formula (I), or a pharmaceutically acceptable salt and/or cocrystal thereof, to the lungs of the subject in which the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; or (e) administering additional doses of the a compound of Formula (I), or a pharmaceutically acceptable salt and/or cocrystal thereof, to the nasal cavity of the subject in which (i) a respiratory rate of <30 breaths per min; (ii) an oxygen saturation at rest of >93%; and (iii) a ratio of partial pressure of arterial oxygen to fractional concentration of oxygen inspired air of >300 mm Hg.

In another aspect, this disclosure features a method of treating a subject having COVID-19, the method comprising: (a) identifying a subject having at least one of (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication; (b) administering one or more doses of a a compound of Formula (I), or a pharmaceutically acceptable salt and/or cocrystal thereof, to the lungs of the subject; (c) after (a) and (b), identifying whether the subject has (i) a respiratory rate of <30 breaths per min; (ii) an oxygen saturation at rest of >93%; and (iii) a ratio of partial pressure of arterial oxygen to fractional concentration of oxygen inspired air of >300 mm Hg; and (d) administering one or more doses of a a compound of Formula (I), or a pharmaceutically acceptable salt and/or cocrystal thereof, to the nasal cavity of the subject in which the subject has (i) a respiratory rate of <30 breaths per min; (ii) an oxygen saturation at rest of >93%; and (iii) a ratio of partial pressure of arterial oxygen to fractional concentration of oxygen inspired air of >300 mm Hg; or (e) administering additional doses of the a compound of Formula (I), or a pharmaceutically acceptable salt and/or cocrystal thereof, to the lungs of the subject in which i(i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication.

In another aspect, this disclosure features methods that include administering a co-crystal that includes a compound of Formula (I) having any one or more or the properties described herein), or a pharmaceutically acceptable salt thereof and a pharmaceutically acceptable coformer. In some embodiments, the cocrystal has a reduced particle size as described anywhere herein (e.g., the cocrystal itself can be reduced to have the reduced particle size range, and/or the reduced particle size distribution described herein for compound of Formula (I).

In another aspect, provided herein is a solid pharmaceutical composition, which comprises: an inner phase which is a wet granulated solid preparation comprising a compound of Formula (I), or a pharmaceutically acceptable salt and/or cocrystal thereof, one or more disintegrants; one or more diluents; and one or more binders; and an external phase comprising one or more glidants and/or one or more lubricants.

In another aspect, provided herein is a solid pharmaceutical composition, which comprises: an inner phase which is a wet granulated solid preparation comprising a compound of Formula (I), or a pharmaceutically acceptable salt and/or cocrystal thereof; crospovidone; lactose monohydrate; and povidone; and an external phase comprising magnesium stearate and talc.

In another aspect, provided herein is an enema preparation comprising a separately contained first component and a separately contained second component, wherein: (i) the first component comprises a solid pharmaceutical composition, which comprises: an inner phase which is a wet granulated solid preparation comprising a compound of Formula (I), or a pharmaceutically acceptable salt and/or cocrystal thereof, one or more disintegrants; one or more diluents; and one or more binders; an external phase comprising one or more glidants and/or one or more lubricants; and (ii) the second component comprises one or more liquids and optionally one or more other pharmaceutically acceptable excipients together forming a liquid carrier.

In another aspect, provided herein is an enema formulation comprising water; methyl cellulose; povidone; methylparaben; propylparaben; sodium dihydrogen phosphate dehydrate; disodium phosphate dodecahydrate; crospovidone; lactose monohydrate; magnesium stearate; talc; and a compound of Formula (I), or a pharmaceutically acceptable salt and/or cocrystal thereof.

In another aspect, provided herein is a method for treating iatrogenic autoimmune colitis in a subject, the method comprising administering by enema an effective amount of an enema formulation comprising a compound of Formula (I). The enema formulation can be described anywhere herein.

In another aspect, this disclosure features highly pure Formula (I) compounds, or pharmaceutically acceptable salts and/or cocrystal thereof.

In some embodiments, the highly pure Formula (I) compounds are useful starting materials, e.g., for preparation of Formula (I) compounds having a reduced particle size range (e.g., as determined by measuring the particle size distribution).

In another aspect, this disclosure features Formula (I) compounds, or a pharmaceutically acceptable salt thereof, having a reduced particle size (e.g., having a reduced particle size range, having a reduced particle size distribution).

In some embodiments, the Formula (I) compounds described herein have a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

In some embodiments, the Formula (I) compounds described herein have a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

In another aspect, this disclosure features highly pure Formula (I) compounds, or a pharmaceutically acceptable salt thereof, having a reduced particle size (e.g., having a reduced particle size range, having a reduced particle size distribution).

In some embodiments, the Formula (I) compounds as described herein have a chemical purity of greater than about 99.0%, a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

In another aspect, provided herein is a composition comprising a compound of Formula (I) (e.g., a compound of Formula (I) having high purity and/or reduced particle size) and one or more pharmaceutically acceptable excipients, wherein the composition is suitable for oral administration. In some embodiments, administration of a single dose of the composition to a subject produces a local concentration of the Formula (I) compound in the GI tract of the subject that is higher than the concentration of the compound in the plasma compartment of the subject.

In another aspect, the disclosure features a method for treating colitis in a subject in need thereof, the method comprising administering an effective amount of a compound of Formula (I) having high purity (e.g., >99%) and/or reduced particle size (e.g., with D(0.9), D(0.5), and D(0.1) values as described supra), a pharmaceutically acceptable salt thereof, a pharmaceutical composition thereof, a dosage form thereof, or a co-crystal containing the same.

Embodiments can include one or more of the following features.

The compound of Formula (I) can be administered orally.

The compound of Formula (I) can be administered rectally (e.g., via enema).

The compound of Formula (I) can be administered locally or topically.

The subject can be a human.

The condition can be associated with unregulated (such as abnormal or elevated) recruitment and/or retention of one or more T cells at the gastrointestinal tract (GI) of the subject.

The condition can be associated with unregulated (such as abnormal or elevated) activation of one or more T cells in the gastrointestinal tract (GI) of the subject.

The condition can be colitis. For example, the condition can be an autoimmune colitis; the condition can be an inflammatory bowel disease (e.g., ulcerative colitis or Crohn's disease). The condition can be colitis. The condition can be iatrogenic autoimmune colitis.

The condition can be colitis (e.g., iatrogenic autoimmune colitis) induced by one or more chemotherapeutic agents.

At least one of the one or more chemotherapeutic agents can be a chemotherapeutic immunomodulator such as an immune checkpoint inhibitor. The immune checkpoint inhibitor can be an inhibitor that targets an immune checkpoint receptor selected from the group consisting of CTLA-4, PD-1, PD-L1, PD-1-PD-L1, PD-1-PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9—TIM3, Phosphatidylserine—TIM3, lymphocyte activation gene 3 protein (LAG3), MHC class II—LAG3, 4-1BB-4-1BB ligand, OX40-OX40 ligand, GITR, GITR ligand—GITR, CD27, CD70-CD27, TNFRSF25, TNFRSF25-TLiA, CD40L, CD40-CD40 ligand, HVEM-LIGHT-LTA, HVEM, HVEM—BTLA, HVEM—CD160, HVEM—LIGHT, HVEM-BTLA-CD160, CD80, CD80—PDL-1, PDL2—CD80, CD244, CD48—CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86—CD28, CD86—CTLA, CD80—CD28, CD39, CD73 Adenosine-CD39-CD73, CXCR4-CXCL12, Phosphatidylserine, TIM3, Phosphatidylserine—TIM3, SIRPA-CD47, VEGF, Neuropilin, CD160, CD30, and CD155. The immune checkpoint inhibitor can be selected from the group consisting of: Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Avelumab (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, and MNRP1685A, and MGA271. The immune checkpoint inhibitor can be an inhibitor that targets CTLA-4. The immune checkpoint inhibitor can be an antibody. The antibody can be is ipilimumab or tremelimumab. The immune checkpoint inhibitor can be an inhibitor that targets PD1 or PD-L1. The immune checkpoint inhibitor can be selected from nivolumab, lambroizumab, and BMS-936559.

The condition can be selected from the group consisting of celiac disease, irritable bowel syndrome, mucositis, uveitis, radiation enteritis, rheumatoid arthritis, lupus, scleroderma, psoriasis, cutaneous T-cell lymphoma, acute graft vs. host disease and chronic graft vs. host disease.

The methods can further include administering one or more additional therapeutic agents.

For example, therapeutic agents useful for treating or preventing inflammatory bowel disease (IBD) (e.g., Crohn's disease, ulcerative colitis), e.g., sphingosine 1-phosphate (S1P) receptor modulators (e.g., etrasimod or ozanimod); steroidal anti-inflammatory agents (e.g, beclomethasone 17 or budesonide); non-steroidal anti-inflammatory agents (e.g., 5-ASA); receptor-interacting protein kinase 1 (RIPK1) inhibitors (e.g., GSK2982772); EP4 modulators (e.g., KAG-308); toll-like receptor (e.g., TLR4, TLR9) modulators (e.g., JKB-122, cobitolimod); Janus kinase (JAK) inhibitors (e.g., TD-1473, tofacitinib, upadacitinib, filgotinib, PF-06651600, and PF-06700841); lanthionine synthetase C-like 2 (LANCL2) modulators (e.g., BT-11); phosphatidylcholine (e.g., LT-02); integrin (e.g., a4 Integrin) modulators (e.g, AJM-300 (carotegrast)); Smad7 modulators (e.g., mongersen); phosphodiesterase 4 (PDE4) modulators (e.g., apremilast); tumor progression locus 2 (TPL2) inhibitors (e.g., GS-4875); tyrosine kinase 2 (TYK2) inhibitors (e.g., BMS-986165, PF-06700841, and PF-06826647); and/or TEC kinase inhibitors (e.g., PF-06651600).

As another example, the one or more therapeutic agents can be: budenoside; epidermal growth factor; corticosteroids; cyclosporine; sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1 monoclonal antibodies; anti-IL-6 monoclonal antibodies (e.g., anti-IL-6 receptor antibodies and anti-IL-6 antibodies); growth factors; elastase inhibitors; pyridinyl-imidazole compounds; TNF antagonists as described herein; IL-4, IL-10, IL-13 and/or TGF.beta. cytokines or agonists thereof (e.g., agonist antibodies); IL-11; glucuronide- or dextran-conjugated prodrugs of prednisolone, dexamethasone or budesonide; ICAM-1 antisense phosphorothioate oligodeoxynucleotides (ISIS 2302; Isis Pharmaceuticals, Inc.); soluble complement receptor 1 (TP10; T Cell Sciences, Inc.); slow-release mesalazine; methotrexate; antagonists of platelet activating factor (PAF); ciprofloxacin; and/or lignocaine.

As a further example, the one or more additional therapeutic agents can be therapeutic agents and/or regimens for treating autoimmune colitis. Non-limiting examples corticosteroids (e.g., budesonide, prednisone, prednisolone, Beclometasone dipropionate), diphenoxylate/atropine, infliximab, loperamide, mesalamine, TIP60 inhibitors (see, e.g., U.S. Patent Application Publication No. 2012/0202848), and vedolizumab.

As a further example, the one or more additional therapeutic agents can be therapeutic agents and/or regimens for treating iatrogenic autoimmune colitis. Non-limiting examples include corticosteroids (e.g., budesonide, prednisone, prednisolone, Beclometasone dipropionate), diphenoxylate/atropine, infliximab, loperamide, TIP60 inhibitors (see, e.g., U.S. Patent Application Publication No. 2012/0202848), and vedolizumab.

As a further example, the one or more additional therapeutic agents can be therapeutic agents and/or regimens for treating colitis induced by one or more chemotherapeutics agents. Non-limiting examples include corticosteroids (e.g., budesonide, prednisone, prednisolone, beclometasone dipropionate), diphenoxylate/atropine, infliximab, loperamide, mesalamine, TIP60 inhibitors (see, e.g., U.S. Patent Application Publication No. 2012/0202848), and vedolizumab.

As a further example, the one or more additional therapeutic agents can be therapeutic agents and/or regimens for treating colitis induced by treatment with adoptive cell therapy. Non-limiting examples include corticosteroids (e.g., budesonide, prednisone, prednisolone, beclometasone dipropionate), diphenoxylate/atropine, infliximab, loperamide, TIP60 inhibitors (see, e.g., U.S. Patent Application Publication No. 2012/0202848), and vedolizumab.

As a further example, the one or more additional therapeutic agents can be therapeutic agents and/or regimens for treating colitis associated with one or more alloimmune diseases. Non-limiting examples include corticosteroids (e.g., budesonide, prednisone, prednisolone, beclometasone dipropionate), sulfasalazine, and eicopentaenoic acid.

As a further example, the one or more additional therapeutic agents can be therapeutic agents and/or regimens for treating radaiation enteritis. Non-limiting examples include teduglutide, amifostine, angiotensin-converting enzyme (ACE) inhibitors (e.g., benazepril, captopril, enalapril, fosinopril, lisinopril, moexipril, perindopril, quinapril, ramipril, and trandolapril), probiotics, selenium supplementation, statins (e.g., atorvastatin, fluvastatin, lovastatin, pravastatin, rosuvastatin, simvastatin, and pitavastatin), sucralfate, and vitamin E.

As a further example, the one or more additional therapeutic agents can be therapeutic agents and/or regimens for treating collagenous colitis. Non-limiting examples include 6-mercaptopurine, azathaioprine, bismuth subsalicate, Boswellia serrata extract, cholestyramine, colestipol, corticosteroids (e.g., budesonide, prednisone, prednisolone, beclometasone dipropionate), loperamide, mesalamine, methotrexate, probiotics, and sulfasalazine.

As a further example, the one or more additional therapeutic agents can be therapeutic agents and/or regimens for treating lyphocytic colitis. Non-limiting examples include 6-mercaptopurine, azathioprine, bismuth subsalicylate, cholestyramine, colestipol, corticosteroids (e.g., budesonide, prednisone, prednisolone, beclometasone dipropionate), loperamide, mesalamine, methotrexate, and sulfasalazine.

As a further example, the one or more additional therapeutic agents can be therapeutic agents and/or regimens for treating microscopic colitis. Non-limiting examples include 6-mercaptopurine, azathioprine, bismuth subsalicylate, Boswellia serrata extract, cholestyramine, colestipol, corticosteroids (e.g., budesonide, prednisone, prednisolone, beclometasone dipropionate), fecal microbial transplantation, loperamide, mesalamine, methotrexate, probiotics, and sulfasalazine.

As a further example, the one or more additional therapeutic agents can be therapeutic agents and/or regimens for treating UC. Non-limiting examples include AbGn-168H, ABT-494, ABX464, apremilast, PF-00547659, PF-06687234, 6-mercaptopurine, adalimumab, azathioprine, bertilimumab, brazikumab (MEDI2070), cobitolimod, certolizumab pegol (Cimzia®), CP-690,550, corticosteroids (e.g., multimax budesonide, Methylprednisolone), cyclosporine, E6007, etrasimod, etrolizumab, fecal microbial transplantation, figlotinib, guselkumab, golimumab, IL-2, IMU-838, infliximab, matrix metalloproteinase 9 (MMP9) inhibitors (e.g., GS-5745), mesalamine, mesalamine, mirikizumab (LY3074828), RPC1063, risankizumab (BI 6555066), SHP647, sulfasalazine, TD-1473, TJ301, tildrakizumab (MK 3222), tofacitinib, tofacitinib, ustekinumab, UTTR1147A, and vedolizumab.

As a further example, the one or more additional therapeutic agents can be therapeutic agents and/or regimens for treating Crohn's Disease (CD). Non-limiting examples include adalimumab, autologous CD34-selected peripheral blood stem cells transplant, 6-mercaptopurine, azathioprine, certolizumab pegol (Cimzia®), corticosteroids (e.g., prednisone), etrolizumab, E6011, fecal microbial transplantation, figlotinib, guselkumab, infliximab, IL-2, JAK inhibitors, matrix metalloproteinase 9 (MMP 9) inhibitors (e.g., GS-5745), MEDI2070, mesalamine, methotrexate, natalizumab, ozanimod, RHB-104, rifaximin, risankizumab, SHP647, sulfasalazine, thalidomide, upadacitinib, V565, and vedolizumab.

As a further example, the one or more additional therapeutic agents can be therapeutic agents and/or regimens for treating IBDs. Non-limiting examples include 6-mercaptopurine, AbGn-168H, ABX464, ABT-494, adalimumab, AJM300, alicaforsen, AMG139, anrukinzumab, apremilast, ATR-107 (PF0530900), autologous CD34-selected peripheral blood stem cells transplant, azathioprine, bertilimumab, BI 655066, BMS-936557, certolizumab pegol (Cimzia®), cobitolimod, corticosteroids (e.g., prednisone, Methylprednisolone, prednisone), CP-690,550, CT-P13, cyclosporine, DIMS0150, E6007, E6011, etrasimod, etrolizumab, fecal microbial transplantation, figlotinib, fingolimod, firategrast (SB-683699) (formerly T-0047), GED0301, GLPG0634, GLPG0974, guselkumab, golimumab, GSK1399686, HMPL-004 (Andrographis paniculata extract), IMU-838, infliximab, Interleukin 2 (IL-2), Janus kinase (JAK) inhibitors, laquinimod, masitinib (AB1010), matrix metalloproteinase 9 (MMP 9) inhibitors (e.g., GS-5745), MEDI2070, mesalamine, methotrexate, mirikizumab (LY3074828), natalizumab, NNC 0142-0000-0002, NNC0114-0006, ozanimod, peficitinib (JNJ-54781532), PF-00547659, PF-04236921, PF-06687234, QAX576, RHB-104, rifaximin, risankizumab, RPC1063, SB012, SHP647, sulfasalazine, TD-1473, thalidomide, tildrakizumab (MK 3222), TJ301, TNF-Kinoid®, tofacitinib, tralokinumab, TRK-170, upadacitinib, ustekinumab, UTTR1147A, V565, vatelizumab, VB-201, vedolizumab, and vidofludimus.

The subject can exhibit a digestive symptom. For example, the subject can exhibit a symptom selected from the group consisting of a lack or loss of appetite, diarrhea, vomiting, abdominal pain, a digestive disease, and combinations thereof. For example, the subject can exhibit a symptom selected from the group consisting of lack or loss of appetite, diarrhea, vomiting, abdominal pain, and combinations thereof. As a non-limiting example, the subject can exhibit a symptom selected from the group consisting of diarrhea.

The subject exhibits no accompanying respiratory symptom.

The subject can exhibit an accompanying respiratory symptom.

The compound of Formula (I) can be administered to the subject at risk of developing COVID-19 prior to exposure to the virus or prior to presumed exposure to the virus (e.g., prior to contact with one or more individuals having or presumed to have COVID-19 and/or prior to contact with one or more articles contaminated with the virus).

The compound of Formula (I) can be administered immediately after or shortly after exposure or presumed exposure to the virus.

The methods herein can further comprises administering a second therapeutic agent. The second therapeutic agent can be an antiviral agent. The second therapeutic agent can be selected from the group consisting of azithromycin, remdesivir, colchicine, hydroxychloroquine, and chloroquine. In certain embodiments, the niclosamide, or a pharmaceutically acceptable salt thereof, is administered by tablet or pill.

In certain embodiments, the method comprises orally administering the niclosamide, or a pharmaceutically acceptable salt thereof, as a pharmaceutical composition, wherein the pharmaceutical composition is capable of local delivery to the lower GI tract.

In certain embodiments, the method comprises orally administering the niclosamide, or a pharmaceutically acceptable salt thereof, as a pharmaceutical composition, wherein the pharmaceutical composition is capable of local delivery to the colon.

In certain embodiments, the method comprises orally administering the niclosamide, or a pharmaceutically acceptable salt thereof, as a pharmaceutical composition, wherein the pharmaceutical composition is capable of local delivery to the small intestine.

In another aspect, the disclosure features a method of reducing the risk of developing COVID-19 in a subject (e.g., a human) at risk thereof, the method comprising administering an effective amount of niclosamide:

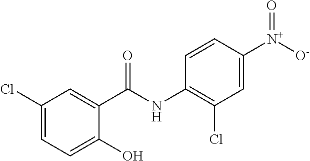 [SMILES: O=C(Nc1ccc([N+](=O)[O-])cc1Cl)c1cc(Cl)ccc1O]

or a pharmaceutically acceptable salt thereof, to the subject (e.g., the human).

In certain of these embodiments, subject is selected from the group consisting of a healthcare worker, a resident of an assisted living facility or nursing home, a patient in a hospital for an unrelated treatment, and a person incarcerated or working in a prison or jail setting. In certain embodiments, the subject is 60 years of age or older. In certain embodiments, the subject suffers from one or more preexisting medical conditions selected from the group consisting of lung disease, cardiovascular disease, cancer, colitis, and an endocrine disease. In certain embodiments, the compound is administered prior to exposure to the coronavirus or immediately after exposure or presumed exposure to the coronavirus. In some embodiments, the methods described herein further comprise one or more of the following: quarantine, self-quarantine, social distancing, frequent handwashing, and frequent environmental sanitization.

In some embodiments of the methods described herein, the subject is a human.

In some embodiments, the methods described herein comprise administering the effective amount of the niclosamide compound, or a pharmaceutically acceptable salt thereof, to the respiratory system of the subject.

In certain embodiments, the methods described herein comprise locally administering an effective amount of the niclosamide compound, or a pharmaceutically acceptable salt thereof, to the respiratory system of the subject.

In certain embodiments, the methods described herein comprise topically administering an effective amount of the niclosamide compound, or a pharmaceutically acceptable salt thereof, to the respiratory system of the subject.

In certain of the foregoing embodiments, the methods described herein comprise locally and topically administering an effective amount of the niclosamide compound, or a pharmaceutically acceptable salt thereof, to the respiratory system of the subject.

In some embodiments, the methods described herein comprise administering the effective amount of the niclosamide compound, or a pharmaceutically acceptable salt thereof, to the lungs of the subject.

In certain embodiments, the methods described herein comprise locally administering an effective amount of the niclosamide compound, or a pharmaceutically acceptable salt thereof, to the lungs of the subject.

In certain embodiments, the methods described herein comprise topically administering an effective amount of the niclosamide compound, or a pharmaceutically acceptable salt thereof, to the lungs of the subject.

In certain embodiments, the methods described herein comprise locally and topically administering an effective amount of the niclosamide compound, or a pharmaceutically acceptable salt thereof, to the lungs of the subject.

In certain embodiments of the methods described herein, the niclosamide compound, or a pharmaceutically acceptable salt thereof, is administered by inhalation.

In another aspect, provided herein is a method for treating COVID-19 in a subject in need thereof, the method comprising administering an effective amount of a niclosamide compound, or a pharmaceutically acceptable salt thereof, to the GI tract of the subject.

In certain embodiments, the method comprises topically administering an effective amount of the niclosamide compound, or a pharmaceutically acceptable salt thereof to the GI tract of the subject.

In certain of these embodiments, the niclosamide compound, or a pharmaceutically acceptable salt thereof, is administered by rectal administration. As a non-limiting example of the foregoing embodiments, the niclosamide compound, or a pharmaceutically acceptable salt thereof, is administered by enema, rectal gel, rectal foam, rectal aerosol, or suppository. For example, the niclosamide compound, or a pharmaceutically acceptable salt thereof, can be administered by enema.

In some embodiments, the method comprises orally administering the niclosamide compound, or a pharmaceutically acceptable salt thereof, as a pharmaceutical composition, wherein the pharmaceutical composition is capable of local delivery to the digestive or GI tract. In certain of these embodiments, the pharmaceutical composition comprises one or more pharmaceutically acceptable excipients that chemically and/or structurally predispose the composition for delivery of the niclosamide compound, or a pharmaceutically acceptable salt thereof, to the lower GI tract. For example, the composition comprises one or more pharmaceutically acceptable excipients that chemically and/or structurally predispose the composition for delivery of the niclosamide compound, or a pharmaceutically acceptable salt thereof, to the colon. As another non-limiting example, the composition comprises one or more pharmaceutically acceptable excipients that chemically and/or structurally predispose the composition for delivery of the niclosamide, or a pharmaceutically acceptable salt thereof, to the ascending colon and/or transverse colon and/or distal colon. As another non-limiting example, the composition comprises one or more pharmaceutically acceptable excipients that chemically and/or structurally predispose the composition for delivery of niclosamide, or a pharmaceutically acceptable salt thereof, to the small intestine (e.g., to the ileum).

The compound of Formula (I) can have a chemical purity of greater than about 99.0%.

The compound of Formula (I) can have a reduced particle size range. For example, the compound can have a particle size range of from about 0.1 μm to about 30 μm, such as a particle size range of from about 0.1 μm to about 20 μm (e.g., a particle size range of from about 0.1 μm to about 10 μm).

The compound of Formula (I) can have a particle size distribution D(0.9) of from about 1.0 μm to about 15.0 μm, such as a particle size distribution D(0.9) of from about 1.0 μm to about 10.0 μm (e.g., a particle size distribution D(0.9) of from about 6.0 μm to about 8.0 μm; or a particle size distribution D(0.9) of from about 2.2 μm to about 3.2 μm).

The compound of Formula (I) can have a particle size distribution D(0.1) of from about 0.1 μm to about 1.5 μm, such as a particle size distribution D(0.1) of from about 0.1 μm to about 1.0 μm (e.g., a particle size distribution D(0.1) of from about 0.3 μm to about 0.9 μm).

The compound of Formula (I) can have a particle size distribution D(0.5) of from about 0.5 μm to about 6.0 μm, such as a particle size distribution D(0.5) of from about 1.0 μm to about 4.0 μm (e.g., a particle size distribution D(0.5) of from about 1.0 μm to about 2.0 μm; or a particle size distribution D(0.5) of from about 2.5 μm to about 3.5 μm).

The compound of Formula (I) can have a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

The compound of Formula (I) can have a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

The compound of Formula (I) can have a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

The compound of Formula (I) can have a chemical purity of greater than about 99.0%, a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

The compound of Formula (I) can have a chemical purity of greater than about 99.0%, a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

The compound of Formula (I) can have a chemical purity of greater than about 99.0%, a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

The compound of Formula (I) can have a specific surface area of from about 5 m²/g to about 10 m²/g.

[A1] The compound of Formula (I) can have a chemical purity of greater than about 99.0%, a particle size range of from about 0.1 μm to about 30 μm, a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

[B1] The compound of Formula (I) can have a chemical purity of greater than about 99.0%, a particle size range of from about 0.1 μm to about 30 μm, a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

[C1] The compound of Formula (I) can have a chemical purity of greater than about 99.0%, a particle size range of from about 0.1 μm to about 30 μm, a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

In certain embodiments of [A1], [B1], and/or [C1] supra, the compound of Formula (I) has a particle size distribution D(0.5) of from about 2.5 μm to about 3.5 μm.

In certain embodiments of [A1], [B1], and/or [C1] supra, the compound of Formula (I) has a particle size distribution D(0.5) of from about 1.0 μm to about 2.0 μm.

In certain embodiments of [A1], [B1], and/or [C1] supra, the compound of Formula (I) has a chemical purity of greater than about 99.5%; or a chemical purity of greater than about 99.7%; or a chemical purity of greater than about 99.8%.

In certain embodiments of [A], [B], and/or [C] supra, the compound of Formula (I) has a specific surface area of from about 5 m²/g to about 10 m²/g.

The compound of Formula (I) can be administered (via respiratory administration) as a pharmaceutical composition, wherein the pharmaceutical composition is capable of local delivery to the respiratory tract. For example, the pharmaceutical composition can comprises one or more pharmaceutically acceptable excipients that chemically and/or structurally predispose the composition for delivery of compound of Formula (I), or a pharmaceutically acceptable salt and/or cocrystal thereof, to the upper respiratory tract (e.g., nose and nasal passages); and or lower respiratory tract (e.g., the lungs).

For example, compound of Formula (I), or a pharmaceutically acceptable salt and/or cocrystal thereof can be administered to the nasal cavity of the subject, wherein compound of Formula (I), or a pharmaceutically acceptable salt and/or cocrystal thereof, is formulated as an intranasal spray, ointment, or gel. As another non-limiting example, compound of Formula (I), or a pharmaceutically acceptable salt and/or cocrystal thereof can be administered to the lungs of the subject, wherein compound of Formula (I), or a pharmaceutically acceptable salt and/or cocrystal thereof, is formulated for delivery by inhalation.

The compound of Formula (I) can be administered (orally or rectally) as a pharmaceutical composition, wherein the pharmaceutical composition is capable of local delivery to the digestive or GI tract. For example, the pharmaceutical composition can comprises one or more pharmaceutically acceptable excipients that chemically and/or structurally predispose the composition for delivery of compound of Formula (I), or a pharmaceutically acceptable salt and/or cocrystal thereof, to e.g., the lower GI tract or the colon (e.g., ascending colon and/or transverse colon and/or distal colon and/or small intestine (e.g., ileum)).

For example, compound of Formula (I), or a pharmaceutically acceptable salt and/or cocrystal thereof can be administered to the GI tract of the subject, wherein compound of Formula (I), or a pharmaceutically acceptable salt and/or cocrystal thereof, is formulated as an oral composition. In one aspect, a cocrystal is provided, which includes: (i) a compound of Formula (I) or a pharmaceutically acceptable salt and/or hydrate thereof, and (ii) one or more pharmaceutically acceptable coformers. In some embodiments, the cocrystal has a reduced particle size as described anywhere herein. In embodiments, the cocrystal coformers can include any coformers described herein, including second therapeutic agents as described above and anywhere herein.

Non-limiting examples of the co-former include sphingosine 1-phosphate (S1P) receptor modulators (e.g., etrasimod or ozanimod); steroidal anti-inflammatory agents (e.g, beclomethasone 17 or budesonide); non-steroidal anti-inflammatory agents (e.g., 5-ASA); receptor-interacting protein kinase 1 (RIPK1) inhibitors (e.g., GSK2982772); EP4 modulators (e.g., KAG-308); toll-like receptor (e.g., TLR4, TLR9) modulators (e.g., JKB-122, cobitolimod); Janus kinase (JAK) inhibitors (e.g., TD-1473, tofacitinib, upadacitinib, filgotinib, PF-06651600, and PF-06700841); lanthionine synthetase C-like 2 (LANCL2) modulators (e.g., BT-11); phosphatidylcholine (e.g., LT-02); integrin (e.g., a4 Integrin) modulators (e.g, AJM-300 (carotegrast)); Smad7 modulators (e.g., mongersen); phosphodiesterase 4 (PDE4) modulators (e.g., apremilast); tumor progression locus 2 (TPL2) inhibitors (e.g., GS-4875); tyrosine kinase 2(TYK2) inhibitors (e.g., BMS-986165, PF-06700841, and PF-06826647); and TEC kinase inhibitors (e.g., PF-06651600).

In some embodiments, the Formula (I) compounds described herein can provide targeted delivery of the Formula (I) compounds to certain regions of the GI tract (e.g., the colon, e.g., the ascending colon and/or the transverse colon and/or the distal colon (e.g., for treatment of an inflammatory bowel disease such as ulcerative colitis); e.g., the small bowel, e.g., the ileum (e.g., for treatment of an inflammatory bowel disease such as Crohn's disease)). In some embodiments, administration (e.g., oral administration; e.g., rectal administration) of a compound of Formula (I) described herein to a subject produces a local concentration of the compound of Formula (I) in the GI tract (e.g., colon, e.g., supra; e.g., the small bowel, e.g., the ileum) of the subject that is higher than the concentration of the compound of Formula (I) in the plasma compartment of the subject, thereby, e.g., more efficiently providing the compound of Formula (I) to diseased tissue in the GI tract (e.g., supra) and reducing risks associated with high systemic the compound of Formula (I) exposure (e.g., toxicity). Moreover, the foregoing can potentially be achieved using a lower dosage with the Formula (I) compounds described herein.

In view of the foregoing advantages and features delineated above, the Formula (I) compounds, methods, and compositions described herein are also expected to be functional in diverse patient populations and/or less sensitive to blocks in cell death mechanisms. Further, the ability to utilize traditional small molecules can help reduce cost and facilitate patient administration.

In some embodiments, the methods and compositions described herein are suitable for use in combination therapy with various other therapeutic regimens (e.g., chemotherapy and/or radiation). In certain embodiments, the chemical entities and methods described herein can be used to treat side effects produced by such therapeutic regimens, e.g., inflammatory bowel diseases induced by chemotherapeutic immunomodulators, e.g., checkpoint inhibitors, which in some cases can be prohibitively severe.

In certain embodiments, the methods and compositions described herein are suitable for use in combination therapy with one or more additional therapeutic agents. For example, therapeutic agents useful for treating or preventing inflammatory bowel disease (IBD) (e.g., Crohn's disease, ulcerative colitis). Non-limiting examples of the additional therapeutic agents include: sphingosine 1-phosphate (S1P) receptor modulators (e.g., etrasimod or ozanimod); steroidal anti-inflammatory agents (e.g, beclomethasone 17 or budesonide); non-steroidal anti-inflammatory agents (e.g., 5-ASA); receptor-interacting protein kinase 1 (RIPK1)inhibitors (e.g., GSK2982772); EP4 modulators (e.g., KAG-308); toll-like receptor (e.g., TLR4, TLR9) modulators (e.g., JKB-122, cobitolimod); Janus kinase (JAK) inhibitors (e.g., TD-1473, tofacitinib, upadacitinib, filgotinib, PF-06651600, and PF-06700841); lanthionine synthetase C-like 2 (LANCL2) modulators (e.g., BT-11); phosphatidylcholine (e.g., LT-02); integrin (e.g., a4 Integrin) modulators (e.g, AJM-300 (carotegrast)); Smad7 modulators (e.g., mongersen); phosphodiesterase 4 (PDE4) modulators (e.g., apremilast); tumor progression locus 2 (TPL2) inhibitors (e.g., GS-4875); tyrosine kinase 2(TYK2)inhibitors (e.g., BMS-986165, PF-06700841, and PF-06826647); and TEC kinase inhibitors (e.g., PF-06651600).

Additionally, the chemical entities, methods, and compositions described herein are also expected to be useful in certain treatment-resistant patient populations, e.g., one that is nonresponsive or resistant to treatment an anti-TNFalpha therapy (e.g., Humira, Enbrel, Remicade) or anti-integrin therapy (e.g., Entyvio, etrolizumab) or corticosteroids.

Any of the foregoing methods can be carried out using any one of the compounds (e.g., a compound of Formula (I)), compositions, formulations (e.g., enema formulaion), solid forms (e.g., with reduced particle size), co-crystals, and pharmaceutical combinations described herein.

### Definitions

To facilitate understanding of the disclosure set forth herein, a number of terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The singular forms “a,” “an,” and “the” may refer to plural articles unless specifically stated otherwise.

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

The term “deuterium enrichment” refers to the percentage of incorporation of deuterium at a given position in a molecule in the place of hydrogen. Because the naturally occurring distribution of deuterium is about 0.0156%, deuterium enrichment at any position in a compound synthesized using non-enriched starting materials is about 0.0156%. The deuterium enrichment can be determined using conventional analytical methods known to one of ordinary skill in the art, including mass spectrometry and nuclear magnetic resonance spectroscopy.

The term “is/are deuterium,” when used to describe a given position in a molecule such as X¹, or the symbol “D”, when used to represent a given position in a drawing of a molecular structure, means that the specified position is enriched with deuterium above the naturally occurring distribution of deuterium. The same is true of the term “contains deuterium.” In one embodiment deuterium enrichment is no less than about 50%, in another no less than about 60%, in another no less than about 70%, in another no less than about 80%, in another no less than about 90%, in another no less than about 95%, in another no less than about 98%, or in another no less than about 99% of deuterium at the specified position.

The term “isotopic enrichment” refers to the percentage of incorporation of a less prevalent isotope of an element at a given position in a molecule in the place of the more prevalent isotope of the element.

The term “non-isotopically enriched” refers to a molecule in which the percentages of the various isotopes are substantially the same as the naturally occurring percentages.

“Niclosamide” refers to a compound having the following chemical structure:

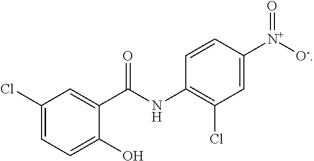

Niclosamide is known by the IUPAC designation: 2′5-dichloro-4′-nitrosalicylanilide and by the CAS designation: CAS: 5-chloro-N-(2-chloro-4-nitrophenyl)-2-hydroxybenzamide. Niclosamide has a relatively low water solubility at about from 5-8 mg/L at 20° C., is sparingly soluble in ether, ethanol and chloroform, and is soluble in acetone. The ethanolamine salt dissolves in distilled water 180-280 mg/L at 20° C.

Niclosamide is available in a various salt or solvated forms. These include, but are not limited to, the ethanolamine salt known by the IUPAC designation 5-chloro-salicyl-(2-chloro-4-nitro) anilide 2-aminoethanol salt or the CAS designation 5-chloro-N-(2-chloro-4-nitrophenyl)-2-hydroxybenzamide with 2-aminoethanol (1:1)—see, e.g., US 2013/0231312; the piperazine salt known by the IUPAC designation 5-chloro-salicyl-(2-chloro-4-nitro) anilide piperazine salt or the CAS designation 5-chloro-N-(2-chloro-4-nitrophenyl)-2-hydroxybenzamide with piperazine (2:1), and niclosamide monohydrate known by the IUPAC designation 5-chloro-salicyl-(2-chloro-4-nitro) anilide monohydrate or the CAS designation 5-chloro-N-(2-chloro-4-nitrophenyl)-2-hydroxybenzamide with monohydrate (1:1).

Niclosamide is commercially available in a variety of formulations including, but not limited to BAYER 73®, BAYER 2353®, BAYER 25 648®, BAYLUSCID®, BAYLUSCIDE®, CESTOCID®, CLONITRALID, DICHLOSALE®, FENASAL®, HL 2447®, IOMESAN®, IOMEZAN®, LINTEX®, MANOSIL®, NASEMO®, NICLOSAMID®, PHENASAL®, TREDEMINE®, SUJLQUI®, VERMITID®, VERMITIN®, YOMESAN®, and the like.

The term “digestive tract” is understood to include the mouth, pharynx, esophagus, stomach, small intestine or small bowel (duodenum, jejunum, ileum), large intestine (colon (cecum, ascending colon, transverse colon, descending colon, sigmoid colon), rectum) and anus.

The term “oral cavity” is understood to include the mouth, the pharynx and the esophagus.

The term “gastrointestinal tract”, or “GI tract” is understood to include the stomach, small intestine (duodenum, jejunum, ileum), large intestine (cecum, colon, rectum) and anus.

The term “acceptable” with respect to a formulation, composition or ingredient, as used herein, means having no persistent detrimental effect on the general health of the subject being treated.

“API” refers to an active pharmaceutical ingredient (e.g., Formula (I) compounds).

The terms “effective amount” or “therapeutically effective amount,” as used herein, refer to a sufficient amount of a chemical entity (e.g., a compound exhibiting activity as a mitochondrial uncoupling agent or a pharmaceutically acceptable salt and/or hydrate and/or cocrystal thereof, e.g., a compound, such a compound of Formula (I) or a pharmaceutically acceptable salt and/or hydrate and/or cocrystal thereof being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The terms “treat,” “treating,” and “treatment,” in the context of treating a disease or disorder, are meant to include alleviating or abrogating a disorder, disease, or condition, or one or more of the symptoms associated with the disorder, disease, or condition; or to slowing the progression, spread or worsening of a disease, disorder or condition or of one or more symptoms thereof. Often, the beneficial effects that a subject derives from a therapeutic agent do not result in a complete cure of the disease, disorder or condition.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C show the components of a representative enema delivery device (FIG. 1A shows the bottle, FIG. 1B shows the breakable capsule, and FIG. 1C shows the rectal cannula (upper arrow) and single flow pack (lower arrow).

### DETAILED DESCRIPTION

This disclosure features chemical entities (e.g., a compound or a pharmaceutically acceptable salt, and/or hydrate, and/or cocrystal, and/or drug combination of the compound) that exhibit activity as mitochondrial uncoupling agents. Said chemical entities are useful, e.g., for treating one or more symptoms of a pathology characterized by an abnormal inflammatory response (e.g., inflammatory bowel diseases) in a subject (e.g., a human). This disclosure also features compositions containing the same as well as methods of using and making the same.

Formula (I) Compounds

In some embodiments provided herein is a compound of Formula (I),

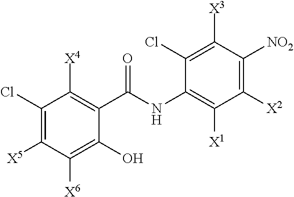

or a pharmaceutically acceptable salt thereof

wherein:

X¹ is hydrogen or deuterium;

X² is hydrogen or deuterium;

X³ is hydrogen or deuterium;

X⁴ is hydrogen or deuterium;

X⁵ is hydrogen or deuterium;

X⁶ is hydrogen or deuterium;

provided that the compound of Formula (I) comprises deuterium.

As used herein, when the compound of Formula (I) is described as “compris[ing] deuterium), one or more (e.g., 1, 2, 3, 4, 5, or 6) of X¹, X², X³, X⁴, X⁵, or X⁶ is independently deuterium.

For example:

if X¹, X², X³, X⁴ and X⁵ are each hydrogen, then X⁶ is deuterium;

if X¹, X², X³, X⁴ and X⁶ are each hydrogen, then X⁵ is deuterium;

if X¹, X², X³, X⁵ and X⁶ are each hydrogen, then X⁴ is deuterium;

if X¹, X², X⁴, X⁵ and X⁶ are each hydrogen, then X³ is deuterium;

if X¹, X³, X⁴, X⁵ and X⁶ are each hydrogen, then X² is deuterium; and

if X², X³, X⁴, X⁵ and X⁶ are each hydrogen, then X¹ is deuterium.

In some embodiments, it is provided that if X¹, X², X³ and X⁴ are each deuterium, then at least one of X⁵ or X⁶ is deuterium.

In some embodiments, the compound of Formula (I) is other than:

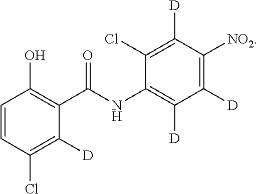 [SMILES: [2H]c1c([2H])c([N+](=O)[O-])c([2H])c(Cl)c1NC(=O)c1c(O)ccc(Cl)c1[2H]]

In some embodiments, the compound of Formula (I) is other than:

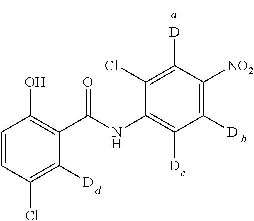 [SMILES: [2H]c1c([2H])c([N+](=O)[O-])c([2H])c(Cl)c1NC(=O)c1c(O)ccc(Cl)c1[2H]]

wherein the level of deuterium incorporation at positions a, b, c, or d is selected from the following table (Table 1):

| Entry | % Dₐ | % D_{b} | % D_{c} | % D_{d} |
|---|---|---|---|---|
| 1 | 66 | 53 | 41 | 66 |
| 2 | 71 | 57 | 18 | 73 |
| 3 | 51 | 11 | 4 | 98 |
| 4 | 97 | 96 | 65 | 96 |
| 5 | 55 | 44 | 40 | 58 |
| 6 | 75 | 70 | 63 | 76 |
| 7 | 83 | 48 | 37 | 87 |
| 8 | 89 | 55 | 28 | 97 |
| 9 | 65 | 55 | 52 | 62 |

In some embodiments, X¹ is deuterium. In one more particular embodiment, X² is hydrogen. In one more particular embodiment, X² is deuterium. In one more particular embodiment, X³ is hydrogen. In one more particular embodiment, X³ is deuterium. In one more particular embodiment, X⁴ is hydrogen. In one more particular embodiment, X⁴ is deuterium. In one more particular embodiment, X⁵ is hydrogen. In one more particular embodiment, X⁵ is deuterium. In one more particular embodiment, X⁶ is hydrogen. In one more particular embodiment, X⁶ is deuterium.

In some embodiments, X¹ is hydrogen. In one more particular embodiment, X² is hydrogen. In one more particular embodiment, X² is deuterium. In one more particular embodiment, X³ is hydrogen. In one more particular embodiment, X³ is deuterium. In one more particular embodiment, X⁴ is hydrogen. In one more particular embodiment, X⁴ is deuterium. In one more particular embodiment, X⁵ is hydrogen. In one more particular embodiment, X⁵ is deuterium. In one more particular embodiment, X⁶ is hydrogen. In one more particular embodiment, X⁶ is deuterium.

In some embodiments, X² is deuterium. In one more particular embodiment, X³ is hydrogen. In one more particular embodiment, X³ is deuterium. In one more particular embodiment, X⁴ is hydrogen. In one more particular embodiment, X⁴ is deuterium. In one more particular embodiment, X⁵ is hydrogen. In one more particular embodiment, X⁵ is deuterium. In one more particular embodiment, X⁶ is hydrogen. In one more particular embodiment, X⁶ is deuterium.

In some embodiments, X² is hydrogen. In one more particular embodiment, X³ is hydrogen. In one more particular embodiment, X³ is deuterium. In one more particular embodiment, X⁴ is hydrogen. In one more particular embodiment, X⁴ is deuterium. In one more particular embodiment, X⁵ is hydrogen. In one more particular embodiment, X⁵ is deuterium. In one more particular embodiment, X⁶ is hydrogen. In one more particular embodiment, X⁶ is deuterium.

In some embodiments, X³ is deuterium. In one more particular embodiment, X⁴ is hydrogen. In one more particular embodiment, X⁴ is deuterium. In one more particular embodiment, X⁵ is hydrogen. In one more particular embodiment, X⁵ is deuterium. In one more particular embodiment, X⁶ is hydrogen. In one more particular embodiment, X⁶ is deuterium.

In some embodiments, X³ is hydrogen. In one more particular embodiment, X⁴ is hydrogen. In one more particular embodiment, X⁴ is deuterium. In one more particular embodiment, X⁵ is hydrogen. In one more particular embodiment, X⁵ is deuterium. In one more particular embodiment, X⁶ is hydrogen. In one more particular embodiment, X⁶ is deuterium.

In some embodiments, X⁴ is deuterium. In one more particular embodiment, X⁵ is hydrogen. In one more particular embodiment, X⁵ is deuterium. In one more particular embodiment, X⁶ is hydrogen. In one more particular embodiment, X⁶ is deuterium.

In some embodiments, X⁴ is hydrogen. In one more particular embodiment, X⁵ is hydrogen. In one more particular embodiment, X⁵ is deuterium. In one more particular embodiment, X⁶ is hydrogen. In one more particular embodiment, X⁶ is deuterium.

In some embodiments, X⁵ is deuterium. In one more particular embodiment, X⁶ is hydrogen. In one more particular embodiment, X⁶ is deuterium.

In some embodiments, X⁵ is hydrogen. In one more particular embodiment, X⁶ is hydrogen. In one more particular embodiment, X⁶ is deuterium.

In some embodiments, X⁶ is deuterium.

In some embodiments, X⁶ is hydrogen.

In one more particular embodiment, X¹ is deuterium, X² is hydrogen, X³ is hydrogen, X⁴ is hydrogen, X⁵ is hydrogen and X⁶ is hydrogen.

In one more particular embodiment, X¹ is deuterium, X² is deuterium, X³ is hydrogen, X⁴ is hydrogen, X⁵ is hydrogen and X⁶ is hydrogen.

In one more particular embodiment, X¹ is deuterium, X² is hydrogen, X³ is deuterium, X⁴ is hydrogen, X⁵ is hydrogen and X⁶ is hydrogen.

In one more particular embodiment, X¹ is deuterium, X² is deuterium, X³ is deuterium, X⁴ is hydrogen, X⁵ is hydrogen and X⁶ is hydrogen.

In one more particular embodiment, X¹ is deuterium, X² is hydrogen, X³ is hydrogen, X⁴ is deuterium, X⁵ is hydrogen and X⁶ is hydrogen.

In one more particular embodiment, X¹ is deuterium, X² is deuterium, X³ is hydrogen, X⁴ is deuterium, X⁵ is hydrogen and X⁶ is hydrogen.

In one more particular embodiment, X¹ is deuterium, X² is hydrogen, X³ is deuterium, X⁴ is deuterium, X⁵ is hydrogen and X⁶ is hydrogen.

In one more particular embodiment, X¹ is deuterium, X² is deuterium, X³ is deuterium, X⁴ is deuterium, X⁵ is hydrogen and X⁶ is hydrogen.

In one more particular embodiment, X¹ is deuterium, X² is hydrogen, X³ is hydrogen, X⁴ is hydrogen, X⁵ is deuterium and X⁶ is hydrogen.

In one more particular embodiment, X¹ is deuterium, X² is deuterium, X³ is hydrogen, X⁴ is hydrogen, X⁵ is deuterium and X⁶ is hydrogen.

In one more particular embodiment, X¹ is deuterium, X² is hydrogen, X³ is deuterium, X⁴ is hydrogen, X⁵ is deuterium and X⁶ is hydrogen.

In one more particular embodiment, X¹ is deuterium, X² is deuterium, X³ is deuterium, X⁴ is hydrogen, X⁵ is deuterium and X⁶ is hydrogen.

In one more particular embodiment, X¹ is deuterium, X² is hydrogen, X³ is hydrogen, X⁴ is deuterium, X⁵ is deuterium and X⁶ is hydrogen.

In one more particular embodiment, X¹ is deuterium, X² is deuterium, X³ is hydrogen, X⁴ is deuterium, X⁵ is deuterium and X⁶ is hydrogen.

In one more particular embodiment, X¹ is deuterium, X² is hydrogen, X³ is deuterium, X⁴ is deuterium, X⁵ is deuterium and X⁶ is hydrogen.

In one more particular embodiment, X¹ is deuterium, X² is deuterium, X³ is deuterium, X⁴ is deuterium, X⁵ is deuterium, and X⁶ is hydrogen.

In one more particular embodiment, X¹ is deuterium, X² is hydrogen, X³ is hydrogen, X⁴ is hydrogen, X⁵ is hydrogen and X⁶ is deuterium.

In one more particular embodiment, X¹ is deuterium, X² is deuterium, X³ is hydrogen, X⁴ is hydrogen, X⁵ is hydrogen and X⁶ is deuterium.

In one more particular embodiment, X¹ is deuterium, X² is hydrogen, X³ is deuterium, X⁴ is hydrogen, X⁵ is hydrogen and X⁶ is deuterium.

In one more particular embodiment, X¹ is deuterium, X² is deuterium, X³ is deuterium, X⁴ is hydrogen, X⁵ is hydrogen and X⁶ is deuterium.

In one more particular embodiment, X¹ is deuterium, X² is hydrogen, X³ is hydrogen, X⁴ is deuterium, X⁵ is hydrogen and X⁶ is deuterium.

In one more particular embodiment, X¹ is deuterium, X² is deuterium, X³ is hydrogen, X⁴ is deuterium, X⁵ is hydrogen and X⁶ is deuterium.

In one more particular embodiment, X¹ is deuterium, X² is hydrogen, X³ is deuterium, X⁴ is deuterium, X⁵ is hydrogen and X⁶ is deuterium.

In one more particular embodiment, X¹ is deuterium, X² is deuterium, X³ is deuterium, X⁴ is deuterium, X⁵ is hydrogen and X⁶ is deuterium.

In one more particular embodiment, X¹ is deuterium, X² is hydrogen, X³ is hydrogen, X⁴ is hydrogen, X⁵ is deuterium and X⁶ is deuterium.

In one more particular embodiment, X¹ is deuterium, X² is deuterium, X³ is hydrogen, X⁴ is hydrogen, X⁵ is deuterium and X⁶ is deuterium.

In one more particular embodiment, X¹ is deuterium, X² is hydrogen, X³ is deuterium, X⁴ is hydrogen, X⁵ is deuterium and X⁶ is deuterium.

In one more particular embodiment, X¹ is deuterium, X² is deuterium, X³ is deuterium, X⁴ is hydrogen, X⁵ is deuterium and X⁶ is deuterium.

In one more particular embodiment, X¹ is deuterium, X² is hydrogen, X³ is hydrogen, X⁴ is deuterium, X⁵ is deuterium and X⁶ is deuterium.

In one more particular embodiment, X¹ is deuterium, X² is deuterium, X³ is hydrogen, X⁴ is deuterium, X⁵ is deuterium and X⁶ is deuterium.

In one more particular embodiment, X¹ is deuterium, X² is hydrogen, X³ is deuterium, X⁴ is deuterium, X⁵ is deuterium and X⁶ is deuterium.

In one more particular embodiment, X¹ is deuterium, X² is deuterium, X³ is deuterium, X⁴ is deuterium, X⁵ is deuterium, and X⁶ is deuterium.

In one more particular embodiment, X¹ is hydrogen, X² is deuterium, X³ is hydrogen, X⁴ is hydrogen, X⁵ is hydrogen and X⁶ is hydrogen.

In one more particular embodiment, X¹ is hydrogen, X² is hydrogen, X³ is deuterium, X⁴ is hydrogen, X⁵ is hydrogen and X⁶ is hydrogen.

In one more particular embodiment, X¹ is hydrogen, X² is deuterium, X³ is deuterium, X⁴ is hydrogen, X⁵ is hydrogen and X⁶ is hydrogen.

In one more particular embodiment, X¹ is hydrogen, X² is hydrogen, X³ is hydrogen, X⁴ is deuterium, X⁵ is hydrogen and X⁶ is hydrogen.

In one more particular embodiment, X¹ is hydrogen, X² is deuterium, X³ is hydrogen, X⁴ is deuterium, X⁵ is hydrogen and X⁶ is hydrogen.

In one more particular embodiment, X¹ is hydrogen, X² is hydrogen, X³ is deuterium, X⁴ is deuterium, X⁵ is hydrogen and X⁶ is hydrogen.

In one more particular embodiment, X¹ is hydrogen, X² is deuterium, X³ is deuterium, X⁴ is deuterium, X⁵ is hydrogen and X⁶ is hydrogen.

In one more particular embodiment, X¹ is hydrogen, X² is hydrogen, X³ is hydrogen, X⁴ is hydrogen, X⁵ is deuterium and X⁶ is hydrogen.

In one more particular embodiment, X¹ is hydrogen, X² is deuterium, X³ is hydrogen, X⁴ is hydrogen, X⁵ is deuterium and X⁶ is hydrogen.

In one more particular embodiment, X¹ is hydrogen, X² is hydrogen, X³ is deuterium, X⁴ is hydrogen, X⁵ is deuterium and X⁶ is hydrogen.

In one more particular embodiment, X¹ is hydrogen, X² is deuterium, X³ is deuterium, X⁴ is hydrogen, X⁵ is deuterium and X⁶ is hydrogen.

In one more particular embodiment, X¹ is hydrogen, X² is hydrogen, X³ is hydrogen, X⁴ is deuterium, X⁵ is deuterium and X⁶ is hydrogen.

In one more particular embodiment, X¹ is hydrogen, X² is deuterium, X³ is hydrogen, X⁴ is deuterium, X⁵ is deuterium and X⁶ is hydrogen.

In one more particular embodiment X¹ is hydrogen, X² is hydrogen, X³ is deuterium, X⁴ is deuterium, X⁵ is deuterium and X⁶ is hydrogen.

In one more particular embodiment, X¹ is hydrogen, X² is deuterium, X³ is deuterium, X⁴ is deuterium, X⁵ is deuterium, and X⁶ is hydrogen.

In one more particular embodiment, X¹ is hydrogen, X² is hydrogen, X³ is hydrogen, X⁴ is hydrogen, X⁵ is hydrogen and X⁶ is deuterium.

In one more particular embodiment, X¹ is hydrogen, X² is deuterium, X³ is hydrogen, X⁴ is hydrogen, X⁵ is hydrogen and X⁶ is deuterium.

In one more particular embodiment, X¹ is hydrogen, X² is hydrogen, X³ is deuterium, X⁴ is hydrogen, X⁵ is hydrogen and X⁶ is deuterium.

In one more particular embodiment, X¹ is hydrogen, X² is deuterium, X³ is deuterium, X⁴ is hydrogen, X⁵ is hydrogen and X⁶ is deuterium.

In one more particular embodiment, X¹ is hydrogen, X² is hydrogen, X³ is hydrogen, X⁴ is deuterium, X⁵ is hydrogen and X⁶ is deuterium.

In one more particular embodiment, X¹ is hydrogen, X² is deuterium, X³ is hydrogen, X⁴ is deuterium, X⁵ is hydrogen and X⁶ is deuterium.

In one more particular embodiment, X¹ is hydrogen, X² is hydrogen, X³ is deuterium, X⁴ is deuterium, X⁵ is hydrogen and X⁶ is deuterium.

In one more particular embodiment, X¹ is hydrogen, X² is deuterium, X³ is deuterium, X⁴ is deuterium, X⁵ is hydrogen and X⁶ is deuterium.

In one more particular embodiment, X¹ is hydrogen, X² is hydrogen, X³ is hydrogen, X⁴ is hydrogen, X⁵ is deuterium and X⁶ is deuterium.

In one more particular embodiment, X¹ is hydrogen, X² is deuterium, X³ is hydrogen, X⁴ is hydrogen, X⁵ is deuterium and X⁶ is deuterium.

In one more particular embodiment, X¹ is hydrogen, X² is hydrogen, X³ is deuterium, X⁴ is hydrogen, X⁵ is deuterium and X⁶ is deuterium.

In one more particular embodiment, X¹ is hydrogen, X² is deuterium, X³ is deuterium, X⁴ is hydrogen, X⁵ is deuterium and X⁶ is deuterium.

In one more particular embodiment, X¹ is hydrogen, X² is hydrogen, X³ is hydrogen, X⁴ is deuterium, X⁵ is deuterium and X⁶ is deuterium.

In one more particular embodiment, X¹ is hydrogen, X² is deuterium, X³ is hydrogen, X⁴ is deuterium, X⁵ is deuterium and X⁶ is deuterium.

In one more particular embodiment, X¹ is hydrogen, X² is hydrogen, X³ is deuterium, X⁴ is deuterium, X⁵ is deuterium and X⁶ is deuterium.

In one more particular embodiment, X¹ is hydrogen, X² is deuterium, X³ is deuterium, X⁴ is deuterium, X⁵ is deuterium, and X⁶ is deuterium.

In some embodiments, X¹ is deuterium.

In one more particular embodiment, X¹ is deuterium and X² is hydrogen.

In one more particular embodiment, X¹ is deuterium and X² is deuterium.

In one more particular embodiment, X¹ is deuterium, X² is hydrogen and X³ is hydrogen.

In one more particular embodiment, X¹ is deuterium, X² is deuterium and X³ is hydrogen.

In one more particular embodiment, X¹ is deuterium, X² is hydrogen and X³ is deuterium.

In one more particular embodiment, X¹ is deuterium, X² is deuterium and X³ is deuterium.

In one more particular embodiment, X¹ is deuterium, X² is hydrogen, X³ is hydrogen and X⁴ is hydrogen.

In one more particular embodiment, X¹ is deuterium, X² is deuterium, X³ is hydrogen and X⁴ is hydrogen.

In one more particular embodiment, X¹ is deuterium, X² is hydrogen, X³ is deuterium and X⁴ is hydrogen.

In one more particular embodiment, X¹ is deuterium, X² is deuterium, X³ is deuterium and X⁴ is hydrogen.

In one more particular embodiment, X¹ is deuterium, X² is hydrogen, X³ is hydrogen and X⁴ is deuterium.

In one more particular embodiment, X¹ is deuterium, X² is deuterium, X³ is hydrogen and X⁴ is deuterium.

In one more particular embodiment, X¹ is deuterium, X² is hydrogen, X³ is deuterium and X⁴ is deuterium.

In one more particular embodiment, X¹ is deuterium, X² is deuterium, X³ is deuterium and X⁴ is deuterium.

In one more particular embodiment, X¹ is deuterium, X² is hydrogen, X³ is hydrogen, X⁴ is hydrogen and X⁵ is hydrogen.

In one more particular embodiment, X¹ is deuterium, X² is deuterium, X³ is hydrogen, X⁴ is hydrogen and X⁵ is hydrogen.

In one more particular embodiment, X¹ is deuterium, X² is hydrogen, X³ is deuterium, X⁴ is hydrogen and X⁵ is hydrogen.

In one more particular embodiment, X¹ is deuterium, X² is deuterium, X³ is deuterium, X⁴ is hydrogen and X⁵ is hydrogen.

In one more particular embodiment, X¹ is deuterium, X² is hydrogen, X³ is hydrogen, X⁴ is deuterium and X⁵ is hydrogen.

In one more particular embodiment, X¹ is deuterium, X² is deuterium, X³ is hydrogen, X⁴ is deuterium and X⁵ is hydrogen.

In one more particular embodiment, X¹ is deuterium, X² is hydrogen, X³ is deuterium, X⁴ is deuterium and X⁵ is hydrogen.

In one more particular embodiment, X¹ is deuterium, X² is deuterium, X³ is deuterium, X⁴ is deuterium and X⁵ is hydrogen.

In one more particular embodiment, X¹ is deuterium, X² is hydrogen, X³ is hydrogen, X⁴ is hydrogen and X⁵ is deuterium.

In one more particular embodiment, X¹ is deuterium, X² is deuterium, X³ is hydrogen, X⁴ is hydrogen and X⁵ is deuterium.

In one more particular embodiment, X¹ is deuterium, X² is hydrogen, X³ is deuterium, X⁴ is hydrogen and X⁵ is deuterium.

In one more particular embodiment, X¹ is deuterium, X² is deuterium, X³ is deuterium, X⁴ is hydrogen and X⁵ is deuterium.

In one more particular embodiment, X¹ is deuterium, X² is hydrogen, X³ is hydrogen, X⁴ is deuterium and X⁵ is deuterium.

In one more particular embodiment, X¹ is deuterium, X² is deuterium, X³ is hydrogen, X⁴ is deuterium and X⁵ is deuterium.

In one more particular embodiment, X¹ is deuterium, X² is hydrogen, X³ is deuterium, X⁴ is deuterium and X⁵ is deuterium.

In one more particular embodiment, X¹ is deuterium, X² is deuterium, X³ is deuterium, X⁴ is deuterium and X⁵ is deuterium.

In some embodiments, X¹ is hydrogen.

In one more particular embodiment, X¹ is hydrogen and X² is hydrogen.

In one more particular embodiment, X¹ is hydrogen and X² is deuterium.

In one more particular embodiment, X¹ is hydrogen, X² is hydrogen and X³ is hydrogen.

In one more particular embodiment, X¹ is hydrogen, X² is deuterium and X³ is hydrogen.

In one more particular embodiment, X¹ is hydrogen, X² is hydrogen and X³ is deuterium.

In one more particular embodiment, X¹ is hydrogen, X² is deuterium and X³ is deuterium.

In one more particular embodiment, X¹ is hydrogen, X² is hydrogen, X³ is hydrogen and X⁴ is hydrogen.

In one more particular embodiment, X¹ is hydrogen, X² is deuterium, X³ is hydrogen and X⁴ is hydrogen.

In one more particular embodiment, X¹ is hydrogen, X² is hydrogen, X³ is deuterium and X⁴ is hydrogen.

In one more particular embodiment, X¹ is hydrogen, X² is deuterium, X³ is deuterium and X⁴ is hydrogen.

In one more particular embodiment, X¹ is hydrogen, X² is hydrogen, X³ is hydrogen and X⁴ is deuterium.

In one more particular embodiment, X¹ is hydrogen, X² is deuterium, X³ is hydrogen and X⁴ is deuterium.

In one more particular embodiment, X¹ is hydrogen, X² is hydrogen, X³ is deuterium and X⁴ is deuterium.

In one more particular embodiment, X¹ is hydrogen, X² is deuterium, X³ is deuterium and X⁴ is deuterium.

In one more particular embodiment, X¹ is hydrogen, X² is hydrogen, X³ is hydrogen, X⁴ is hydrogen and X⁵ is hydrogen.

In one more particular embodiment, X¹ is hydrogen, X² is deuterium, X³ is hydrogen, X⁴ is hydrogen and X⁵ is hydrogen.

In one more particular embodiment, X¹ is hydrogen, X² is hydrogen, X³ is deuterium, X⁴ is hydrogen and X⁵ is hydrogen.

In one more particular embodiment, X¹ is hydrogen, X² is deuterium, X³ is deuterium, X⁴ is hydrogen and X⁵ is hydrogen.

In one more particular embodiment, X¹ is hydrogen, X² is hydrogen, X³ is hydrogen, X⁴ is deuterium and X⁵ is hydrogen.

In one more particular embodiment, X¹ is hydrogen, X² is deuterium, X³ is hydrogen, X⁴ is deuterium and X⁵ is hydrogen.

In one more particular embodiment X¹ is hydrogen, X² is hydrogen, X³ is deuterium, X⁴ is deuterium and X⁵ is hydrogen.

In one more particular embodiment, X¹ is hydrogen, X² is deuterium, X³ is deuterium, X⁴ is deuterium and X⁵ is hydrogen.

In one more particular embodiment, X¹ is hydrogen, X² is hydrogen, X³ is hydrogen, X⁴ is hydrogen and X⁵ is deuterium.

In one more particular embodiment X¹ is hydrogen, X² is deuterium, X³ is hydrogen, X⁴ is hydrogen and X⁵ is deuterium.

In one more particular embodiment, X¹ is hydrogen, X² is hydrogen, X³ is deuterium, X⁴ is hydrogen and X⁵ is deuterium.

In one more particular embodiment, X¹ is hydrogen, X² is deuterium, X³ is deuterium, X⁴ is hydrogen and X⁵ is deuterium.

In one more particular embodiment, X¹ is hydrogen, X² is hydrogen, X³ is hydrogen, X⁴ is deuterium and X⁵ is deuterium.

In one more particular embodiment, X¹ is hydrogen, X² is deuterium, X³ is hydrogen, X⁴ is deuterium and X⁵ is deuterium.

In one more particular embodiment, X¹ is hydrogen, X² is hydrogen, X³ is deuterium, X⁴ is deuterium and X⁵ is deuterium.

In one more particular embodiment, X¹ is hydrogen, X² is deuterium, X³ is deuterium, X⁴ is deuterium and X⁵ is deuterium.

In some embodiments, X² is hydrogen.

In one more particular embodiment, X² is hydrogen and X³ is hydrogen.

In one more particular embodiment, X² is hydrogen and X³ is deuterium.

In one more particular embodiment, X² is hydrogen, X³ is hydrogen and X⁴ is hydrogen.

In one more particular embodiment, X² is hydrogen, X³ is deuterium and X⁴ is hydrogen.

In one more particular embodiment, X² is hydrogen, X³ is hydrogen and X⁴ is deuterium.

In one more particular embodiment, X² is hydrogen, X³ is deuterium and X⁴ is deuterium.

In one more particular embodiment, X² is hydrogen, X³ is hydrogen, X⁴ is hydrogen and X⁵ is hydrogen.

In one more particular embodiment, X² is hydrogen, X³ is deuterium, X⁴ is hydrogen and X⁵ is hydrogen.

In one more particular embodiment, X² is hydrogen, X³ is hydrogen, X⁴ is deuterium and X⁵ is hydrogen.

In one more particular embodiment, X² is hydrogen, X³ is deuterium, X⁴ is deuterium and X⁵ is hydrogen.

In one more particular embodiment, X² is hydrogen, X³ is hydrogen, X⁴ is hydrogen and X⁵ is deuterium.

In one more particular embodiment, X² is hydrogen, X³ is deuterium, X⁴ is hydrogen and X⁵ is deuterium.

In one more particular embodiment, X² is hydrogen, X³ is hydrogen, X⁴ is deuterium and X⁵ is deuterium.

In one more particular embodiment, X² is hydrogen, X³ is deuterium, X⁴ is deuterium and X⁵ is deuterium.

In one more particular embodiment, X² is hydrogen, X³ is hydrogen, X⁴ is hydrogen, X⁵ is hydrogen and X⁶ is hydrogen.

In one more particular embodiment, X² is hydrogen, X³ is deuterium, X⁴ is hydrogen, X⁵ is hydrogen and X⁶ is hydrogen.

In one more particular embodiment, X² is hydrogen, X³ is hydrogen, X⁴ is deuterium, X⁵ is hydrogen and X⁶ is hydrogen.

In one more particular embodiment, X² is hydrogen, X³ is deuterium, X⁴ is deuterium, X⁵ is hydrogen and X⁶ is hydrogen.

In one more particular embodiment, X² is hydrogen, X³ is hydrogen, X⁴ is hydrogen, X⁵ is deuterium and X⁶ is hydrogen.

In one more particular embodiment, X² is hydrogen, X³ is deuterium, X⁴ is hydrogen, X⁵ is deuterium and X⁶ is hydrogen.

In one more particular embodiment, X² is hydrogen, X³ is hydrogen, X⁴ is deuterium, X⁵ is deuterium and X⁶ is hydrogen.

In one more particular embodiment, X² is hydrogen, X³ is deuterium, X⁴ is deuterium, X⁵ is deuterium and X⁶ is hydrogen.

In one more particular embodiment, X² is hydrogen, X³ is hydrogen, X⁴ is hydrogen, X⁵ is hydrogen and X⁶ is deuterium.

In one more particular embodiment, X² is hydrogen, X³ is deuterium, X⁴ is hydrogen, X⁵ is hydrogen and X⁶ is deuterium.

In one more particular embodiment, X² is hydrogen, X³ is hydrogen, X⁴ is deuterium, X⁵ is hydrogen and X⁶ is deuterium.

In one more particular embodiment, X² is hydrogen, X³ is deuterium, X⁴ is deuterium, X⁵ is hydrogen and X⁶ is deuterium.

In one more particular embodiment, X² is hydrogen, X³ is hydrogen, X⁴ is hydrogen, X⁵ is deuterium and X⁶ is deuterium.

In one more particular embodiment, X² is hydrogen, X³ is deuterium, X⁴ is hydrogen, X⁵ is deuterium and X⁶ is deuterium.

In one more particular embodiment, X² is hydrogen, X³ is hydrogen, X⁴ is deuterium, X⁵ is deuterium and X⁶ is deuterium.

In one more particular embodiment, X² is hydrogen, X³ is deuterium, X⁴ is deuterium, X⁵ is deuterium and X⁶ is deuterium.

In some embodiments, X² is deuterium.

In one more particular embodiment, X² is deuterium and X³ is hydrogen.

In one more particular embodiment, X² is deuterium and X³ is deuterium.

In one more particular embodiment, X² is deuterium, X³ is hydrogen and X⁴ is hydrogen.

In one more particular embodiment, X² is deuterium, X³ is deuterium and X⁴ is hydrogen.

In one more particular embodiment, X² is deuterium, X³ is hydrogen and X⁴ is deuterium.

In one more particular embodiment, X² is deuterium, X³ is deuterium and X⁴ is deuterium.

In one more particular embodiment, X² is deuterium, X³ is hydrogen, X⁴ is hydrogen and X⁵ is hydrogen.

In one more particular embodiment, X² is deuterium, X³ is deuterium, X⁴ is hydrogen and X⁵ is hydrogen.

In one more particular embodiment, X² is deuterium, X³ is hydrogen, X⁴ is deuterium and X⁵ is hydrogen.

In one more particular embodiment, X² is deuterium, X³ is deuterium, X⁴ is deuterium and X⁵ is hydrogen.

In one more particular embodiment, X² is deuterium, X³ is hydrogen, X⁴ is hydrogen and X⁵ is deuterium.

In one more particular embodiment, X² is deuterium, X³ is deuterium, X⁴ is hydrogen and X⁵ is deuterium.

In one more particular embodiment, X² is deuterium, X³ is hydrogen, X⁴ is deuterium and X⁵ is deuterium.

In one more particular embodiment, X² is deuterium, X³ is deuterium, X⁴ is deuterium and X⁵ is deuterium.

In one more particular embodiment, X² is deuterium, X³ is hydrogen, X⁴ is hydrogen, X⁵ is hydrogen and X⁶ is hydrogen.

In one more particular embodiment, X² is deuterium, X³ is deuterium, X⁴ is hydrogen, X⁵ is hydrogen and X⁶ is hydrogen.

In one more particular embodiment, X² is deuterium, X³ is hydrogen, X⁴ is deuterium, X⁵ is hydrogen and X⁶ is hydrogen.

In one more particular embodiment, X² is deuterium, X³ is deuterium, X⁴ is deuterium, X⁵ is hydrogen, and X⁶ is hydrogen.

In one more particular embodiment, X² is deuterium, X³ is hydrogen, X⁴ is hydrogen, X⁵ is deuterium and X⁶ is hydrogen.

In one more particular embodiment, X² is deuterium, X³ is deuterium, X⁴ is hydrogen, X⁵ is deuterium and X⁶ is hydrogen.

In one more particular embodiment, X² is deuterium, X³ is hydrogen, X⁴ is deuterium, X⁵ is deuterium and X⁶ is hydrogen.

In one more particular embodiment, X² is deuterium, X³ is deuterium, X⁴ is deuterium, X⁵ is deuterium and X⁶ is hydrogen.

In one more particular embodiment, X² is deuterium, X³ is hydrogen, X⁴ is hydrogen, X⁵ is hydrogen and X⁶ is deuterium.

In one more particular embodiment, X² is deuterium, X³ is deuterium, X⁴ is hydrogen, X⁵ is hydrogen and X⁶ is deuterium.

In one more particular embodiment, X² is deuterium, X³ is hydrogen, X⁴ is deuterium, X⁵ is hydrogen and X⁶ is deuterium.

In one more particular embodiment, X² is deuterium, X³ is deuterium, X⁴ is deuterium, X⁵ is hydrogen, and X⁶ is deuterium.

In one more particular embodiment, X² is deuterium, X³ is hydrogen, X⁴ is hydrogen, X⁵ is deuterium and X⁶ is deuterium.

In one more particular embodiment, X² is deuterium, X³ is deuterium, X⁴ is hydrogen, X⁵ is deuterium and X⁶ is deuterium.

In one more particular embodiment, X² is deuterium, X³ is hydrogen, X⁴ is deuterium, X⁵ is deuterium and X⁶ is deuterium.

In one more particular embodiment, X² is deuterium, X³ is deuterium, X⁴ is deuterium, X⁵ is deuterium and X⁶ is deuterium.

In some embodiments, X³ is hydrogen.

In one more particular embodiment, X³ is hydrogen and X⁴ is hydrogen.

In one more particular embodiment, X³ is hydrogen and X⁴ is deuterium.

In one more particular embodiment, X³ is hydrogen, X⁴ is hydrogen and X⁵ is hydrogen.

In one more particular embodiment, X³ is hydrogen, X⁴ is hydrogen and X⁵ is deuterium.

In one more particular embodiment, X³ is hydrogen, X⁴ is deuterium and X⁵ is hydrogen.

In one more particular embodiment, X³ is hydrogen, X⁴ is deuterium and X⁵ is deuterium.

In one more particular embodiment, X³ is hydrogen, X⁴ is hydrogen, X⁵ is hydrogen, and X⁶ is hydrogen.

In one more particular embodiment, X³ is hydrogen, X⁴ is deuterium, X⁵ is hydrogen and X⁶ is hydrogen.

In one more particular embodiment, X³ is hydrogen, X⁴ is hydrogen, X⁵ is deuterium and X⁶ is hydrogen.

In one more particular embodiment, X³ is hydrogen, X⁴ is deuterium, X⁵ is deuterium and X⁶ is hydrogen.

In one more particular embodiment, X³ is hydrogen, X⁴ is hydrogen, X⁵ is hydrogen, and X⁶ is deuterium.

In one more particular embodiment, X³ is hydrogen, X⁴ is deuterium, X⁵ is hydrogen and X⁶ is deuterium.

In one more particular embodiment, X³ is hydrogen, X⁴ is hydrogen, X⁵ is deuterium and X⁶ is deuterium.

In one more particular embodiment, X³ is hydrogen, X⁴ is deuterium, X⁵ is deuterium and X⁶ is hydrogen.

In one more particular embodiment, X³ is hydrogen, X⁴ is deuterium, X⁵ is deuterium and X⁶ is deuterium.

In some embodiments, X³ is deuterium.

In one more particular embodiment, X³ is deuterium and X⁴ is hydrogen.

In one more particular embodiment, X³ is deuterium and X⁴ is deuterium.

In one more particular embodiment, X³ is deuterium, X⁴ is hydrogen and X⁵ is hydrogen.

In one more particular embodiment, X³ is deuterium, X⁴ is hydrogen and X⁵ is deuterium.

In one more particular embodiment, X³ is deuterium, X⁴ is deuterium and X⁵ is hydrogen.

In one more particular embodiment, X³ is deuterium, X⁴ is deuterium and X⁵ is deuterium.

In one more particular embodiment, X³ is deuterium, X⁴ is hydrogen, X⁵ is hydrogen, and X⁶ is hydrogen.

In one more particular embodiment, X³ is deuterium, X⁴ is deuterium, X⁵ is hydrogen and X⁶ is hydrogen.

In one more particular embodiment, X³ is deuterium, X⁴ is hydrogen, X⁵ is deuterium and X⁶ is hydrogen.

In one more particular embodiment, X³ is deuterium, X⁴ is deuterium, X⁵ is deuterium and X⁶ is hydrogen.

In one more particular embodiment, X³ is deuterium, X⁴ is hydrogen, X⁵ is hydrogen and X⁶ is deuterium.

In one more particular embodiment, X³ is deuterium, X⁴ is deuterium, X⁵ is hydrogen and X⁶ is deuterium.

In one more particular embodiment, X³ is deuterium, X⁴ is hydrogen, X⁵ is deuterium and X⁶ is deuterium.

In one more particular embodiment, X³ is deuterium, X⁴ is deuterium, X⁵ is deuterium and X⁶ is deuterium.

In some embodiments, X⁴ is hydrogen.

In one more particular embodiment, X⁴ is hydrogen and X⁵ is hydrogen.

In one more particular embodiment, X⁴ is hydrogen and X⁵ is deuterium.

In one more particular embodiment, X⁴ is hydrogen, X⁵ is hydrogen, and X⁶ is hydrogen.

In one more particular embodiment, X⁴ is hydrogen, X⁵ is deuterium and X⁶ is hydrogen.

In one more particular embodiment, X⁴ is hydrogen, X⁵ is hydrogen, and X⁶ is deuterium.

In one more particular embodiment, X⁴ is hydrogen, X⁵ is deuterium and X⁶ is deuterium.

In some embodiments, X⁴ is deuterium.

In one more particular embodiment, X⁴ is deuterium and X⁵ is hydrogen.

In one more particular embodiment, X⁴ is deuterium and X⁵ is deuterium.

In one more particular embodiment, X⁴ is deuterium, X⁵ is hydrogen, and X⁶ is hydrogen.

In one more particular embodiment, X⁴ is deuterium, X⁵ is deuterium and X⁶ is hydrogen.

In one more particular embodiment, X⁴ is deuterium, X⁵ is hydrogen, and X⁶ is deuterium.

In one more particular embodiment, X⁴ is deuterium, X⁵ is deuterium and X⁶ is deuterium.

In some embodiments, X⁵ is hydrogen.

In one more particular embodiment, X⁵ is hydrogen, and X⁶ is hydrogen.

In one more particular embodiment, X⁵ is hydrogen, and X⁶ is deuterium.

In some embodiments, X⁵ is deuterium.

In one more particular embodiment, X⁵ is deuterium and X⁶ is hydrogen.

In one more particular embodiment, X⁵ is deuterium and X⁶ is deuterium.

In some embodiments, X⁶ is hydrogen. In some embodiments, X⁶ is deuterium.

As non-limiting examples, the compound of Formula (I) can be selected from the group consisting of:

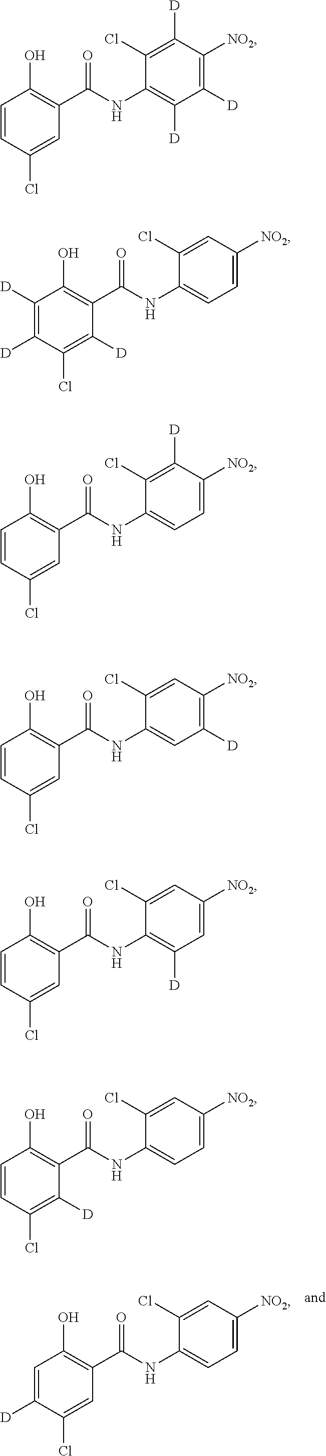

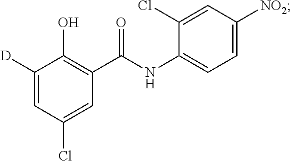 [SMILES: [2H]c1cc(Cl)cc(C(=O)Nc2ccc([N+](=O)[O-])cc2Cl)c1O]

or a pharmaceutically acceptable salt thereof.

Chemical Purity

In some embodiments, the Formula (I) compounds has a chemical purity of greater than about 99.0%; e.g., greater than about 99.5%; or greater than about 99.7%; or greater than about 99.8%.

In some embodiments, Formula (I) compounds have less than about 45 ppm of 5-chloro-salicylic acid (including deuterated 5-chloro-salicyclic acid); e.g., less than about 30 ppm of 5-chloro-salicylic acid (including deuterated 5-chloro-salicylic acid).

In some embodiments, the compound has less than about 50 ppm of 2-chloro-4 nitro-aniline (including deuterated 2-chloro-4 nitro-aniline). In certain embodiments, the compound has less than about 10 ppm of 2-chloro-4 nitro-aniline (including deuterated 2-chloro-4 nitro-aniline).

In some embodiments, the compound has less than about 45 ppm of 5-chloro-salicylic acid (including deuterated 5-chloro-salicylic acid) and less than about 50 ppm of 2-chloro-4 nitro-aniline (including deuterated 2-chloro-4 nitro-aniline).

In some embodiments, the compound has less than about 30 ppm of 5-chloro-salicylic acid (including deuterated 5-chloro-salicylic acid) and less than about 10 ppm of 2-chloro-4 nitro-aniline (including deuterated 2-chloro-4 nitro-aniline).

In some embodiments, the compound has less than about 0.05% water. In certain embodiments, the compound is substantially free of hydrated Formula (I) compound solid forms. As a non-limiting example, the compound can be an anhydrous compound of Formula (I).

In some embodiments, purification can be carried out according to the following process. Acetone and crude Formula (I) compound are mixed in a vessel and heated to reflux (−56° C.) until solids dissolve. The solution is clarified by filtration and transferred to a second vessel, heated to 45° C. to 55° C. to dissolve the solids, cooled to −5° C. to 5° C. and stirred at this temperature for at least 2 hours. The solids are filtered and washed with acetone. Crystallized Formula (I) compound is obtained after vacuum drying of the solids at 70° C. IPC LOD testing is performed on the dry solids with a specification of <1.0%. If the LOD results are >1.0% the drying step may be repeated two additional times. IPC testing is also performed to ensure the level of the starting material 2-chloro-4-nitroaniline (including deuterated 2-chloro-4 nitro-aniline) is <100 ppm. If the level of 2-chloro-4-nitroaniline (including deuterated 2-chloro-4 nitro-aniline) is >100 ppm, a second crystallization may be performed.

In some embodiments, purity analysis can be achieved according to the following procedure. Chromatograph: UPLC system consisting pump, diode array; detector, autosampler, auto injector, and column cooler/heater, or equivalent. Column: Agilent Poroshell 120 EC-C18 column, 4.6×50 mm, 2.7 μm or equivalent. Column Temperature: 35° C. Mobile phase A: 20 mM ammonium acetate (pH 5.50). Mobile phase B: MeOH:ACN (70:30, v/v). Diluent: MeOH:DMSO (70:30, v/v).

| Time | Solvent A | Solvent B |
|---|---|---|
| (min.) | (%) | (%) |
| 0.1 | 75 | 25 |
| 1.0 | 75 | 25 |
| 21.0 | 30 | 70 |

Flow rate: 1.0 ml/min. Injected volume: 3.00 μl. Preparation of standard and sample solutions. Niclosamide Standard Solutions: Concentration of this solution is nominally 0.8 mg/mL. Retention times: 5-Chlorosalicylic acid (2.9 minutes); 2-Chloro-4-nitroaniline (7.0 minutes); and Niclosamide (18.8 minutes).

Particle Size

In some embodiments, the compound has a reduced particle size (e.g., as achieved by techniques including but not limited to milling).

In some embodiments, Formula (I) compounds having reduced particle size can be prepared by jet milling, e.g., using CMTI equipment NGMP-Mill-A, a 2-inch, pancake micronizer manufactured by Sturtevant; a flexible containment unit was used during the milling process (Mill and Venturi pressure both=50 psi; feed rate 96.0 g/hour).

In some embodiments of the foregoing, the compound has a particle size range of from about 0.1 μm to about 30 μm. In certain embodiments, the compound has a particle size range of from about 0.1 μm to about 20 μm. In certain embodiments, the compound has a particle size range of from about 0.1 μm to about 10 μm.

The term “particle size distribution” of a powder, or granular material, or particles dispersed in fluid, as used within this application, is a list of values or a mathematical function that defines the relative amounts of particles present, sorted according to size. The d(0.1), d(0.5) and d(0.9) values indicate that 10%, 50% and 90% of the particles measured were less than or equal to the size stated. For example, values of d(0.1)=0.6, d(0.5)=3.1 and d(0.9)=7.3 mean that 10% of the particles were less than or equal to 0.6 μm, 50% were less than or equal to 3.1 μm, and 90% were less than or equal to 7.3 μm.

Particle Size Distribution (PSD) can be determined by laser diffraction technique, e.g., using a “MALVERN MASTERSIZER 2000” (standard range between 0.020 and 2000.0 microns), model “APA 2000”, equipped with “Hydro 2000 μm” as dispersing unit. A representative procedure includes: approximately 50 mg of a compound of Formula (I) is dispersed manually into 25 ml of water; after dispersion the sample was sonicated with external ultrasound for two minutes (Ultrasonic frequency; 37 kHz—Elmasonic S100 (H)—Elma Schmidbauer GmbH, Germany); the following operative conditions/machine parameters are taken into account: Dispersant: Water+3 drops of Tyloxapol 1.5%; Background measurement time: 10 seconds; Number of measurements cycles: 3 (to obtain average value); Stir speed (dispersing unit): 1500 rpm.

In some embodiments, the compound has a particle size distribution D(0.9) of from about 1.0 μm to about 15.0 μm. In certain embodiments, the compound has a particle size distribution D(0.9) of from about 1.0 μm to about 10.0 μm. In certain embodiments, the compound has a particle size distribution D(0.9) of from about 6.0 μm to about 8.0 μm (e.g., about 7.3 μm (e.g., 7.3 μm)). In other embodiments, the compound has a particle size distribution D(0.9) of from about 2.2 μm to about 3.2 μm.

In some embodiments, the compound has a particle size distribution D(0.1) of from about 0.1 μm to about 1.5 μm. In certain embodiments, the compound has a particle size distribution D(0.1) of from about 0.1 μm to about 1.0 μm. In certain embodiments, the compound has a particle size distribution D(0.1) of from about 0.3 μm to about 0.9 μm. In certain embodiments, the compound has a particle size distribution D(0.1) of from about 0.45 μm to about 0.75 μm (e.g., about 0.6 μm (e.g., 0.6 μm)).

In some embodiments, the compound has a particle size distribution D(0.5) of from about 0.5 μm to about 6.0 μm. In certain embodiments, the compound has a particle size distribution D(0.5) of from about 1.0 μm to about 4.0 μm. In certain embodiments, the compound has a particle size distribution D(0.5) of from about 1.0 μm to about 2.0 μm. In certain other embodiments, the compound has a particle size distribution D(0.5) of from about 2.5 μm to about 3.5 μm (e.g., about 3.1 μm (e.g., 3.1 μm)).

The parameter D(0.1) as used herein refers to the mesh size of a single notional sieve allowing 10% of the total of all particles of the sample to pass. Thus D(0.1)=0.1-1.5 μm means that the upper limit of the particle size range defining the 10% of smallest particles in the sample is between 0.1 μm to 1.5 μm. Thus 10% of the total particles have a particle size of not more than D(0.1) meaning in this case that they have a maximum size of 0.1 μm to 1.5 μm.

The parameter D(0.5) refers to the mesh size of a single notional sieve allowing 50% of the total of all particles of the sample to pass. Thus D(0.5)=0.5-6.0 μm means that the upper limit of the particle size range defining the notional half of the sample containing the smaller particles is between 0.5 μm to 6.0 μm. Thus, 50% of the total of all particles have a particle size of not more than D(0.5) meaning in this case that they have a maximum size of 0.5 μm to 6.0 μm.

The parameter D(0.9) refers to the mesh size of a single notional sieve allowing 90% of the total of all particles of the sample to pass i.e. only 10% of the sample is retained. Thus, D(0.9)=1.0-15.0 μm means that the lower limit of the particle size range defining the 10% of largest particles in the sample is between 1.0 μm to 15.0 μm. Thus 90% of all particles have a particle size of not more than D(0.9) meaning in this case that they have a maximum size of 1.0 μm to 15.0 μm.

In some embodiments, the compound has less than about 0.05% water (e.g., as determined by Karl Fisher technique). In certain embodiments, the compound is substantially free of hydrated Formula (I) compound solid forms. As a non-limiting example, the compound can be an anhydrous compound of Formula (I).

In some embodiments, the compound is crystalline.

In some embodiments, the compound has a specific surface area of from about 5 m²/g to about 10 m²/g.

### Non-Limiting Combination

### Non-Limiting Combinations [A]

In some embodiments, the compound has a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

In some embodiments, the compound has a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

In some embodiments, the compound has a particle size distribution D(0.9) of from about 7.0 μm to about 7.5 μm (e.g., about 7.3 μm), a particle size distribution D(0.5) of from about 2.5 μm to about 4.0 μm (e.g., about 3.1 μm), and a particle size distribution D(0.1) of from about 0.45 μm to about 0.75 μm (e.g., about 0.6 μm).

In some embodiments, the compound has a particle size distribution D(0.9) of about 7.3 μm, a particle size distribution D(0.5) of about 3.1 μm, and a particle size distribution D(0.1) of about 0.6 μm.

In some embodiments, the compound has a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

In some embodiments, the compound has a chemical purity of greater than about 99.0%, a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

In some embodiments, the compound has a chemical purity of greater than about 99.0%, a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm. n some embodiments, the compound has a chemical purity of greater than about 99.0%, a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

In some embodiments, the compound has a chemical purity of greater than about 99.0%, a particle size range of from about 0.1 μm to about 30 μm, a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

In some embodiments, the compound has a chemical purity of greater than about 99.0%, a particle size range of from about 0.1 μm to about 30 μm, a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

In some embodiments, the compound has a chemical purity of greater than about 99.0%, a particle size range of from about 0.1 μm to about 30 μm, a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

In certain embodiments of [A], the compound has a particle size distribution D(0.5) of from about 2.5 μm to about 3.5 μm.

In certain embodiments of [A], the compound has a particle size distribution D(0.5) of from about 1.0 μm to about 2.0 μm.

In certain embodiments of [A], the compound has a chemical purity of greater than about 99.5%; or a chemical purity of greater than about 99.7%; or a chemical purity of greater than about 99.8%.

In certain embodiments of [A], the compound has less than about 45 ppm of 5-chloro-salicylic acid (including deuterated 5-chloro-salicylic acid); or less than about 30 ppm of 5-chloro-salicylic acid (including deuterated 5-chloro-salicylic acid).

In certain embodiments of [A], the compound has less than about 50 ppm of 2-chloro-4 nitro-aniline (including deuterated 2-chloro-4 nitro-aniline); or less than about 10 ppm of 2-chloro-4 nitro-aniline (including deuterated 2-chloro-4 nitro-aniline).

In certain embodiments of [A], the compound has less than about 45 ppm of 5-chloro-salicylic acid (including deuterated 5-chloro-salicylic acid) and less than about 50 ppm of 2-chloro-4 nitro-aniline (including deuterated 2-chloro-4 nitro-aniline); or less than about 30 ppm of 5-chloro-salicylic acid (including deuterated 5-chloro-salicylic acid) and less than about 10 ppm of 2-chloro-4 nitro-aniline (including deuterated 2-chloro-4 nitro-aniline).

In certain embodiments of [A], the compound has less than about 0.05% water.

In certain embodiments of [A], the compound is substantially free of hydrated Formula (I) compound solid forms.

In certain embodiments of [A], the compound is an anhydrous compound of Formula (I).

In certain embodiments of [A], the compound is crystalline.

In certain embodiments of [A], the compound has a specific surface area of from about 5 m²/g to about 10 m²/g.

Cocrystals of Compounds of Formula (I)

Overview

In some embodiments, the Formula (I) compounds can be in the form of a cocrystal that includes (i) a compound of Formula (I) or a pharmaceutically acceptable salt thereof, and (ii) one or more pharmaceutically acceptable coformers. The term “co-crystal” as used herein refers to a crystalline material comprised of two or more unique solids at room temperature in a stoichiometric or non-stoichiometric ratio, which are held together in the crystal lattice by one or more non-covalent interactions (e.g., hydrogen bonds, pi-stacking, guest-host complexation and van der Waals interactions).

In some embodiments, at least one of the one or more non-covalent interactions is a hydrogen bond. In certain of these embodiments, the chemical entity is the hydrogen bond donor, and one of one or more coformers is the hydrogen bond acceptor. In other embodiments, the chemical entity is the hydrogen bond acceptor, and one of one or more coformers is the hydrogen bond donor.

The co-crystals described herein can include one or more solvate (e.g., water or an organic solvent containing one or more hydroxyl groups, e.g., a C₁-C₆ alcohol or diol, e.g., a C₁-C₆ alcohol or diol, e.g., ethanol or propylene glycol) molecules in the crystalline lattice. However, solvates of chemical entities that do not further comprise a coformer (e.g., a solid conformer) are not encompassed by the co-crystal definition set forth in this disclosure.

In some embodiments, the cocrystal includes more than one coformer. For example, two, three, four, five, or more co formers can be incorporated in a co-crystal with the chemical entity. The ratio of the chemical entity to each of the one or more pharmaceutically acceptable coformers may be stoichiometric or non-stoichiometric. As a non-limiting example, 1:1, 1:1.5 and 1:2 ratios of chemical entity:coformer are contemplated.

The Formula (I) compounds and each of the one or more pharmaceutically acceptable coformers may each be independently specified as a free form, or more specifically, a free acid, free base, or zwitter ion; a salt, or more specifically for example, an inorganic base addition salt such as sodium, potassium, lithium, calcium, magnesium, ammonium, aluminum salts or organic base addition salts, or an inorganic acid addition salts such as HBr, HCl, sulfuric, nitric, or phosphoric acid addition salts or an organic acid addition salt such as acetic, proprionic, pyruvic, malanic, succinic, malic, maleic, fumaric, tartaric, citric, benzoic, methanesulfonic, ethanesulforic, stearic or lactic acid addition salt; an anhydrate or hydrate of a free form or salt, or more specifically, for example, a hemihydrate, monohydrate, dihydrate, trihydrate, quadrahydrate, pentahydrate; or a solvate of a free form or salt.

Coformers

In some embodiments, at least one of the one or more pharmaceutically acceptable coformers can form one or more hydrogen bonds with the chemical entity in the cocrystal. In some embodiments, at least one of the one or more pharmaceutically acceptable coformers can accept one or more hydrogen bonds from the chemical entity in the cocrystal. In some embodiments, at least one of the one or more pharmaceutically acceptable coformers can form one or more hydrogen bonds with the chemical entity in the cocrystal, and at least one of the one or more pharmaceutically acceptable coformers can accept one or more hydrogen bonds from the chemical entity in the cocrystal.

In some embodiments, at least one of the one or more pharmaceutically acceptable coformers comprises one or more functional groups selected from the group consisting of: ether, thioether, hydroxy, sulfhydryl, aldehyde, ketone, thioketone, nitrate ester, phosphate ester, thiophosphate ester, ester, thioester, sulfate ester, carboxylic acid, phosphonic acid, phosphinic acid, sulfonic acid, amido, primary amine, secondary amine, ammonia, tertiary amino, sp2 amino, thiocyanate, cyanamide, oxime, nitrile, diazo, haloalkyl, nitro, heterocyclic ring, heteroaryl ring, epoxide, peroxide, and hydroxamic acid.

In certain embodiments, each of the one of the one or more pharmaceutically acceptable coformers is independently selected from acetamide, benzamide, (+/−)-limonene, 1-(phenylazo)-2-naphthylamine, 1,2,6-hexanetriol, 1,2-dimyristoyl-sn-glycero-3-(phospho-s-(1-glycerol)), 1,2-dimyristoyl-sn-glycero-3-phosphocholine, 1,2-dioleoyl-sn-glycero-3-phosphocholine, 1,2-dipalmitoyl-sn-glycero-3-(phospho-rac-(1-glycerol)), 1,2-distearoyl-sn-glycero-3-(phospho-rac-(1-glycerol)), 1,2-distearoyl-sn-glycero-3-phosphocholine, 1,5-naphthalene-disulfonic acid, 1-hydroxy-2-naphthoic acid, 1-o-tolylbiguanide, 2-ethyl-1,6-hexanediol, 4-aminobenzoic acid, 4-aminopyridine, 4-aminosalicylic acid, 4-chlorobenzene-sulfonic acid, 4-ethoxyphenyl urea, 7-oxo-dhea, acacia, acacia mucilage, acacia syrup, acesulfame, acesulfame potassium, acetohydroxamic acid, acetone sodium bisulfite, acetylated lanolin alcohols, acetylated monoglycerides, acetylcysteine, acetyltributyl citrate, acrylates copolymer, acrylic acid-isooctyl acrylate copolymer, adenine, adipic acid, alanine, albumin aggregated, albumin colloidal, albumin human, albumins, alginic acid, alkyl ammonium sulfonic acid betaine, alkyl aryl sodium sulfonate, allantoin, allopurineol, allyl alpha-ionone, alpha-terpineol, alpha-tocopherol, alpha-tocopherol acetate, aminobenzoate sodium, amyl acetate, anethole, anhydrous citric acid, anhydrous dextrose, anhydrous lactose, anhydrous tribasic sodium phosphate, anhydrous trisodium citrate, arginine, arlacel, asafetida, ascorbic acid, ascorbyl palmitate, asparagine, aspartame, aspartic acid, bacteriostatic sodium chloride injection, barium sulfate, benzalkonium chloride, benzenesulfonic acid, benzethonium chloride, benzododecinium bromide, benzoic acid, benzyl acetate, benzyl alcohol, benzyl benzoate, benzyl chloride, beta-carotene, betanaphthol, betose, bibapcitide, bismuth subcarbonate, bismuth subgallate, boric acid, brocrinat, butyl stearate, butylated hydroxyanisole, butylated hydroxytoluene, butylparaben, butyric acid, C-11-1-aminocyclohexanecarboxylic acid, C12-15 alkyl lactate, caffeine, calcobutrol, caldiamide sodium, caloxetate trisodium, calteridol calcium, camphoric acid, capric acid, captan, captisol, carboxypolymethylene, carmine, carnauba wax, carnauba yellow wax, carrageenan, carrageenan calcium, carrageenan salt, carrageenan sodium, ceresin, ceteareth-12, ceteareth-15, ceteareth-30, cetearyl alcohol/ceteareth-20, cetearyl ethylhexanoate, ceteth-10, ceteth-2, ceteth-20, ceteth-23, cetostearyl alcohol, cetrimonium chloride, cetyl alcohol, cetyl esters wax, cetyl palmitate, cetylpyridinium chloride, chlorocresol, chloroxylenol, cholesterol, chrysin, cinnamaldehyde, cinnamic acid, citrate, citric acid, citric acid monohydrate, clemizole, cocamide ether sulfate, cocamine oxide, coco betaine, coco diethanolamide, coco monoethanolamide, coco-caprylate, coco-glycerides, creatine, creatinine, cresol, cupric sulfate, cyclamic acid, cyclomethicone, cyclomethicone 5, cysteine, dalfampridine, decyl methyl sulfoxide, dehydroacetic acid, denatonium benzoate, deoxycholic acid, dextran, dextran 40, dextrates, dextrin, dextrose, dextrose monohydrate, diacetylated monoglycerides, diatrizoic acid, dibasic anhydrous sodium phosphate, dibasic sodium phosphate, dibasic sodium phosphate dihydrate, dibasic sodium phosphate dodecahydrate, dibasic sodium phosphate heptahydrate, dibutyl phthalate, dibutyl sebacate, diethyl phthalate, diethyl pyrocarbonate, diethyl sebacate, diethylaminoethyl stearamide phosphate, diethylene glycol monoethyl ether, diethylene glycol monomethyl ether, diethylhexyl phthalate, diisopropyl adipate, diisopropyl dilinoleate, diisopropylbenzothiazyl-2-sulfenamide, dimethicone medical fluid 360, dimethyl isosorbide, dimethyl phthalate, dimethyl sulfoxide, dimethyldioctadecylammonium bentonite, dimethylglycine, dimethylsiloxane/methylvinylsiloxane copolymer, dinoseb-ammonium, dipropylene glycol, disodium cocoamphodiacetate, disodium hydrogen citrate, disodium laureth sulfosuccinate, disodium lauryl sulfosuccinate, disodium oleamido monoethanolamine sulfosuccinate, disodium sulfosalicylate, disofenin, dl-a350 lactic acid, dl-acetyltryptophan, dl-alpha-tocopherol, dl-alpha-tocopherol acetate, dl-dipalmitoylphosphatidylglycerol, dl-distearoylphosphatidylcholine, dl-glutamic acid, dl-tartaric acid, d-mannose, dmdm hydantoin, docosanol, docusate sodium, d-ribose, edetate calcium disodium, edetate disodium, edetate sodium, edetic acid, egg phosphatidyl glycerol, egg phospholipids, entsufon, entsufon sodium, epilactose, epitetracycline hydrochloride, erythorbic acid, erythritol, ethanolamine hydrochloride, ethyl maltol, ethyl oleate, ethyl vanillate, ethyl vanillin, ethylenediamine dihydrochloride, ethylhexyl hydroxystearate, ethylparaben, eucalyptol, eugenol, exametazime, fatty acid esters, fatty acid glycerides, fatty acid pentaerythriol ester, fatty acids, fatty alcohol citrate, fatty alcohols, ferric chloride, ferric oxide, ferrosoferric oxide, ferrous fumarate, ferrous oxide, fluorescein, fructose, fumaric acid, fumaryl diketopiperazine, gadolinium oxide, galactaric acid, galactose, gamma cyclodextrin, genistein, gentisic acid, gentisic acid ethanolamide, gentisic acid ethanolamine, gluceptate sodium, gluconic acid, gluconolactone, glucosamine, glucose, glucuronic acid, glutamic acid, glutamic acid hydrochloride, glutamine, glutaric acid, glutathione, glyceryl caprylate, glyceryl dibehenate, glyceryl distearate, glyceryl isostearate, glyceryl laurate, glyceryl monostearate, glyceryl oleate, glyceryl palmitate, glyceryl palmitostearate, glyceryl ricinoleate, glyceryl stearate, glyceryl stearate—laureth-23, glyceryl stearate/peg stearate, glyceryl stearate/peg-100 stearate, glyceryl stearate/peg-40 stearate, glyceryl stearate-stearamidoethyl diethylamine, glyceryl trioleate, glycine, glycine hydrochloride, glycol distearate, glycol stearate, glycolic acid, glycyrrhizin, guanidine hydrochloride, hexylresorcinol, hippuric acid, histidine, hyaluronate sodium, hydrocortisone, hydroquinone, hydrous-citric acid, hydroxyethylpiperazine ethane sulfonic acid, hydroxyoctacosanyl hydroxystearate, hydroxyprogesterone caproate, hydroxypropyl beta-cyclodextrin, hystrene, illicium anisatum, imidazole, imidurea, indigotindisulfonate sodium, iodoxamic acid, iofetamine hydrochloride, ipriflavone, isoleucine, isopropyl isostearate, isopropyl myristate, isopropyl myristate—myristyl alcohol, isopropyl palmitate, isopropyl stearate, isostearic acid, isostearyl alcohol, lactate, lactitol monohydrate, lactobionic acid, lactose, landalgine, lanolin, lauralkonium chloride, lauramine oxide, laureth sulfate, lauric acid, lauric diethanolamide, lauric myristic diethanolamide, lauroyl sarcosine, lauryl lactate, lauryl sulfate, lecithin, leucine, levomenthol, levulinic acid, lidofenin, 1-sodium lactate, lysine, maleic acid, malic acid, malonic acid, maltitol, maltodextrin, maltol, maltose anhydrous, mandelic acid, mannitol, maprofix, mebrofenin, medium-chain triglycerides, medronate disodium, medronic acid, menthol, metacresol, methionine, methyl salicylate, methyl stearate, methylchloroisothiazolinone, methylisothiazolinone, methylparaben, methylparaben sodium, miripirium chloride, mono and diglyceride, monobasic sodium phosphate, monobasic sodium phosphate anhydrous, monobasic sodium phosphate dihydrate, monobasic sodium phosphate monohydrate, monoglyceride citrate, monoglycerides, monosodium citrate, monosodium glutamate, monostearyl citrate, monothioglycerol, myristic acid, myristyl alcohol, myristyl lactate, niacinamide, nicotinamide, nicotinic acid, N-methyl glucamine, octanoic acid, oleth-20, oleyl alcohol, oleyl oleate, orotic acid, oxalic acid, oxidronate disodium, oxyquinoline, palmitamine oxide, palmitic acid, pamoic acid, pentadecalactone, pentaerythritol cocoate, pentasodium pentetate, pentetate calcium trisodium, pentetic acid, phenol, phenonip, phenoxyethanol, phenylalanine, phenylethyl alcohol, phospholipid, piperazine, piperazine hexahydrate, procaine, product wat, proline, propenyl guaethol, propyl gallate, propylene carbonate, propylene glycol, propylene glycol—lecithin, propylene glycol alginate, propylene glycol diacetate, propylene glycol dicaprylate, propylene glycol monolaurate, propylene glycol monopalmitostearate, propylene glycol palmitostearate, propylene glycol ricinoleate, propylene glycol/diazolidinyl urea/methylparaben/propylparben, propylparaben, propylparaben sodium, p-toluenesulfonic acid, pyridoxamine, pyridoxine (4-pyridoxic acid), quercetin, resveratrol, riboflavin, saccharin, saccharin calcium, saccharin sodium, saccharin sodium anhydrous, salicylic acid, saturated fatty acid esters, sebacic acid, serine, sodium 1,2-ethanedisulfonate, sodium 2-naphthalenesulfonate, sodium acetate, sodium acetate anhydrous, sodium alginate, sodium alkyl sulfate, sodium aluminium silicate, sodium ascorbate, sodium benzoate, sodium bicarbonate, sodium bisulfate, sodium bisulfate acetone, sodium bisulfite, sodium bitartrate, sodium borate, sodium borate decahydrate, sodium carbonate, sodium carbonate decahydrate, sodium carbonate monohydrate, sodium carboxymethyl beta-glucan (ds 065-085), sodium caseinate, sodium cellulose, sodium cetostearyl sulfate, sodium chlorate, sodium chloride, sodium chloride injection, sodium cholesteryl sulfate, sodium citrate, sodium citrate hydrous, sodium cocoyl sarcosinate, sodium cyclamate, sodium desoxycholate, sodium dithionite, sodium dodecylbenzenesulfonate, sodium ethylparaben, sodium formaldehyde sulfoxylate, sodium gluconate, sodium hydroxide, sodium hypochlorite, sodium iodide, sodium lactate, sodium laureth-2 sulfate, sodium laureth-3 sulfate, sodium laureth-5 sulfate, sodium lauroyl sarcosinate, sodium lauryl sulfate, sodium lauryl sulfoacetate, sodium metabisulfite, sodium nitrate, sodium oleate, sodium phosphate, sodium phosphate dihydrate, sodium phosphite, sodium polyacrylate, sodium polyacrylate (2500000 MW), sodium polymetaphosphate, sodium propionate, sodium pyrophosphate, sodium pyrrolidone carboxylate, sodium starch glycolate, sodium starch glycolate type a corn, sodium starch glycolate type a potato, type B potato sodium starch glycolate, sodium stearate, sodium stearyl fumarate, sodium succinate hexahydrate, sodium sulfate, sodium sulfate anhydrous, sodium sulfate decahydrate, sodium sulfite, sodium sulfosuccinated undecyclenic monoalkylolamide, sodium tartrate, sodium thioglycolate, sodium thiomalate, sodium thiosulfate, sodium thiosulfate anhydrous, sodium trimetaphosphate, sodium tripolyphosphate, sodium xylenesulfonate, sorbic acid, sorbitan, sorbitan isostearate, sorbitan monolaurate, sorbitan monooleate, sorbitan monopalmitate, sorbitan monostearate, sorbitan sesquioleate, sorbitan trioleate, sorbitan tristearate, sorbitol, squalane, stannous 2-ethylhexanoate, stearalkonium chloride, stearalkonium hectorite/propylene carbonate, stearamidoethyl diethylamine, stearates, stearic acid, stearic diethanolamide, stearoxytrimethylsilane, stearyl alcohol, succinic acid, sucralose, sucrose, sucrose distearate, sucrose laurate, sucrose palmitate, sucrose polyesters, sucrose stearate, sucrose syrup, sulfacetamide sodium, sulfobutylether beta-cyclodextrin, tagatose, tartaric acid, tegacid, tert-butylhydroquinone, tetrofosmin, theophylline, thimerosal, threonine, thymol, tocopherol, tocophersolan, tragacanth, triacetin, tribasic sodium phosphate, tribasic sodium phosphate monohydrate, tribehenin, tricaprylin, triceteareth-4 phosphate, triethanolamine lauryl sulfate, triethyl citrate, trihydroxystearin, trilaneth-4 phosphate, trilaureth-4 phosphate, trimyristin, tris, trisodium citrate dihydrate, trisodium hedta, tristearin, trolamine, tromantadine, tromethamine, tryptophan, tyloxapol, tyrosine, undecylenic acid, urea, urethane, ursodiol, valine, vanillin, versetamide, viscarin, vitamin E, vitamin E acetate, vitamin K5, xylitol, and zinc sulfate. See also U.S. Pat. No. 7,927,613, which is incorporated herein by reference in its entirety. Other pharmaceutically acceptable coformers include those delineated in the “Generally Regarded as Safe” (“GRAS”) and/or the US FDA “Everything Added to Food in the United States” (“EAFUS”) lists.

In certain embodiments, at least one of the one or more pharmaceutically acceptable coformers is selected from the group consisting of caffeine, urea, p-aminobenzoic acid, theophylline, benzyl benzoate, and nicotinamide. In other embodiments, the one or more pharmaceutically acceptable coformers is other than those selected from the group consisting of caffeine, urea, p-aminobenzoic acid, theophylline, benzyl benzoate, and nicotinamide. In other embodiments, the one or more pharmaceutically acceptable coformers is other than those selected from the group consisting of acetamide, benzamide, 2-aminothiazole, and isoniazide. In still other embodiments, the one or more pharmaceutically acceptable coformers is an amino acid (e.g., proline, e.g., D-proline or L-proline, or racemic proline). In another embodiment, the one or more pharmaceutically acceptable coformers is a 5-10 (e.g., 5-9, 5-6, or 5) membered heteroaryl, e.g., a nitrogen-containing heteroaryl, e.g., imidazole.

In certain embodiments, at least one of the one or more pharmaceutically acceptable coformers is a second API. In certain of these embodiments, the second API is independently selected from (−)-amlodipine, (−)-halofenate, (R)-salbutamol, (R)-salbutamol, (R,R)-formoterol, (S)-doxazosin, (S)-fluoxetine, (S)-oxybutynin, 1,2-naphthoquinone, 17-methyltestosterone, 17α-hydroxyprogesterone, 195mPt-cisplatin, 1-naphthyl salicylate, 1-naphthylamine-4-, 1-theobromineacetic, 1α-hydroxycholecalciferol, 2,4,6-tribromo-m-cresol, 2,6-diamino-2′-butyloxy-3,5′-azopyridine, 2-[[[(1r)-2-(1h-imidazol-4-yl)-1-methylethyl]imino]phenylmethyl]-phenol, 21-acetoxypregnenolone, 2-amino-4-picoline, 2-aminothiazole, 2-ethoxybenzoic acid, 2-naphthol, 2-naphthyl benzoate, 2-naphthyl lactate, 2-naphthyl salicylate, 2-p-sulfanilylanilinoethanol, 2-thiouracil, 3′,3″,5′,5″-tetra-bromophenolphthalein, 3-amino-4-hydroxybutyric acid, 3-Bromo-D-camphor, 3-Hydroxycamphor, 3-O-Lauroylpyridoxol Diacetate, 3-pentadecylcatechol, 3-quinuclidinol, 4,4′-oxydi-2-butanol, 4,4′-sulfinyldianiline, 4-amino-3-hydroxybutyric acid, 4-amino-3-phenylbutyric acid, 4-aminosalicylic acid, 4-chloro-m-cresol, 4-hexylresorcinol, 4-salicyloylmorpholine, 5′-nitro-2′-propoxyacetanilide, 5-aminolevulinic acid, 5-azacitidine, 5-bromosalicyl-hydroxamic acid, 5F-DF-203, 5-FU, 5-HT3 antagonists, 6-azauridine, 6-mercaptopurine, 8-hydroxyquinoline, 9-aminocamptothecin, A-151892, A-5021, abacavir, abaperidone, abarelix, abciximab, abecarnil, abetimus, abiraterone, ABLC, ABT-751, AC-5216, acadesine, acamprosate, acamprosate, acarbose, acebrophylline, acebutolol, acecainide, acecarbromal, aceclofenac, acedapsone, acediasulfone, acefylline, aceglutamide, aceglutamide, acemetacin, acenocoumarol, aceponate, acetal, acetamidoeugenol, acetaminophen, acetaminosalol, acetanilide, acetarsone, acetazolamide, acetiamine, acetohexamide, acetohydroxamic acid, acetophenazine, acetophenide, acetophenone, acetosulfone, acetoxolone, acetrizoat, acetyl, acetylcarnitine, acetylcholine, acetylcholine, acetylcysteine, acetylleucine, acetylpheneturide, acetylsalicylate, acetylsalicylic acid, aciclovir, acifran, acipimox, acitazanolast, acitretin, aclarubicin, aclatonium, aconitine, Acranil®, acriflavine, acrisorcin, acrivastine, acrivastine, actagardine derivative, actarit, ACTH, acyclovir, adapalene, ADCON-L, adefovir, adefovir dipivoxil, adenoscan, adenosine triphosphate, ADEPT, adinazolam, adiphenine, ADL-10-0101, adrafinil, adrenalone, adrenochrome, adrogolide, AEOL-10150, aesthinol, AET, AF-2259, afloqualone, AG-041R, AG-2037, AGN-194310, agomelatine, ahistan, AHL-157, AIT-034, AIT-202, AJ-9677, AJG-049, ajmaline, akzo desogestrel, alacepril, alapivoxil, albaconazole, albendazole, albuterol, albutoin, alclofenac, alclometasone, alcuronium, aldioxa, aldol, aldosterone, alendronate, alendronic acid, alexidine, alfacalcidol, alfadolone, alfaxalone, alfentanil, alfimeprase, alfuzosin, alfuzosin, algestone, algestone, algin, alglucerase, alibendol, aliskiren, alitertinoin, alizapride, alkannin, alkofanone, allantoin, allobarbital, allopurinol, allyl isothiocyanate, allylestrenol, almagate, alminoprofen, almitrine, almotriptan, aloe-emodin, aloin, alosetron, alovudine, aloxiprin, alpha-, alpha-1 protease, alphaprodine, alpidem, alpiropride, alprazolam, alprenolol, alsactide, ALT-711, Althiazid, altinicline, altretamine, aluminium chloride hexahydrate, aluminon, aluminum acetate solution, aluminum chlorate, aluminum hydroxychloride, aluminum potassium sulfate, aluminum sodium sulfate, alusulf, alverine, alvimopan, alvocidib, ALX-0646, AM-24, AM-36, AM-477, amantadine, amantanium, ambazon, ambenonium, ambrisentan, ambroxol, ambucaine, ambuphylline, ambusid, ambutonium bromide, amcinonide, AMD-3100, amdinocillin, amdinocillin pivoxil, amdoxovir, amelubant, americaine, amezinium, amfenac, amidephrine, amidinomycin, amifostine, amiglumide, amikacin, amiloride, aminacrine, amineptine, aminitrozole, amino acid preparations, aminocaproic acid, aminoglutethimide, aminoguanidine, aminohippurate, aminometradine, aminopentamide, aminophylline, aminopromazine, aminopyrine, aminoquinuride, aminorex, amiodarone, amiodipine, amiphenazole, amiprilose, amisulpride, amitriptyline, amitriptyline+ketamine, amitriptylinoxide, amlexanox, ammoniacum, ammoniated mercuric chloride, ammonium benzoate, ammonium mandelate, ammonium salicylate, ammonium valerate, amobarbital, amocarzine, amodiaquin, amorolfine, amoscanat, amosulalol, amotriphene, amoxapine, amoxicillin, amoxicillin+potassium clavulan, AMPAlex, amphetamine, amphetaminil, amphotericin B, ampicillin, ampiroxicam, ampligen, amprenavir, amrinose, amrubicin, amsacrine, amtolmetin guacil, amylocaine, AN-152, anabolic steroids, anagestone, anagrelide, anastrozole, anazolene, ancitabine, ancrod, andolast, androisoxazole, androstenediol, anecortave, anethole, anethole trithione, angiogenix, angiotensin, anhydrovinblastine, anidulafungin, anilerdine, aniracetam, anisindione, anisomycin, anisotropine, anistreplase, antazoline, anthiolimine, anthralin, anthramycin, anthrarobin, anthrax inhibitor, antiangiogenic, anticort, antidepressants, anti-invasins, antimony potassium tartrate, antimony sodium thioglycollate, antimony thioglycollamide, antiprogestin, antipyrine, antipyrine salicylate, antithrombin III, anxiolytics, AP-521, AP-5280, apalcillin, apaziquone, apazone, apocodeine, apomine, apomorphine, apraclonidine, aprepitant, aprindine, aprobarbital, apronalide, aprotinin, aptiganel, AQ4N, aquavan, AR-116081, AR-A2, arachidonic acid, aranidipine, arbekacin, arbidol, arbutamine, arcitumomab, ardeparin, arecoline, argatroban, arginine, Ariflo®, aripiprazole, arofylline, arotinolol, arsacetin, arsenic trioxide, arsphenamine, arteether, arteflene, artemether, artemisinin, artemotil, artesunate, arzoxifene, AS-3201, ASA, ascaridole, ascorbic acid, asenapine, asimadoline, asocarboxazid, asoprisnil, asoxime, aspartic acid, aspidin, aspidinol, aspirin, aspirin dipyridamole, aspoxicillin, AST-120, astemizole, asulacrine, AT-1015, atamestane, atazanavir, atenolol, atenolol+chlorthalidone, atenolol+nifedipine, atevirdine, atipamezole, atiprimod dimaleate, ATL-146e, atomoxetine, atorvastatin, atosiban, atovaquone, atovaquone+proguanil, atracurium, atrasentan, atrial natriuretic, atrolactamide, atropine, augmentin, auranofin, aurothioglucose, avasimibe, avobenzone, AWD-12-281, azacitidine, azacyclonol, azanidazole, azapropazone, azaserine, azasertron, azatadine, azathipprine, AZD-4282, AZD-6140, azelaic acid, azelastine, azelnidipine, azidamfenicol, azidocillin, azimilide, azintamide, azithromycin, azlocillin, azosemide, aztreonam, azulene, bacampicillin, bacitracin, baclofen, baicalein, balofloxacin, balsalazide, bambuterol, bamethan, bamifylline, bamipine, barbital, barnidipine, BAS-118, basic alumina, baslilximab, batimastat, batroxobin, Bay-41-2272, Bay-41-8543, BAY-43-9006, BAY-57-1293, bazedoxifen, BBR-3464, BBR-3576, BBR-3610, BCH-1868, bebeerine, beclamide, beclometasone, befloxatone, befunolol, bemegride, benactyzine, benazepril, bencyclane, bendazac, bendroflumethiazide, benetonide, benexate, benfluorex, benfotiamine, benfurodil, benidipine, benorylate, benoxaprofen, benoxinate, benperidol, benproperine, benserazide, bentazepam, bentiromide, bentoquatam, benzafibrate, benzalkonium, benzarone, benzathine, benzbromarone, benzethonium, benzetimide, benzilonium, benziodarone, benznidazole, benzocaine, benzoctamine, benzonatate, benzoxonium chloride, benzoyl peroxide, benzoylpas, benzphetamine, benzpiperylon, benzquinamide, benzthiazide, benztropine, benzydamine, benzyl benzoate, benzylhydrochloro-thiazide, benzylmorphine, bephenium, bepotastine, bepridil, beraprost, berberine, bergapten, bermoprofen, besipirdine, betahistine, betaine, betamethasone, betamipron, betasine, betaxolol, betazole, bethanechol, bethanidine, betoxycaine, bevantolol, bevonium, bexarotene, bezitramide, BG-9928, BIA-2-024, BIA-2-093, BIA-3-202, bialamicol, biapenem, bibenzonium, bibrocathol, bicalutamide, bicifadine, bicisate, bicyclic, bidisomide, bietamiverine, bietanautine, bietaserpine, bifermelane, bifluranol, bifonazole, bimatoprost, bimoclomol, bimosiamose, binifibrate, binodenoson, biomed-101, biotin, biperiden, biriperone, birlcodar, bisacodyl, bisantrene, bisbentiamine, bisdequalinium, bismuth, bismuth, bismuth, bismuth aluminate, bismuth ethyl, bismuth sodium, bismuth sodium triglycollamate, bismuth subcarbonate, bismuth subgallate, bismuth subnitrate, bismuth subsalicylate, bisoprolol, bisoprolol+HCTZ, bisoprolol+trichloromethiazide, bisoxatin, bithionol, bitolterol, bitoscanat, BL-3875, bleomycin, blonanserin, BMS-184476, BMS-387032, BN-82451, BNP-7787, BO-653, bolandiol, bolasterone, boldenone, bopindolol, bornyl chloride, bornyl salicylate, bortezomib, bosentan, bradycor, brain natriuretic, brallobarbital, brasofensine, brequinar, bretylium, brilliant green, brimonidine, brinzolamide, brivudin, brodimoprim, bromazepam, bromfenac, bromhexine bromide, bromindione, bromisovalum, bromocriptine, bromo-diphenhydramine, bromoform, bromopride, bromo-salicychloranilide, bromperidol, brompheniramine, broparoestrol, bropirimine, brostallicin, brotizolam, brovincamine, broxyquinoline, brozuridine, brucine, bucetin, bucillamine, bucindolol, bucladesine, buclizine, buclosamide, bucolome, bucricaine, bucumolol, budesonide, budesonide+formoterol, budipine, budralazine, bufeniode, bufetolol, bufexamac, buflomedil, buformin, bufuralol, bumadizon, bumetanide, bunaftine, bunamiodyl sodium, bunazosin, bunitrolol, bupivacaine, bupranolol, buprenorphine, bupropion, buramate, buserelin, buspirone, busulfan, busulfan, butabarbital, butacaine, butacetin, butalamine, butalbital, butallylonal, butamben, butamirate, butanilicaine, butaperazine, butaverine, butazolamide, butedronic acid, butenafine, butethal, butethamate, butethamine, buthalital, buthiazide, butibufen, butidrine, butobendine, butoconazole, butoctamide, butofilolol, butorphanol, butoxycaine, butriptyline, butropium, butylthiolaurate, butyrate propio, buzepide, BVT-5182, BXT-51072, C-1311, cabergoline, cabergoline, cacodylic acid, cactinomycin, cadexomer iodine, cadmium salicylate, cadralazine, cafaminol, caffeine, calcifediol, calcipotriene, calcipotriol, calcipotriol+beclometasone, calcitriol, calcium 3-aurothio-2-propanol-1-sulfonate, calcium acetylsalicylate, calcium bromolactobionate, calcium carbonate, calcium gluconate, calcium glycerophosphate, calcium hopantothenate, calcium iodobehenate, calcium iodosterate, calcium lactate, calcium levulinate, calcium mesoxalate, calcium N-carbamoylaspartate, calcium polycarbophil, calcium propionate, calcium succinate, caldaret, calusterone, camazepam, camostat, camphor, camphorate, camphotamide, camptothecin, candesartan, candesartan cilexetil, candoxatril, canertinib, canrenone, cantharidin, cantuzumab mertansine, capecitabine, capobenic acid, capravirine, capromab, capsaicin cream, captodiamine, captopril, captopril+HCTZ, capuride, carabersat, caramiphen, carazolol, carbachol, carbamazepine, carbamide peroxide, carbarsone, carbaryl, carbazochrome, carbendazim, carbenicillin, carbenoxolone, carbetapentane, carbicarb, carbidopa, carbidopa+levodopa-1, carbimazole, carbinoxamine, carbocloral, carbocysteine, carbon tetrachloride, carbonate gel, carboplatin, carboprost, carboprost, carboquone, carbromal, carbubarb, carbutamide, carbuterol, carfimate, carglumic acid, cargutocin, carindacillin, cariporide, cariporide, carisoprodol, carmofur, carmoxirole, carmustine, carnitine, caroverine, caroxazone, carphenazine, carpipramine, carprofen, carsalam, carteolol, carticaine, carubicin, carumonam, carvacrol, carvedilol, carvone, cascarillin, caspofungin, catechin, cathepsin K inhibitors, cathepsin S inhibitors, CC-401, CCI-779, CCR5 antagonists, CDC-394, CDC-801, CEE-03-310, cefactor, cefadroxil, cefalexin, cefalexin pivoxil, cefamandole, cefatrizine, cefazedone, cefazolin, cefbuperazone, cefcapene pivoxil, cefclidin, cefdinir, cefditoren pivoxil, cefepime, cefetamet, cefetamet pivoxil, cefixime, cefmenoxime, cefmetazole, cefininox, cefodizime, cefonicid, cefoperazone, cefoperazone+sulbactam, ceforanide, cefoselis, cefotazime, cefotetan, cefotiam, cefotiam hexetil, cefoxitin, cefozopran, cefpimizole, cefpiramide, cefpirome, cefpodoxime, cefprozil, cefroxadine, cefsulodin, ceftazidime, cefteram, ceftezole, ceftibuten, ceftizoxime, ceftizoxime, ceftriaxone, cefuroxime, cefuroxime axetil, cefuzonam, celecoxib, celgosivir, celiprolol, cellulose ethyl, CEP-1347, CEP-701, cephacetrile, cephaeline, cephalexin, cephaloglycin, cephaloridine, cephalosporin C, cephalothin, cephapirin, cephradine, cerivastatin, ceronapril, certoparin, ceruletide, cerviprost, cetalkonium, cetamolol, cethexonium, cethromycin, cetiedil, cetirizine, cetirizine, cetirizine+pseudoephedrine, cetotiamine, cetoxime, cetraxate, cetrimonium, cetrorelix, cetyldimethylethyl-ammonium, cetylpyridinium, cevimeline, CG-1521, chaulmoogric acid, chenodiol, CHF-3381, chlophedianol, chloracizine, chloral, chlorambucil, chloramine-B, chloramine-T, chloramino-chloramphenicol, chlorazanil, chlorbenzoxamine, chlorbetamide, chlorcyclizine, chlordantoin, chlorguanide, chlorhexadol, chlorhexidine, chloriazepoxide, chlorisondamine, chlormadinone, chlormerodrin, chlormezanone, chlormidazole, chlornaphazine, chloroazodin, chlorophyll, chloroprednisone, chloroprocaine, chloropyramine, chloroquine, chlorothen, chlorothiazide, chlorotrianisene, chloroxine, chloroxylenol, chlorozotocin, chlorphenamine, chlorphenesin, chlorpheniramine, chlorphenoxamide, chlorphenoxamine, chlorphentermine, chlorproethazine, chlorproguanil, chlorproguanil+dapsone, chlorpromazine, chlorpropamide, chlorprothixene, chlorquinaldol, chlortetracycline, chlorthalidone, chlorthenoxazine(e), chlorzoxazone, cholic acid, choline, choline theophyllinate, choline-L-alfoscerate, chromocarb, chromonar, chrysoidine, CHS-828, CI-1031, CI-1040, cibenzoline, ciclesonide, cicletanine, ciclonicate, ciclopirox, ciclosidomine, ciclosporin A, cidofovir, cifenline, cilansetron, cilastatin, cilazapril, cilengitide, cilnidipine, cilomilast, cilostazol, cimetidine, cimetropium, cinacalcet, cinchonidine, cinchonine, cinchophen, cinepazet, cinepazide, cinepazide, cinitapride, cinmetacin, cinnamedrine, cinnarizine, cinolazepam, cinoxacin, cinoxate, cinromide, cioteronel, cipamfylline, cipralisant, ciprofibrate, ciprofloxacin, ciprofloxacin+ciramadol, cisapride, cisatracurium, cisplatin, citalopram, citicoline, Citiolone, citrate, citric acid, citrulline, cizolirtine, CJ-13610, CKD-602, cladribine, clanobutin, clarithromycin, clavulan, clavulanate disodium, clavulanic acid, clebopride, clemastine, clemizol, clenbuterol, clentiazem, clevidipine, clevudine, clidanac, clidinium, clinafloxacin, clindamycin, clindamycin, clindamycin+tretinoin, clinofibrate, clinprost, clobazam, clobenfurol, clobenoside, clobenzepam, clobenzorex, clobenztropine, clobetasol, clobetasone, clobutinol, clocapramine, clocinizine, cloconazole, clocortolone, clodronate, clodronic acid, clofarabine, clofazimine, clofenamide, clofibrat, clofibric acid, cloflucarban, clofoctol, cloforex, clomacran, clomestrone, clometacin, clomethiazole, clometocillin, clomiphene, clomipramine, clomocycline, clonazepam, clonidine, clonitazene, clonitrate, clonixin, clopamid, clopenthixol, cloperastine, clopidogrel, clopirac, cloprednol, cloranolol, clorazepic acid, clorexolone, cloricromene, clorindione, clorprenaline, clortermine, clospirazine, clostebol, clothiapine, clotiazepam, clotrimazole, clotrimazole+betamethasone, cloxacillin, cloxazolam, cloxotestosterone, cloxyquin, clozapine, CMI-392, CMT-3, CNI-1493, CNS-5161, cobamamide, cocaethylene, cocaine, codeine, cofactor, colchicine, colesevelam, colestilan, colestipol, colforsin daropate, colfosceril, collagraft, colocynthin, colpormon, coluracetam, combretastatin A-4 prodrug, compound B, conivaptin conjugate, connettivina, convallatoxin, coparaffinate, corticorelin ovine, corticosterone, cortisone, cortivazol, cosyntropin, cotarnine, cotinine, co-trimazine, coumetarol, CP-248, CP-461, CPC-211, CPI-1189, CRA-0450, creatinol-O-phosphate, CRL-5861, crobenetine, croconazole, cromoglicic acid, cromolyn, cropropamide, crotamiton, crotethamide, crystacide, CS-502, CS-758, CS-834, CT-052923, CT-32228, cupric citrate, cuproxoline, CVT-2584, CX-659S, cyacetacide, cyamemazine, cyanidin, CYC400, cyclacillin, cyclandelate, cyclazocine, cyclexanone, cyclexedrine, cyclidrol, cyclin D1 inhibitors, cyclizine, cyclobarbital, cyclobendazole, cyclobenzaprine, cyclobutyrol, cyclocumarol, cyclodrine, cyclofenil, cycloguanil, cyclomethycaine, cycloniumelodide, cyclopentamine, cyclopenthiazide, cyclopentobarbital, cyclopentolate, cyclophosphamide, cyclopiroxalamine, cycloserine, cyclothiazide, cyclovalone, cymarin, cymserine, cynarin(e), cyp26 inhibitors, cyproheptadine, cyproterone, cysteamine, cystic fibrosis ther, cytarabine, D-24851, D-4418, DA-5018, DA-6034, DA-7867, DA-7911, DA-8159, dacarbazine, daclizumab, dactinomycin, dalbavancin, dalfopristin, dalfopristin+quinupristin, dalteparin, daltroban, danaparoid, danazol, danthron, dantrolene, dapiprazole, dapivirine, dapoxetine, dapsone, daptomycin, darbepoetin alfa, darifenacin, daunorubicin, DAX<SciClone, DB-67, D-camphocarboxylic, DCF-987, DDT, deaminooxytocin, deanol, debrisoquin, decamethonium, decimemide, decitabine, declopramide, deferiprone, deferoxamine, deflazacort, defosfamide, degarelix, dehydroascorbic acid, dehydroemetine, dehyrdocholic acid, delapri+manidipine, delapril, delavirdine, delmadinone, delmopinol, delorazepam, delucemine, demanyl, demecarium, demeclocycline, demecolcine, demegestone, demexiptilline, denaverine, dendrimers, denileukin diftitox, denopamine, denopterin, deoxycholic acid, deoxycorticosterone, deoxydihydro-streptomycin, deoxyepinephrine, depreotide, depsipeptide, deptropine, dequalinium, dersalazine, deserpidine, desferrioxamine, desflurane, desipramine, deslanoside, desloratadine, deslorelin, desmopressin, desogestrel, desogestrel+estradiol, desogestrel+ethinylestrad (1), desomorphine, desonide, desoximetasone, detaxtran, devacade, dexamethasone, dexanabinol, dexecadotril, dexefaroxan, dexetimide, dexibuprofen, dexketoprofen, dexloxiglumide, dexmedetomidine, dexmethylphenidate, dexpanthenol, dexrazoxane, dextran-1, dextranomer, dextroamphetamine, dextromethorphan, dextromoramide, dextropropoxyphene, dezocine, DF-1012, DFA-IV, D-fenchone, D-glucuronolactone, Diab II, diacerein, diampromide, diamthazole, diathymosulfone, diatrizoate, diazepam, diaziquone, diazoxide, dibekacin, dibenzepin, dibromopropamidine, dibucaine, dichloralphenazone, dichloramine T, dichlorisone, dichlorobenzyl alcohol, dichlorohydrin, dichlorophen, dichlorophenarsine, dichlorphenamide, diclofenac, diclofenac+HA, dicloxacillin, dicoumarol, dicumarol, dicyclomine, didanosine, dideoxyadenosine, didox, dienestrol, dienogest, dienogest+estradiol, diethadione, diethazine, diethylamide, diethylbromo-acetamide, diethylcarbamazine, diethylpropion, diethylstilbestrol, difemerine, difenamizole, difenoxin, difenpiramide, diflomotecan, diflorasone, difloxacin, diflucortolone, diflunisal, difluprednate, digitalin, digitoxin, digoxin, dihexyverine, dihydralazine, dihydrocodeine, dihydrocodeinone enol, dihydroergocryptine, dihydroergocryptine, dihydroergotamine, dihydromorphine, dihydrostreptomycin, dihydrotachysterol, dihydroxyaluminum, diisopromine, diisopropyl paraoxon, diisopropylamine, dilazep, dilevalol, diloxanide, diltiazem, dimecrotic acid, dimefline, dimeglumine, dimemorfan, dimenhydrinate, dimenoxadol, dimepheptanol, dimercaprol, dimetacrine, dimethadione, dimethazan, dimethindene, dimethisoquin, dimethisterone, dimethocaine, dimethoxanate, dimethyl sulfoxide, dimethylthiambutene, dimetofrine, dimorpholamine, dinoprostone, diosmectite, diosmin, dioxadrol, dioxaphetyl, dioxethedrine, dioxybenzone, diphemanil, diphenadione, diphencyprone, diphenhydramine, diphenidol, diphenoxylate, diphenylpyraline, diphetarsone, diphtheria & tetanus toxoids and acellular pertussis vaccine adsorbed, dipipanone, dipivefrin, dipyridamole, dipyridamole, dipyrocetyl, dipyrone, diquafosol, dirithromycin, disodium pamidronate, disofenin, disopyramide, distigmine, disulfamide, disulfiram, ditazol, dithiazanine, dithranol, ditiocarb, dixanthogen, dixyrazine, DJ-927, DK-507k, DL-Lactic Acid, DMDC, DMXAA, DNA Stealth, dobesilate, dobutamine, docarpamine, docetaxel, docosahexaenoic acid, docosanol, docusate, dofetilide, dolasetron mesilate, domiodol, domiphen, domitroban, domperidone, donepezil, donitriptan, dopamine, dopexamine, doramapimod, doranidazole, doripenem, dorzolamide, dorzolamide+timolol, dosmalfate, dosulepine, dotarizine, dothiepin, doxacurium, doxapram, doxazosin, doxefazepam, doxenitoin, doxepin, doxercalciferol, doxifluridine, doxofylline, doxorubicin, doxycycline, doxylamine, DPC-817, DPI-3290, DQ-113, drofenine, droloxifene, drometrizole, dromostanolone, dronabinol, dronedarone, droperidol, droprenilamine, dropropizine, drospirenone, drotaverine, drotebanol, droxicam, droxidopa, droxidopa, DU-125530, duloxetine, duramycin, durapatite, dutasteride, DW-1141, DW-286a, DW-471, DX-9065a, DY-9760e, dyclonine, dydrogesterone, dymanthine, dyphyllin, E-1010, E-2101, E2F antagonists, E-3620, E-5564, E-5842, E-6259, EAA-90, ebastine, eberconazole, ebrotidine, ebselen, eburnamonine, ecabapide, ecabet, ecadotril, ecgonidine, ecgonine, echothiophate, econazole, ecopipam, ecraprost, ectylurea, ED-71, edaravone, edatrexate, edetate calcium disodium, edetate disodium, edetate sodium, edetate trisodium, edonentan, edotreotide, edoxudine, edrecolomab, edrophonium, efalith, efaproxiral, efavirenz, efletirizine, eflornithine, efloxate, eflucimibe, efonidipine, EGIS-7229, eglumegad, egualen, elarofiban, elcatonin, elcosapentaenoic acid, eledoisin, eletriptan, elgodipine, ellagic acid, elliptinium, eltoprazine, elvucitabine, elzasonan, embelin, embramine, emedastine, emepronium, emetine, emitefur, EMM-210525, emodin, emorfazone, EMR-62203, emtricitabine, emylcamate, enalapril, enalaprilat, enallylpropymal, encainide, enciprazine, endralazine, enfenamic acid, enflurane, enilconazole, eniluracil, ENMD-0995, enocitabine, enol-3-IPA, enoxacin, enoxaparin, enoximone, enoxolone, enprostil, enrasentan, entacapone, entecavir, enviomycin, eoinephrine, epalrestat, epavir, EPC-K1, eperisone, epervudine, ephedrine, epicillin, epimestrol, epinastine, epirizole, epirubicin, epitiostanol, eplerenone, eplivanserin, epoprostenol, epostane, eprazinone, epristeride, eprosartan, eprozinol, eptapirone, eptaplatin, eptastigmine, eptazocine, eptifibatide, equilenin, equilin, ERA-923, erdosteine, ergocornine, ergocorninine, ergoloid mesylates, ergonovine, ergosterol, ergotamine, eritadenine, erlotinib, ertapenem, erythrityl tetranitrate, erythrocentaurin, erythromycin acistrate, erythromycin erythrophleine, erythromycin estolate, erythromycin glucoheptonate, erythromycin lactobionate, erythromycin propionate, erythromycin stearate, erythromycin stinoprate, esaprazole, escitalopram, esculin, eseridine, esmolol, esomeprazole, estazolam, ester, estradiol, estradiol, estramustine, estriol, estrogen, estrone, eszopiclone, etafedrine, etafenone, etamiphyllin, etanercept, etanidazole, etaqualone, eterobarb, ethacridine, ethacrynic acid, ethadion, ethambutol, ethamivan, ethamsylate, ethanolamine, ethaverine, ethchlorvynol, ethenzamide, ethiazide, ethinamate, ethinyl estradiol, ethinyl estradiol, ethinyl estradiol, ethionamide, ethisterone, ethoheptazine, ethopropazine, ethosuximide, ethotoin, ethoxzolamide, ethybenztropine, ethyl alcohol, ethyl biscoumacetate, ethyl chloride, ethyl dibunate, ethyl ether, ethyl icosapentate, ethyl loflazepate, ethyl loflazepate, ethylamine, ethylene, ethylestrenol, ethylidene, ethylmethyl-thiambutene, ethylmorphine, ethylnorepinephrine, ethynodiol, ethynylcytidine, etidocaine, etidronate, etidronic acid, etifelmin, etifoxine, etilefrin, etilevodopa, etiprednol, etiroxate, etizolam, etodolac, etodroxizine, etofenamate, etofibrate, etofylline, etofylline clofibrate, etofylline nicotinate, etoglucid, etomidate, etomidoline, etonitazene, etonogestrel, etoperidone, etoposide, etoposide phosphate, etoricoxib, etoxadrol, etozolin, etretinate, etryptamine, etymemazine, eucatropine, eugenol, EUK-134, EUK-189, evans blue, everolimus, exalamide, exametazime, exatecan, exemestane, exifone, exisulind, Exosurf®, ezetimibe, Factor IX, Factor VIII, Factor XIII, fadolmidine, fadrozole, falecalcitriol, famciclovir, famotidine, fampridine, fandofloxacin, fantofarone, faropenem, faropenem daloxate, fasidotril, fasudil, fazadinium bromide, febarbamate, febuprol, febuxostat, fedotozine, felbamate, felbinac, felodipine, felypressin, femoxetine, fenbenicillin, fenbufen, fenbutrazate, fencamfamine, fencamine, fenclozic acid, fendiline, fendosal, fenethylline, fenfluramine, fenipentol, fenofibrate, fenoldopam, fenoprofen, fenoterol, fenoverine, fenoxazoline, fenoxedil, fenozolone, fenpentadiol, fenpiprane, fenpiverinium, fenproporex, fenquizone, fenretinide, fenspiride, fentanyl, fentiazac, fenticlor, fenticonazole, fentonium bromide, fepradinol, feprazone, ferric sodium edetate, ferrioxamine B, ferrocholinate, ferrous gluconate, ferumoxytol, fesoterodine, fexofenadine, fibrostat, fidarestat, fiduxosin, finasteride, finrozole, fipexide, FK-960, flavopiridol, flavoxate, flecainide, fleroxacin, flesinoxan, flibanserin, floctafenine, flomoxef, flopropione, florantyrone, flosequinan, floxacillin, floxuridine, fluacizine, fluanisone, fluarizine, fluasterone, fluazacort, flucloronide, flucloxacillin, fluconazole, flucytosine, fludarabine, fludeoxyglucose F18, fludiazepam, fludrocortisone, flufenamic acid, fluindione, flumazenil, flumecinol, flumequine, flumethasone, flumethiazide, flunisolide, flunitrazepam, flunoxaprofen, fluocinolone acetonide, fluocinolone SAL, fluocinonide, fluocortin butyl, fluocortolone, fluorescein, fluoresone, fluorometholone, fluorosalan, fluorouracil, fluoxetine, fluoxymesterone, flupentixol, fluperolone, fluphenazine, flupirtine, fluprednidene acetate, fluprednisolone, fluproquazone, flurandrenolide, flurazepam, flurbiprofen, flurithromycin, flurogestone, flurothyl, fluroxen, fluspirilene, flutamide, flutazolam, fluticasone, flutoprazepam, flutrimazole, flutropium bromide, fluvastatin, fluvoxamine, folic acid, folinic acid, fomepizole, fominoben, fomivirsen, fomocaine, fonazine, fondaparinux, formebolone, formestane, formocortal, formoterol, fosamprenavir, foscarnet, fosfestrol, fosfluconazole, fosfomycin, fosfomycin, fosfosal, fosinopril, fosphenytoin, fotemustine, fropenem, frovatriptan, fructose, fructose-1,6-diphosphate, FTC, FTY-720, fudosteine, fulvestrant, fumagiline, fumagillin, furaltadone, furazabol, furazolidone, furazolium chloride, furonazide, furosemide, fursultiamine, furtrethonium, fusidic acid, G1, YM BioSciences, G25, GABA-A Alpha5, gabapentin, gabexate, gaboxadol, gadobenat, gadobutrol, gadodiamide, gadolinium, gadopentetic acid, gadoteridol, gadoversetamide, gadoxetic acid, galantamine, galanthamine, galarubicin, gallamine triethiodide, gallic acid, gallium maltolate, gallium nitrate, gallopamil, ganaxolone, ganciclovir, ganirelix, ganstigmine, gantofiban, garenoxacin, garnocestim, gatifloxacin, gefarnate, gefitinib, gemcabene, gemcitabine, gemeprost, gemfibrozil, gemifloxacin, gentamicin, gentian violet, gentiopicrin, gentisic acid, gepefrine, gepirone, gestodene, gestodene+ethinylest, gestonorone caproate, gestrinone, gimatecan, giractide, gitoxin, GL-406349, Glafenine, glatiramer, Glibornuride, gliclazide, glimepiride, glipizide, gliquidone, glisolamide, glisoxepid, globulin (human), glucametacin, glucoheptonic acid, gluconic acid, glucosamine, glucosulfone, glufosfamide, glutamic acid, glutaraldehyde, glutethimide, glyburide, glybuthiazol(e), glybuzole, glycerol, glycerophosphate, glycocyamine, glycol salicylate, glyconiazide, glycopyrrolate, glyhexamide, glymidine, glypinamide, GMDP, gold sodium, goserelin, GPI-1485, GPI-5693, graftskin, granisetron, grepafloxacin, griseofulvin, guaiacol, guaiapate, guaiazulene, guaifenesin, guaimesal, gualacolsulfonate, guamecycline, guanabenz, guanadrel, guanethidine, guanfacine, guanoxabenz, guanoxan, gugulipid, gusperimus, GW-280430A, GW-320659, GYKI-16084, hachimycin, halazepam, halcinonide, halobetasol, halofantrine, halometasone, haloperidol, halopredone, haloprogin, halopropane, halothane, haloxazolam, harkoseride, HE-2000, healos, hematoporphyrin, hepronicate, heptabarbital, heptaminol, hetacillin, hetastarch, hexacetonide, hexachlorophene, hexadimethrine, hexafluorenium, hexamethonium, hexamidine, hexapropymate, hexedine, hexestrol, hexestrol Bis(β-diethylaminoethyl ether), hexethal, hexetidine, hexobarbital, hexobendine, hexocyclium methyl sulfate, hexoprenaline, hextend, hexylcaine, HF-0299, HGP-2, HGP-6A, hidrosmin, histamine, Histapyrrodine, histrelin, HM-101, HMN-214, homatropine, homocamfin, homochlorcyclizine, hopantenic acid, HP-228, huperzine A, hyaluronan, hycanthone, hydnocarpic acid, hydralazine, hydrastine, hydrastinine, hydrochlorothiazide, hydrocodone, hydrocortamate, hydrocortisone, hydrocortisone, hydroflumethiazide, hydromorphone, hydroquinidine, hydroquinine, hydroquinone, hydroxid, hydroxocobalamin, hydroxyamphetamine, hydroxychloroquine, hydroxydione, hydroxyethyl ether, hydroxynaphthoate, hydroxypethidine, hydroxyphenamate, hydroxypropyl cellulose, hydroxystilbamidine, hydroxytetracaine, hydroxyzine, Hylan G-F 20, hymecromone, hyoscyamine, hypericin, IACFT, ibandronic acid, ibopamine, ibopamine, Ibritumomab, ibrolipim, ibudilast, Ibufenac, ibuprofen, ibuprofen piconol, ibuproxam, ibutilide, ICA-17043, icodextrin, idarubicin, Idazoxan, IdB-1016, idebenone, IDN-5109, idoxifen, idraparinux, idrocilamide, ifenprodil, ifosfamide, iguratimod, ilaprazole, ilomastat, iloperidone, iloprost trometamol, ILX23-7553, imatinib, imidapril, imidazole salicylate, imipenem, imipramine, imipramine N-Oxide, imiquimod, imolamine, implitapide, improsulfan, inactivated, inaperisone, incadronate, incadronic acid, indalpine, indanazoline, indapamide, indecainid, indeloxazine, indeloxazine, indenolol, indinavir, indiplon, indisetron, indisulam, indobufen, indocyanine green, indometacin, indoprofen, indoramin, induclem, infliximab, inhibitor, inhibitors, inosine pranobex, inositol, inositol niacinate, inverse agonist Mer, iobenguane, iobenzamic acid, iobitridol, iocarmic acid, iocetamic acid, iodamide, iodide, iodine, iodipamide, iodixanol, iodoalphionic acid, iodochlorhydroxyquin, iodoform, iodopyracet, iodopyrrole, iodoquinol, iodosubgallate, iofetamine 1231, ioglycamic acid, iohexol, iomeglamic acid, iomeprol, iopamidol, iopanoic acid, iopentol, iophendylate, iophenoxic acid, iopromide, iopronic acid, iopydol, iopydone, iothalamic acid, iotrolan, ioversol, ioxaglic acid, ioxilan, IP-751, ipidacrine, IPL-576092, ipodate, iponiazid, ipratpopium, ipratropium, ipratropium bromide, iprazochrome, ipriflavone, iprindole, iproclozid, ipsapiron, irbesartan, IRFI-042, IRFI-165, iridomyrmecin, irindalone, irinotecan, irofulven, iron sorbitex, irsogladine, IS-741, isaglitazone, ISAtx-247, isbogrel, isepamicin, isoaminile, isobutyl p-aminobenzoate, isoconazole, isoetharine, isofloxythepin, isoflurane, isoflurophate, isoladol, isomethadone, isometheptene, isoniazid, isonixin, isopromethazine, isopropamide iodide, isopropyl alcohol, isopropyl unoprostone, isoproterenol, isosorbide, isosorbide dinitrate, isosorbide mononitrate, isothipendyl, isotretinoin, isovaleryl, isoxepac, isoxicam, isoxsuprine, isradipine, israpafant, ISV-403, itasetron, ITF-282, itopride, itraconazole, itramin, itriglumide, iturelix, ivabradine, ixabepilone, J-104132, J-107088, J-113397, Janex-1, josamycin, JTV-519, K-777, kainic acid, kalimate, kallidin, KB-130015, KCB-328, kebuzone, ketamine, ketanserin, ketazolam, kethoxal, ketobemidone, ketoconazole, ketoprofen, ketorolac, ketorolac, ketotifen, khellin, kinetin, KNI-272, KP-103, KP-157, KP-544, KRN-5500, KT-136, KUL-7211, KW-2170, KW-6002, KW-7158, L-365260, L-5-hydroxy-tryptophan, L-745337, L-758298, L-826141, labetalol, lacidipine, lactic acid, lactitol, lactulose, lafutidine, lamifiban, lamivudine, lamotrigine, landiolol, lanicemine, laniquidar, lanoconazole, lanoteplase, lanreotide, lansoprazole, lanthanum carbonate, lapatinib, laquinimod, lasofoxifene, latamoxef, latanoprost, lauroguadine, laurolinium acetate, lawsone, LAX-111, lazabemide, LB-30057, L-cysteine, lefetamine, leflunomide, leflunomide, leiopyrrole, lenampicillin, lentinan, lepirudin, lercanidipine, lerisetron, lesopitron, leteprinim, letosteine, letrozole, leucocyanidin, leuprolide, leuprolide acetate, leuprorelin, levallorphan, levaminsole, levcromakalim, levetiracetam, levobetaxolol, levobunolol, levobupivacaine, levocabastine, levocetirizine, levodopa, levodropropizine, levofloxacin, levomethadyl acetate, levomoprolol, levonorgestrel, levophacetoperane, levopropoxyphene, levorphanol, levosimendan, levosulpride, levothyroxine, levovirin, lexidronam, lexipafant, LF-15-0195, LF-16-0687, LGD-1550, LH, LH-RH, liarozote, licofelone, licostinel, lidadronate, lidamidine, lidocaine, lidofenin, lidoflazine, limaprost, lincomycin, lindan, linezolid, linoleic acid, linolenic acid, liothyronine, lipase, lipo-dexamethasone, lipo-flurbiprofen, Lipogel HA, LiquiVent, liranaftate, lisinopril, lisofyllin, lisuride, lithium, lithium citrate, lixivaptan, LJP-1082, LLUAlpha, LMP-160, LMP-420, loanzapine, lobaplatin, lobeline, lobenzarit, lodoxamide, lofentanil, lofepramine, lofexidine, loflucarban, lomefloxacin, lomerizine, lomifylline, lomustine, lonafarnib, lonapalene, lonazolac, lonidamine, loperamide, loperamide oxide, loprazolam, loprinone, loracarbef, lorajmine, loratadine, lorazepam, lorcainide, lormetazepam, lornoxicam, losartan, loteprednol, lotrafiban, lovastatin, loxapine, loxiglumide, loxoprofen, Lu-35-138, lubeluzole, lubiprostone, lucanthone, lucanthone, lumefantrine, lumiracoxib, lurtotecan, lutetium texaphyrin, LV-216, LX-104, LY-156735, LY-293111, LY-293558, LY-355703, lyapolate, lymecycline, lynestrenol, lypressin, lysine acetylsalicylate, lysine salicylate, lysophospholipids, M-40403, mabuprofen, mabuterol, macrophage colony-stimulating factor, MADU, mafenide, mafosfamide, magaldrate, magenta I, magnesium, magnesium carbonate, magnesium chloride, magnesium citrate, magnesium gluconate, magnesium lactate, magnesium salicylate, malathion, malotilate, mandelic acid, mandelic acid isoamyl, mangafodipir, manidipine, mannomustine, mannose-6-phosphate, maprotilline, maribavir, marimastat, maxacalcitol, mazindol, mazipredone, MC-5723, MCC-478, MCI-154, m-cresyl acetate, MDAM, MDI-101, MDI-403, MDL-100907, mebendazole, mebeverine, mebhydroline, mebrofenin, mebutamate, mecamylamine, mechlorethamine, mechlorethamine oxide, mecillinam, meclizine, meclocycline, meclofenamate, meclofenamic acid, meclofenoxate, mecloqualone, mecysteine, medazepam, medifoxamine, medrogestone, medronic acid, medroxyprogesterone, medrysone, mefenamic acid, mefenorex, mefexamide, mefloquine, mefruside, megestrol, meglumin, meglutol, melagatran, melanocortin-4 agonist, melarsoprol, melengestrol, melevodopa, melinamide, melitracen, meloxicam, melperone, melphalan, meluadrine, memantine, MEN-10700, MEN-10755, menadiol, menadione, menadoxime, menbutone, menogaril, MENT, menthol, menthyl valerate, meobentine, meparfynol, mepartricin, mepazine, mepenzolate bromide, meperidine, mephenesin, mephenoxalone, mephentermine, mephenytoin, mephobarbital, mepindolol, mepitiostane, mepivacaine, mepixanox, meprednisone, meprobamate, meproscillarin, meptazinol, mequitazine, meralein, meralluride, merbromin, mercaptomerin, mercumallylic acid, mercuric oleate, mercuric oxycyanide, merimepodib, meropenem, mersalyl, mertiatide, mesalamine, mesalazine, mesna, mesoridazine, mestanolone, mesterolone, mestranol, mesulfen, metaclazepam, metampicillin, metapramine, metaproterenol, metaraminol, metazocine, metergoline, metformin, methacholine, methacycline, methadone, methafurylene, methamphetamine, methandriol, methandrostenolone, methantheline, methapyrilene, methaqualone, metharbital, methazolamide, methdilazine, methenamine, methenolone, methestrol, methetoin, methicillin, methimazole, methiodal, methionic acid, methionine, methisazone, methitural, methixene, methocarbamol, methohexital, methotrexate, methotrimeprazine, methoxamine, methoxsalen, methoxycinnamate, methoxyflurane, methoxyphenamine, methoxypromazine, methscopolamine, methsuximide, methyclothiazide, methyl blue, methyl nicotinate, methyl propyl ether, methyl salicylate, methyl tert-butyl ether, methylbenzethonium chloride, methylbromide, methylcobalamin, methyldopa, methylene blue, methylergonovine, methylhexaneamide, methylphenidate, methylprednisolone, methylprednisolone, methylprednisolone, methylthiouracil, methyltrienolone, methyprylon, methysergide, metiazinic acid, metipranolol, metoclopramide, metocurine iodide, metofenazate, metolazone, metopimazine, metopon, metoprolol, metralindole, metrizamide, metrizoic acid, metron s, metyrapone, metyrosine, mexazolam, mexenone, mexiletine, mezlocillin, MFH-244, mianserin, mibefradil, miboplatin, micafungin, miconazole, micronomicin, midaxifyline, midazolam, midecamycin, midecamycin acetate, midesteine, midodrine, midostaurin, mifepristone, miglitol, miglustat, mildronate, milnacipran, miloxacin, milrinone, miltefosine, minaprine, minocycline, minodronic acid, minoxidil, miokamycin, mirtazapine, misoprostol, mitemcinal, mitiglinide, mitobronitol, mitoguazone, mitolactol, mitomycin, mitotane, mitoxantrone, mitoxantrone, MIV-210, mivacurium, mivazerol, mizolastine, mizoribine, MKC-733, MLN-519, MLN-576, moclobemide, modafinil, moexipril, mofarotene, mofebutazone, mofegiline, mofetil, mofezolac, MOL-6131, molindone, molsidomine, mometasone, monatepil, monobenzone, monoethanolamine, monolaurin, monoterpene diols, montelukast, monteplase, moperone, mopidamol, moprolol, moracizine, morazone, moricizine, moroxydine, morphazinamide, morphine, morphine-6-glucuronide, mosapramine, mosapride, motexafin, motretinide, moveltipril, moxalactam, moxastine, moxaverine, moxestrol, moxifloxacin, moxisylyte, moxonidine, M-PGA, MPI-5010, MPI-5020, MPL, MRS-1754, MS-209, MS-275, MS-325, MS-377, mupirocin, muscarin, muzolimine, MX-1013, mycophenolate, mycophenolic acid, myrophine, N-(hydroxymethyl)-nicotinamide, N,N,N′,N′-tetraethylphthalamide, N-[4-[4-(2-methoxyphenyl)-1-piperazinyl]butyl]naphthalene-2-carboxamide, N2-formyl-sulfisomidine, N4-sulfanilylsulfanilamide, N4-β-D-glucosylsulfanilamide, nabilone, nabumetone, N-acetylcysteine, N-acetylmethionine, nadifloxacin, nadolol, nadoxolol, nafamostat, nafarelin, nafcillin, nafronyl, naftidofuryl, naftifine, naftopidil, nalbuphine, nalidixic acid, nalmefene, nalorphine, naloxone, naltrexone, NAMI, naminidil, nandrolone, napadisilate, naphazoline, naphthalene, naproxen, naproxen betainate, naratriptan, narceine, narcobarbital, natamycin, nateglinide, N-butyldeoxy-nojirimycin, N-butylscopolammonium Bromide, NC-503, NC-531, NCX-1000, NCX-4016, NCX-456, NCX-950, n-docosanol, NE-100, nealbarbital, nebivolol, nebostinel, nebracetam, nedaplatin, nedocromil, nefazodone, nefiracetam, nefopam, negamycin, nelfinavir, nemonapride, neostigmine, nepadutant, neramexane, neridronic acid, neriifolin, N-ethylamphetamine, neticonazole, netilmicin, nevirapine, NGD-98-2, nialamide, niaprazine, nicametate, nicaraven, nicardipine, nicergoline, niceritrol, niclosamide, nicoclonate, nicofuranose, nicomol, nicomorphine, nicorandil, nicotinamide, nicotine, nicotinic acid, nicotinic acid benzyl ester, nicotinyl alcohol, nifedipine, nifekalant, nifenalol, niflumic acid, nifuratel, nifurfoline, nifuroxazide, nifuroxime, nifurpirinol, nifurprazine, nifurtimox, nifurtoinol, nifurzide, NIK-254, nikethamide, nilutamide, nilvadipine, nimesulide, nimetazepam, nimodipine, nimorazole, nimustine, ninopterin, NIP-142, NIP-531, niperotidine, nipradilol, niridazole, nisoldipine, nitazoxanide, nitisinone, nitracrine, nitrazepam, nitrendipine, nitroflurbiprofen, nitrofurantoin, nitrofurazone, nitroglycerin, nitromersol, nitronaproxen, nitroxazepine, nitroxoline, nizatidine, nizofenone, NM-3, NM-702, N-methylephedrine, N-methylepinephrine, N-methylglucamine, NN-414, NNC-05-1869, nobel, nogalamycin, nolatrexed, nolomirole, nolpitantium, nomegestrol, nomifensine, noprylsulfamide, norbolethone, nordazepam, nordefrin, nordihydroguaiaretic acid, norelgestromin, norepinephrine, norethandrolone, norethindrone, norethynodrel, norfenefrine, norfloxacin, norgesterone, norgestimate, norgestrel, norgestrienone, norlevorphanol, normethadone, normethandrone, normorphine, norphenazone, norpipanone, norpseudoephedrine, nortriptyline, norvinisterone, noscapine, novembichin, novobiocin, noxiptillin, noxythiolin, NS-1209, NS-1231, NS-126, NS-220, NS-2330, NS5A inhibitors, NS-7, NS-8, NSC-330507, NSC-619534, NSC-697726, N-sulfanilyl-3,4-xylamide, NU-6027 nucleosides, NV-07, NVP-SRA880, NW-1029, NXY-059, Nylidrin, NZ-314, NZ-419, obidoxime chloride, OC-108, ocinaplon, octabenzone, octacaine, octamoxin, octaverine, octenidine, octodrine, octopamine, octotiamine, octreotide, octyl, ofloxacin, oleandrin, oleic acid, olmesartan—medoxomil, o-lodohippurate, olopatadine, olpadronic acid, olsalazine, oltipraz, OM-294DP, omacor, omapatrilat, omeprazole, omiloxetine, omoconazole, onapristone, ondansetron, ONO-3403, ONO-4128, ONO-8815 Ly, ONT-093, OPC-14523, OPC-31260, OPC-51803, OPC-6535, opiniazide, opioid analgesics, opipramol, orazamide, orazipone, Org-12962, Org-24448, oritavancin, orlistat, ormeloxifene, ornidazole, ornipressin, ornithine, ornoprostil, orotic acid, orphenadrine, orthocaine, osalmid, osanetant, osaterone, oseltamivir, OSI-7836, OSI-7904, ospemifene, otilonium bromide, ouabain, oxaceprol, oxacillin, oxaflozane, oxaliplatin, oxalyt-C, oxamarin, oxametacine, oxamniquine, oxandrolone, oxantel, oxapropanium, oxaprozin, oxatomide, oxazepam, oxazolam, oxcarbazepine, oxeladin, oxendolone, oxethazaine, oxetoron, oxiconazole, oxidronic acid, oxiniacic acid, oxiracetam, oxitropium, oxolamin, oxolinix acid, oxophenarsine, oxprenolol, oxybenzone, oxybutynin, oxycinchophen, oxycodone, oxygent, oxymesterone, oxymetazoline, oxymetholone, oxymethurea, oxymorphone, oxypendyl, oxypertine, oxyphenbutazone, oxyphencyclimine, oxyphenisatin, oxyphenonium, oxypinocamphone, oxypurinol, oxytedrine, oxytetracycline, ozagrel, p-(benzylsulfonamido)-benzoic acid, P-100, P-1202, P32/98, PA-824, PACAP 38, pactitaxel, PADRE, pagoclone, PAI inhibs, palindore, palivizumab, palonosetron, pamabrom, pamaquine, pamicogral, pamidronate, p-aminobenzoic acid, p-aminohippuric acid, p-amino-propiophenone, p-aminosalicylic acid, panavir, pancuronium, panipenem, pantethine, pantoprazole, pantothenic acid, papain, papaverine, paracetamol, paraflutizide, paraldehyde, paramethadione, paramethasone, paranyline, parathyroid hormone, parecoxib, parethoxycaine, pargyline, paricalcitol, paromomycin, paroxetine, paroxypropione, parsalmide, patrin-2, pazinaclone, pazufloxacin, p-bromoacetanilide, PC-NSAIDs, PD-0166285, pecilocin, pefloxacin, pegvisomant, pelletierine, pemetrexed, pemirolast, pemoline, pempidine, PEN-203, penamecillin, penbutolol, penciclovir, penethamate, penfluridol, penicillamine, penicillin G, penicillin G Procaine, penicillin N, penicillin O, penicillin V, penimepicycline, penntuss, pentaerythritol, pentaerythritol, pentaerythritol chloral, pentagastrin, pentagestrone, pentalyte, pentam thonium, pentamidine, pentazocine, pentetate, pentetic acid, pentetreotide, penthienate, pentifyllin, pentigetide, pentisomide, pentobarbital, pentolinium, pentorex, pentosan, pentostatin, pentoxifylline, pentoxyl, pentrinitrol, pentylenetetrazole, peplomycin, peptide, peptide, perazine, perfiromycin, perflubron, perfosfamide, pergolide, perhexiline, pericyazine, perifosine, perillyl alcohol, perimethazine, perindopril, periodyl, perisoxal, perlapine, permanganate, permethrin, perospirone, perphenazine, petroleum benzin, PH-10, phanquinone, pharmacor, pharmaprojects no. 6362, pharmaprojects no. 4994, pharmaprojects no. 5325, pharmaprojects no. 5972, pharmaprojects no. 6446, pharmaprojects no. 6590, pharmaprojects no. 6656, pharmaprojects no. 6691, pharmaprojects no. 6743, pharmaprojects no. 6748, phenacaine, phenacemide, phenacetin, phenadoxone, phenallymal, phenamet, phenamide, phenazocine, phenazopyridine, phenbutamide, phencyclidine, phendimetrazine, phenelzine, phenesterine, phenetharbital, phenethicillin, pheneturide, phenformin, phenglutarimide, phenindamine, phenindione, pheniprazine, pheniramine, phenmetrazine, phenobarbital, phenobutiodil, phenocoll, phenoctide, phenolphthalein, phenolphthalol, phenolsulfonphthalein, phenol-tetrachlorophthalein, phenoperidine, phenosulfazole, phenoxybenzamine, phenoxypropazine, phenprobamate, phenprocoumon, phenserine, phensuximide, phentermine, phentetiothalein, phentolamine, phenyl acetylsalicylate, phenyl aminosalicylate, phenyl salicylate, phenylbutazone, phenylephrine, phenylethanolamine, phenylmercury, phenylmethylbarbituric acid, phenylpropanolamine, phenylpropyl-methylamine, phenyltoloxamine, phenyramidol, phenytoin, phethenylate, phloroglucinol, pholcodine, pholedrine, phoramide, phosphate, phosphate, phosphocreatine, phosphocysteamine, phosphorylcholine, phthalylsulfathiazole, phthalysulfacetamide, p-hydroxyephedrine, phylloquinone, physostigmine, phytic acid, PI-88, piberaline, piboserod, picilorex, picloxydine, picoperine, picosulfate, picotamide, picumast, pidotimod, pifarnine, piketoprofen, pildralazine, pilocarpine, piloplex, pilsicainide, pimeclone, pimecrolimus, pimefylline, pimilprost, piminodine, pimobendan, pimozide, pinacidil, pinaverium, pinazepam, pindolol, pioglitazone, pipacycline, pipamazine, pipamperone, pipazethate, pipebuzone, pipecurium, pipecuronium, pipemidic acid, pipenzolate bromide, piperacetazine, piperacillin, piperazine adipate, piperidione, piperidolate, piperilate, piperine analogues, piperocaine, piperonal, piperoxan, piperylone, pipobroman, piposulfan, pipotiazine, pipoxolan, pipradrol, piprozolin, piracetam, pirarubicin, pirazolac, pirbuterol, pirenoxine, pirenzepine, piretanide, pirfenidone, piribedil, piridocaine, pirifibrate, piritramide, piritrexim, pirlindole, pirmenol, piroctone, piroheptine, piromidic acid, piroxicam, piroxicam betadex, piroxicam cinnamate, pirozadil, pirprofen, pitavastatin, pivagabine, pivaloyloxymethyl, pivalylbenzhydrazine, pivampicillin, pivampicillin/pivmecillinam, pivcefalexin, pivmecillinam, pixantrone, pizotifen, pizotyline, PKI-166, p-lactophenetide, plafibride, plasminogen activator, plasmocid, platonin, plaunotol, PLD-118, PLD-147, pleconaril, plicamycin, p-methyldiphenhydramine, PMS-601, Pneumococcal, PNU-288034, podophyllotoxin, polaprezinc, poldine methylsulfate, policresulen, polidexide, polidocanol, poliovirus vaccine, poly-ADPRT inhibitors, polyestradiol, polyphenon E, polythiazide, porfimer, posaconazole, posatirelin, potassium, potassium, potassium, potassium chloride, potassium gluconate, potassium p-aminobenzoate, povidone, povidone-iodine, PP-117, PR-2699, PR-608, practolol, prajmaline, pralidoxime, pralnacasan, pramipexole, pramiracetam, pramiverin, pramlintide, pramoxine, pranidipine, pranlukast, pranoprofen, prasterone, pratosartan, pravastatin, prazepam, praziquantel, prazosin, prednicarbate, prednimustine, prednisolone, prednisolone 21-diethylaminoacetate, prednisolone farnesil, prednisolone sodium, prednisone, prednival, prednylidene, pregabalin, pregnan-3α-ol-20-one, premarin+trimegestone, prenalterol, prenoxdiazine, prenylamine, prezatide, pridinol, prifinium, prilocaine, primaquine, primidone, prinomastat, PRO-2000, probenecid, probucol, procainamide, procaine, procarbazine, procaterol, prochlorperazine, procodazol, procyclidine, procymate, prodipine, proflavine, progabide, progesterone, proglumetacin, proglumide, proheptazine, prolactin, prolintane, prolonium, promazine, promedol, promegestone, promestriene, promethazine, pronethalol, propacetamol, propafenone, propagermanium, propallylonal, propamidine, propane-1,2-diol, propanidid, propantheline, proparacaine, propatyl, propenidazole, propentofylline, propicillin, propiomazine, propionic acid, propionyl 1-carnitine, propipocaine, propiram, propiverine, propizepine, propofol, propoxycaine, propoxyphene, propranolol, propylhexedrine, propyliodone, propylthiouracil, propyphenazone, proquazone, proscillaridin, prostacyclin, prostaglandin E1, prostaglandin E2, prostaglandin F2α, prosultiamine, protein C, protheobromine, prothipendyl, protiofate, protionamide, protizinic acid, protoanemonin, protoklol, protoporphyrin IX, protriptyline, pro-urokinase, proxazole, proxetil, proxibarbal, proxigermanium, proxyphylline, prozapine, prucalopride, prulifloxacin, pseudococaine, pseudoephedrine, pseudoephedrine, pseudoephedrine+triprolidine, psilocybin, PSK-3841, p-sulfanilyl-benzylamine, PT-141, pteropterin, puromycin, PX-12, pyrantel, pyrazinamide, pyridinol carbamate, pyridostigmine, pyridoxal 5-phosphate, pyridoxine, pyrilamine, pyrimethamine, pyrinoline, pyrisuccideanol, pyrithione, pyrithyldione, pyritinol, pyrocatechol, pyrogallol, pyronaridine, pyrophosphate, pyrovalerone, pyroxylin, pyrrobutamine, pyrrocaine, pyrrointrin, pyrvinium pamoate, quazepam, quercetin, quetiapine, quinacillin, quinacrine, quinagolide, quinapril, quinaprilat, quinapyramine, quinbolone, quinestradiol, quinestrol, quinethazone, quinfamide, quinidine, quinine, quinocide, quinupramine, quinupristin, R-107500, R-667, rabeprazole, racecadotril, racemethorphan, raloxifene, raltitrexed, ramatroban, ramifenazone, ramipril, ramosetron, Ramot project No. 1097, ranimustine, ranitidine, ranitidine bismuth, ranolazine, ranpirnase, rapacuronium, rasagiline, raubasine, ravuconazole, raxofelast, razoxane, RC-529, rebamipide, rebimastat, reboxetime, remacemide, remifentanil, reminetant, remoxipride, renzapride, repaglinide, repertaxin L-lysine salt, repinotan, repirinast, reposal, reproterol, rescimetol, rescinnamine, reserpiline, reserpine, resibufogenin, resiquimod, resorcinol, reteplase, retigabine, retinoic acid, revimid, R-flurbiprofen, rho (D) immune, rho-kinase inhibitors, ribavirin, riboflavin, ribostamycin, ricinoleic acid, ridogrel, rifabutin, rifalazil, rifametane, rifamide, rifampicin+trimethoprim, rifampin, rifamycin SV, rifapentine, rifaximin, rifaximine cream, rilmazafone, rilmenidine, riluzole, rimantadine, rimazolium, rimexolone, rimiterol, rimonabant, riodoxol, rioprostil, risedronate, risedronic acid, risperidone, ritanserin, ritipenem, ritodrine, ritonavir, rituximab, rivastigmine, rizatriptan, RJR-2403, RNA Stealth, Ro-0094889, Ro-61-1790, rociverine, rocuronium, rofecoxib, roflumilast, rokitamycin, rolipram, rolitetracycline, romurtide, ronifibrate, ropinirole, ropivacaine, roquinimex, rosaprostol, rosaramicin, rose bengal, rosiglitazone, rosoxacin, rostaporfin, rosuvastatin, rotigotine, rotraxate, roxarsone, roxatidine, roxifiban, roxindol, roxithromycin, RPR-109881A, RPR-130401, R-roscovitine, RS-0406, RSR-13, rubijervine, rubitecan, ruboxistaurin, rufinamide, rufloxacin, rupatadine, rutin, RWJ-54428, S-0139, S-15535, S-18886, S-34730, S-3578, S-36496, S-36527, S-5751, S-8510, S-8921, sabcomeline, sabeluzole, S-adenosylmethionine, safinamide, salacetamide, salazosulfadimidine, salbutamol, salicin, salicyl alcohol, salicylamide, salicylamide O-acetic acid, salicylanilide, salicylic acid, salicylsilfuric acid, salinazid, salmeterol, salsalate, salverine, samarium ¹⁵³Sm, sampatrilat, sancycline, saperconazole, sapropterin, saquinavir, saralasin, saredutant, saredutant, sarizotan, sarizotan, sarpogrelate, sarpogrelate, satigrel, satigrel, satraplatin, satraplatin, satumomab, satumomab, SB-237376, SB-237376, SB-238039, SB-238039, SB-277011, SB-277011, scarlet red, SCH-00013, SCH-00013, Sch-23863, Sch-23863, Sch-57790, Sch-63390, scillarenin, scopolamine, scopolamine, scopolamine N-oxide, SCS technology, secalciferol, secnidazole, secobarbital, selegiline, selenomethionine, sematilide, semotiadil, seocalcitol, sepimostat, seratrodast, sertaconazole, sertaconazole, sertindole, sertindole, sertraline, sertraline, sestamibi, setastine, setastine, sevelamer, sevelamer, sevoflurane, sevoflurane, SG-210, sibutramine, siccanin, sildenafil, silodosin, silprostone, silver lactate, silver picrate, silver sulfadiazine, simetride, simfibrate, simvastatin, sincalide, sintropium bromide, sisomicin, sitafloxacin, sitamaquine, sitaxsentan, sivelestat, SJA-6017, SL-65-1498, SLV-306, SLV-308, Sm153 lexidronam, S-methylmethionine, SMP-300, SN-38, SNAP-7941, SOA-132, soblidotin, sobrerol, sobuzoxane, sodium arsanilate, sodium arsphenamine, sodium chloride, sodium dibunate, sodium folate, sodium formaldehydesulfoxylate, sodium hyaluronate, sodium iodomethamate, sodium nitrite, sodium nitroprusside, sodium oxybate, sodium phenol-sulfonate, sodium phenylbutyrate, sodium phosphate, sodium prasterone sulfate, sodium propionate, sodium salicylate, sodium tetradecyl sulfate, sofalcone, solasulfone, solifenacin, sorbinicate, sorbitol, sorivudine, sotalol, soterenol, sozoiodolic acid, spaglumic acid, sparfloxacin, sparteine, SPA-S-843, spasmolytol, SPD-754, spectinomycin, SPI-339, spiperone, spirapril, spirogermanium, spironolactone, SR-121463, SR-144190, SR-146131, SR-271425, SR-27897, SR-31747, SR-58611, SS732, SS-750, SSR-149415, SSR-180575, SSR-181507, SSR-591813, SST-101, SSY-726, ST-200, stachyfilin, stallimycin, stampidine, stannous, stannsoporfin, stanolone, stanozolol, staph aureus ther, STAT4 inhibitors, stavudine, stenbolone, stepronim, stibocaptate, stibophen, stilbamidine, stiripentol, streptodornase, streptomycin, streptonicozid, streptonigrin, streptozocin, strontium ranelate, strontium-89 chloride, succimer, succinimide, succinylcholine, succinylcholine, succinylsulfathiazole, succisulfone, suclofenide, sucralfate, sufentanil, sulbactam, sulbactam+ampicillin, sulbenicillin, sulbentine, sulbutiamine, sulconazole, suleptanate, sulesomab, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanole, sulfalene, sulfaloxic acid, sulfamerazine, sulfameter, sulfamethazine, sulfamethizole, sulfamethomidine, sulfamethoxazole, sulfamethoxypyrazine, sulfamethoxypyridazine, sulfametrole, sulfamidochrysoidine, sulfamoxole, sulfanilamide, sulfanilic acid, sulfanilylurea, sulfaperine, sulfaphenazole, sulfaproxyline, sulfapyrazine, sulfapyridine, sulfarside, sulfarsphenamine, sulfasalazine, sulfasomizole, sulfasymazine, sulfathiazole, sulfathiourea, sulfinalol, sulfinpyrazone, sulfiram, sulfisomidine, sulfisoxazole, sulfobromophthalein, sulfonethylmethane, sulfoniazide, sulfonic acid, sulfonmethane, sulforidazine, sulfoxone, sulindac, sulisatin, sulisobenzone, sulmarin, sulmazole, suloctidil, sulphan blue, sulpiride, sultamicillin, sulthiame, sultopride, sultosilic acid, sumanirole, sumatriptan, SUN-N8075, suplatast, suprofen, suramin, surfactant TA, suriclone, suxibuzone, SYM-1010, SYM-2081, SYM-2207, symclosene, Syn-1253, Syn-2190, Syn-2869, synephrine, syrosingopine, T-1095, T-1249, T-3912, T-588, T-67, T-82, TA-2005, TA-2005, TA-993, tabimorelin, tacalcitol, tacedinaline, tacrine, tacrolimus, tadalafil, tafenoquine, tafluposide, TAK-375, TAK-427, TAK-559, taka-diastase, talampanel, talampicillin, talaporfin, talastine, talbutal, talinolol, talipexole, talnetant, talniflumate, taltirelin, tamoxifen, tamsulosin, tandospirone, tannoform, taprostene, tariquidar, TAS-103, tasosartan, taurocholic acid, taurolidine, tazanolast, tazarotene, tazobactam, tazobactam+piperacillin, TBC-3711, TCH-346, tebipenem, teboroxime, tecadenoson, tecastemizole, Technetium ⁹⁹Tc, teclothiazide, teclozan, tedisamil, teflurane, tegafur, tegafur+uracil, tegaserod, teicoplanin, telbivudine, telenzepine, telithromycin, telmesteine, telmisartan, telomerase inhibs, temazepam, temiverine, temocapril, temocillin, temoporfin, temozolomide, tenatoprazole, tenecteplase, tenidap, teniposide, tenofovir, tenofovir disoproxil, tenonitrozole, tenoxicam, tenuazonic acid, teprenone, terazosin, terbinafine, terbutaline, terconazole, terfenadine, terguride, terlipressin, terodiline, terofenamate, terpin, tertalolol, tert-pentyl alcohol, tesaglitazar, tesmilifene, testolactone, testosterone, tetrabamate, tetrabarbital, tetrabenazine, tetracaine, tetrachloroethylene, tetracine, tetracycline, tetrahydrozoline, tetrandrine, tetrantoin, tetrazepam, tetrofosmin, tetroxoprim, Tevenel®, tezacitabine, tezosentan, thalidomide, thenaldine, thenyldiamine, theobromine, theofibrate, theophylline, thiabendazole, thiacetazone, thiacymserine, thialbarbital, thiamine, thiamiprine, thiamphenicol, thiamylal, thiazesim, thiazinamium, thiazolinobutazone, thiazolsulfone, thibenzazoline, thiemalat, thiethylperazine, thimerfonate, thimerosal, thiobarbital, thiobutabarbital, thiocarbamizine, thiocarbarsone, thiocolchicine, thiocresol, thioctic acid, thioglycerol, thioguanine, thioimrag, thiopental, thiophosphoramide, thiopropazate, thioproperazine, thioridazine, thiosulfate, thiothixene, thiovir, thiphenamil, thiram, thonzylamine, thozalinone, thromboplastin, thurfyl nicotinate, thymectacin, thymol, thymopentin, thymyl N-isoamylcarbamate, thyropropic acid, thyroxine, tiadenol, tiagabine, tiamenidine, tianeptine, tiapride, tiaprofenic acid, tiaramide, tiazofurin, tibezonium, tibolone, ticarcillin, ticlopidine, ticrynafen, tiemonium, tigecycline, tigemonam, tigloidine, tilidine, tilisolol, tilmacoxib, tiludronic acid, timentin, timepidium, timiperone, timolol, timonacic, tin ethyl etiopurpurin, tinazoline, tinidazole, tinoridine, tiocarlide, tioclomarol, tioconazole, tiopronin, tiotropium, tioxolone, tipepidine, tipifarnib, tipranavir, tiquizium, tirapazamine, tiratricol, tirilazad, tirofiban, tiropramide, titanium sulfate, tiuxetan, tixocortol, tizanidine, TLK-199, TLK-286, TNF-β analogue, TNP-470, TO-186, tobramycin, tocainide, tocamphyl, tocladesine, tocoretinate, todralazine, tofenacin, tofimilast, tofisopam, tolazamid, tolazolin, tolbutamide, tolcapone, tolciclate, tolcyclamide, tolevamer, tolfenamic acid, tolindate, toliprolol, tolmetin, tolnaftate, tolonidine, tolonium, toloxatone, tolperisone, tolpropamine, tolrestat, tolserine, tolterodine, tolvaptan, tolycaine, topiramate, topoisomerase, topotecan, torasemide, torcetapib, torcitabine, toremifene, torsemide, tositumomab, tosulfloxacin, tramadol, tramazoline, trandolapril, tranexamic acid, tranilast, trans-retinoic acid, tranylcypromine, trapidil, trastuzumab, travoprost, traxanox, traxoprodil, trazodone, tremacamra, trenbolone, trengestone, treosulfan, trepibutone, treprostinol, tretinoin, tretoquinol, TRH, TRI-50b, triacetin, triamcinolone, triamcinolone, triamcinolone, triamcinolone acetonide, triamterene, triapine, triaziquone, triazolam, tribenoside, tribromophenate, trichlorfon, trichlormethiazide, trichlormethine, trichloroethylene, triclobisonium, triclocarban, triclofenol piperazine, triclofos, triclosan, tricromyl, tridihexethyl iodide, trientine, triethanolamine, triethylenemelamine, trifluoperazine, trifluperidol, triflupromazine, trifluridine, triflusal, triflutate, trihexyphenidyl, trimazosin, trimebutine, trimecaine, trimeprazine, trimetazidine, trimethadione, trimethaphan, trimethobenzamide, trimethoprim, trimetozine, trimetrexate, trimipramine, trimoprostil, triolstane, trioxsalen, tripamide, triparanol, tripelennamine, triprolidine, triptorelin, tritiozine, tritoqualine, TRK-530, TRK-820, troclosene, trofosfamide, troglitazone, troleandomycin, trolnitrate, tromantadine, trometamol, trometamol, tromethamine, tromethamine, tropacine, tropesin, tropicamide, tropine, tropisetron, trospectomycin, trospium, trovafloxacin, troxacitabine, troxerutin, troxipide, trypan red, tryparsamide, tryptophan, TSH, TSN-09, TU-2100, tuaminoheptane, tubercidin, tubocurarine chloride, tulobuterol, TV-3326, TY-11223, TY-12533, TYB-3215, tybamate, tyloxapol, tymazoline, tyramine, tyropanoate, ubenimex, ufenamate, undecylenic acid, unoprostone, UR-8880, uracil mustard, uralyt-U, urapidil, urea, uredepa, urethan, uridine 5′-triphosphate, urinastatin, ursodeoxycholic acid, ursodiol, ushercell, uzarin, vaccine, Diphtheria Vaccine, Polyvalent Vaccine, valacyclovir, valdecoxib, valdetamide, valethamate, valganciclovir, valnoctamide, valomaciclovir, valproate, valproic acid, valpromide, valrocemide, valrubicin, valsartan, valspodar, vardenafil, varespladib, varicella virus, vatanidipine, VEA, vecuronium, velnacrine, venlafaxine, veralipride, verapamil, verteporfin, vesnarinone, vetrabutine, VF-233, VI-0134, vidarabine, vigabatrin, vilazodone, viloxazine, viminol, vinbarbital, vinblastine, vinburnine, vincamine, vinconate, vincristine, vindesine, vinflunine, vinorelbine, vinpocetine, vinyl ether, vinylbital, viquidil, viridin, visnadine, vitamin A, vitamin B12, vitamin C, vitamin D2, vitamin D3, vitamin K5, prenatal vitamins, VLA-4 antagonists, VNP-4010M, voglibose, voriconazole, vorozole, VUF-K-8788, warfarin, WF-10, WMC-79, wound healing matrix, WP-170, xaliproden, xamoterol, xanomeline, xanthinol niacinate, xemilofiban, xenbucin, xibenolol, xibornol, ximelagatran, ximoprofen, xipamide, xorphanol, XR-5118, XR-5944, xylometazoline, xylose, YH-1885, YM-511, YM-598, yohimbine, YT-146, Z-321, Z-335, zafirlukast, zalcitabine, zaldaride, zaleplon, zaltoprofen, zanamivir, zanapezil, zatebradine, ZD-0473, ZD-0947, ZD-6126, ZD-9331, zebularine, zelandopam, zenarestat, ziconotide, zidovudine, zileuton, zimeldine, zinc acetate, zinc acexamate, zinc ibuprofenate, zinc p-phenolsulfonate, zinc salicylate, zinostatin, zinostatin stimalamer, zipeprol, ziprasidone, zofenopril, zofenpril+HCTZ, zoledronic acid, zolimidine, zolmitriptan, zolpidem, zomepirac, zonampanel, zoniporide, zonisamide, zopiclone, zopolrestat, zorubicin, zosuquidar, zotepine, ZP-123, Z-tamoxifen, zuclopenthixol, α1-antitrypsin, α-bisabolol, α-chloralose, α-ethylbenzyl alcohol, α-glucose-1-phosphate, α-phenylbutyramide, α-santonin, α-terpineol, α-tocopherol, β-alethine, β-benzalbutyramide, β-carotene, β-eucaine, β-propiolactone, β-sitosterol, γ-aminobutyric acid, γ-hydroxybutyrate, γ-linolenic acid, δ-aminolevulinic acid, ε-acetamidocaproic, and ε-aminocaproic acid. See also U.S. Pat. No. 7,927,613, which is incorporated herein by reference in its entirety. Other pharmaceutically acceptable coformers include those delineated in the “Generally Regarded as Safe” (“GRAS”) and/or the US FDA “Everything Added to Food in the United States” (“EAFUS”) lists.

In some of these embodiments, at least one of the one or more pharmaceutically acceptable coformers can be a compound having any one of formulas (I), (XVIII)-(XXV), and XXVII, (e.g., formula XXIV or XXV) as described in U.S. Pat. No. 10,292,951 which is incorporated herein by reference in its entirety; or any one of the compounds delineated above. In certain of these embodiments, at least one of the one or more pharmaceutically acceptable coformers can be a niclosamide analogue having any one of formulas (I), (XVIII)-(XXV), and XXVII (e.g., formula XXIV or XXV; or XXVI) as described in U.S. Pat. No. 10,292,951 which is incorporated herein by reference in its entirety; or any one of the compounds specifically delineated above.

In some embodiments, the coformer can be any one or more additional therapeutic agents as described herein.

In some embodiments, the co-former is selected from the group consisting of: a sphingosine 1-phosphate (S1P) receptor modulator; a steroidal anti-inflammatory agent; a non-steroidal anti-inflammatory agent; a receptor-interacting protein kinase 1 (RIPK1) inhibitor; an EP4 modulator; a toll-like receptor (e.g., TLR4, TLR9) modulator; a Janus kinase (JAK) inhibitor; a lanthionine synthetase C-like 2 (LANCL2) modulator; a phosphatidylcholine; an integrin (e.g., a4 Integrin) modulator; a Smad7 modulator; a phosphodiesterase 4 (PDE4) modulator; a tumor progression locus 2 (TPL2) inhibitor; a tyrosine kinase 2 (TYK2) inhibitor; and a TEC kinase inhibitor.

In certain embodiments, the co-former is a sphingosine 1-phosphate (S1P) receptor modulator.

In certain embodiments, the co-former is etrasimod or ozanimod.

In certain embodiments, the co-former is a steroidal anti-inflammatory agent. As a non-limiting example, the co-former can be beclomethasone 17 or budesonide.

In certain embodiments, the co-former is a non-steroidal anti-inflammatory agent such as 5-ASA.

In certain embodiments, the co-former is a receptor-interacting protein kinase 1 (RIPK1) inhibitor such as GSK2982772.

In certain embodiments, the co-former is an EP4 modulator such as KAG-308.

In certain embodiments, the co-former is a toll-like receptor (e.g., TLR4, TLR9) modulator. In certain of these embodiments, the co-former is a TLR4 modulator such as JKB-122. In certain embodiments, the co-former is a TLR9 modulator such as cobitolimod.

In certain embodiments, the co-former is a Janus kinase (JAK) inhibitor. In certain of these embodiments, the co-former is selected from the group consisting of TD-1473, tofacitinib, upadacitinib, filgotinib, PF-06651600, and PF-06700841.

In certain embodiments, the co-former is a lanthionine synthetase C-like 2 (LANCL2) modulator such as BT-11.

In certain embodiments, the co-former is a phosphatidylcholine such as LT-02.

In certain embodiments, the co-former is an integrin modulator. In certain of these embodiments, the co-former is an a4 Integrin modulator such as AJM-300 (carotegrast).

In certain embodiments, the co-former is a Smad7 antisense oligonucleotide such as mongersen.

In certain embodiments, the co-former is a phosphodiesterase 4 (PDE4) modulator such as apremilast.

In certain embodiments, the co-former is a tumor progression locus 2 (TPL2) inhibitor such as GS-4875.

In certain embodiments, the co-former is a tyrosine kinase 2 (TYK2) inhibitor.

In certain of these embodiments, the co-former is BMS-986165, PF-06700841, or PF-06826647.

In certain embodiments, the co-former is a TEC kinase inhibitor such as PF-06651600.

Non-Limiting Combinations

In some embodiments, the cocrystal includes (i) a compound of Formula (I); and (ii) a pharmaceutically acceptable salt of the compound of Formula (I); or a pharmaceutically acceptable salt and/or hydrate of the compound of Formula (I).

In some embodiments, the cocrystal includes (i) a compound of Formula (I); and (ii) a second API.

In some embodiments, the cocrystal includes (i) a pharmaceutically acceptable salt of a compound of Formula (I); and (ii) a second API.

In some embodiments, the cocrystal includes (i) a compound of Formula (I); and (ii) a second API.

In some embodiments, the cocrystal includes (i) a pharmaceutically acceptable salt of a compound of Formula (I); and (ii) an amino acid (e.g., proline, e.g., D-proline, or L-proline, or racemic proline).

In some embodiments, the cocrystal includes (i) a compound of Formula (I); and (ii) an amino acid (e.g., proline, e.g., D-proline, or L-proline, or racemic proline).

In some embodiments, the cocrystal includes (i) a pharmaceutically acceptable salt of a compound of Formula (I); and (ii) a 5-10 (e.g., 5-9, 5-6, or 5) membered heteroaryl, e.g., a nitrogen-containing heteroaryl, e.g., imidazole.

In some embodiments, the cocrystal includes (i) a compound of Formula (I); and (ii) a 5-10 (e.g., 5-9, 5-6, or 5) membered heteroaryl, e.g., a nitrogen-containing heteroaryl, e.g., imidazole.

For examples, see Sanphui, P. Cryst. Growth Des. 2012, 12, 4588; Imramovský, A. Crystals 2012, 2, 349-361; and Grifasi, F. Cryst. Growth Des. 2015, 15, 4588.

The Formula (I) Compound of the Co-Crystal

In some embodiments, the chemical purity of the Formula (I) compound of the co-crystal can be as defined anywhere herein.

Particle Size of the Co-Crystal

In some embodiments, the co-crystal can have a reduced particle size as defined anywhere herein for the Formula (I) compounds.

In some embodiments, co-crystals having reduced particle size can be prepared by jet milling, e.g., using CMTI equipment NGMP-Mill-A, a 2-inch, pancake micronizer manufactured by Sturtevant.

Particle Size Distribution (PSD) can be determined by laser diffraction technique, e.g., using a “MALVERN MASTERSIZER 2000” (standard range between 0.020 and 2000.0 microns), model “APA 2000”, equipped with “Hydro 2000 sm” as dispersing unit.

In some embodiments, the co-crystal has a reduced particle size range.

In some embodiments, co-crystal has a particle size range of from about 0.1 μm to about 30 μm. In certain embodiments, the co-crystal has a particle size range of from about 0.1 μm to about 20 μm. In certain embodiments, the co-crystal has a particle size range of from about 0.1 μm to about 10 μm.

In some embodiments, the co-crystal has a particle size distribution D(0.9) of from about 1.0 μm to about 15.0 μm. In certain embodiments, the co-crystal has a particle size distribution D(0.9) of from about 1.0 μm to about 10.0 μm. In certain embodiments, the co-crystal has a particle size distribution D(0.9) of from about 6.0 μm to about 8.0 μm. In certain embodiments, the co-crystal has a particle size distribution D(0.9) of from about 2.2 μm to about 3.2 μm.

In some embodiments, the co-crystal has a particle size distribution D(0.1) of from about 0.1 μm to about 1.5 μm. In certain embodiments, the co-crystal has a particle size distribution D(0.1) of from about 0.1 μm to about 1.0 μm. In certain embodiments, the co-crystal has a particle size distribution D(0.1) of from about 0.3 μm to about 0.9 μm.

In some embodiments, the co-crystal has a particle size distribution D(0.5) of from about 0.5 μm to about 6.0 μm. In certain embodiments, the co-crystal has a particle size distribution D(0.5) of from about 1.0 μm to about 4.0 μm. In certain embodiments, the co-crystal has a particle size distribution D(0.5) of from about 1.0 μm to about 2.0 μm. In certain embodiments, the co-crystal has a particle size distribution D(0.5) of from about 2.5 μm to about 3.5 μm.

In some embodiments, the co-crystal has a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

In some embodiments, the co-crystal has a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

In some embodiments, the co-crystal has a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

In some embodiments, the compound of Formula (I) has a chemical purity of greater than about 99.0%; and the co-crystal has a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

In some embodiments, the compound of Formula (I) has a chemical purity of greater than about 99.0%; and the co-crystal has a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

In some embodiments, the compound of Formula (I) has a chemical purity of greater than about 99.0%; and the co-crystal has a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

In some embodiments, the compound of Formula (I) has a chemical purity of greater than about 99.0%; and the co-crystal has a particle size range of from about 0.1 μm to about 30 μm, a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

In some embodiments, the compound of Formula (I) has a chemical purity of greater than about 99.0%; and the co-crystal has a particle size range of from about 0.1 μm to about 30 μm, a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

In some embodiments, the compound of Formula (I) has a chemical purity of greater than about 99.0%; and the co-crystal has a particle size range of from about 0.1 μm to about 30 μm, a particle size distribution D(0.9) of from about 2.2 μm to about 3.2 μm, a particle size distribution D(0.5) of from about 1.0 μm to about 4.0 μm, and a particle size distribution D(0.1) of from about 0.3 μm to about 0.9 μm.

In certain of the foregoing embodiments, the co-crystal has a particle size distribution D(0.5) of from about 2.5 μm to about 3.5 μm.

In certain other of the foregoing embodiments, the co-crystal has a particle size distribution D(0.5) of from about 1.0 μm to about 2.0 μm.

Pharmaceutical Compositions and Administration

General

A compound of Formula (I), or a pharmaceutically acceptable salt and/or cocrystal thereof is administered to a subject in need thereof by any route which makes the compound bioavailable (e.g., locally bioavailable). In certain embodiments, the route is oral administration. In certain embodiments, the route is rectal administration.

In some embodiments, a compound of Formula (I), or a pharmaceutically acceptable salt and/or cocrystal thereof is administered as a pharmaceutical composition that includes the chemical entity and one or more pharmaceutically acceptable excipients, and optionally one or more other therapeutic agents as described herein.

In some embodiments, the Formula (I) compounds can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

In some embodiments, the Formula (I) compounds described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumor, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Local Administration

In some embodiments, the Formula (I) compounds described herein or a pharmaceutical composition thereof are suitable for local administration, e.g., local administration by way of administering the Formula (I) compounds or composition thereof at a particular treatment site, (e.g., the digestive tract, the gastrointestinal (“GI”) tract, e.g., colon) so as to provide local administration of the chemical entity to the area in need of treatment (e.g., oral cavity; GI tract, e.g., the colon; eye; skin; e.g., respiratory tract (e.g., nasal passage or the lungs) or joint). In certain embodiments, relatively low systemic exposure of the Formula (I) compounds occurs during said local administration. Examples of such compositions include, e.g., compositions for oral administration, administration by inhalation. Examples of such compositions also include e.g., compositions for rectal administration. In some embodiments, the Formula (I) compounds described herein or a pharmaceutical composition thereof are suitable for local administration to the GI tract, e.g., colon, local administration to the respiratory tract, e.g., the upper respiratory tract (e.g., nose or nasal passage) or lower respiratory tract (e.g., lungs). In certain embodiments, upon administration, the local concentration of the Formula (I) compounds in the GI tract is higher (e.g., from about 2 times higher to about 1,000 times higher; from about 2 times higher to about 900 times higher; from about 2 times higher to about 800 times higher; from about 2 times higher to about 700 times higher; from about 2 times higher to about 500 times higher; from about 2 times higher to about 400 times higher; from about 2 times higher to about 300 times higher; from about 2 times higher to about 200 times higher; from about 2 times higher to about 100 times higher; from about 2 times higher to about 50 times higher, from about 5 times higher to about 1,000 times higher; from about 5 times higher to about 900 times higher; from about 5 times higher to about 800 times higher; from about 2 times higher to about 700 times higher; from about 5 times higher to about 500 times higher; from about 5 times higher to about 400 times higher; from about 5 times higher to about 300 times higher; from about 5 times higher to about 200 times higher; from about 5 times higher to about 100 times higher; from about 5 times higher to about 50 times higher; from about 5 times higher to about 25 times higher; from about 5 times higher to about 15 times higher; e.g., about 1,000 times higher, about 900 times higher, about 800 times higher, about 700 times higher, about 600 times higher, about 500 times higher, about 400 times higher, about 300 times higher, about 200 times higher, about 100 times higher, about 50 times higher, about 25 time higher, about 20 times higher, about 15 times higher, about 10 times higher, about 5 times higher) than the concentration of the chemical entity in the plasma compartment. In certain of these embodiments, the chemical entity in the plasma compartment is subject to first pass metabolism.

In some embodiments, the Formula (I) compounds described herein or a pharmaceutical composition thereof are suitable for local administration to one or more specific locations within the digestive or GI tract, e.g., colon, local administration to one or more specific locations within the respiratory tract. For example, at least some of the compound of Formula (I) is present in the upper GI tract (e.g., stomach); is present in the upper respiratory tract (e.g., nose and nasal passages, paranasal sinuses, the pharynx, and the portion of the larynx above the vocal folds (cords) (e.g., nose and nasal passages or at least some of the compound of Formula (I) is present in the lower GI tract (e.g., the large intestine, e.g., the colon, e.g., the ascending colon and/or transverse colon and/or distal colon; or the small bowel); is present in the lower respiratory tract (e.g., portion of the larynx below the vocal folds, trachea, bronchi, and lungs (e.g., lungs). As a further example, at least some of the compound of Formula (I) is present in the ascending colon and/or the transverse colon and/or the distal colon and/or the small bowel and/or the stomach. Methods of said local administration can include, without limitation, oral administration, respiratory administration such as inhalation or intranasal administration, and/or rectal administration.

In one aspect, provided herein is a composition comprising a compound of Formula (I) or co-crystal as described anywhere herein and one or more pharmaceutically acceptable excipients, wherein the composition is suitable for oral administration.

In one aspect, provided herein is a composition comprising a compound of Formula (I) or co-crystal as described anywhere herein and one or more pharmaceutically acceptable excipients, wherein the composition is suitable for local, topical administration.

In certain embodiments, the chemical entities described herein or a pharmaceutical composition thereof are suitable for rectal administration. Rectal compositions include, without limitation, enemas, rectal gels, rectal foams, rectal aerosols, suppositories, jelly suppositories, and enemas (e.g., retention enemas).

Pharmacologically acceptable excipients usable in the rectal composition as a gel, cream, enema, or rectal suppository, include, without limitation, any one or more of cocoa butter glycerides, synthetic polymers such as polyvinylpyrrolidone, PEG (like PEG ointments), glycerine, glycerinated gelatin, hydrogenated vegetable oils, poloxamers, mixtures of polyethylene glycols of various molecular weights and fatty acid esters of polyethylene glycol Vaseline, anhydrous lanolin, shark liver oil, sodium saccharinate, menthol, sweet almond oil, sorbitol, sodium benzoate, anoxid SBN, vanilla essential oil, aerosol, parabens in phenoxyethanol, sodium methyl p-oxybenzoate, sodium propyl p-oxybenzoate, diethylamine, carbomers, carbopol, methyloxybenzoate, macrogol cetostearyl ether, cocoyl caprylocaprate, isopropyl alcohol, propylene glycol, liquid paraffin, xanthan gum, carboxy-metabisulfite, sodium edetate, sodium benzoate, potassium metabisulfite, grapefruit seed extract, methyl sulfonyl methane (MSM), lactic acid, glycine, vitamins, such as vitamin A and E and potassium acetate.

In certain embodiments, suppositories can be prepared by mixing the chemical entities described herein with suitable non-irritating excipients or carriers such as cocoa butter, polyethylene glycol or a suppository wax which are solid at ambient temperature but liquid at body temperature and therefore melt in the rectum and release the active compound. In other embodiments, compositions for rectal administration are in the form of an enema.

In some embodiments, administration of a single dose of the composition to a subject produces a local concentration of the compound of Formula (I) in the GI tract (e.g., colon) of the subject that is higher than the concentration of the compound in the plasma compartment of the subject.

In some embodiments, administration of a single dose of the composition to a subject produces a local concentration of the compound of Formula (I) in the GI tract (e.g., colon) of the subject that is at least about 200 times higher than the concentration of the compound in the plasma compartment of the subject.

In some embodiments, administration of a single dose of the composition to a subject produces a local concentration of the compound of Formula (I) in the GI tract (e.g., colon) of the subject that is at least about 300 times higher than the concentration of the compound in the plasma compartment of the subject.

In some embodiments, administration of a single dose of the composition to a subject produces a local concentration of the compound of Formula (I) in the GI tract (e.g., colon) of the subject that is at least about 500 times higher than the concentration of the compound in the plasma compartment of the subject.

In some embodiments, administration of a single dose of the composition to a subject produces a local concentration of the compound of Formula (I) in the GI tract (e.g., colon) of the subject that is at least about 700 times higher than the concentration of the compound in the plasma compartment of the subject.

In some embodiments, the local concentration of the compound of Formula (I) in the GI tract (e.g., colon) of the subject is higher than a local concentration produced by oral administration of a single dose of a second composition comprising a second compound of Formula (I), wherein the second compound of Formula (I) has a higher particle size than the first compound of Formula (I).

In some embodiments, the local concentration of the compound of Formula (I) in the GI tract (e.g., colon) of the subject is at least about 2 times higher than a local concentration produced by oral administration of a single dose of a second composition comprising a second compound of Formula (I), wherein the second compound of Formula (I) has a higher particle size than the first compound of Formula (I).

In some embodiments, the local concentration of the compound of Formula (I) in the GI tract (e.g., colon) of the subject is at least about 5 times higher than a local concentration produced by oral administration of a single dose of a second composition comprising a second compound of Formula (I), wherein the second compound of Formula (I) has a higher particle size than the first compound of Formula (I).

In some embodiments, the local concentration of the compound of Formula (I) in the GI tract (e.g., colon) of the subject is at least about 10 times higher than a local concentration produced by oral administration of a single dose of a second composition comprising a second compound of Formula (I), wherein the second compound of Formula (I) has a higher particle size than the first compound of Formula (I).

In some embodiments, the local concentration of the compound of Formula (I) in the GI tract (e.g., colon) of the subject is at least about 25 times higher than a local concentration produced by oral administration of a single dose of a second composition comprising a second compound of Formula (I), wherein the second compound of Formula (I) has a higher particle size than the first compound of Formula (I).

In some embodiments, the local concentration of the compound of Formula (I) in the GI tract (e.g., colon) of the subject is at least about 50 times higher than a local concentration produced by oral administration of a single dose of a second composition comprising a second compound of Formula (I), wherein the second compound of Formula (I) has a higher particle size than the first compound of Formula (I).

In some embodiments, the local concentration of the compound of Formula (I) in the GI tract (e.g., colon) of the subject is at least about 100 times higher than a local concentration produced by oral administration of a single dose of a second composition comprising a second compound of Formula (I), wherein the second compound of Formula (I) has a higher particle size than the first compound of Formula (I).

In some embodiments, the second compound of Formula (I) has a particle size distribution D(0.9) of from about 25.0 μm to about 65.0 μm.

In some embodiments, the second compound of Formula (I) has a particle size distribution D(0.1) of from about 4.0 μm to about 10.0 μm.

In another aspect, provided herein is a dosage form (e.g., a unit dosage form) comprising a composition as described anywhere herein, wherein the dosage form is suitable for oral administration.

In another aspect, provided herein is a dosage form (e.g., a unit dosage form) comprising a composition as described anywhere herein, wherein the dosage form is suitable for rectal administration.

In some embodiments, the dosage form further comprises one or more components that chemically and/or structurally predispose the dosage form for delivery of the compound to the ascending colon.

In some embodiments, the dosage form further comprises one or more components that chemically and/or structurally predispose the dosage form for delivery of the compound to the transverse colon.

In some embodiments, the dosage form further comprises one or more components that chemically and/or structurally predispose the dosage form for delivery of the compound to the distal colon.

In some embodiments, the dosage form further comprises one or more components that chemically and/or structurally predispose the dosage form for delivery of the compound to the small bowel.

In one aspect, provided herein is a composition comprising compound of Formula (I) or co-crystal as described anywhere herein and one or more pharmaceutically acceptable excipients, wherein the composition is suitable for respiratory administration (e.g., inhalation).

In some embodiments, administration of a single dose of the composition to a subject produces a local concentration of the compound of Formula (I) in the lower respiratory tract (e.g., lungs) of the subject that is higher than the concentration of the compound in the upper respiratory tract of the subject.

In certain embodiments, upon administration, the local concentration of the compound of Formula (I) in the respiratory tract is higher (e.g., from about 2 times higher to about 1,000 times higher; from about 2 times higher to about 900 times higher; from about 2 times higher to about 800 times higher; from about 2 times higher to about 700 times higher; from about 2 times higher to about 500 times higher; from about 2 times higher to about 400 times higher; from about 2 times higher to about 300 times higher; from about 2 times higher to about 200 times higher; from about 2 times higher to about 100 times higher; from about 2 times higher to about 50 times higher, from about 5 times higher to about 1,000 times higher; from about 5 times higher to about 900 times higher; from about 5 times higher to about 800 times higher; from about 2 times higher to about 700 times higher; from about 5 times higher to about 500 times higher; from about 5 times higher to about 400 times higher; from about 5 times higher to about 300 times higher; from about 5 times higher to about 200 times higher; from about 5 times higher to about 100 times higher; from about 5 times higher to about 50 times higher; from about 5 times higher to about 25 times higher; from about 5 times higher to about 15 times higher; e.g., about 1,000 times higher, about 900 times higher, about 800 times higher, about 700 times higher, about 600 times higher, about 500 times higher, about 400 times higher, about 300 times higher, about 200 times higher, about 100 times higher, about 50 times higher, about 25 time higher, about 20 times higher, about 15 times higher, about 10 times higher, about 5 times higher) than the concentration of the chemical entity in the plasma compartment. In certain of these embodiments, the chemical entity in the plasma compartment is subject to first pass metabolism.

Oral Delivery

In other embodiments, the chemical entities described herein or a pharmaceutical composition thereof are suitable for local delivery to the digestive or GI tract by way of oral administration (e.g., solid or liquid dosage forms.).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

Liquid dosage forms for oral administration include pharmaceutically acceptable emulsions, microemulsions, solutions, suspensions, syrups and elixirs. In addition to the chemical entities described herein, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments, the liquid dosage form is a mouthwash. In certain embodiments, such liquid oral dosage forms are useful for local and topical administration to the digestive or GI tract, e.g., digestive tract, e.g., oral cavity.

Orally administered compound of Formula (I) can be delivered to the digestive tract (e.g., the colon) using one or more delivery systems. Non-limiting examples of delivery systems include: prodrugs (e.g., azo-conjugates, pectin prodrugs, or prodrugs formed by conjugation (e.g., through azo-bond) to one or more carrier molecules such as cyclodextrin, glucuronide, dextran, amino acids (e.g., sodium alginate), and HM4PC); biodegradable delivery systems (e.g., colon-specific biodegradable delivery systems) (e.g., biodegradable delivery systems using guar gum and derivatives thereof (e.g., AcGGM), azo-aromatic polymers); matrix-based systems (e.g., by embedding compound of Formula (I) in polymer matrices such as starch derived polymer matrices (e.g., pH-sensitive and/or biodegradable matrices such as Assam Bora rice starch matrices)); time-released systems (e.g., using pH sensitive polymers); bioadhesive systems (e.g., using polymers such as polycarbophils, polyurethanes, polyethylene oxide, Assam Bora rice starch); multiparticulate systems (e.g., using microspheres (e.g., biodegradable microspheres) such as chitosan microspheres (e.g., coated with Eudragit), guar gum base microspheres, polysaccharide pectins, pectin-4-aminothiophenol (Pec-ATP) conjugates, calcium alginate-carboxymethyl cellulose (CA-CMC), nanoparticles (e.g., with MMT-K10 clay), each of which can be optionally coated with a pH sensitive polymer (e.g., Eudragit)); polysaccharide-based delivery systems (e.g., with xanthan gum, guar gum, pectin (e.g., mixture with an insoluble polymer such as ethyl cellulose), chitosan, HPMC derivatives, chondroitin sulfate, galactomannan, amylose, or combinations of polysaccharides such as combinations of cellulose derivatives (e.g., combinations of non-enteric cellulose esters such as cellulose acetate and enteric cellulose esters such as CAP and HPMCP; e.g., pectin-HPMC, chitosan-HPMC, chitosan-pectin, guar gum-chitosan, and dextran-chitosan)); coatings (e.g., with pH sensitive polymers such as enteric-soluble polymers such as methacrylic-acid based polymers (e.g., Eudragit, Eudragit L, or Eudragit S) or Landolphia owariensis latex (LOL), with acid-soluble polymers such as Eudragit E, with pulsatile coatings, with rupturable film coatings, with permeable or semi-permeable film coatings, and optionally using compression-coating systems wherein the core tablet comprising compound of Formula (I) and one or more polymer coatings is further coated with a coating excipient (e.g., almond-gum matrix); pressure-controlled delivery systems; osmotic controlled delivery systems (e.g., OROS-CT); and pulsincap systems. Additional examples are described in Amido, AAPS PharmSciTech, Vol. 16, No. 4, August 2015, which is incorporated herein by reference in its entirety.

Non-limiting examples of oral dosage forms suitable for the selective delivery to the digestive tract (e.g., to the colon) include: delayed release tablets; timed release capsules; immediate release tablets; immediate release capsules (e.g., soft gelatin immediate release capsules); multi-matrix tablets; extended release tablets; gastro-resistant prolonged-release tablets; oral colon-targeted pellets; oral solutions; and oral powders.

Additional examples are described in Amido, AAPS PharmSciTech, Vol. 16, No. 4, August 2015, which is incorporated herein by reference in its entirety.

Further non-limiting examples of dosage forms suitable for selective delivery to the digestive tract (e.g., to the colon) include those described in U.S. Pat. Nos. 9,192,583; 6,224,910; 5,914,132; 9,237,760; 9,023,368; 6,228,396; 10,588,864; Int. J. Appl. Res. Nat. Prod., 2012, 5, 1-16; Carbohydrate Polymers, 2013, 92, 367-373; and J. Controlled Release, 1996, 38, 75-94, each of which is incorporated herein by reference in its entirety.

Enema Formulations

In some embodiments, enema formulations containing the chemical entities described herein are provided in “ready-to-use” form.

In some embodiments, enema formulations containing the chemical entities described herein are provided in one or more kits or packs. In certain embodiments, the kit or pack includes two or more separately contained/packaged components, e.g. two components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and optionally one or more pharmaceutically acceptable excipients (e.g., together formulated as a solid preparation, e.g., together formulated as a wet granulated solid preparation); and (ii) the second component (e.g., contained in a vial or bottle) includes one or more liquids and optionally one or more other pharmaceutically acceptable excipients together forming a liquid carrier. Prior to use (e.g., immediately prior to use), the contents of (i) and (ii) are combined to form the desired enema formulation, e.g., as a suspension. In other embodiments, each of component (i) and (ii) is provided in its own separate kit or pack.

In some embodiments, each of the one or more liquids is water, or a physiologically acceptable solvent, or a mixture of water and one or more physiologically acceptable solvents. Typical such solvents include, without limitation, glycerol, ethylene glycol, propylene glycol, polyethylene glycol and polypropylene glycol. In certain embodiments, each of the one or more liquids is water. In other embodiments, each of the one or more liquids is an oil, e.g. natural and/or synthetic oils that are commonly used in pharmaceutical preparations.

Further pharmaceutical excipients and carriers that may be used in the pharmaceutical products herein described are listed in various handbooks (e.g. D. E. Bugay and W. P. Findlay (Eds) Pharmaceutical excipients (Marcel Dekker, New York, 1999), E-M Hoepfner, A. Reng and P. C. Schmidt (Eds) Fiedler Encyclopedia of Excipients for Pharmaceuticals, Cosmetics and Related Areas (Edition Cantor, Munich, 2002) and H. P. Fielder (Ed) Lexikon der Hilfsstoffe für Pharmazie, Kosmetik and angrenzende Gebiete (Edition Cantor Aulendorf, 1989)).

In some embodiments, each of the one or more pharmaceutically acceptable excipients can be independently selected from thickeners, viscosity enhancing agents, bulking agents, mucoadhesive agents, penetration enhancers, buffers, preservatives, diluents, binders, lubricants, glidants, disintegrants, fillers, solubilizing agents, pH modifying agents, preservatives, stabilizing agents, anti-oxidants, wetting or emulsifying agents, suspending agents, pigments, colorants, isotonic agents, chelating agents, emulsifiers, and diagnostic agents.

In certain embodiments, each of the one or more pharmaceutically acceptable excipients can be independently selected from thickeners, viscosity enhancing agents, mucoadhesive agents, buffers, preservatives, diluents, binders, lubricants, glidants, disintegrants, and fillers.

In certain embodiments, each of the one or more pharmaceutically acceptable excipients can be independently selected from thickeners, viscosity enhancing agents, bulking agents, mucoadhesive agents, buffers, preservatives, and fillers.

In certain embodiments, each of the one or more pharmaceutically acceptable excipients can be independently selected from diluents, binders, lubricants, glidants, and disintegrants.

Examples of thickeners, viscosity enhancing agents, and mucoadhesive agents include without limitation: gums, e.g. xanthan gum, guar gum, locust bean gum, tragacanth gums, karaya gum, ghatti gum, cholla gum, psyllium seed gum and gum arabic; poly(carboxylic acid-containing) based polymers, such as poly (acrylic, maleic, itaconic, citraconic, hydroxyethyl methacrylic or methacrylic) acid which have strong hydrogen-bonding groups, or derivatives thereof such as salts and esters; cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof, clays such as manomorillonite clays, e.g. Veegun, attapulgite clay; polysaccharides such as dextran, pectin, amylopectin, agar, mannan or polygalactonic acid or starches such as hydroxypropyl starch or carboxymethyl starch; polypeptides such as casein, gluten, gelatin, fibrin glue; chitosan, e.g. lactate or glutamate or carboxymethyl chitin; glycosaminoglycans such as hyaluronic acid; metals or water soluble salts of alginic acid such as sodium alginate or magnesium alginate; schleroglucan; adhesives containing bismuth oxide or aluminium oxide; atherocollagen; polyvinyl polymers such as carboxyvinyl polymers; polyvinylpyrrolidone (povidone); polyvinyl alcohol; polyvinyl acetates, polyvinylmethyl ethers, polyvinyl chlorides, polyvinylidenes, and/or the like; polycarboxylated vinyl polymers such as polyacrylic acid as mentioned above; polysiloxanes; polyethers; polyethylene oxides and glycols; polyalkoxys and polyacrylamides and derivatives and salts thereof. Preferred examples can include cellulose derivatives, such as methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, hydroxypropylmethyl cellulose or cellulose esters or ethers or derivatives or salts thereof (e.g., methyl cellulose); and polyvinyl polymers such as polyvinylpyrrolidone (povidone).

Examples of preservatives include without limitation: benzalkonium chloride, benzoxonium chloride, benzethonium chloride, cetrimide, sepazonium chloride, cetylpyridinium chloride, domiphen bromide (Bradosol®), thiomersal, phenylmercuric nitrate, phenylmercuric acetate, phenylmercuric borate, methylparaben, propylparaben, chlorobutanol, benzyl alcohol, phenyl ethyl alcohol, chlorohexidine, polyhexamethylene biguanide, sodium perborate, imidazolidinyl urea, sorbic acid, Purite®), Polyquart®), and sodium perborate tetrahydrate and the like.

In certain embodiments, the preservative is a paraben, or a pharmaceutically acceptable salt thereof. In some embodiments, the paraben is an alkyl substituted 4-hydroxybenzoate, or a pharmaceutically acceptable salt or ester thereof. In certain embodiments, the alkyl is a C1-C4 alkyl. In certain embodiments, the preservative is methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof.

Examples of buffers include without limitation: phosphate buffer system (sodium dihydrogen phospahate dehydrate, disodium phosphate dodecahydrate, bibasic sodium phosphate, anhydrous monobasic sodium phosphate), bicarbonate buffer system, and bisulfate buffer system.

Examples of disintegrants include, without limitation: carmellose calcium, low substituted hydroxypropyl cellulose (L-HPC), carmellose, croscarmellose sodium, partially pregelatinized starch, dry starch, carboxymethyl starch sodium, crospovidone, polysorbate 80 (polyoxyethylenesorbitan oleate), starch, sodium starch glycolate, hydroxypropyl cellulose pregelatinized starch, clays, cellulose, alginine, gums or cross linked polymers, such as cross-linked PVP (Polyplasdone XL from GAF Chemical Corp). In certain embodiments, the disintegrant is crospovidone.

Examples of glidants and lubricants (aggregation inhibitors) include without limitation: talc, magnesium stearate, calcium stearate, colloidal silica, stearic acid, aqueous silicon dioxide, synthetic magnesium silicate, fine granulated silicon oxide, starch, sodium laurylsulfate, boric acid, magnesium oxide, waxes, hydrogenated oil, polyethylene glycol, sodium benzoate, stearic acid glycerol behenate, polyethylene glycol, and mineral oil. In certain embodiments, the glidant/lubricant is magnesium stearate, talc, and/or colloidal silica; e.g., magnesium stearate and/or talc.

Examples of diluents, also referred to as “fillers” or “bulking agents” include without limitation: dicalcium phosphate dihydrate, calcium sulfate, lactose (e.g., lactose monohydrate), sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar. In certain embodiments, the diluent is lactose (e.g., lactose monohydrate).

Examples of binders include without limitation: starch, pregelatinized starch, gelatin, sugars (including sucrose, glucose, dextrose, lactose and sorbitol), polyethylene glycol, waxes, natural and synthetic gums such as acacia tragacanth, sodium alginate cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (povidone). In certain embodiments, the binder is polyvinylpyrrolidone (povidone).

In some embodiments, enema formulations containing the chemical entities described herein include water and one or more (e.g., all) of the following excipients:
One or more (e.g., one, two, or three) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof,
One or more (e.g., one or two; e.g., two) buffers, such as phosphate buffer system (e.g., sodium dihydrogen phospahate dehydrate, disodium phosphate dodecahydrate);
One or more (e.g., one or two, e.g., two) glidants and/or lubricants, such as magnesium stearate and/or talc;
One or more (e.g., one or two; e.g., one) disintegrants, such as crospovidone; and
One or more (e.g., one or two; e.g., one) diluents, such as lactose (e.g., lactose monohydrate).

In certain of these embodiments, the chemical entity is a compound of Formula (I), or a pharmaceutically acceptable salt and/or hydrate and/or cocrystal thereof.

In certain embodiments, enema formulations containing the chemical entities described herein include water, methyl cellulose, povidone, methylparaben, propylparaben, sodium dihydrogen phospahate dehydrate, disodium phosphate dodecahydrate, crospovidone, lactose monohydrate, magnesium stearate, and talc. In certain of these embodiments, the chemical entity is a compound of Formula (I), or a pharmaceutically acceptable salt and/or hydrate and/or cocrystal thereof.

In certain embodiments, enema formulations containing the chemical entities described herein are provided in one or more kits or packs. In certain embodiments, the kit or pack includes two separately contained/packaged components, which when mixed together, provide the desired formulation (e.g., as a suspension). In certain of these embodiments, the two component system includes a first component and a second component, in which: (i) the first component (e.g., contained in a sachet) includes the chemical entity (as described anywhere herein) and one or more pharmaceutically acceptable excipients (e.g., together formulated as a solid preparation, e.g., together formulated as a wet granulated solid preparation); and (ii) the second component (e.g., contained in a vial or bottle) includes one or more liquids and one or more one or more other pharmaceutically acceptable excipients together forming a liquid carrier. In other embodiments, each of component (i) and (ii) is provided in its own separate kit or pack.

In certain of these embodiments, component (i) includes the chemical entity (e.g., a compound of Formula (I), or a pharmaceutically acceptable salt and/or hydrate and/or cocrystal thereof) and one or more (e.g., all) of the following excipients:
(a) One or more (e.g., one) binders (e.g., a polyvinyl polymer, such as polyvinylpyrrolidone (povidone);
(b) One or more (e.g., one or two, e.g., two) glidants and/or lubricants, such as magnesium stearate and/or talc;
(c) One or more (e.g., one or two; e.g., one) disintegrants, such as crospovidone; and
(d) One or more (e.g., one or two; e.g., one) diluents, such as lactose (e.g., lactose monohydrate).

In certain embodiments, component (i) includes from about 40 weight percent to about 80 weight percent (e.g., from about 50 weight percent to about 70 weight percent, from about 55 weight percent to about 70 weight percent; from about 60 weight percent to about 65 weight percent; e.g., about 62.1 weight percent) of the chemical entity (e.g., a compound of Formula (I), or a pharmaceutically acceptable salt and/or hydrate and/or cocrystal thereof).

In certain embodiments, component (i) includes from about 0.5 weight percent to about 5 weight percent (e.g., from about 1.5 weight percent to about 4.5 weight percent, from about 2 weight percent to about 3.5 weight percent; e.g., about 2.76 weight percent) of the binder (e.g., povidone).

In certain embodiments, component (i) includes from about 0.5 weight percent to about 5 weight percent (e.g., from about 0.5 weight percent to about 3 weight percent, from about 1 weight percent to about 3 weight percent; about 2 weight percent e.g., about 1.9 weight percent) of the disintegrant (e.g., crospovidone).

In certain embodiments, component (i) includes from about 10 weight percent to about 50 weight percent (e.g., from about 20 weight percent to about 40 weight percent, from about 25 weight percent to about 35 weight percent; e.g., about 31.03 weight percent) of the diluent (e.g., lactose, e.g., lactose monohydrate).

In certain embodiments, component (i) includes from about 0.05 weight percent to about 5 weight percent (e.g., from about 0.05 weight percent to about 3 weight percent) of the glidants and/or lubricants.

In certain embodiments (e.g., when component (i) includes one or more lubricants, such as magnesium stearate), component (i) includes from about 0.05 weight percent to about 1 weight percent (e.g., from about 0.05 weight percent to about 1 weight percent; from about 0.1 weight percent to about 1 weight percent; from about 0.1 weight percent to about 0.5 weight percent; e.g., about 0.27 weight percent) of the lubricant (e.g., magnesium stearate).

In certain embodiments (when component (i) includes one or more lubricants, such as talc), component (i) includes from about 0.5 weight percent to about 5 weight percent (e.g., from about 0.5 weight percent to about 3 weight percent, from about 1 weight percent to about 3 weight percent; from about 1.5 weight percent to about 2.5 weight percent; from about 1.8 weight percent to about 2.2 weight percent; about 1.93 weight percent) of the lubricant (e.g., talc).

In certain of these embodiments, each of (a), (b), (c), and (d) above is present.

In certain embodiments, component (i) includes the ingredients and amounts as shown in Table 7.

TABLE 7

| Ingredient | Weight Percent |
|---|---|
| Formula (I) compound | 40 weight percent to about 80 weight |
| | percent (e.g., from about 50 weight percent |
| | to about 70 weight percent, from about 55 |
| | weight percent to about 70 weight percent; |
| | from about 60 weight percent to about 65 |
| | weight percent; e.g., about 62.1 weight |
| | percent) |
| Crospovidone | 0.5 weight percent to about 5 weight |
| (Kollidon CL) | percent (e.g., from about 0.5 weight |
| | percent to about 3 weight percent, from |
| | about 1 weight percent to about 3 weight |
| | percent; about 1.93 weight percent |
| lactose monohydrate | about 10 weight percent to about 50 weight |
| (Pharmatose 200M) | percent (e.g., from about 20 weight percent |
| | to about 40 weight percent, from about 25 |
| | weight percent to about 35 weight percent; |
| | e.g., about 31.03 weight percent |
| Povidone | about 0.5 weight percent to about 5 weight |
| (Kollidon K30) | percent (e.g., from about 1.5 weight |
| | percent to about 4.5 weight percent, from |
| | about 2 weight percent to about 3.5 weight |
| | percent; e.g., about 2.76 weight percent |
| talc | 0.5 weight percent to about 5 weight |
| | percent (e.g., from about 0.5 weight |
| | percent to about 3 weight percent, from |
| | about 1 weight percent to about 3 weight |
| | percent; from about 1.5 weight percent to |
| | about 2.5 weight percent; from about 1.8 |
| | weight percent to about 2.2 weight |
| | percent; e.g., about 1.93 weight percent |
| Magnesium stearate | about 0.05 weight percent to about 1 |
| | weight percent (e.g., from about 0.05 |
| | weight percent to about 1 weight percent; |
| | from about 0.1 weight percent to about 1 |
| | weight percent; from about 0.1 weight |
| | percent to about 0.5 weight percent; e.g., |
| | about 0.27 weight percent |

In certain embodiments, component (i) includes the ingredients and amounts as shown in Table 8.

TABLE 8

| Ingredient | Weight Percent |
|---|---|
| Formula (I) compound | About 62.1 weight percent) |
| Crospovidone (Kollidon CL) | About 1.93 weight percent |
| lactose monohydrate (Pharmatose 200M) | About 31.03 weight percent |
| Povidone (Kollidon K30) | About 2.76 weight percent |
| talc | About 1.93 weight percent |
| Magnesium stearate | About 0.27 weight percent |

In certain embodiments, component (i) is formulated as a wet granulated solid preparation. In certain of these embodiments an internal phase of ingredients (the chemical entity, disintegrant, and diluent) are combined and mixed in a high-shear granulator. A binder (e.g., povidone) is dissolved in water to form a granulating solution. This solution is added to the Inner Phase mixture resulting in the development of granules. While not wishing to be bound by theory, granule development is believed to be facilitated by the interaction of the polymeric binder with the materials of the internal phase. Once the granulation is formed and dried, an external phase (e.g., one or more lubricants—not an intrinsic component of the dried granulation), is added to the dry granulation. It is believed that lubrication of the granulation is important to the flowability of the granulation, in particular for packaging. See, e.g., Example 8.

In certain of the foregoing embodiments, component (ii) includes water and one or more (e.g., all) of the following excipients:
(a′) One or more (e.g., one, two; e.g., two) thickeners, viscosity enhancing agents, binders, and/or mucoadhesive agents (e.g., cellulose or cellulose esters or ethers or derivatives or salts thereof (e.g., methyl cellulose); and polyvinyl polymers such as polyvinylpyrrolidone (povidone);
(b′) One or more (e.g., one or two; e.g., two) preservatives, such as a paraben, e.g., methyl 4-hydroxybenzoate (methylparaben), or a pharmaceutically acceptable salt or ester thereof, propyl 4-hydroxybenzoate (propylparaben), or a pharmaceutically acceptable salt or ester thereof, or a combination thereof, and
(c′) One or more (e.g., one or two; e.g., two) buffers, such as phosphate buffer system (e.g., sodium dihydrogen phosphate dihydrate, disodium phosphate dodecahydrate);

In certain of the foregoing embodiments, component (ii) includes water and one or more (e.g., all) of the following excipients:
(a″) a first thickener, viscosity enhancing agent, binder, and/or mucoadhesive agent (e.g., a cellulose or cellulose ester or ether or derivative or salt thereof (e.g., methyl cellulose));
(a′″) a second thickener, viscosity enhancing agent, binder, and/or mucoadhesive agent (e.g., a polyvinyl polymer, such as polyvinylpyrrolidone (povidone));
(b″) a first preservative, such as a paraben, e.g., propyl 4-hydroxybenzoate (propylparaben), or a pharmaceutically acceptable salt or ester thereof,
(b″) a second preservative, such as a paraben, e.g., methyl 4-hydroxybenzoate (methylparaben), or a pharmaceutically acceptable salt or ester thereof,
(c″) a first buffer, such as phosphate buffer system (e.g., disodium phosphate dodecahydrate);
(c′″) a second buffer, such as phosphate buffer system (e.g., sodium dihydrogen phospahate dehydrate),

In certain embodiments, component (ii) includes from about 0.05 weight percent to about 5 weight percent (e.g., from about 0.05 weight percent to about 3 weight percent, from about 0.1 weight percent to about 3 weight percent; e.g., about 1.4 weight percent) of (a″).

In certain embodiments, component (ii) includes from about 0.05 weight percent to about 5 weight percent (e.g., from about 0.05 weight percent to about 3 weight percent, from about 0.1 weight percent to about 2 weight percent; e.g., about 1.0 weight percent) of (a′″).

In certain embodiments, component (ii) includes from about 0.005 weight percent to about 0.1 weight percent (e.g., from about 0.005 weight percent to about 0.05 weight percent; e.g., about 0.02 weight percent) of (b″).

In certain embodiments, component (ii) includes from about 0.05 weight percent to about 1 weight percent (e.g., from about 0.05 weight percent to about 0.5 weight percent; e.g., about 0.20 weight percent) of (b′″).

In certain embodiments, component (ii) includes from about 0.05 weight percent to about 1 weight percent (e.g., from about 0.05 weight percent to about 0.5 weight percent; e.g., about 0.15 weight percent) of (c″).

In certain embodiments, component (ii) includes from about 0.005 weight percent to about 0.5 weight percent (e.g., from about 0.005 weight percent to about 0.3 weight percent; e.g., about 0.15 weight percent) of(c′″).

In certain of these embodiments, each of (a″)-(c′″) is present.

In certain embodiments, component (ii) includes water (up to 100) and the ingredients and amounts as shown in Table 9.

TABLE 9

| Ingredient | Weight Percent |
|---|---|
| methyl cellulose | 0.05 weight percent to about 5 weight |
| (Methocel A15C premium) | percent (e.g., from about 0.05 weight |
| | percent to about 3 weight percent, from |
| | about 0.1 weight percent to about 3 weight |
| | percent; e.g., about 1.4 weight percent |
| Povidone (Kollidon K30) | 0.05 weight percent to about 5 weight |
| | percent (e.g., from about 0.05 weight |
| | percent to about 3 weight percent, from |
| | about 0.1 weight percent to about 2 weight |
| | percent; e.g., about 1.0 weight percent |
| propyl 4-hydroxybenzoate | about 0.005 weight percent to about 0.1 |
| | weight percent (e.g., from about 0.005 |
| | weight percent to about 0.05 weight |
| | percent; e.g., about 0.02 weight percent) |
| methyl 4-hydroxybenzoate | about 0.05 weight percent to about 1 |
| | weight percent (e.g., from about 0.05 |
| | weight percent to about 0.5 weight percent; |
| | e.g., about 0.20 weight percent) |
| disodium phosphate | about 0.05 weight percent to about 1 |
| dodecahydrate | weight percent (e.g., from about 0.05 |
| | weight percent to about 0.5 weight percent; |
| | e.g., about 0.15 weight percent) |
| sodium dihydrogen | about 0.005 weight percent to about 0.5 |
| phosphate dihydrate | weight percent (e.g., from about 0.005 |
| | weight percent to about 0.3 weight percent; |
| | e.g., about 0.15 weight percent) |

In certain embodiments, component (ii) includes water (up to 100%) and the ingredients and amounts as shown in Table 10.

TABLE 10

| Ingredient | Weight Percent |
|---|---|
| methyl cellulose | about 1.4 weight percent |
| (Methocel A15C premium) | |
| Povidone (Kollidon K30) | about 1.0 weight percent |
| propyl 4-hydroxybenzoate | about 0.02 weight percent |
| methyl 4-hydroxybenzoate | about 0.20 weight percent |
| disodium phosphate dodecahydrate | about 0.15 weight percent |
| sodium dihydrogen phosphate dihydrate | about 0.15 weight percent |

Ready-to-use” enemas are generally be provided in a “single-use” sealed disposable container of plastic or glass. Those formed of a polymeric material preferably have sufficient flexibility for ease of use by an unassisted patient. Typical plastic containers can be made of polyethylene. These containers may comprise a tip for direct introduction into the rectum. Such containers may also comprise a tube between the container and the tip. The tip is preferably provided with a protective shield which is removed before use. Optionally the tip has a lubricant to improve patient compliance.

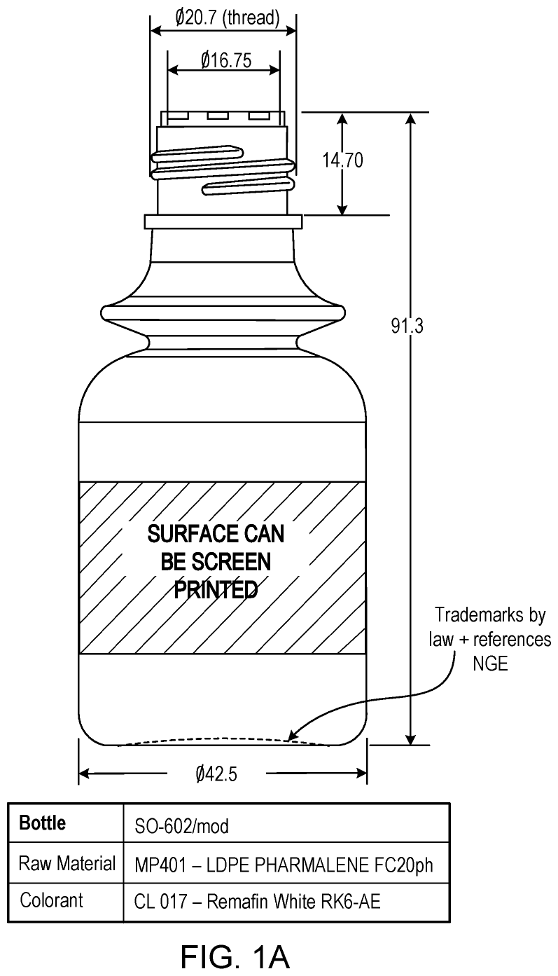

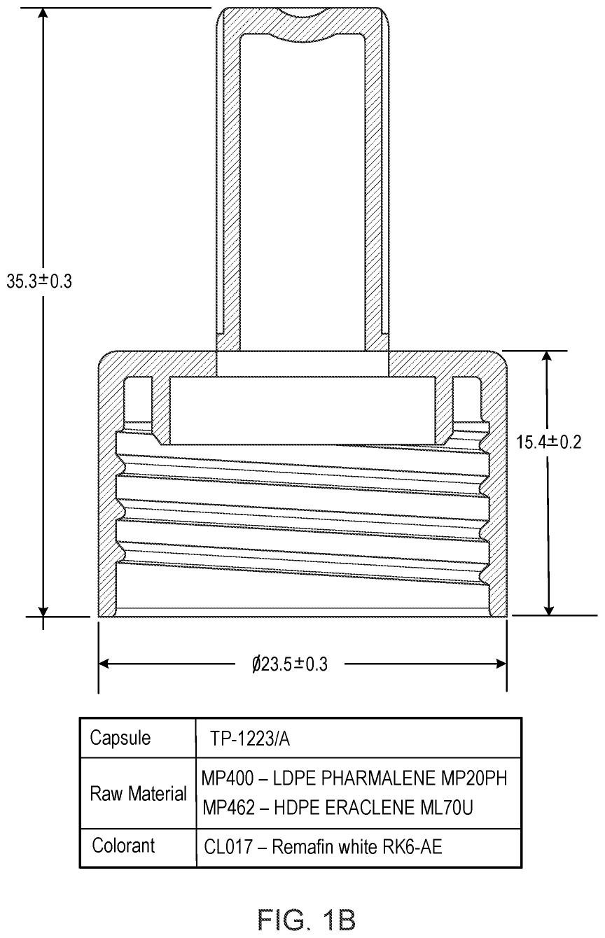

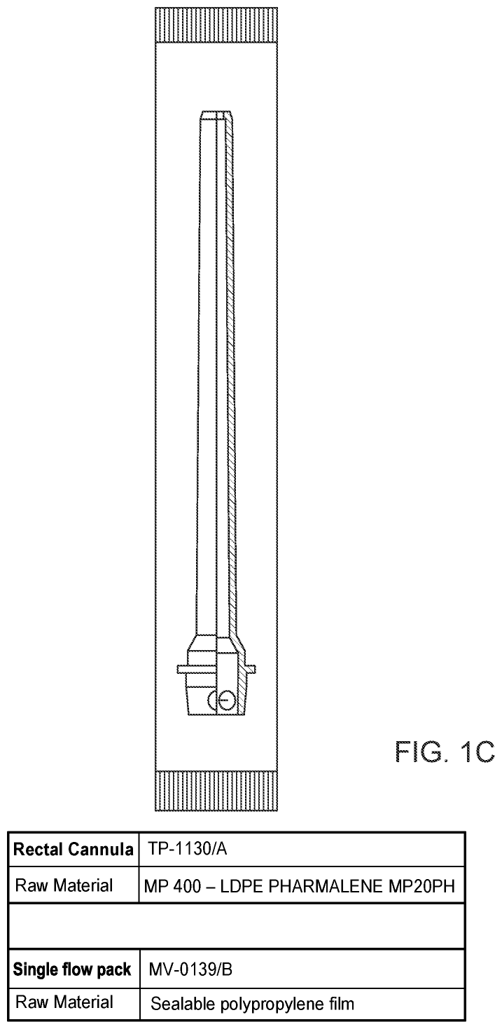

In some embodiments, the enema formulation (e.g., suspension) is poured into a bottle for delivery after it has been prepared in a separate container. In certain embodiments, the bottle is a plastic bottle (e.g., flexible to allow for delivery by squeezing the bottle), which can be a polyethylene bottle (e.g., white in color). In some embodiments, the bottle is a single chamber bottle, which contains the suspension or solution. In other embodiments, the bottle is a multichamber bottle, where each chamber contains a separate mixture or solution. In still other embodiments, the bottle can further include a tip or rectal cannula for direct introduction into the rectum. In some embodiments, the enema formulation can be delivered in the device shown in FIGS. 1A-1C, which includes a plastic bottle, a breakable capsule, and a rectal cannula and single flow pack.

Inhalation and Intranasal Therapy

In some embodiments, compound of Formula (I) or a pharmaceutically acceptable salt and/or cocrystal thereof can be formulated into any suitable dosage form. Non-limiting examples of such dosage forms include aerosols, dispersions (e.g., aqueous oral dispersions, self-emulsifying dispersions, liposomal dispersions, dispersions with colloidal silica or nanospheres such as hydroxypropylmethylcellulose phthalate (HPMCP) nanospheres), pegylated liposomes, liquids, elixirs, suspensions (e.g., nanosuspensions), aerosols, controlled release formulations, lyophilized formulations, powders, delayed release formulations, extended release formulations, multiparticulate formulations, and mixed immediate release formulations. In some embodiments, compound of Formula (I) or a pharmaceutically acceptable salt and/or cocrystal thereof, can be formulated for administration intranasally and/or by inhalation, e.g., using an inhalation device.

An “inhalation device,” as used herein, refers to any device that is capable of administering a drug formulation to the respiratory airways of a subject. Inhalation devices include conventional inhalation devices such as nebulizers, metered dose inhalers (MDIs), dry powder inhalers (DPIs), heat vaporizers, soft mist inhalers, thermal aerosol inhalers, or electrohydrodynamic-based solution misting inhalers. Inhalation devices also include nebulizers. “Nebulizer,” as used herein, refers to a device that turns medications, compositions, formulations, suspensions, and mixtures, etc. into a fine aerosol mist for delivery to the lungs. Non-limiting examples of nebulizers include jet nebulizers, mesh nebulizers, and ultrasonic wave nebulizers. Nebulizers, metered dose inhalers, and soft mist inhalers deliver pharmaceuticals by forming an aerosol which includes droplet sizes that can easily be inhaled. The aerosol can be used by a subject within the bounds of an inhalation therapy, whereby the compound of Formula (I) or a pharmaceutically-acceptable salt and/or cocrystal thereof reaches the subject's respiratory tract upon inhalation. In some embodiments, the methods disclosed herein comprise administering to a subject a nominal dose of compound of Formula (I) or a pharmaceutically-acceptable salt and/or cocrystal thereof by an inhalation device, such as a nebulizer.

In some embodiments of the methods disclosed herein, administration of a composition comprising compound of Formula (I) or a pharmaceutically acceptable salt and/or cocrystal thereof, to a subject with an inhalation device, e.g., a nebulizer, a metered dose inhaler, a dry powder inhaler (DPI), a jet nebulizer, an ultrasonic wave nebulizer, a heat vaporizer, a soft mist inhaler, a thermal aerosol inhaler, or an electrohydrodynamic-based solution misting inhaler, is effective for the treatment or prophylaxis of COVID-19 in a subject.

Inhalation devices may be mechanical or electrical, and include, for example, jet nebulizers and ultrasonic nebulizers. Jet nebulizers generally utilize compressors to generate compressed air, which breaks the liquid medication into small breathable droplets, which form an aerosolized (atomized) mist. In some embodiments, when the subject breathes in, a valve at the top opens, which then allows air into the apparatus, thereby increasing the rate of mist generation; when the subject breathes out, the top valve closes, thereby slowing down mist generation while simultaneously permitting the subject to breathe out through the opening of a mouthpiece flap. Some nebulizers may provide the aerosol in a continuous mode (e.g., the eFlow from PARI Pharma Starnberg), by a breath enhanced mode (e.g., the PART LC Plus or Sprint from PARI Starnberg), by breath actuated mode dependent on the breathing pattern of the subject (e.g., the AeroEclipse from Trudell, Canada or the I-Neb from Philips Respironics), or according to given inhalation profile (e.g., the Akita from Activaero, Gmuenden, Germany).

Some conventional inhalation devices are disclosed in U.S. Pat. Nos. 9,566,399, 6,513,727, 6,513,519, 6,176,237, 6,085,741, 6,000,394, 5,957,389, 5,740,966, 5,549,102, 5,461,695, 5,458,136, 5,312,046, 5,309,900, 5,280,784, and 4,496,086, and International Publication Nos. WO 2018/191776 and WO 2018/213834, each of which is hereby incorporated by reference in its entirety. Commercial conventional inhalation devices are available from: PARI (Germany) under the trade names PARI LC Plus®., LC Star®., and PARI-Jet®; A & H Products, Inc. (Tulsa, Okla.) under the trade name AquaTower®; Hudson RCI (Temecula, Calif.) under the trade name AVA-NEB®; Intersurgical, Inc. (Liverpool, N.Y.) under the trade name Cirrus®; Salter Labs (Arvin, Calif.) under the trade name Salter 8900®; Respironics (Murrysville, Pa.) under the trade name Sidestream®; Bunnell (Salt Lake City, Utah) under the trade name Whisper Jet®; Smiths-Medical (Hyth Kent, UK) under the trade name Downdraft®, and DeVilbiss (Somerset, Pa.) under the trade name DeVilbiss®; or Trudell, Canada under the trade name AeroEclipse®.

In some embodiments of the methods disclosed herein, compositions comprising compound of Formula (I) or a pharmaceutically acceptable salt and/or cocrystal thereof are administered with a dry powder inhaler. Compositions administered with dry powder inhalers can include nanoparticles, spray dried materials, engineered porous particles with low mass median diameter but a high geometric diameter, liposomes, stealth (or PEGylated) liposomes, or combinations thereof. In some embodiments, compositions administered by dry powder inhalers administered in the methods disclosed herein comprise nanoparticle clusters that aggregate into micrometer sized particles at neutral or basic pH but dissociate into nanoparticles at the pH encountered in the lung. In some embodiments the nanoparticle clusters comprise fumaryl diketopiperazine. In some embodiments, compositions administered with dry powder inhalers comprise lactose.

In some embodiments, a dry powder inhaler used to administer an inhalation formulation in the methods disclosed herein comprises a pre-metered dose. For example a pre-metered dose inhaler can comprise a capsule pre-filled with a powder (e.g., a Plastiape Monodose inhaler). In some embodiments, a dry powder inhaler used to administer an inhalation formulation in the methods disclosed herein has a device-metered system such as Twisthaler, sold by Schering Plough, which comprises a reservoir to store a powder and a twisting top to dispense each dose. Inhalation formulations for administration with a dry powder inhaler may be prepared by blending compound of Formula (I) or a pharmaceutically acceptable salt and/or cocrystal thereof, with lactose, or spray drying compound of Formula (I) or a pharmaceutically acceptable salt and/or cocrystal thereof, or by pelletizing compound of Formula (I) or a pharmaceutically acceptable salt and/or cocrystal thereof, to form free-flowing spherical agglomerates.

In some embodiments of the methods disclosed herein, compositions comprising compound of Formula (I) or a pharmaceutically acceptable salt and/or cocrystal thereof are administered with a metered dose inhaler. In some embodiments, a composition administered with a metered dose inhaler in the methods disclosed herein comprises one or more of nanoparticles, spray dried materials, engineered porous particles with low mass median diameter but a high geometric diameter, liposomes, and stealth (or PEGylated) liposomes.

In some embodiments of the methods disclosed herein, compositions comprising compound of Formula (I) or a pharmaceutically acceptable salt and/or cocrystal thereof are administered with a thermal aerosol inhaler.

In some embodiments of the methods disclosed herein, compositions comprising compound of Formula (I) or a pharmaceutically acceptable salt and/or cocrystal thereof are administered with an electrohydrodynamic-based solution misting inhaler. In some embodiments, the aerosol in the electrohydrodynamic-based solution-misting inhaler is generated by subjecting a solution of compound of Formula (I) or a pharmaceutically acceptable salt and/or cocrystal thereof, or a liposome or pegylated liposome comprising compound of Formula (I) or a pharmaceutically acceptable salt and/or cocrystal thereof, to electrohydrodynamic forces through electrostatic energy.

Nebulizers are inhalation devices that comprise a micro-perforated membrane through which a liquid solution is converted through electrical or mechanical means into aerosol droplets suitable for inhalation. Nebulizers can deliver a large fraction of a loaded dose to a subject. In some embodiments, the nebulizer also utilizes one or more actively or passively vibrating microperforated membranes. In some embodiments, the nebulizer contains one or more oscillating membranes. In some embodiments, the nebulizer contains a vibrating mesh or plate with multiple apertures and optionally a vibration generator with an aerosol mixing chamber. In some such embodiments, the mixing chamber functions to collect (or stage) the aerosol from the aerosol generator. In some embodiments, an inhalation valve is also used to allow an inflow of ambient air into the mixing chamber during an inhalation phase and is closed to prevent escape of the aerosol from the mixing chamber during an exhalation phase. In some such embodiments, the exhalation valve is arranged at a mouthpiece which is removably mounted at the mixing chamber and through which the subject inhales the aerosol from the mixing chamber. Still yet, in some embodiments, the nebulizer contains a pulsating membrane. In some embodiments, the nebulizer is continuously operating.

In some embodiments, the nebulizer contains a vibrating micro-perforated membrane of tapered nozzles that generates a plume of droplets without the need for compressed gas. In these embodiments, a solution in the micro-perforated membrane nebulizer is in contact with a membrane, the opposite side of which is open to the air. The membrane is perforated by a large number of nozzle orifices of an atomizing head. An aerosol is created when alternating acoustic pressure in the solution is built up in the vicinity of the membrane causing the fluid on the liquid side of the membrane to be emitted through the nozzles as uniformly sized droplets.

Some embodiments of nebulizers use passive nozzle membranes and a separate piezoelectric transducer that stimulates the membrane. In contrast, some nebulizers employ an active nozzle membrane, which use the acoustic pressure in the nebulizer to generate very fine droplets of solution via the high frequency vibration of the nozzle membrane.

Some nebulizers can contain a resonant system. For example, in such nebulizers, the membrane is driven by a frequency for which the amplitude of the vibrational movement at the center of the membrane is particularly large, resulting in a focused acoustic pressure in the vicinity of the nozzle; the resonant frequency may be about 100 kHz. A flexible mounting is used to keep unwanted loss of vibrational energy to the mechanical surroundings of the atomizing head to a minimum. In some embodiments, the vibrating membrane of the nebulizer may be made stainless steel, or of a nickel-palladium alloy by electroforming.

In some embodiments, a nebulizer may be adapted or adaptable to operate in conjunction with a unit dosage form, such as an ampule or vial, which contains a single dose of a composition comprising compound of Formula (I), or a pharmaceutically-acceptable salt and/or cocrystal thereof, for the treatment of COVID-19. The unit dosage form comprises a container that contains an inhalation formulation comprising the compound of Formula (I), or a pharmaceutically-acceptable salt and/or cocrystal thereof. The container is adapted to cooperate with the nebulizer device in such a way as to permit administration of the nominal dose of the inhalation formulation to a subject. In some embodiments, the nebulizer and the unit dosage form are configured so that they are useable together, but not with other devices or dosage forms. In some particular embodiments, the unit dosage form is configured such that it fits into a keyhole-like structure in the nebulizer, but will not operate with other nebulizer devices. In such embodiments, the nebulizer is configured such that it will accept and properly operate with the unit dosage form containing the compound of Formula (I), or a pharmaceutically-acceptable salt and/or cocrystal thereof, but not with other dosage forms.

Commercial high efficiency nebulizers are available from: PARI (Germany) under the trade name eFlow®; Aerogen, Ltd. (Ireland) under the trade names AeroNeb® Go and AeroNeb® Pro, AeroNeb® Solo, and other nebulizers utilizing the OnQ® nebulizer technology; Respironics (Murrysville, Calif.) under the trade names I-Neb®; Omron (Bannockburn, Ill.) under the trade name Micro-Air®; Activaero (Germany) under the trade name Akita®, and AerovectRx (Atlanta, Ga.) under the trade name AerovectRx®.

In some embodiments, a composition comprising compound of Formula (I), or a pharmaceutically acceptable salt thereof, is formulated as an inhalable nanosuspension (see, e.g., Costabile et al. Mol Pharm. 2015 August 3; 12(8):2604-17.) In some embodiments, an inhalable nanosuspension of compound of Formula (I), or a pharmaceutically acceptable salt and/or cocrystal thereof, is administered to a subject using a nebulizer.

In some embodiments, devices for intranasal administration of compound of Formula (I), or a pharmaceutically acceptable salt and/or cocrystal thereof, include one or more features present in any inhalation device described herein. In some embodiments, devices for intranasal administration of compound of Formula (I), or a pharmaceutically acceptable salt and/or cocrystal thereof, are spray devices. Suitable commercially available nasal spray devices include Accuspray™ (Becton Dickinson). In some embodiments, spray devices for intranasal use are devices for which the performance of the device is not dependent upon the pressure applied by the user. These devices are known as pressure threshold devices. Pressure threshold device release liquid from the nozzle only when a threshold pressure is applied. These devices make it easier to achieve a spray with a regular droplet size. Pressure threshold devices suitable for use with the present invention are known in the art and are described for example in WO 91/13281, EP 311863, and EP 516636. Pressure threshold devices are commercially available from Pfeiffer GmbH and are also described in Bommer, R. Pharmaceutical Technology Europe, September 1999.

In some embodiments, the intranasal devices can administer compound of Formula (I), or a pharmaceutically acceptable salt thereof, by means of bi-dose delivery. Bi-dose devices contain two sub-doses of a single dose, one sub-dose for administration to each nostril. Generally, the two sub-doses are present in a single chamber and the construction of the device allows for efficient delivery of a single sub-dose at a time. Alternatively, a monodose device may be used for administering the vaccines according to the invention.

In some embodiments, compound of Formula (I), or a pharmaceutically acceptable salt and/or cocrystal thereof, is formulated as an ointment or gel for intranasal delivery.

In some embodiments, the compositions disclosed herein can include one or pharmaceutical excipients that provide suitable properties for intranasal administration and/or administration by inhalation of compound of Formula (I), or a pharmaceutically acceptable salt and/or cocrystal thereof. See, e.g., Labiris and Dolovich, Br J Clin Pharmacol. 2003 December; 56(6): 600-612. Non-limiting examples of such pharmaceutical excipients can include surfactants, suspending agents, viscosity enhancing agents, wetting agents, and propellants.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional examples of surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans, and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

Non-limiting examples of propellants include chlorofluorocarbons (CFC) and hydrofluoroalkanes (HFAs).

Compositions and formulations of the disclosure may have a surface tension effective for deposition, penetration or retention of the composition primarily in the peripheral lung regions, including the bronchioles and alveoli.

Other Forms of Delivery In some embodiments, the chemical entities described herein or a pharmaceutical composition thereof are suitable for local and topical administration to the eye (e.g., eye drops). Ocular compositions can include, without limitation, one or more of any of the following: viscogens (e.g., Carboxymethylcellulose, Glycerin, Polyvinylpyrrolidone, Polyethylene glycol); Stabilizers (e.g., Pluronic (triblock copolymers), Cyclodextrins); Preservatives (e.g., Benzalkonium chloride, ETDA, SofZia (boric acid, propylene glycol, sorbitol, and zinc chloride; Alcon Laboratories, Inc.), Purite (stabilized oxychloro complex; Allergan, Inc.)).

In some embodiments, the chemical entities described herein or a pharmaceutical composition thereof are suitable for local and topical administration to skin (e.g., ointments and creams). Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. As with other carriers or vehicles, an ointment base should be inert, stable, nonirritating and non-sensitizing.

Dosages

The dosages may be varied depending on the requirement of the patient, the severity of the condition being treating and the particular compound being employed.

Determination of the proper dosage for a particular situation can be determined by one skilled in the medical arts. The total daily dosage may be divided and administered in portions throughout the day or by means providing continuous delivery.

In some embodiments, compound of Formula (I) is administered is administered at a dosage of from about 0.01 mg/Kg to about 200 mg/Kg (e.g., from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg).

In certain embodiments, the compound of Formula (I) is administered at a dosage of from about 15 mg/Kg to about 100 mg/Kg (e.g., from about 15 mg/Kg to about 90 mg/Kg, from about 20 mg/Kg to about 100 mg/Kg; from about 20 mg/Kg to about 90 mg/Kg; from about 20 mg/Kg to about 80 mg/Kg; from about 30 mg/Kg to about 90 mg/Kg; from about 30 mg/Kg to about 80 mg/Kg; from about 35 mg/Kg to about 75 mg/Kg; from about 10 mg/Kg to about 50 mg/Kg; from about 15 mg/Kg to about 45 mg/Kg; e.g., about 35 mg/Kg or about 75 mg/Kg). In other embodiments, the chemical entity is administered at a dosage of from about 0.1 mg/Kg to about 10 mg/Kg (e.g., from about 0.1 mg/Kg to about 5 mg/Kg; from about 1 mg/Kg to about 10 mg/Kg; from about 1 mg/Kg to about 5 mg/Kg).

In some embodiments, formulations include from about 0.5 mg to about 2500 mg (e.g., from about 0.5 mg to about 2000 mg, from about 0.5 mg to about 1000 mg, from about 0.5 mg to about 750 mg, from about 0.5 mg to about 600 mg, from about 0.5 mg to about 500 mg, from about 0.5 mg to about 400 mg, from about 0.5 mg to about 300 mg, from about 0.5 mg to about 200 mg; e.g., from about 5 mg to about 2500 mg, from about 5 mg to about 2000 mg, from about 5 mg to about 1000 mg; from about 5 mg to about 750 mg; from about 5 mg to about 600 mg; from about 5 mg to about 500 mg; from about 5 mg to about 400 mg; from about 5 mg to about 300 mg; from about 5 mg to about 200 mg; e.g., from about 50 mg to about 2000 mg, from about 50 mg to about 1000 mg, from about 50 mg to about 750 mg, from about 50 mg to about 600 mg, from about 50 mg to about 500 mg, from about 50 mg to about 400 mg, from about 50 mg to about 300 mg, from about 50 mg to about 200 mg; e.g., from about 100 mg to about 2500 mg, from about 100 mg to about 2000 mg, from about 100 mg to about 1000 mg, from about 100 mg to about 750 mg, from about 100 mg to about 700 mg, from about 100 mg to about 600 mg, from about 100 mg to about 500 mg, from about 100 mg to about 400 mg, from about 100 mg to about 300 mg, from about 100 mg to about 200 mg; e.g., from about 150 mg to about 2500 mg, from about 150 mg to about 2000 mg, from about 150 mg to about 1000 mg, from about 150 mg to about 750 mg, from about 150 mg to about 700 mg, from about 150 mg to about 600 mg, from about 150 mg to about 500 mg, from about 150 mg to about 400 mg, from about 150 mg to about 300 mg, from about 150 mg to about 200 mg; e.g., from about 150 mg to about 500 mg; e.g., from about 300 mg to about 2500 mg, from about 300 mg to about 2000 mg, from about 300 mg to about 1000 mg, from about 300 mg to about 750 mg, from about 300 mg to about 700 mg, from about 300 mg to about 600 mg; e.g., from about 400 mg to about 2500 mg, from about 400 mg to about 2000 mg, from about 400 mg to about 1000 mg, from about 400 mg to about 750 mg, from about 400 mg to about 700 mg, from about 400 mg to about 600 from about 400 mg to about 500 mg; e.g., 150 mg or 450 mg) of the compound of Formula (I).

In certain embodiments, formulations include from about 50 mg to about 250 mg (e.g., from about 100 mg to about 200; e.g., about 150 mg) of the compound of Formula (I).

In certain embodiments, enema formulations include from about 350 mg to about 550 mg (e.g., from about 400 mg to about 500; e.g., about 450 mg) of the compound of Formula (I).

The foregoing dosages can be administered on a daily basis (e.g., as a single dose per day; or as two or more divided doses per day; or a two or more doses; e.g., two doses per day) or non-daily basis (e.g., every other day, every two days, every three days, once weekly, twice weeks, once every two weeks, once a month). In certain embodiments, dosages can be administered for about 1 week, about 2 weeks, about 3 weeks, about 4 weeks, about 5 weeks, about 6 weeks, about 7 weeks, about 8 weeks, about 3 months, about 6 months, about 1 year, or beyond. For example, dosages (e.g., about 2.5 mg/mL or about 7.5 mg/mL) of the chemical entity in liquid carrier can be administered twice a day on a daily basis for about 6 weeks. For example, about 2.5 mg/mL or about 7.5 mg/mL of a compound of Formula (I) in liquid carrier can be administered twice a day on a daily basis for about 6 weeks. Representative liquid carriers include, e.g., those previously described in conjunction with component (ii).

Methods of Treatment

In some embodiments, methods for inducing cell death of one or more T cells (e.g., in the digestive and/or gastrointestinal tract (GI), skin, eyes, or joints), of a subject are provided. The methods include contacting the one or more T cells with an effective amount of a compound of Formula (I), or a pharmaceutically acceptable salt and/or cocrystal thereof as defined anywhere herein. In certain embodiments, the methods consist essentially or consist of the contacting step described above in this paragraph.

In some embodiments, methods for treating a subject having a condition associated with unregulated (abnormal, elevated) recruitment and/or retention of one or more T cells (e.g., at the digestive and/or gastrointestinal tract (GI), e.g., colon, e.g., skin, eyes, or joints) of the subject are provided. The methods include contacting the one or more T cells with an effective amount of a compound of Formula (I), or a pharmaceutically acceptable salt and/or cocrystal thereof as defined anywhere herein. In certain embodiments, the methods consist essentially of or consist of the contacting step described above in this paragraph.

In some embodiments, methods for treating a subject having a condition associated with unregulated (abnormal, elevated) activation of one or more T cells (e.g., in the digestive and/or gastrointestinal tract (GI), e.g., colon) of the subject are provided. The methods include contacting the one or more activated T cells with an effective amount of a cocrystal comprising (i) a compound of Formula (I), or a pharmaceutically acceptable salt thereof; and (ii) one or more pharmaceutically acceptable coformers as defined anywhere herein. In certain embodiments, the methods consist essentially of or consist of the contacting step described above in this paragraph.

In some embodiments, inducing cell death of the one or more T cells includes one or more of the following pathways: Programmed cell death, Necroptosis, Apoptosis, Necrosis, Pyroptosis, Ferroptosis, Anoikis, Mitotic cathastrophe, Paraptosis, Pyronecrosis, Entosis, Netosis, Parthanatos, Autophagic cell death, RGD: regulated cell death, Non-apoptotic programmed cell-death, Caspase-independent programmed cell-death inducing necrosis or apoptosis of the one or more T cells, e.g., necrosis or apoptosis of the one or more T cells. In certain embodiments, the effective amount is an amount sufficient to induce cell death of at least one of the one or more T cells (e.g., by any one or more of the pathways described above, e.g., necrosis or apoptosis of the one or more T cells).

In some embodiments, the one or more T cells include one or more activated T cells, e.g., one or more activated T cells is independently selected from the group consisting of:

CD45+CD3+TCRαβ+CD62L−;

CD45+CD3+TCRαβ+CD62L−CCR7−;

CD45+CD3+TCRαβ+CD62L−CD69+;

CD45+CD3+TCRαβ+CD62L−CD69+PD-1+;

CD45+CD3+TCRαβ+CD62L−CTLA4+;

CD45+CD3+TCRαβ+CD62L−PD-1++CTLA4+;

CD45+CD3+TCRγδ+CD62L−;

CD45+CD3+TCRγδ+CD62L−CCR7−;

CD45+CD3+TCRγδ+CD62L−CD69+;

CD45+CD3+TCRγδ+CD62L−CD69+PD-1+;

CD45+CD3+CD62L−TCRγδ+CTLA4+; and

CD45+CD3+TCRγδ+CD62L−PD-1++CTLA4+.

In certain embodiments, the effective amount is an amount sufficient to induce cell death of at least one of the one or more activated T cells (e.g., by any one or more of the pathways described above, e.g., necrosis or apoptosis of the one or more activated T cells).

In some embodiments, the one or more T cells are present within the intestinal epithelium and/or within the lamina propria and/or within the Peyer's patches (PP) and/or within the GALT (gut associated lymphoid tissue) and/or within the intestinal mucosa and/or within the intestinal submucosa and/or within the intestinal muscular layer and/or within the intestinal serosa.

In some embodiments, the one or more T cells comprise one or more gut tropic T cells. In certain embodiments, each of the one or more gut tropic T cells independently expresses one or more gut-homing receptors selected from the group consisting of:

(CD3+CCR9+;

CD3+α4+ or CD3+β7+;

CD3+α4+β7+;

CD3+β1+;

CD3+α4+β1+;

CD3+LFA1;

CD3+CCR4+; and

CD3+CCR10+.

In some embodiments, methods for treating a condition (or one or more symptoms thereof) characterized by an abnormal inflammatory response in a subject in need thereof are provided (e.g., an autoimmune disorder, e.g., colitis, e.g., autoimmune colitis, e.g., an inflammatory bowel disease; e.g., Crohn's disease or ulcerative colitis). The methods include administering to the subject an effective amount of a chemical entity (e.g., a compound of Formula (I), or a pharmaceutically acceptable salt and/or cocrystal thereof) as defined anywhere herein. In certain embodiments, the methods consist essentially of or consist of the administering step described above in this paragraph.

In some embodiments, methods for treating a condition (or one or more symptoms thereof) characterized by an abnormal inflammatory response in a subject in need thereof are provided (e.g., an autoimmune disorder, e.g., colitis, e.g., autoimmune colitis, e.g., an inflammatory bowel disease; e.g., Crohn's disease or ulcerative colitis). The methods include topically and locally administering to the subject an effective amount of a chemical entity (e.g., a compound of Formula (I), or a pharmaceutically acceptable salt and/or cocrystal thereof) as defined anywhere herein. In certain embodiments, the methods consist essentially of or consist of the administering step described above in this paragraph.

In certain of these embodiments, the condition is an autoimmune disease. Non-limiting examples of autoimmune diseases include: arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis (e.g., Hashimoto's thyroiditis), dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

In some embodiments, methods for treating colitis, e.g., autoimmune colitis, e.g., an inflammatory bowel disease; e.g., Crohn's disease or ulcerative (or one or more symptoms thereof) in a subject are provided. The methods include administering to the subject an effective amount of a chemical entity (e.g., a compound of Formula (I), or a pharmaceutically acceptable salt and/or cocrystal thereof) as defined anywhere herein. In certain embodiments, the methods consist essentially of or consist of the administering step described above in this paragraph.

In some embodiments, methods for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject are provided. The methods include administering to the subject an effective amount of a chemical entity (e.g., a compound of Formula (I), or a pharmaceutically acceptable salt and/or cocrystal thereof) as defined anywhere herein. In certain embodiments, the methods consist essentially of or consist of the administering step described above in this paragraph.

In some embodiments, the condition is colitis, e.g., autoimmune colitis. For example, the autoimmune colitis can be an inflammatory bowel disease. The inflammatory bowel disease can be Crohn's disease. The inflammatory bowel disease can be ulcerative colitis. The colitis (e.g., autoimmune colitis) can be iatrogenic autoimmune colitis, e.g., colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease). In other embodiments, the iatrogenic autoimmune colitis can result from Clostridium dificile infection, which is among the leading cause of nosocomial diarrhea and colitis in the industrialized world and typically occurs in subjects taking broad spectrum antibiotics. The colitis can be collagenous colitis, lymphocytic colitis, or microscopic colitis.

In certain embodiments, the condition is autoimmune colitis, e.g., an inflammatory bowel disease (e.g., Crohn's disease or ulcerative colitis). In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis.

In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In certain embodiments, the condition is autoimmune colitis.

In certain of these embodiments, the autoimmune colitis is induced by one or more chemotherapeutic agents, e.g., a chemotherapeutic immunomodulator, e.g., an immune checkpoint inhibitor. In certain of these embodiments, the immune checkpoint inhibitor targets an immune checkpoint receptor selected from the group consisting of CTLA-4, PD-1, PD-L1, PD-1—PD-L1, PD-1—PD-L2, interleukin-2 (IL-2), indoleamine 2,3-dioxygenase (IDO), IL-10, transforming growth factor-β (TGFβ), T cell immunoglobulin and mucin 3 (TIM3 or HAVCR2), Galectin 9—TIM3, Phosphatidylserine—TIM3, lymphocyte activation gene 3 protein (LAG3), MHC class II—LAG3, 4-1BB-4-1BB ligand, OX40-OX40 ligand, GITR, GITR ligand—GITR, CD27, CD70-CD27, TNFRSF25, TNFRSF25-TLiA, CD40L, CD40-CD40 ligand, HVEM-LIGHT-LTA, HVEM, HVEM—BTLA, HVEM—CD160, HVEM—LIGHT, HVEM-BTLA-CD160, CD80, CD80—PDL-1, PDL2—CD80, CD244, CD48—CD244, CD244, ICOS, ICOS-ICOS ligand, B7-H3, B7-H4, VISTA, TMIGD2, HHLA2-TMIGD2, Butyrophilins, including BTNL2, Siglec family, TIGIT and PVR family members, KIRs, ILTs and LIRs, NKG2D and NKG2A, MICA and MICB, CD244, CD28, CD86—CD28, CD86—CTLA, CD80—CD28, CD39, CD73 Adenosine-CD39-CD73, CXCR4-CXCL12, Phosphatidylserine, TIM3, Phosphatidylserine—TIM3, SIRPA-CD47, VEGF, Neuropilin, CD160, CD30, and CD155; e.g., CTLA-4 or PD1 or PD-L1). See, e.g., Postow, M. J. Clin. Oncol. 2015, 33, 1.

In certain of these embodiments, the immune checkpoint inhibitor is selected from the group consisting of: Urelumab, PF-05082566, MEDI6469, TRX518, Varlilumab, CP-870893, Pembrolizumab (PD1), Nivolumab (PD1), Atezolizumab (formerly MPDL3280A) (PDL1), MEDI4736 (PD-L1), Avelumab (PD-L1), PDR001 (PD1), BMS-986016, MGA271, Lirilumab, IPH2201, Emactuzumab, INCB024360, Galunisertib, Ulocuplumab, BKT140, Bavituximab, CC-90002, Bevacizumab, and MNRP1685A, and MGA271.

In certain of these embodiments, the immune checkpoint inhibitor targets CTLA-4, e.g., an antibody, e.g., ipilimumab or tremelimumab.

In certain of these embodiments, the immune checkpoint inhibitor targets PD1 or PD-L1, e.g., nivolumab, lambroizumab, or BMS-936559.

In certain embodiments, the condition is mucositis, also known as stomatitits, which can occur as a result of chemotherapy or radiation therapy, either alone or in combination as well as damage caused by exposure to radiation outside of the context of radiation therapy. Chemotherapeutic agents which may induce mucositis when used alone or in combination include, but are not limited to, platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide and doxorubicin. Additional agents include inhibitors of mTOR (mammalian target of rapamycin), including but not limited to rapamycin, everolimus, temsirolimus and deforolimus.

In certain embodiments, the condition is uveitis, which is inflammation of the uvea (e.g., anterior uveitis, e.g., iridocyclitis or iritis; intermediate uveitis (also known as pars planitis); posterior uveitis; or chorioretinitis, e.g., pan-uveitis).

In some embodiments, the condition is cancer. Non-limiting examples of cancer include: multiple myeloma, leukemias (HTLV-1 dependent, erythroleukemia, acute myelogenous leukemia (AML), chronic myelogenous leukemia (CML), and large granular lymphocyte leukemia (LGL), lymphomas (EBV-related/Burkitt's, mycosis fungoides, cutaneous T-cell lymphoma, non-Hodgkins lymphoma (NHL), anaplastic large-cell lymphoma (ALCL), breast cancers, triple-negative breast cancers, head and neck cancers, melanoma, ovarian cancers, lung cancers, pancreatic cancers, prostate cancers, sarcomas, osteosarcoma, Kaposi's sarcoma, Ewing's sarcoma, hepatocellular cancers, glioma, neuroblastoma, astrocytoma, colorectal cancers, Wilm's tumors, renal cancers, bladder cancers, endometrial cancers, cervical cancers, esophageal cancers, cutaneous squamous cell cancers, basal cell cancers, and metastatic cancers.

In some embodiments, the condition is a metabolic disorder. As used herein, the term “metabolic disorder” refers to a disorder, disease, or condition which is caused or characterized by an abnormal metabolism (i.e., the chemical changes in living cells by which energy is provided for vital processes and activities) in a subject. Metabolic disorders include diseases, disorders, or conditions associated with aberrant thermogenesis or aberrant adipose cell (e.g., brown or white adipose cell) content or function. Metabolic disorders can be characterized by a misregulation (e.g., downregulation or upregulation) of PGC-1 activity. Metabolic disorders can detrimentally affect cellular functions such as cellular proliferation, growth, differentiation, or migration, cellular regulation of homeostasis, inter- or intra-cellular communication; tissue function, such as liver function, muscle function, or adipocyte function; systemic responses in an organism, such as hormonal responses (e.g., insulin response). Examples of metabolic disorders include obesity, including insulin resistant obesity, diabetes, hyperphagia, endocrine abnormalities, triglyceride storage disease, Bardet-Biedl syndrome, Lawrence-Moon syndrome, Prader-Labhart-Willi syndrome, anorexia, and cachexia. Non-limiting examples of metabolic disorders include obesity, metabolic syndrome, insulin resistance, dyslipidemia (e.g., diabetic dyslipidemia), hyperlipidemia, hypertension, diabetes (e.g., type 2 diabetes, type 1 diabetes, pediatric diabetes), hyperglycemia, gout, LADA, prandial hyperglycemia, hyperlipoproteinemia, micro-/macroalbuminuria, impaired glucose tolerance (IGT), impaired fasting glucose (IFG), increased hepatic glucose production, hyperinsulinemia, non-alcoholic fatty liver diseases (NAFD), non-alcoholic steatohepatitis (NASH), nephropathy, retinopathy, neuropathy, and diabetic complications (including cardiovascular diseases, cardiovascular disorders, disorders of lipid metabolism, neurodegenerative and psychiatric disorders, dysregulation of intraocular pressure including glaucoma, atherosclerosis, hypertension, coronary heart disease, gallbladder disease, nephropathy, retinopathy, or diabetic ulcers).

In some embodiments, the condition is a cardiovascular disease, such as myocardial infarction and cardiac hypertrophy. Cardiovascular disease may further include coronary heart disease (including heart attack and angina pectoris or chest pain); stroke; congestional heart failure; hypertension (e.g., pulmonary artery hypertension), high blood pressure; heart failure; rheumatic fever/rheumatic heart disease; congenital cardiovascular defects; arrhythmias (disorders of heart rhythm); diseases of the arteries, arterioles, and capillaries (including atherosclerosis and Kawasaki disease); bacterial endocarditis; cardiomyopathy; valvular heart disease; diseases of pulmonary circulation; diseases of veins and lymphatics; and other diseases of the circulatory system. In certain embodiments, one or more of the methods herein result in a beneficial effect on infarct healing, increased angiogenesis, and/or an attenuated hypertrophic response in the heart.

In some embodiments, the condition is a condition associated with microbial infection (e.g., viral, bacterial, fungi, and/or parasite infection). Non-limiting examples include: tuberculosis (e.g. pulmonary tuberculosis, non-pulmonary tuberculosis (such as tuberculosis lymph glands, genito-urinary tuberculosis, tuberculosis of bone and joints, tuberculosis meningitis) and miliary tuberculosis), anthrax, abscesses, acne vulgaris, actinomycosis, asthma, bacilliary dysentry, bacterial conjunctivitis, bacterial keratitis, bacterial vaginosis, botulism, Buruli ulcer, bone and joint infections, bronchitis (acute or chronic), brucellosis, burn wounds, cat scratch fever, cellulitis, chancroid, cholangitis, cholecystitis, cutaneous diphtheria, cystic fibrosis, cystitis, diffuse panbronchiolitis, diphtheria, dental caries, diseases of the upper respiratory tract, eczema, empymea, endocarditis, endometritis, enteric fever, enteritis, epididymitis, epiglottitis, erysipelis, erysipelas, erysipeloid, erythrasma, eye infections, furuncles, gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leprosy, leptospirosis, listeriosis, liver abscesses, Lyme disease, lymphogranuloma venerium, mastitis, mastoiditis, meningitis and infections of the nervous system, mycetoma, nocardiosis (e.g. Madura foot), non-specific urethritis, opthalmia (e.g. opthalmia neonatorum), osteomyelitis, otitis (e.g. otitis extema and otitis media), orchitis, pancreatitis, paronychia, pelveoperitonitis, peritonitis, peritonitis with appendicitis, pharyngitis, phlegmons, pinta, plague, pleural effusion, pneumonia, postoperative wound infections, postoperative gas gangrene, prostatitis, pseudo-membranous colitis, psittacosis, pulmonary emphysema, pyelonephritis, pyoderma (e.g. impetigo), Q fever, rat-bite fever, reticulosis, ricin poisoning, Ritter's disease, salmonellosis, salpingitis, septic arthritis, septic infections, septicameia, sinusitis, skin infections (e.g. skin granulomas, impetigo, folliculitis and furunculosis), syphilis, systemic infections, tonsillitis, toxic shock syndrome, trachoma, tularaemia, typhoid, typhus (e.g. epidemic typhus, murine typhus, scrub typhus and spotted fever), urethritis, wound infections, yaws, aspergillosis, candidiasis (e.g. oropharyngeal candidiasis, vaginal candidiasis or balanitis), cryptococcosis, favus, histoplasmosis, intertrigo, mucormycosis, tinea (e.g. tinea corporis, tinea capitis, tinea cruris, tinea pedis and tinea unguium), onychomycosis, Pityriasis versicolor, ringworm and sporotrichosis; or infections with MSSA, MRSA, Staph. epidermidis, Strept. agalactiae, Strept. pyogenes, Escherichia coli, Klebs. pneumoniae, Klebs. oxytoca, Pr. mirabilis, Pr. rettgeri, Pr. vulgaris, Haemophilis injluenzae, Enterococcus faecalis and Enterococcus faecium. Non-limiting examples also include DNA and RNA viral diseases caused by infection for example, by orthomyxoviruses (influenza viruses types A and B), paramyxoviruses (respiratory syncytial virus (RSV), subacute sclerosing panencephalitis (SSPE) virus) measles and parainfluenza type 3), herpes viruses (HSV-1, HSV-2, HHV-6, HHV-7, HHV-8, Epstein Barr Virus (EBV), cytomegalovirus (HCMV) and varicella zoster virus (VZV)), retroviruses (HIV-1, HIV-2, HTLV-1, HTLV-2), flavi- and pestiviruses (yellow fever virus (YFV), hepatitis C virus (HCV), dengue fever virus, bovine viral diarrhea virus (BVDV), hepa-totrophic viruses (hepatitis A virus (HAV), hepatitis B virus (HBV), HCV, hepatitis D virus (HDV), hepatitis E virus (HEV), hepatitis G virus (HGV), Crimean-Congo hemorrhagic fever virus (CCHF), bunyaviruses (Punta Toro virus, Rift Valley fever virus (RVFV), and sandfly fever Sicilian virus), Hantaan virus, Caraparu virus), human papilloma viruses, encephalitis viruses (La Crosse virus), arena viruses (Junin and Tacaribe virus), reovirus, vesicular stomatitis virus, rhinbviruses, enteroviruses (polio virus, coxsackie viruses, encephalomyocarditis virus (EMC)), Lassa fever virus, togaviruses (Sindbis and Semlike forest viruses), coronaviruses, and poxvir. Examples of coronaviruses include SARS-CoV (causing Severe Acute Respiratory Syndrome (SARS)), MERS-CoV (Middle East Respiratory Syndrome (MERS), also sometimes called camel flu). In late 2019, human infection by the coronavirus SARS-CoV-2 (sometimes also called 2019-nCoV) was first recorded in Wuhan, China and quickly spread throughout the globe. The corresponding outbreak of disease is often called COVID-19 (coronavirus disease 2019, sometimes also called Wuhan coronavirus). Further non-limiting examples include infections by fungi such as Candida, Sporothrix schenkii, Histoplasma, Paracoccidioides, Aspergillus, etc.; or parasites such as Leishmania, malaria, acanthomoeba, cestodes, etc.

In another aspect, this disclosure features methods useful for preventing the progression of COVID-19 in a subject (e.g., methods for reducing the likelihood of a subject's developing COVID-19 as well as methods for reducing or slowing the progression of COVID-19 in a subject, e.g., reducing the likelihood that a subject will experience one or more severe or life-threating COVID-19 symptoms), wherein the method includes administering to the subject an effective amount of a compound of Formula (I) or a pharmaceutically acceptable salt and/or cocrystal thereof.

In a further aspect, provided herein is a method for treating a condition associated with viral infection (e.g., COVID-19-related viral infection). The method includes administering to the subject an effective amount of a compound of Formula (I) or a pharmaceutically acceptable salt and/or cocrystal thereof. In some embodiments, the method includes orally administering the compound of Formula (I) so as to treat the COVID-19-related viral infection.

In another aspect, a method of reducing the risk of developing COVID-19 in a subject at risk thereof is provided. The method includes administering an effective amount (e.g., a prophylactically effective amount) of the compound of Formula (I), or a pharmaceutically acceptable salt and/or cocrystal thereof, to the subject.

In another aspect, a method for treating COVID-19 in a subject in need thereof is provided. The method includes administering an effective amount of the compound of Formula (I), or a pharmaceutically acceptable salt and/or cocrystal thereof, to the subject, such as to the GI tract of the subject. In another aspect, a method for treating one or more digestive symptoms in a subject having COVID-19 is provided. The method includes administering the compound of Formula (I), or a pharmaceutically acceptable salt and/or cocrystal thereof, to the subject.

In another aspect, a method for treating mild COVID-19 in a subject in need thereof is provided. The method includes administering an effective amount of the compound of Formula (I), or a pharmaceutically acceptable salt and/or cocrystal thereof, to the subject. In another aspect, a method for treating severe COVID-19 in a subject in need thereof is provided. The method includes administering an effective amount of the compound of Formula (I), or a pharmaceutically acceptable salt and/or cocrystal thereof, to the subject.

In a further aspect, provided herein is a method for reducing SARS-CoV-2 transmission from a subject. The method includes administering the compound of Formula (I), or a pharmaceutically acceptable salt and/or cocrystal thereof, to a subject having COVID-19. In another aspect, a method for reducing the viral load of SARS-CoV-2 in a subject is provided. The method includes administering the compound of Formula (I), or a pharmaceutically acceptable salt and/or cocrystal thereof, to a subject having COVID-19.

In another aspect, this disclosure features a method of treating a subject having COVID-19, the method comprising: identifying a subject that has: (i) a respiratory rate of <30 breaths per min; (ii) an oxygen saturation at rest of >93%; and (iii) a ratio of partial pressure of arterial oxygen to fractional concentration of oxygen inspired air of >300 mm Hg; and administering to the nasal cavity of the identified subject a treatment that includes a compound of Formula (I), or a pharmaceutically acceptable salt and/or cocrystal thereof.

In certain embodiments, a compound of Formula (I), or a pharmaceutically acceptable salt and/or cocrystal thereof, is formulated for delivery by inhalation.

In another aspect, this disclosure features a method of treating a subject having COVID-19, the method comprising: identifying a subject that has at least one of (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication; and administering to the lungs of the identified subject a treatment that includes a a compound of Formula (I), or a pharmaceutically acceptable salt and/or cocrystal thereof. In certain embodiments, the compound of Formula (I), or a pharmaceutically acceptable salt and/or cocrystal thereof, is formulated for delivery by inhalation.

In another aspect, this disclosure features a method for treating severe COVID-19 in a subject in need thereof, the method comprising administering an effective amount of a a compound of Formula (I), or a pharmaceutically acceptable salt and/or cocrystal thereof, to the GI tract of the subject (e.g., by oral delivery).

In another aspect, this disclosure features a method of treating a subject having COVID-19, the method comprising: (a) identifying a subject having (i) a respiratory rate of <30 breaths per min; (ii) an oxygen saturation at rest of >93%; and (iii) a ratio of partial pressure of arterial oxygen to fractional concentration of oxygen inspired air of >300 mm Hg; (b) administering one or more doses of a a compound of Formula (I), or a pharmaceutically acceptable salt and/or cocrystal thereof, to the nasal cavity of the subject; (c) after (a) and (b), identifying whether the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; and (d) administering one or more doses of a a compound of Formula (I), or a pharmaceutically acceptable salt and/or cocrystal thereof, to the lungs of the subject in which the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication; or (e) administering additional doses of the a compound of Formula (I), or a pharmaceutically acceptable salt and/or cocrystal thereof, to the nasal cavity of the subject in which (i) a respiratory rate of <30 breaths per min; (ii) an oxygen saturation at rest of >93%; and (iii) a ratio of partial pressure of arterial oxygen to fractional concentration of oxygen inspired air of >300 mm Hg.

In another aspect, this disclosure features a method of treating a subject having COVID-19, the method comprising: (a) identifying a subject having (i) a respiratory rate of <30 breaths per min; (ii) an oxygen saturation at rest of >93%; and (iii) a ratio of partial pressure of arterial oxygen to fractional concentration of oxygen inspired air of >300 mm Hg; (b) administering one or more doses of a a compound of Formula (I), or a pharmaceutically acceptable salt and/or cocrystal thereof, to the nasal cavity of the subject; (c) after (a) and (b), identifying whether the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication; and (d) administering one or more doses of a a compound of Formula (I), or a pharmaceutically acceptable salt and/or cocrystal thereof, to the GI tract of the subject in which the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; or (e) administering additional doses of the compound of Formula (I), or a pharmaceutically acceptable salt and/or cocrystal thereof, to the nasal cavity of the subject in which (i) a respiratory rate of <30 breaths per min; (ii) an oxygen saturation at rest of >93%; and (iii) a ratio of partial pressure of arterial oxygen to fractional concentration of oxygen inspired air of >300 mm Hg.

In another aspect, this disclosure features a method of treating a subject having COVID-19, the method comprising: (a) identifying a subject having (i) a respiratory rate of <30 breaths per min; (ii) an oxygen saturation at rest of >93%; and (iii) a ratio of partial pressure of arterial oxygen to fractional concentration of oxygen inspired air of >300 mm Hg; (b) administering one or more doses of a a compound of Formula (I), or a pharmaceutically acceptable salt and/or cocrystal thereof, to the nasal cavity of the subject; (c) after (a) and (b), identifying whether the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication; and (d) administering one or more doses of a a compound of Formula (I), or a pharmaceutically acceptable salt and/or cocrystal thereof, to the GI tract of the subject and administering one or more doses of a a compound of Formula (I), or a pharmaceutically acceptable salt and/or cocrystal thereof, to the lungs of the subject in which the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; or (e) administering additional doses of the a compound of Formula (I), or a pharmaceutically acceptable salt and/or cocrystal thereof, to the nasal cavity of the subject in which (i) a respiratory rate of <30 breaths per min; (ii) an oxygen saturation at rest of >93%; and (iii) a ratio of partial pressure of arterial oxygen to fractional concentration of oxygen inspired air of >300 mm Hg.

In another aspect, this disclosure features a method of treating a subject having COVID-19, the method comprising: (a) identifying a subject having at least one of (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication; (b) administering one or more doses of a a compound of Formula (I), or a pharmaceutically acceptable salt and/or cocrystal thereof, to the lungs of the subject; (c) after (a) and (b), identifying whether the subject has (i) a respiratory rate of <30 breaths per min; (ii) an oxygen saturation at rest of >93%; and (iii) a ratio of partial pressure of arterial oxygen to fractional concentration of oxygen inspired air of >300 mm Hg; and (d) administering one or more doses of a a compound of Formula (I), or a pharmaceutically acceptable salt and/or cocrystal thereof, to the nasal cavity of the subject in which the subject has (i) a respiratory rate of <30 breaths per min; (ii) an oxygen saturation at rest of >93%; and (iii) a ratio of partial pressure of arterial oxygen to fractional concentration of oxygen inspired air of >300 mm Hg; or (e) administering additional doses of the a compound of Formula (I), or a pharmaceutically acceptable salt and/or cocrystal thereof, to the lungs of the subject in which i(i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication.

In another aspect, this disclosure features methods that include administering a co-crystal that includes a compound of Formula (I) having any one or more or the properties described herein), or a pharmaceutically acceptable salt thereof and a pharmaceutically acceptable coformer. In some embodiments, the cocrystal has a reduced particle size as described anywhere herein (e.g., the cocrystal itself can be reduced to have the reduced particle size range, and/or the reduced particle size distribution described herein for compound of Formula (I). Accordingly, also provided herein is a method for treating one or more digestive symptoms in a subject having COVID-19, the method comprising administering an effective amount of a compound of Formula (I), or a pharmaceutically acceptable salt and/or cocrystal thereof, to the subject.

In certain embodiments, the subject suffers from one or more preexisting medical conditions selected from the group consisting of lung disease, cardiovascular disease, cancer, hypertension, and an endocrine disease.

In certain embodiments, the subject suffers from, or is predisposed to suffer from, colitis.

In certain embodiments, the colitis is an autoimmune colitis. In certain embodiments, the colitis is an inflammatory bowel disease. In certain embodiments, the colitis is ulcerative colitis or Crohn's disease. In certain embodiments, the colitis is iatrogenic autoimmune colitis. In certain embodiments, the colitis is selected from the group consisting of colitis induced by treatment with adoptive cell therapy, colitis associated by one or more alloimmune diseases, collagenous colitis, lymphocytic colitis, and microscopic colitis.

In some embodiments, the one or more digestive symptoms are the result of autoimmune colitis. For example, the autoimmune colitis can be the result of inflammation in the GI tract. In some embodiments, the autoimmune colitis is the result of overresponsiveness and/or hyperreactivity of the subject's immune system to SARS-CoV-2. In certain embodiments when the subject exhibits a digestive symptom, the digestive symptom appears from about 2 to about 14 days (e.g., about 2-3 days, about 4-5 days, about 6-7 days, about 8-10 days, or about 11-14 days) after the subject's exposure to coronavirus. The methods disclosed herein can further comprise a step of identifying a subject having COVID-19. Identification of a subject as having COVID-19 can include the detection of RNA from severe acute respiratory syndrome coronavirus 2 (SARS-CoV-2) in a biological sample from the subject. In some embodiments, the biological sample is a respiratory sample. Non-limiting examples of respiratory samples that can be used to detect SARS-CoV-2 include a nasopharyngeal swab sample, an oropharyngeal swab sample, a sputum sample, a bronchoalveolar lavage sample, a nasopharyngeal aspirate, a nasopharyngeal wash, a nasal aspirate, a nasal wash, and a lower respiratory tract aspirate.

In some embodiments, the biological sample is a fecal sample and/or an anal/rectal swab sample. In some embodiments, polymerase chain reaction (PCR) is used to detect RNA from SARS-CoV-2 in a sample from a subject. Non-limiting examples of types of PCR that can be used to identify a subject as having COVID-19 include reverse transcription PCR (RT-PCR), real-time PCR (e.g., quantitative PCR (qPCR)), and real-time RT-PCR (rRT-PCR). In some embodiments, a specific gene from SARS-CoV-2 is detected. For example, the E gene, RNA-dependent RNA polymerase gene (RdRp) gene, ORF1a gene, ORF1b gene, N gene, or a combination thereof can be detected using primers or probes specific to the gene or a portion thereof. In some embodiments, detection of RNA from SARS-CoV-2 can include using a kit comprising, for example, PCR reagents and primers and/or probes for detecting RNA from SARS-CoV-2 (e.g., primers and/or probes specific to the E gene, RNA-dependent RNA polymerase gene (RdRp) gene, ORF1a gene, ORF1b gene, N gene, or a combination thereof). Many commercial kits are available to detect RNA from SARS-CoV-2. Non-limiting examples of such kits include PowerChek™ 2019-nCov RT-PCR kit (Kogene Biotech), RT-PCR Allplex 2019-nCoV Assay (Seegene); STANDARD M n-CoV Real-Time Detection Kit (SD Biosensor); rRT-PCR XPERT® Xpress SARS-CoV-2 (Cepheid Innovation); and Primerdesign Ltd COVID-19 GENESIG® Real-Time PCR assay. See also, the kits approved by the Food and Drug Administration (fda.gov/medical-devices/emergency-situations-medical-devices/emergency-use-authorizations#covid19ivd).

In some embodiments, the E gene and RdRp gene specific to SARS-CoV-2 is detected (see, e.g., PowerChek™ 2019-nCov RT-PCR kit; RT-PCR Allplex 2019-nCoV Assay; and STANDARD M n-CoV Real-Time Detection Kit (SD Biosensor)). In some embodiments, the ORF1a gene and N gene are detected (see, e.g., DiaPlexQ™ Novel Coronavirus Detection Kit (2019-nCoV) (SolGent Co., Ltd.)) In some embodiments, the RdRP gene, E gene, and N gene are detected (see, e.g., Corman et al. Eurosurveillance, 25, 2000045 (2020)). In some embodiments, the ORF1 ab genome region is detected (see, e.g., Primerdesign Ltd COVID-19 GENESIG® Real-Time PCR assay). In some embodiments, the N2 gene and E gene are detected (see, e.g., rRT-PCR XPERT® Xpress SARS-CoV-2 (Cepheid Innovation)). In some embodiments, primers and probes to detect the RdRp gene spanning nucleotides 12621-12727 and 14010-14116 (positions according SARS-CoV, NC_004718) can be used to detect SARS-CoV-2. See, e.g. the World Health Organization Protocol from the National Reference Center for Respiratory Viruses, Institut Pasteur, Paris (www.who.int/docs/default-source/coronaviruse/real-time-rt-pcr-assays-for-the-detection-of-sars-cov-2-institut-pasteur-paris.pdf?sfvrsn=3662fcb6_2). In some embodiments, a first gene is detected in a screening test and a second gene is detected for confirmation. For example, the N gene from SARS-CoV-2 can be detected in a screening assay and ORF1b from SARS-CoV-2 can be detected as a confirmatory assay.

In some embodiments, rRT-PCR can be used to monitor a subject with COVID-19. For example, prior to starting a therapy as described herein (e.g., a compound of Formula (I), or a pharmaceutically acceptable salt and/or cocrystal thereof as described herein), a biological sample can be obtained from the subject and the level of SARS-CoV-2 RNA (e.g., the level of RNA corresponding to an SARS-CoV-2 gene described herein) determined in the biological sample. This sample can be considered a base-line sample.

The subject can then be administered one or more doses of a therapy as described herein (e.g., a compound of Formula (I), or a pharmaceutically acceptable salt and/or cocrystal thereof as described herein) and the levels of SARS-CoV-2 RNA can be monitored (e.g., after the first dose, second dose, third dose, etc. or after one week, two weeks, three weeks, four weeks, etc.). If the level of SARS-CoV-2 RNA is lower than the baseline sample (e.g., a 1% to about a 99% reduction, a 1% to about a 95% reduction, a 1% to about a 90% reduction, a 1% to about a 85% reduction, a 1% to about a 80% reduction, a 1% to about a 75% reduction, a 1% reduction to about a 70% reduction, a 1% reduction to about a 65% reduction, a 1% reduction to about a 60% reduction, a 1% reduction to about a 55% reduction, a 1% reduction to about a 50% reduction, a 1% reduction to about a 45% reduction, a 1% reduction to about a 40% reduction, a 1% reduction to about a 35% reduction, a 1% reduction to about a 30% reduction, a 1% reduction to about a 25% reduction, a 1% reduction to about a 20% reduction, a 1% reduction to about a 15% reduction, a 1% reduction to about a 10% reduction, a 1% to about a 5% reduction, about a 5% to about a 99% reduction, about a 10% to about a 99% reduction, about a 15% to about a 99% reduction, about a 20% to about a 99% reduction, about a 25% to about a 99% reduction, about a 30% to about a 99% reduction, about a 35% to about a 99% reduction, about a 40% to about a 99% reduction, about a 45% to about a 99% reduction, about a 50% to about a 99% reduction, about a 55% to about a 99% reduction, about a 60% to about a 99% reduction, about a 65% to about a 99% reduction, about a 70% to about a 99% reduction, about a 75% to about a 95% reduction, about a 80% to about a 99% reduction, about a 90% reduction to about a 99% reduction, about a 95% to about a 99% reduction, about a 5% to about a 10% reduction, about a 5% to about a 25% reduction, about a 10% to about a 30% reduction, about a 20% to about a 40% reduction, about a 25% to about a 50% reduction, about a 35% to about a 55% reduction, about a 40% to about a 60% reduction, about a 50% reduction to about a 75% reduction, about a 60% reduction to about 80% reduction, or about a 65% to about a 85% reduction etc.), this is indicative of responsiveness to therapy. In some embodiments, the level of SARS-CoV-2 RNA in a biological sample obtained from the subject (n) is compared to the sample taken just previous (n−1). If the level of SARS-CoV-2 RNA in the n sample is lower than the n−1 sample (e.g., a 1% to about a 99% reduction, a 1% to about a 95% reduction, a 1% to about a 90% reduction, a 1% to about a 85% reduction, a 1% to about a 80% reduction, a 1% to about a 75% reduction, a 1% reduction to about a 70% reduction, a 1% reduction to about a 65% reduction, a 1% reduction to about a 60% reduction, a 1% reduction to about a 55% reduction, a 1% reduction to about a 50% reduction, a 1% reduction to about a 45% reduction, a 1% reduction to about a 40% reduction, a 1% reduction to about a 35% reduction, a 1% reduction to about a 30% reduction, a 1% reduction to about a 25% reduction, a 1% reduction to about a 20% reduction, a 1% reduction to about a 15% reduction, a 1% reduction to about a 10% reduction, a 1% to about a 5% reduction, about a 5% to about a 99% reduction, about a 10% to about a 99% reduction, about a 15% to about a 99% reduction, about a 20% to about a 99% reduction, about a 25% to about a 99% reduction, about a 30% to about a 99% reduction, about a 35% to about a 99% reduction, about a 40% to about a 99% reduction, about a 45% to about a 99% reduction, about a 50% to about a 99% reduction, about a 55% to about a 99% reduction, about a 60% to about a 99% reduction, about a 65% to about a 99% reduction, about a 70% to about a 99% reduction, about a 75% to about a 95% reduction, about a 80% to about a 99% reduction, about a 90% reduction to about a 99% reduction, about a 95% to about a 99% reduction, about a 5% to about a 10% reduction, about a 5% to about a 25% reduction, about a 10% to about a 30% reduction, about a 20% to about a 40% reduction, about a 25% to about a 50% reduction, about a 35% to about a 55% reduction, about a 40% to about a 60% reduction, about a 50% reduction to about a 75% reduction, about a 60% reduction to about 80% reduction, or about a 65% to about a 85% reduction.

In certain embodiments, the subject is 60 years of age or older. In certain embodiments, the subject suffers from one or more preexisting medical conditions selected from the group consisting of lung disease, cardiovascular disease, and diabetes. In certain embodiments, the subject is unresponsive to treatment with remdesivir.

In another aspect, provided herein is a method of reducing the risk of developing COVID-19 in a subject at risk thereof, the method comprising administering an effective amount (e.g., a prophylactically effective amount) of a compound of Formula (I), or a pharmaceutically acceptable salt and/or cocrystal thereof, to the subject.

In certain embodiments, the subject at risk of developing COVID-19 is a healthcare worker (e.g., emergency room physician or nurse, first responder).

In certain embodiments, the subject at risk of developing COVID-19 is 60 years of age or older.

In certain embodiments, the subject at risk of developing COVID-19 suffers from one or more preexisting medical conditions selected from the group consisting of lung disease, cardiovascular disease, and diabetes.

In certain embodiments, the subject at risk of developing COVID-19 is a resident of an assisted living facility or nursing home, a patient in a hospital for an unrelated treatment (i.e., not related to treatment for COVID-19), or a person incarcerated or working in a prison or jail setting.

In certain embodiments, the subject at risk of developing COVID-19 is unresponsive to treatment with remdesivir.

In certain embodiments, the subject at risk of developing COVID-19 has been exposed to the virus or presumed to have been exposed to the virus.

In certain embodiments, the compound is administered prior to exposure to the virus or prior to presumed exposure to the virus (e.g., prior to contact with one or more individuals having or presumed to have COVID-19 and/or prior to contact with one or more articles contaminated with the virus). For example, the compound can be administered immediately after or shortly after exposure or presumed exposure to the virus.

In another aspect, provided herein is a method for treating COVID-19 in a subject in need thereof, the method comprising orally administering an effective amount of niclosamide:

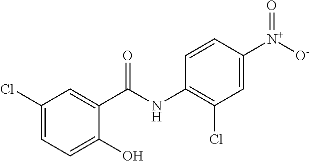 [SMILES: O=C(Nc1ccc([N+](=O)[O-])cc1Cl)c1cc(Cl)ccc1O]

or a pharmaceutically acceptable salt thereof, to the subject.

In certain of these embodiments, the method comprises administering niclosamide.

In certain embodiments, subject exhibits a digestive symptom. In certain embodiments, the subject exhibits a symptom selected from the group consisting of a lack or loss of appetite, diarrhea, vomiting, abdominal pain, a digestive disease, and combinations thereof. In certain embodiments, the subject exhibits a symptom selected from the group consisting of lack or loss of appetite, diarrhea, vomiting, abdominal pain, and combinations thereof. As a non-limiting example of the foregoing embodiments, the subject exhibits.

Also provided herein are methods for treating mild COVID-19 in a subject in need thereof, the method comprising administering an effective amount of a compound of Formula (I), or a pharmaceutically acceptable salt and/or cocrystal thereof, to the subject. In some embodiments, a subject having mild COVID-19 has: (i) a respiratory rate of <30 breaths per min; (ii) an oxygen saturation at rest of >93%; and (iii) a ratio of partial pressure of arterial oxygen to fractional concentration of oxygen inspired air of >300 mm Hg. In some embodiments, a subject having mild COVID-19 does not have a severe disease complication. Severe disease complications can include, but are not limited to, respiratory failure, requirement of mechanical ventilation, septic shock, and non-respiratory organ failure. In some embodiments, a subject having mild COVID-19 does not have digestive symptoms. For example, the subject does not have diarrhea, abdominal pain, or vomiting. In some embodiments, a subject having mild COVID-19 has a low viral load. Viral load can be estimated using the ΔCt method (Ctₛₐₘₚₗₑ−Ct_{ref}). In some embodiments, a sample from a subject with a low viral load has a ΔCt>3. For example, a sample from a subject with a low viral load can have a ΔCt of about 3 to about 15. In some embodiments, the compound of Formula (I), or a pharmaceutically acceptable salt thereof, is administered in combination with one or more additional therapeutic agents (e.g., any of the additional therapeutic agents described herein).

Also provided herein are methods for treating severe COVID-19 in a subject in need thereof, the method comprising administering an effective amount of compound of Formula (I), or a pharmaceutically acceptable salt and/or cocrystal thereof, to the subject. In some embodiments, a subject having severe COVID-19 has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein. In some embodiments, a subject having severe COVID-19 has one or more digestive symptoms. For example, the subject has one or more of diarrhea, abdominal pain, and vomiting. In some embodiments, a subject having severe COVID-19 has one or more of an elevated liver enzyme level, lower monocyte count, and longer prothrombin time. For example, an elevated liver enzyme level can include an AST or ALT level of >50 U/L. In some embodiments, a subject having severe COVID-19 a high viral load. In some embodiments, a sample from a subject with a high viral load has a ΔCt≤2. For example, a sample from a subject with a low viral load can have a ΔCt of about 2 to about −10. In some embodiments, the compound of Formula (I), or a pharmaceutically acceptable salt and/or cocrystal thereof, is administered in combination with one or more additional therapeutic agents (e.g., any of the additional therapeutic agents described herein). Also provided herein are methods for treating mild COVID-19 in a subject in need thereof, the method comprising administering an effective amount of a compound of Foruls (I), or a pharmaceutically acceptable salt and/or cocrystal thereof, to the nasal cavity of the subject. In some embodiments, a subject having mild COVID-19 has: (i) a respiratory rate of ≤30 breaths per min; (ii) an oxygen saturation at rest of 93%; and (iii) a ratio of partial pressure of arterial oxygen to fractional concentration of oxygen inspired air of ≥300 mm Hg. In some embodiments, the subject does not have a severe disease complication. In some embodiments, the subject having mild COVID-19 has a low viral load (e.g., a ΔCt of about 3 to about 15). In some embodiments, the compound of Formula (I), or a pharmaceutically acceptable salt and/or cocrystal thereof, is formulated as an intranasal spray, ointment, or gel (e.g., any of the intranasal compositions described herein).

Also provided herein are methods of treating a subject having COVID-19 that include: identifying a subject that has: (i) a respiratory rate of <30 breaths per min; (ii) an oxygen saturation at rest of >93%; and (iii) a ratio of partial pressure of arterial oxygen to fractional concentration of oxygen inspired air of >300 mm Hg; and administering to the nasal cavity of the identified subject a treatment that includes a compound Formula (I), or a pharmaceutically acceptable salt and/or cocrystal thereof. In some embodiments, the subject does not have a severe disease complication. In some embodiments, the subject has a low viral load (e.g., a ΔCt of about 3 to about 15). In some embodiments, the compound Formula (I), or a pharmaceutically acceptable salt and/or cocrystal thereof, is formulated as an intranasal spray, ointment, or gel (e.g., any of the intranasal compositions described herein).

Also provided herein are methods for treating severe COVID-19 in a subject in need thereof, the method comprising administering an effective amount of a compound Formula (I), or a pharmaceutically acceptable salt and/or cocrystal thereof, to the lungs of the subject. In some embodiments, a subject having severe COVID-19 has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein. In some embodiments, the subject has one or more digestive symptoms (e.g., any of the digestive symptoms described herein). In some embodiments, the subject has one or more of an elevated liver enzyme level, lower monocyte count, and a longer prothrombin time. In some embodiments, the subject has a high viral load (e.g., a ΔCt of about 2 to about −10). In some embodiments, the compound of Formula (I), or a pharmaceutically acceptable salt and/or cocrystal thereof, is formulated for delivery by inhalation (e.g., any of the compositions for inhalation described herein).

Also provided herein are methods of treating a subject having COVID-19 that include: identifying a subject that has at least one of(i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; and administering to the lungs of the identified subject a treatment that includes a compound Formula (I), or a pharmaceutically acceptable salt and/or cocrystal salt thereof. In some embodiments, the subject has one or more digestive symptoms (e.g., any of the digestive symptoms described herein). In some embodiments, the subject has one or more of an elevated liver enzyme level, lower monocyte count, and a longer prothrombin time. In some embodiments, the subject has a high viral load (e.g., a ΔCt of about 2 to about −10).

In some embodiments, the compound Formula (I), or a pharmaceutically acceptable salt and/or cocrystal thereof, is formulated for delivery by inhalation (e.g., any of the compositions for inhalation described herein).

Also provided herein are methods for treating severe COVID-19 in a subject in need thereof, the method comprising administering an effective amount of a compound Formula (I), or a pharmaceutically acceptable salt and/or cocrystal thereof, to the GI tract of the subject. In some embodiments, a subject having severe COVID-19 has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein. In some embodiments, the subject has one or more digestive symptoms (e.g., any of the digestive symptoms described herein). In some embodiments, the subject has one or more of an elevated liver enzyme level, lower monocyte count, and a longer prothrombin time. In some embodiments, the subject has a high viral load (e.g., a ΔCt of about 2 to about −10). In some embodiments, the compound Formula (I), or a pharmaceutically acceptable salt and/or cocrystal thereof, is formulated for oral delivery.

Also provided herein are methods of treating a subject having COVID-19 that include: (a) identifying a subject having (i) a respiratory rate of <30 breaths per min; (ii) an oxygen saturation at rest of >93%; and (iii) a ratio of partial pressure of arterial oxygen to fractional concentration of oxygen inspired air of >300 mm Hg; and (b) administering one or more doses of a compound Formula (I), or a pharmaceutically acceptable salt and/or cocrystal thereof, to the nasal cavity of the subject; (c) after (a) and (b), identifying whether the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; and (d) administering one or more doses of a compound Formula (I), or a pharmaceutically acceptable salt and/or cocrystal thereof, to the lungs of the subject in which the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; or (e) administering additional doses of the compound Formula (I), or a pharmaceutically acceptable salt and/or cocrystal thereof, to the nasal cavity of the subject in which (i) a respiratory rate of <30 breaths per min; (ii) an oxygen saturation at rest of >93%; and (iii) a ratio of partial pressure of arterial oxygen to fractional concentration of oxygen inspired air of >300 mm Hg. In some embodiments, the subject identified in step (a) does not have a severe disease complication. In some embodiments, wherein the compound Formula (I), or a pharmaceutically acceptable salt and/or cocrystal thereof is administered to the nasal cavity of the subject, compound Formula (I), or a pharmaceutically acceptable salt and/or cocrystal thereof, is formulated as an intranasal spray, ointment, or gel (e.g., any of the intranasal compositions described herein). In some embodiments, wherein the compound Formula (I), or a pharmaceutically acceptable salt and/or cocrystal thereof is administered to the lungs of the subject, the compound Formula (I), or a pharmaceutically acceptable salt and/or cocrystal thereof, is formulated for delivery by inhalation (e.g., any of the compositions for inhalation described herein).

Also provided herein are methods of treating a subject having COVID-19 that include: (a) identifying a subject having (i) a respiratory rate of <30 breaths per min; (ii) an oxygen saturation at rest of >93%; and (iii) a ratio of partial pressure of arterial oxygen to fractional concentration of oxygen inspired air of >300 mm Hg; and (b) administering one or more doses of a compound Formula (I), or a pharmaceutically acceptable salt and/or cocrystal thereof, to the nasal cavity of the subject; (c) after (a) and (b), identifying whether the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; and (d) administering one or more doses of a compound Formula (I), or a pharmaceutically acceptable salt and/or cocrystal thereof, to the GI tract of the subject in which the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; or (e) administering additional doses of the compound Formula (I), or a pharmaceutically acceptable salt and/or cocrystal thereof, to the nasal cavity of the subject in which (i) a respiratory rate of <30 breaths per min; (ii) an oxygen saturation at rest of >93%; and (iii) a ratio of partial pressure of arterial oxygen to fractional concentration of oxygen inspired air of >300 mm Hg. In some embodiments, the subject identified in step (a) does not have a severe disease complication. In some embodiments, wherein the compound Formula (I), or a pharmaceutically acceptable salt and/or cocrystal thereof is administered to the nasal cavity of the subject, the compound Formula (I), or a pharmaceutically acceptable salt and/or cocrystal thereof, is formulated as an intranasal spray, ointment, or gel (e.g., any of the intranasal compositions described herein). In some embodiments, wherein the compound Formula (I), or a pharmaceutically acceptable salt and/or cocrystal thereof is administered to the GI tract of the subject, the compound Formula (I), or a pharmaceutically acceptable salt and/or cocrystal thereof, is formulated as an oral composition (e.g., any of the oral compositions described herein).

Also provided herein are methods of treating a subject having COVID-19 that include: (a) identifying a subject having (i) a respiratory rate of <30 breaths per min; (ii) an oxygen saturation at rest of >93%; and (iii) a ratio of partial pressure of arterial oxygen to fractional concentration of oxygen inspired air of >300 mm Hg; and (b) administering one or more doses of a compound Formula (I), or a pharmaceutically acceptable salt and/or cocrystal thereof, to the nasal cavity of the subject; (c) after (a) and (b), identifying whether the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication; and (d) administering one or more doses of a compound Formula (I), or a pharmaceutically acceptable salt and/or cocrystal thereof, to the GI tract of the subject and administering one or more doses of a compound Formula (I), or a pharmaceutically acceptable salt and/or cocrystal thereof, to the lungs of the subject in which the subject has at least one of: (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein; or (e) administering additional doses of the compound Formula (I), or a pharmaceutically acceptable salt and/or cocrystal thereof, to the nasal cavity of the subject in which (i) a respiratory rate of <30 breaths per min; (ii) an oxygen saturation at rest of >93%; and (iii) a ratio of partial pressure of arterial oxygen to fractional concentration of oxygen inspired air of >300 mm Hg. In some embodiments, the subject identified in step (a) does not have a severe disease complication. In some embodiments, wherein the compound Formula (I), or a pharmaceutically acceptable salt and/or cocrystal thereof is administered to the nasal cavity of the subject, the compound Formula (I), or a pharmaceutically acceptable salt and/or cocrystal thereof, is formulated as an intranasal spray, ointment, or gel (e.g., any of the intranasal compositions described herein). In some embodiments, wherein the compound Formula (I), or a pharmaceutically acceptable salt and/or cocrystal thereof is administered to the GI tract of the subject, the compound Formula (I), or a pharmaceutically acceptable salt and/or cocrystal thereof, is formulated as an oral composition (e.g., any of the oral compositions described herein). In some embodiments, wherein the n compound Formula (I), or a pharmaceutically acceptable salt and/or cocrystal thereof is administered to the lungs of the subject, the compound Formula (I), or a pharmaceutically acceptable salt and/or cocrystal thereof, is formulated for delivery by inhalation (e.g., any of the compositions for inhalation described herein).

Also provided herein are methods of treating a subject having COVID-19 that include: (a) identifying a subject having at least one of (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication; and (b) administering one or more doses of a compound Formula (I), or a pharmaceutically acceptable salt and/or cocrystal thereof, to the lungs of the subject; (c) after (a) and (b), identifying whether the subject has (i) a respiratory rate of <30 breaths per min; (ii) an oxygen saturation at rest of >93%; and (iii) a ratio of partial pressure of arterial oxygen to fractional concentration of oxygen inspired air of >300 mm Hg; and (d) administering one or more doses of a compound Formula (I), or a pharmaceutically acceptable salt and/or cocrystal thereof, to the nasal cavity of the subject in which the subject has (i) a respiratory rate of <30 breaths per min; (ii) an oxygen saturation at rest of >93%; and (iii) a ratio of partial pressure of arterial oxygen to fractional concentration of oxygen inspired air of >300 mm Hg; or (e) administering additional doses of the compound Formula (I), or a pharmaceutically acceptable salt and/or cocrystal thereof, to the lungs of the subject in which the subject has at least one of (i) respiratory distress (i.e., ≥30 breaths per min); (ii) an oxygen saturation at rest of ≤93%; (iii) a ratio of partial pressure of arterial oxygen to fractional concentration of oxygen inspired air of ≤300 mm Hg; and (iv) a severe disease complication, e.g., a severe disease complication as described herein. In some embodiments, the subject identified in step (c) does not have a severe disease complication. In some embodiments, wherein the compound Formula (I), or a pharmaceutically acceptable salt and/or cocrystal thereof is administered to the GI tract of the subject, compound Formula (I), or a pharmaceutically acceptable salt and/or cocrystal thereof, is formulated as an oral composition (e.g., any of the oral compositions described herein). In some embodiments, wherein the compound Formula (I), or a pharmaceutically acceptable salt and/or cocrystal thereof is administered to the lungs of the subject, the compound Formula (I), or a pharmaceutically acceptable salt and/or cocrystal thereof, is formulated for delivery by inhalation (e.g., any of the compositions for inhalation described herein). Also provided herein are methods of treating a subject having COVID-19 that include: (a) identifying a subject having a low viral load (e.g., a ΔCt of about 3 to about 15); and (b) administering one or more doses of a compound Formula (I), or a pharmaceutically acceptable salt and/or cocrystal thereof, to the nasal cavity of the subject; (c) after (a) and (b), identifying whether the subject has a high viral load (e.g., a ΔCt of about 2 to about −10); and (d) administering one or more doses of a compound Formula (I), or a pharmaceutically acceptable salt and/or cocrystal thereof, to the lungs of the subject in which the subject has a high viral load (e.g., a ΔCt of about 2 to about −10); or (e) administering additional doses of the compound Formula (I), or a pharmaceutically acceptable salt and/or cocrystal thereof, to the nasal cavity of the subject in which the subject has a low viral load (e.g., a ΔCt of about 3 to about 15). In some embodiments, the subject identified in step (a) does not have a severe disease complication. In some embodiments, wherein the compound Formula (I), or a pharmaceutically acceptable salt and/or cocrystal thereof is administered to the nasal cavity of the subject, the compound Formula (I), or a pharmaceutically acceptable salt and/or cocrystal thereof, is formulated as an intranasal spray, ointment, or gel (e.g., any of the intranasal compositions described herein). In some embodiments, wherein the compound Formula (I), or a pharmaceutically acceptable salt and/or cocrystal thereof is administered to the lungs of the subject, the compound Formula (I), or a pharmaceutically acceptable salt and/or cocrystal thereof, is formulated for delivery by inhalation (e.g., any of the compositions for inhalation described herein).

Also provided herein are methods of treating a subject having COVID-19 that include: (a) identifying a subject having a low viral load (e.g., a ΔCt of about 3 to about 15); and (b) administering one or more doses of compound Formula (I), or a pharmaceutically acceptable salt and/or cocrystal thereof, to the nasal cavity of the subject; (c) after (a) and (b), identifying whether the subject has a high viral load (e.g., a ΔCt of about 2 to about −10); and (d) administering one or more doses of a compound Formula (I), or a pharmaceutically acceptable salt and/or cocrystal thereof, to the GI tract of the subject in which the subject has a high viral load (e.g., a ΔCt of about 2 to about −10); or (e) administering additional doses of the compound Formula (I), or a pharmaceutically acceptable salt and/or cocrystal thereof, to the nasal cavity of the subject in which the subject has a low viral load (e.g., a ΔCt of about 3 to about 15). In some embodiments, the subject identified in step (a) does not have a severe disease complication. In some embodiments, wherein the compound Formula (I), or a pharmaceutically acceptable salt and/or cocrystal thereof is administered to the nasal cavity of the subject, the compound Formula (I), or a pharmaceutically acceptable salt and/or cocrystal thereof, is formulated as an intranasal spray, ointment, or gel (e.g., any of the intranasal compositions described herein). In some embodiments, wherein the compound Formula (I), or a pharmaceutically acceptable salt and/or cocrystal thereof is administered to the GI tract of the subject, the compound Formula (I), or a pharmaceutically acceptable salt and/or cocrystal thereof, is formulated as an oral composition (e.g., any of the oral compositions described herein).

Also provided herein are methods of treating a subject having COVID-19 that include: (a) identifying a subject having a low viral load (e.g., a ΔCt of about 3 to about 15); and (b) administering one or more doses of a compound Formula (I), or a pharmaceutically acceptable salt and/or cocrystal thereof, to the nasal cavity of the subject; (c) after (a) and (b), identifying whether the subject has a high viral load (e.g., a ΔCt of about 2 to about −10); and (d) administering one or more doses of a compound Formula (I), or a pharmaceutically acceptable salt and/or cocrystal thereof, to the GI tract of the subject and administering one or more doses of a compound Formula (I), or a pharmaceutically acceptable salt and/or cocrystal thereof, to the lungs of the subject in which the subject has a high viral load (e.g., a ΔCt of about 2 to about −10); or (e) administering additional doses of the compound Formula (I), or a pharmaceutically acceptable salt and/or cocrystal thereof, to the nasal cavity of the subject in which the subject has a low viral load (e.g., a ΔCt of about 3 to about 15). In some embodiments, the subject identified in step (a) does not have a severe disease complication. In some embodiments, wherein the compound Formula (I), or a pharmaceutically acceptable salt and/or cocrystal thereof is administered to the nasal cavity of the subject, the compound Formula (I), or a pharmaceutically acceptable salt and/or cocrystal thereof, is formulated as an intranasal spray, ointment, or gel (e.g., any of the intranasal compositions described herein). In some embodiments, wherein the compound Formula (I), or a pharmaceutically acceptable salt and/or cocrystal thereof is administered to the GI tract of the subject, the compound Formula (I), or a pharmaceutically acceptable salt and/or cocrystal thereof, is formulated as an oral composition (e.g., any of the oral compositions described herein). In some embodiments, wherein the compound Formula (I), or a pharmaceutically acceptable salt and/or cocrystal thereof is administered to the lungs of the subject, the compound Formula (I), or a pharmaceutically acceptable salt and/or cocrystal thereof, is formulated for delivery by inhalation (e.g., any of the compositions for inhalation described herein).

Also provided herein are methods of treating a subject having COVID-19 that include: (a) identifying a subject having COVID-19 has a high viral load (e.g., a ΔCt of about 2 to about −10); and (b) administering one or more doses of a compound Formula (I), or a pharmaceutically acceptable salt and/or cocrystal thereof, to the lungs of the subject; (c) after (a) and (b), identifying whether the subject has a low viral load (e.g., a ΔCt of about 3 to about 15); and (d) administering one or more doses of a compound Formula (I), or a pharmaceutically acceptable salt and/or cocrystal thereof, to the nasal cavity of the subject in which the subject has a low viral load (e.g., a ΔCt of about 3 to about 15); or (e) administering additional doses of the compound Formula (I), or a pharmaceutically acceptable salt and/or cocrystal thereof, to the lungs of the subject in which the subject has a high viral load (e.g., a ΔCt of about 2 to about −10). In some embodiments, the subject identified in step (c) does not have a severe disease complication. In some embodiments, wherein the compound Formula (I), or a pharmaceutically acceptable salt and/or cocrystal thereof is administered to the GI tract of the subject, the n compound Formula (I), or a pharmaceutically acceptable salt and/or cocrystal thereof, is formulated as an oral composition (e.g., any of the oral compositions described herein). In some embodiments, wherein the compound Formula (I), or a pharmaceutically acceptable salt and/or cocrystal thereof is administered to the lungs of the subject, the compound Formula (I), or a pharmaceutically acceptable salt and/or cocrystal thereof, is formulated for delivery by inhalation (e.g., any of the compositions for inhalation described herein).

In some embodiments, the condition is an allergic disease. Non-limiting examples include urticaria, food allergy, anaphylactic shock, hypereosinophilic syndrome, asthma, allergic rhinitis, allergic conjunctivitis, and atopic dermatitis.

In some embodiments, the condition is a condition associated with NF-κB activation and/or inflammatory cytokine production. Non-limiting examples include autoimmune diseases such as chronic rheumatism, osteoarthritis, systematic lupus erythematosus, systematic scleroderma, polymyositis, Sjoegren's syndrome, vasculitis syndrome, antiphospholipid syndrome, Still's disease, Behcet's disease, periarteritis nodosa, ulcerative colitis, Crohn's disease, active chronic hepatitis, glomerulonephritis, and chronic nephritis, chronic pancreatitis, gout, atherosclerosis, multiple sclerosis, arteriosclerosis, endothelial hypertrophy, psoriasis, psoriatic arthritis, contact dermatitis, atopic dermatitis, allergic disease such as pollinosis, asthma, bronchitis, interstitial pneumonia, lung disease involving granuloma, chronic obstructive lung disease, chronic pulmonary thromboembolism, inflammatory colitis, insulin resistance, obesity, diabetes and its complications (nephropathy, retinopathy, neurosis, hyperinsulinemia, arteriosclerosis, hypercentiona, peripheral vessel obstruction, etc.) diseases involving abnormal vascular proliferation such as hyperlipemia, retinopathy, and pneumonia, Alzheimer's disease, encephalomyelitis, acute hepatitis, chronic hepatitis, drug induced toxic hepatopathy, alcoholic hepatitis, viral hepatitis, icterus, cirrhosis, hepatic insufficiency, atrial myxoma, Caslemann's syndrome, mesangial nephritis, kidney cancer, lung cancer, liver cancer, breast cancer, uterine cancer, pancreatic cancer, other solid cancer, sarcoma, osteosarcoma, metastatic invasion of cancer, carceration of inflammatory focus, cancerous cachexia, metastasis of cancer, leukemia, such as acute myeloblastic leukemia, multiple myeloma, Lennert's lymphoma, malignant lymphoma, development of carcinostatic resistance of cancer, carciration of foci such as viral hepatitis and cirrhosis, carciration from polyp of colon, brain tumor, nervous tumor, endotoxic shock, sepsis, cytome, galoviral pneumonia, cytomegaloviral retinopathy, adenoviral cold, adenoviral pool fever, adenoviral ophthalmia, conjunctivitis, AIDS, uveitis, diseases or complications provoked by infections of other bacteria, viruses, and mycetes, complications after surgery such as generalized inflammatory symptoms, restenosis after percutaneous tubal coronary artery plastic surgery, reperfusion disorders after vascular occulusion opening such as ischemia reperfusion disorders, organ transplantation rejection and perfusion disorders of heart, liver, kidney, or the like, itch, anorexia, malaise, chronic fatigue syndrome, and the like.

In some embodiments, the condition is a condition mediated by an aquaporin, e.g., diseases or conditions of water imbalance. Non-limiting examples include: edema of the brain or spinal cord (e.g., cerebral edema, e.g. cerebral edema consequent to head trauma), ischemic stroke, glioma, meningitis, acute mountain sickness, epileptic seizures, infections, metabolic disorders, hypoxia (including general systemic hypoxia and hypoxia due to cardiac arrest), water intoxication, hepatic failure, hepatic encephalopathy, diabetic ketoacidosis, abscess, eclampsia, Creutzfeldt-Jakob disease, lupus cerebritis, or invasive central nervous system procedure (e.g., neurosurgery, endovascular clot removal, spinal tap, aneurysm repair, or deep brain stimulation or, e.g., spinal cord edema consequent to spinal cord trauma, e.g., spinal cord compression), or cerebral and/or optical nerve edema consequent to microgravity and/or radiation exposure; retinal edema; hyponatremia excessive fluid retention, e.g., consequent to heart failure (HF), liver cirrhosis, nephrotic disorder, or syndrome of inappropriate anti diuretic hormone secretion (SIADH); epilepsy, retinal ischemia or other diseases of the eye associated with abnormalities in intraocular pressure and/or tissue hydration, myocardial ischemia, myocardial ischemia/reperfusion injury, myocardial infarction, myocardial hypoxia, congestive heart failure, sepsis, or neuromyelitis optical; and migraines.

In some embodiments, the condition is a neurodegenerative or psychiatric disease.

Non-limiting examples of neurodegenerative or psychiatric diseases include: amyotrophic lateral sclerosis, Parkinson's disease, Alzheimer's disease, neuronal death, impaired cognitive function, depression, anxiety, eating disorders, appetite regulation, migraine, epilepsia, and addiction to chemical substances.

In some embodiments, the condition is erectile dysfunction (male or female), myopathy, loss of muscle tissue, muscle wasting, muscle catabolism, osteoporosis, or decreased linear growth. In some embodiments, the condition is systemic sclerosis.

In some embodiments, monotherapy includes administering (e.g., topically and locally) to a subject an effective amount of a chemical entity (e.g., a compound of Formula (I), or a pharmaceutically acceptable salt and/or cocrystal thereof) as defined anywhere herein, but excludes the administration of other therapeutic agents (e.g., the active compounds, e.g., peptides, disclosed in U.S. Pat. No. 8,148,328, which is incorporated herein by reference in its entirety).

In some embodiments, the methods described herein can further include administering a second therapeutic agent or regimen.

In certain embodiments, the second therapeutic agent or regimen is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the second therapeutic agent or regimen is administered to the subject at about the same time as contacting with or administering the chemical entity.

By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the second therapeutic agent or regimen is administered to the subject after contacting with or administering the chemical entity (e.g., about one hour after, or about 6 hours after, or about 12 hours after, or about 24 hours after, or about 48 hours after, or about 1 week after, or about 1 month after).

In certain embodiments, the second therapeutic agent is a chemotherapeutic immunomodulator, e.g., an immune checkpoint inhibitor, which can be as defined anywhere herein. In other embodiments, the second therapeutic agent or regimen is one or more anti-inflammatory agents or immunomodulator acting locally in the GI tract. In other embodiments, the second therapeutic agent or regimen is 5-ASA (and associated delivery systems), anti-SMAD7 antisense, orally formulated anti-TNFs, anti-integrins, sulfasalazine, balsalazide, steroids, azathioprine, and methotrexate. In further embodiments, the second therapeutic agent or regimen is radiation or surgery.

In certain embodiments, the second therapeutic agent is platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide and doxorubicin. Additional agents include inhibitors of mTOR (mammalian target of rapamycin), including but not limited to rapamycin, everolimus, temsirolimus and deforolimus.

Combination Therapy

In some embodiments, the methods and compositions described herein are suitable for use in combination therapy with various other therapeutic regimens (e.g., chemotherapy and/or radiation). In certain embodiments, the chemical entities and methods described herein can be used to treat side effects produced by such therapeutic regimens, e.g., inflammatory bowel diseases induced by chemotherapeutic immunomodulators, e.g., checkpoint inhibitors, which in some cases can be prohibitively severe.

In some embodiments, the methods and compositions described herein are suitable for use in combination therapy with one or more additional therapeutic agents.

In certain embodiments, the one or more additional therapeutic agents is administered to the subject prior to contacting with or administering the chemical entity (e.g., about one hour prior, or about 6 hours prior, or about 12 hours prior, or about 24 hours prior, or about 48 hours prior, or about 1 week prior, or about 1 month prior).

In other embodiments, the one or more additional therapeutic agents is administered to the subject at about the same time as contacting with or administering the chemical entity. By way of example, the second therapeutic agent or regimen and the chemical entity are provided to the subject simultaneously in the same dosage form. As another example, the second therapeutic agent or regimen and the chemical entity are provided to the subject concurrently in separate dosage forms.

In still other embodiments, the one or more additional therapeutic agents is administered to the subject after contacting with or administering the chemical entity (e.g., about one hour after, or about 6 hours after, or about 12 hours after, or about 24 hours after, or about 48 hours after, or about 1 week after, or about 1 month after).

As another example, the one or more therapeutic agents can be: budenoside; epidermal growth factor; corticosteroids; cyclosporine; sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1 monoclonal antibodies; anti-IL-6 monoclonal antibodies (e.g., anti-IL-6 receptor antibodies and anti-IL-6 antibodies); growth factors; elastase inhibitors; pyridinyl-imidazole compounds; TNF antagonists as described herein; IL-4, IL-10, IL-13 and/or TGF.beta. cytokines or agonists thereof (e.g., agonist antibodies); IL-11; glucuronide- or dextran-conjugated prodrugs of prednisolone, dexamethasone or budesonide; ICAM-1 antisense phosphorothioate oligodeoxynucleotides (ISIS 2302; Isis Pharmaceuticals, Inc.); soluble complement receptor 1 (TP10; T Cell Sciences, Inc.); slow-release mesalazine; methotrexate; antagonists of platelet activating factor (PAF); ciprofloxacin; and/or lignocaine.

In some embodiments, the methods and compositions described herein are suitable for use in combination therapy with one or more additional therapeutic agents for treating or preventing inflammatory bowel disease (IBS) (e.g., Crohn's disease, ulcerative colitis).

Non-limiting examples of the additional therapeutic agents include: sphingosine 1-phosphate (S1P) receptor modulators (e.g., etrasimod or ozanimod); steroidal anti-inflammatory agents (e.g, beclomethasone 17 or budesonide); non-steroidal anti-inflammatory agents (e.g., 5-ASA); receptor-interacting protein kinase 1 (RIPK1) inhibitors (e.g., GSK2982772); EP4 modulators (e.g., KAG-308); toll-like receptor (e.g., TLR4, TLR9) modulators (e.g., JKB-122, cobitolimod); Janus kinase (JAK) inhibitors (e.g., TD-1473, tofacitinib, upadacitinib, filgotinib, PF-06651600, and PF-06700841); lanthionine synthetase C-like 2 (LANCL2) modulators (e.g., BT-11); phosphatidylcholine (e.g., LT-02); integrin (e.g., a4 Integrin) modulators (e.g, AJM-300 (carotegrast)); Smad7 modulators (e.g., mongersen); phosphodiesterase 4 (PDE4) modulators (e.g., apremilast); tumor progression locus 2 (TPL2) inhibitors (e.g., GS-4875); tyrosine kinase 2 (TYK2) inhibitors (e.g., BMS-986165, PF-06700841, and PF-06826647); and TEC kinase inhibitors (e.g., PF-06651600).

In some embodiments, the one or more additional therapeutic agents is selected from an agent/regimen for treating rheumatoid arthritis. Non-limiting examples include non-steroidal anti-inflammatory drugs (NSAIDs; e.g., ibuprofen and naproxen), corticosteroids (e.g, prednisone), disease-modifying antirheumatic drugs (DMARDs; e.g., methotrexate (Trexall®, Otrexup®, Rasuvo®, Rheumatrex®), leflunomide (Arava®), hydroxychloroquine (Plaquenil), PF-06650833, iguratimod, tofacitinib (Xeljanz®), ABBV-599, evobrutinib, and sulfasalazine (Azulfidine®)), and biologics (e.g., abatacept (Orencia®), adalimumab (Humira®), anakinra (Kineret®), certolizumab (Cimzia®), etanercept (Enbrel®), golimumab (Simponi®), infliximab (Remicade®), rituximab (Rituxan®), tocilizumab (Actemra®), vobarilizumab, sarilumab (Kevzara®), secukinumab, ABP 501, CHS-0214, ABC-3373, and tocilizumab (ACTEMRA®)).

In some embodiments, the one or more additional therapeutic agents is selected from an agent/regimen for treating lupus. Non-limiting examples include steroids, topical immunomodulators (e.g., tacrolimus ointment (Protopic®) and pimecrolimus cream (Elidel®)), thalidomide (Thalomid®), non-steroidal anti-inflammatory drugs (NSAIDs; e.g., ibuprofen and naproxen), antimalarial drugs (e.g., Hydroxychloroquine (Plaquenil)), corticosteroids (e.g, prednisone) and immunomodulators (e.g., evobrutinib, iberdomide, voclosporin, cenerimod, azathioprine (Imuran®), cyclophosphamide (Cytoxan®, Neosar®, Endoxan®), and cyclosporine (Neoral, Sandimmune®, GengrafM), and mycophenolate mofetil) baricitinb, iguratimod, filogotinib, GS-9876, rapamycin, and PF-06650833), and biologics (e.g., belimumab (Benlysta®), anifrolumab, prezalumab, MEDIO700, obinutuzumab, vobarilizumab, lulizumab, atacicept, PF-06823859, and lupizor, rituximab, BT063, BI655064, B1B059, aldesleukin (Proleukin®), dapirolizumab, edratide, IFN-α-kinoid, OMS721, RC18, RSLV-132, theralizumab, XmAb5871, and ustekinumab (Stelara®)). For example, non-limiting treatments for systemic lupus erythematosus include non-steroidal anti-inflammatory drugs (NSAIDs; e.g., ibuprofen and naproxen), antimalarial drugs (e.g., Hydroxychloroquine (Plaquenil)), corticosteroids (e.g, prednisone) and immunomodulators (e.g., iberdomide, voclosporin, azathioprine (Imuran®), cyclophosphamide (Cytoxan®, Neosar®, Endoxan®), and cyclosporine (Neoral, Sandimmune®, GengrafS), and mycophenolate mofetil, baricitinb, filogotinib, and PF-06650833), and biologics (e.g., belimumab (Benlysta®), anifrolumab, prezalumab, MEDIO700, vobarilizumab, lulizumab, atacicept, PF-06823859, lupizor, rituximab, BT063, BI655064, B1B059, aldesleukin (Proleukin®), dapirolizumab, edratide, IFN-α-kinoid, RC18, RSLV-132, theralizumab, XmAb5871, and ustekinumab (Stelara®)). As another example, non-limiting examples of treatments for cutaneous lupus include steroids, immunomodulators (e.g., tacrolimus ointment (Protopic®) and pimecrolimus cream (Elidel®)), GS-9876, filogotinib, and thalidomide (Thalomid®). Agents and regimens for treating drug-induced and/or neonatal lupus can also be administered.

In some embodiments, the one or more additional therapeutic agents is selected from an agent/regimen for treating IBDs. Non-limiting examples include 6-mercaptopurine, AbGn-168H, ABX464, ABT-494, adalimumab, AJM300, alicaforsen, AMG139, anrukinzumab, apremilast, ATR-107 (PF0530900), autologous CD34-selected peripheral blood stem cells transplant, azathioprine, bertilimumab, BI 655066, BMS-936557, certolizumab pegol (Cimzia®), cobitolimod, corticosteroids (e.g., prednisone, Methylprednisolone, prednisone), CP-690,550, CT-P13, cyclosporine, DIMS0150, E6007, E6011, etrasimod, etrolizumab, fecal microbial transplantation, figlotinib, fingolimod, firategrast (SB-683699) (formerly T-0047), GED0301, GLPG0634, GLPG0974, guselkumab, golimumab, GSK1399686, HMPL-004 (Andrographis paniculata extract), IMU-838, infliximab, Interleukin 2 (IL-2), Janus kinase (JAK) inhibitors, laquinimod, masitinib (AB1010), matrix metalloproteinase 9 (MMP 9) inhibitors (e.g., GS-5745), MEDI2070, mesalamine, methotrexate, mirikizumab (LY3074828), natalizumab, NNC 0142-0000-0002, NNC0114-0006, ozanimod, peficitinib (JNJ-54781532), PF-00547659, PF-04236921, PF-06687234, QAX576, RHB-104, rifaximin, risankizumab, RPC1063, SB012, SHP647, sulfasalazine, TD-1473, thalidomide, tildrakizumab (MK 3222), TJ301, TNF-Kinoid®, tofacitinib, tralokinumab, TRK-170, upadacitinib, ustekinumab, UTTR1147A, V565, vatelizumab, VB-201, vedolizumab, and vidofludimus.

In some embodiments, the one or more additional therapeutic agents is selected from an agent/regimen for treating irritable bowel syndrome. Non-limiting examples include alosetron, bile acid sequesterants (e.g., cholestyramine, colestipol, colesevelam), chloride channel activators (e.g., lubiprostone), coated peppermint oil capsules, desipramine, dicyclomine, ebastine, eluxadoline, farnesoid X receptor agonist (e.g., obeticholic acid), fecal microbiota transplantation, fluoxetine, gabapentin, guanylate cyclase-C agonists (e.g., linaclotide, plecanatide), ibodutant, imipramine, JCM-16021, loperamide, lubiprostone, nortriptyline, ondansetron, opioids, paroxetine, pinaverium, polyethylene glycol, pregabalin, probiotics, ramosetron, rifaximin, and tanpanor.

In some embodiments, the one or more additional therapeutic agents is selected from an agent/regimen for treating scleroderma. Non-limiting examples include non-steroidal anti-inflammatory drugs (NSAIDs; e.g., ibuprofen and naproxen), corticosteroids (e.g, prednisone), immunomodulators (e.g., azathioprine, methotrexate (Trexall®, Otrexup®, Rasuvo®, Rheumatrex®), cyclophosphamide (Cytoxan®, Neosar®, Endoxan®), and cyclosporine (Neoral®, Sandimmune®, GengrafS), antithymocyte globulin, mycophenolate mofetil, intravenous immunoglobulin, rituximab, sirolimus, and alefacept), calcium channel blockers (e.g., nifedipine), alpha blockers, serotonin receptor antagonists, angiotensin II receptor inhibitors, statins, local nitrates, iloprost, phosphodiesterase 5 inhibitors (e.g., sildenafil), bosentan, tetracycline antibiotics, endothelin receptor antagonists, prostanoids, and tyrosine kinase inhibitors (e.g., imatinib, nilotinib and dasatinib).

In some embodiments, the one or more additional therapeutic agents is selected from an agent/regimen for treating Crohn's Disease (CD). Non-limiting examples include adalimumab, autologous CD34-selected peripheral blood stem cells transplant, 6-mercaptopurine, azathioprine, certolizumab pegol (Cimzia®), corticosteroids (e.g., prednisone), etrolizumab, E6011, fecal microbial transplantation, figlotinib, guselkumab, infliximab, IL-2, JAK inhibitors, matrix metalloproteinase 9 (MMP 9) inhibitors (e.g., GS-5745), MEDI2070, mesalamine, methotrexate, natalizumab, ozanimod, RHB-104, rifaximin, risankizumab, SHP647, sulfasalazine, thalidomide, upadacitinib, V565, and vedolizumab.

In some embodiments, the one or more additional therapeutic agents is selected from an agent/regimen for treating UC. Non-limiting examples include AbGn-168H, ABT-494, ABX464, apremilast, PF-00547659, PF-06687234, 6-mercaptopurine, adalimumab, azathioprine, bertilimumab, brazikumab (MEDI2070), cobitolimod, certolizumab pegol (Cimzia®), CP-690,550, corticosteroids (e.g., multimax budesonide, Methylprednisolone), cyclosporine, E6007, etrasimod, etrolizumab, fecal microbial transplantation, figlotinib, guselkumab, golimumab, IL-2, IMU-838, infliximab, matrix metalloproteinase 9 (MMP9) inhibitors (e.g., GS-5745), mesalamine, mesalamine, mirikizumab (LY3074828), RPC1063, risankizumab (BI 6555066), SHP647, sulfasalazine, TD-1473, TJ301, tildrakizumab (MK 3222), tofacitinib, tofacitinib, ustekinumab, UTTR1147A, and vedolizumab.

In some embodiments, the one or more additional therapeutic agents is selected from an agent/regimen for treating autoimmune colitis. Non-limiting examples include corticosteroids (e.g., budesonide, prednisone, prednisolone, Beclometasone dipropionate), diphenoxylate/atropine, infliximab, loperamide, mesalamine, TIP60 inhibitors (see, e.g., U.S. Patent Application Publication No. 2012/0202848), and vedolizumab.

In some embodiments, the one or more additional therapeutic agents is selected from an agent/regimen for treating iatrogenic autoimmune colitis. Non-limiting examples include corticosteroids (e.g., budesonide, prednisone, prednisolone, Beclometasone dipropionate), diphenoxylate/atropine, infliximab, loperamide, TIP60 inhibitors (see, e.g., U.S. Patent Application Publication No. 2012/0202848), and vedolizumab.

In some embodiments, the one or more additional therapeutic agents is selected from an agent/regimen for treating colitis induced by one or more chemotherapeutics agents. Non-limiting examples include corticosteroids (e.g., budesonide, prednisone, prednisolone, beclometasone dipropionate), diphenoxylate/atropine, infliximab, loperamide, mesalamine, TIP60 inhibitors (see, e.g., U.S. Patent Application Publication No. 2012/0202848), and vedolizumab.

In some embodiments, the one or more additional therapeutic agents is selected from an agent/regimen for treating colitis induced by treatment with adoptive cell therapy. Non-limiting examples include corticosteroids (e.g., budesonide, prednisone, prednisolone, beclometasone dipropionate), diphenoxylate/atropine, infliximab, loperamide, TIP60 inhibitors (see, e.g., U.S. Patent Application Publication No. 2012/0202848), and vedolizumab.

In some embodiments, the one or more additional therapeutic agents is selected from an agent/regimen for treating colitis associated with one or more alloimmune diseases. Non-limiting examples include corticosteroids (e.g., budesonide, prednisone, prednisolone, beclometasone dipropionate), sulfasalazine, and eicopentaenoic acid.

In some embodiments, the one or more additional therapeutic agents is selected from an agent/regimen for treating radaiation enteritis. Non-limiting examples include teduglutide, amifostine, angiotensin-converting enzyme (ACE) inhibitors (e.g., benazepril, captopril, enalapril, fosinopril, lisinopril, moexipril, perindopril, quinapril, ramipril, and trandolapril), probiotics, selenium supplementation, statins (e.g., atorvastatin, fluvastatin, lovastatin, pravastatin, rosuvastatin, simvastatin, and pitavastatin), sucralfate, and vitamin E.

In some embodiments, the one or more additional therapeutic agents is selected from an agent/regimen for treating collagenous colitis. Non-limiting examples include 6-mercaptopurine, azathaioprine, bismuth subsalicate, Boswellia serrata extract, cholestyramine, colestipol, corticosteroids (e.g., budesonide, prednisone, prednisolone, beclometasone dipropionate), loperamide, mesalamine, methotrexate, probiotics, and sulfasalazine.

In some embodiments, the one or more additional therapeutic agents is selected from an agent/regimen for treating lyphocytic colitis. Non-limiting examples include 6-mercaptopurine, azathioprine, bismuth subsalicylate, cholestyramine, colestipol, corticosteroids (e.g., budesonide, prednisone, prednisolone, beclometasone dipropionate), loperamide, mesalamine, methotrexate, and sulfasalazine.

In some embodiments, the one or more additional therapeutic agents is selected from an agent/regimen for treating microscopic colitis. Non-limiting examples include 6-mercaptopurine, azathioprine, bismuth subsalicylate, Boswellia serrata extract, cholestyramine, colestipol, corticosteroids (e.g., budesonide, prednisone, prednisolone, beclometasone dipropionate), fecal microbial transplantation, loperamide, mesalamine, methotrexate, probiotics, and sulfasalazine.

In some embodiments, the one or more additional therapeutic agents is selected from an agent/regimen for treating alloimmune disease. Non-limiting examples include intrauterine platelet transfusions, intravenous immunoglobin, maternal steroids, abatacept, alemtuzumab, alpha1-antitrypsin, AMG592, antithymocyte globulin, barcitinib, basiliximab, bortezomib, brentuximab, cannabidiol, corticosteroids (e.g., methylprednisone, prednisone), cyclosporine, dacilzumab, defribrotide, denileukin diftitox, glasdegib, ibrutinib, IL-2, infliximab, itacitinib, LBH589, maraviroc, mycophenolate mofetil, natalizumab, neihulizumab, pentostatin, pevonedistat, photobiomodulation, photopheresis, ruxolitinib, sirolimus, sonidegib, tacrolimus, tocilizumab, and vismodegib.

In some embodiments, the one or more additional therapeutic agents is selected from an agent/regimen for treating multiple sclerosis (MS). Non-limiting examples include alemtuzumab (Lemtrada®), ALKS 8700, amiloride, ATX-MS-1467, azathioprine, baclofen (Lioresal®), beta interferons (e.g., IFN-β-1a, IFN-β-1b), cladribine, corticosteroids (e.g., methylprednisolone), daclizumab, dimethyl fumarate (Tecfidera®), fingolimod (Gilenya®), fluoxetine, glatiramer acetate (Copaxone®), hydroxychloroquine, ibudilast, idebenone, laquinimod, lipoic acid, losartan, masitinib, MD1003 (biotin), mitoxantrone, montelukast, natalizumab (Tysabri®), NeuroVax™, ocrelizumab, ofatumumab, pioglitazone, and RPC1063.

In some embodiments, the one or more additional therapeutic agents is selected from an agent/regimen for treating graft-vs-host disease. Non-limiting examples include abatacept, alemtuzumab, alpha1-antitrypsin, AMG592, antithymocyte globulin, barcitinib, basiliximab, bortezomib, brentuximab, cannabidiol, corticosteroids (e.g., methylprednisone, prednisone), cyclosporine, dacilzumab, defribrotide, denileukin diftitox, glasdegib, ibrutinib, IL-2, imatinib, infliximab, itacitinib, LBH589, maraviroc, mycophenolate mofetil, natalizumab, neihulizumab, pentostatin, pevonedistat, photobiomodulation, photopheresis, ruxolitinib, sirolimus, sonidegib, tacrolimus, tocilizumab, and vismodegib.

In some embodiments, the one or more additional therapeutic agents is selected from an agent/regimen for treating acute graft-vs-host disease. Non-limiting examples include alemtuzumab, alpha-1 antitrypsin, antithymocyte globulin, basiliximab, brentuximab, corticosteroids (e.g., methylprednisone, prednisone), cyclosporine, dacilzumab, defribrotide, denileukin diftitox, ibrutinib, infliximab, itacitinib, LBH589, mycophenolate mofetil, natalizumab, neihulizumab, pentostatin, photopheresis, ruxolitinib, sirolimus, tacrolimus, and tocilizumab.

In some embodiments, the one or more additional therapeutic agents is selected from an agent/regimen for treating chronic graft vs. host disease. Non-limiting examples include abatacept, alemtuzumab, AMG592, antithymocyte globulin, basiliximab, bortezomib, corticosteroids (e.g., methylprednisone, prednisone), cyclosporine, dacilzumab, denileukin diftitox, glasdegib, ibrutinib, IL-2, imatinib, infliximab, mycophenolate mofetil, pentostatin, photobiomodulation, photopheresis, ruxolitinib, sirolimus, sonidegib, tacrolimus, tocilizumab, and vismodegib.

In some embodiments, the one or more additional therapeutic agents is selected from an agent/regimen for treating celiac disease. Non-limiting examples include AMG 714, AMY01, Aspergillus niger prolyl endoprotease, BL-7010, CALY-002, GBR 830, Hu-Mik-Beta-1, IMGX003, KumaMax, Larazotide Acetate, Nexvan2®, pancrelipase, TIMP-GLIA, vedolizumab, and ZED1227.

In some embodiments, the one or more additional therapeutic agents is selected from an agent/regimen for treating psoriasis. Non-limiting examples include topical corticosteroids, topical crisaborole/AN2728, topical SNA-120, topical SAN021, topical tapinarof, topical tocafinib, topical IDP-118, topical M518101, topical calcipotriene and betamethasone dipropionate (e.g., MC2-01 cream and Taclonex®), topical P-3073, topical LEO 90100 (Enstilar®), topical betamethasone dipropriate (Sernivo®), halobetasol propionate (Ultravate®), vitamin D analogues (e.g., calcipotriene (Dovonex®) and calcitriol (Vectical®)), anthralin (e.g., Dritho-Scalp® and Dritho-Creme®), topical retinoids (e.g., tazarotene (e.g., Tazorac® and Avage®)), calcineurin inhibitors (e.g., tacrolimus (Prograf®) and pimecrolimus (Elidel®)), salicylic acid, coal tar, moisturizers, phototherapy (e.g., exposure to sunlight, UVB phototherapy, narrow band UVB phototherapy, Goeckerman therapy, psoralen plus ultraviolet A (PUVA) therapy, and excimer laser), retinoids (e.g., acitretin (Soriatane®)), methotrexate (Trexall®, Otrexup®, Rasuvo®, Rheumatrex®), Apo805K1, baricitinib, FP187, KD025, prurisol, VTP-43742, XP23829, ZPL-389, CF101 (piclidenoson), LAS41008, VPD-737 (serlopitant), upadacitinib (ABT-494), aprmilast, tofacitibin, cyclosporine (Neoral®, Sandimmune®, GengrafS), biologics (e.g., etanercept (Enbrel®), entanercept-szzs (Elrezi®), infliximab (Remicade®), adalimumab (Humira®), adalimumab-adbm (Cyltezo®), ustekinumab (Stelara®), golimumab (Simponi®), apremilast (Otezla®), secukinumab (Cosentyx®), certolixumab pegol, secukinumab, tildrakizumab-asmn, infliximab-dyyb, abatacept, ixekizumab (Taltz®), ABP 710, BCD-057, BI695501, bimekizumab (UCB4940), CHS-1420, GP2017, guselkumab (CNTO 1959), HD203, M923, MSB11022, Mirikizumab (LY3074828), PF-06410293, PF-06438179, risankizumab (BI655066), SB2, SB4, SB5, siliq (brodalumab), namilumab (MT203, tildrakizumab (MK-3222), and ixekizumab (Taltz®)), thioguanine, and hydroxyurea (e.g., Droxia® and Hydrea®).

In some embodiments, the one or more additional therapeutic agents is selected from an agent/regimen for treating cutaneous T-cell lymphoma. Non-limiting examples include phototherapy (e.g., exposure to sunlight, UVB phototherapy, narrow band UVB phototherapy, Goeckerman therapy, psoralen plus ultraviolet A (PUVA) therapy, and excimer laser), extracorporeal photopheresis, radiation therapy (e.g., spot radiation and total skin body electron beam therapy), stem cell transplant, corticosteroids, imiquimod, bexarotene gel, topical bis-chloroethyl-nitrourea, mechlorethamine gel, vorinostat (Zolinza®), romidepsin (Istodax®), pralatrexate (Folotyn®) biologics (e.g., alemtuzumab (Campath®), brentuximab vedotin (SGN-35), mogamulizumab, and IPH4102).

In some embodiments, the one or more additional therapeutic agents is selected from an agent/regimen for treating uveitis. Non-limiting examples include corticosteroids (e.g., intravitreal triamcinolone acetonide injectable suspensions), antibiotics, antivirals (e.g., acyclovir), dexamethasone, immunomodulators (e.g., tacrolimus, leflunomide, cyclophosphamide (Cytoxan®, Neosar®, Endoxan®), and cyclosporine (Neoral®, Sandimmune®, Gengrafg), chlorambucil, azathioprine, methotrexate, and mycophenolate mofetil), biologics (e.g., infliximab (Remicade®), adalimumab (Humira®), etanercept (Enbrel®), golimumab (Simponi®), certolizumab (Cimzia®), rituximab (Rituxan®), abatacept (Orencia®), basiliximab (Simulect®), anakinra (Kineret®), canakinumab (Ilaris®), gevokixumab (XOMA052), tocilizumab (Actemra®), alemtuzumab (Campath®), efalizumab (Raptiva®), LFG316, sirolimus (Santen®), abatacept, sarilumab (Kevzara®), and daclizumab (Zenapax®)), cytotoxic drugs, surgical implant (e.g., fluocinolone insert), and vitrectomy.

In some embodiments, the one or more additional therapeutic agents is selected from an agent/regimen for treating mucositis. Non-limiting examples include AG013, SGX942 (dusquetide), amifostine (Ethyol®), cryotherapy, cepacol lonzenges, capsaicin lozenges, mucoadhesives (e.g., MuGard®) oral diphenhydramine (e.g., Benadry® elixir), oral bioadherents (e.g., polyvinylpyrrolidone-sodium hyaluronate gel (Gelclair®)), oral lubricants (e.g., Oral Balance®), caphosol, Chamomilla recutita mouthwash, edible grape plant exosome, antiseptic mouthwash (e.g., chlorhexidine gluconate (e.g., Peridex® or Periogard®), topical pain relievers (e.g., lidocaine, benzocaine, dyclonine hydrochloride, xylocaine (e.g., viscous xylocaine 2%), and Ulcerease® (0.6% phenol)), corticosteroids (e.g., prednisone), pain killers (e.g., ibuprofen, naproxen, acetaminophen, and opioids), GC4419, palifermin (keratinocyte growth factor; Kepivance®), ATL-104, clonidine lauriad, IZN-6N4, SGX942, rebamipide, nepidermin, soluble β-1,3/1,6 glucan, P276, LP-0004-09, CR-3294, ALD-518, IZN-6N4, quercetin, granules comprising Vaccinium myrtillus extract, Macleaya cordata alkaloids and Echinacea angustifolia extract (e.g., SAMITAL®), and gastrointestinal cocktail (an acid reducer such aluminum hydroxide and magnesium hydroxide (e.g., Maalox), an antifungal (e.g., nystatin), and an analgesic (e.g., hurricane liquid)). For example, non-limiting examples of treatments for oral mucositis include AG013, amifostine (Ethyol®), cryotherapy, cepacol lonzenges, mucoadhesives (e.g., MuGard®) oral diphenhydramine (e.g., Benadry® elixir), oral bioadherents (e.g., polyvinylpyrrolidone-sodium hyaluronate gel (Gelclair®)), oral lubricants (e.g., Oral Balance®), caphosol, Chamomilla recutita mouthwash, edible grape plant exosome, antiseptic mouthwash (e.g., chlorhexidine gluconate (e.g., Peridex® or Periogard®), topical pain relievers (e.g., lidocaine, benzocaine, dyclonine hydrochloride, xylocaine (e.g., viscous xylocaine 2%), and Ulcerease® (0.6% phenol)), corticosteroids (e.g., prednisone), pain killers (e.g., ibuprofen, naproxen, acetaminophen, and opioids), GC4419, palifermin (keratinocyte growth factor; Kepivance®), ATL-104, clonidine lauriad, IZN-6N4, SGX942, rebamipide, nepidermin, soluble β-1,3/1,6 glucan, P276, LP-0004-09, CR-3294, ALD-518, IZN-6N4, quercetin, and gastrointestinal cocktail (an acid reducer such aluminum hydroxide and magnesium hydroxide (e.g., Maalox), an antifungal (e.g., nystatin), and an analgesic (e.g., hurricane liquid)). As another example, non-limiting examples of treatments for esophageal mucositis include xylocaine (e.g., gel viscous Xylocaine 2%). As another example, treatments for intestinal mucositis, treatments to modify intestinal mucositis, and treatments for intestinal mucositis signs and symptoms include gastrointestinal cocktail (an acid reducer such aluminum hydroxide and magnesium hydroxide (e.g., Maalox), an antifungal (e.g., nystatin), and an analgesic (e.g., hurricane liquid)).

In certain embodiments, the one or more additional therapeutic agents is a chemotherapeutic immunomodulator, e.g., an immune checkpoint inhibitor, which can be as defined anywhere herein. In other embodiments, the second therapeutic agent or regimen is one or more anti-inflammatory agents or immunomodulator acting locally in the GI tract. In other embodiments, the second therapeutic agent or regimen is 5-ASA (and associated delivery systems), anti-SMAD7 antisense, orally formulated anti-TNFs, anti-integrins, sulfasalazine, balsalazide, steroids, azathioprine, and methotrexate. In further embodiments, the second therapeutic agent or regimen is radiation or surgery.

In certain embodiments, the one or more additional therapeutic agents is platinum, cisplatin, carboplatin, oxaliplatin, mechlorethamine, cyclophosphamide, chlorambucil, azathioprine, mercaptopurine, vincristine, vinblastine, vinorelbine, vindesine, etoposide and teniposide, paclitaxel, docetaxel, irinotecan, topotecan, amsacrine, etoposide, etoposide phosphate, teniposide, 5-fluorouracil, leucovorin, methotrexate, gemcitabine, taxane, leucovorin, mitomycin C, tegafur-uracil, idarubicin, fludarabine, mitoxantrone, ifosfamide and doxorubicin. Additional agents include inhibitors of mTOR (mammalian target of rapamycin), including but not limited to rapamycin, everolimus, temsirolimus and deforolimus.

In certain embodiments, the one or more additional therapeutic agents can be selected from those delineated above (see U.S. Pat. No. 7,927,613, which is incorporated herein by reference in its entirety).

In certain embodiments, the one or more additional therapeutic agents can be selected from the compounds that are disclosed generically, sub generically and specifically in any one or more of WO 2004/006906: WO 2006/120178; US 2009/0062396; WO 2012/143377; WO 2012/068274, U.S. Pat. Nos. 7,132,546, 7,989,498; and 8,263,857; each of which is incorporated herein by reference in its entirety.

In certain embodiments, the one or more additional therapeutic agent can be an anthelminthic agent selected from nitazoxanide, closantel, pyrvinium pamoate, and salinomycin. See, e.g., Senkowski, W., et al., Mol. Cancer Ther. 2015, 14, 1504.

In some embodiments, the methods and compositions described herein for treating a subject having COVID-19 are suitable for use in combination therapy with various other therapeutic regimens. In certain embodiments, the compound of Formula (I), or pharmaceutically acceptable salts and/or cocrystal thereof, and methods described herein can be used to treat side effects produced by such therapeutic regimens.

In some embodiments, the methods and compositions described herein are suitable for use in combination therapy with one or more additional therapeutic agents.

In certain embodiments, the one or more additional therapeutic agents is administered to the subject prior to contacting with or administering the compound of Formula (I), or a pharmaceutically acceptable salt and/or cocrystal thereof, (e.g., about one hour prior, or about 6 hours prior, or about 12 hours prior, or about 24 hours prior, or about 48 hours prior, or about 1 week prior, or about 1 month prior).

In other embodiments, the one or more additional therapeutic agents is administered to the subject at about the same time as contacting with or administering the compound of Formula (I), or a pharmaceutically acceptable salt and/or cocrystal thereof. By way of example, the second therapeutic agent or regimen and the compound of Formula (I), or a pharmaceutically acceptable salt and/or cocrystal thereof, are provided to the subject simultaneously in the same dosage form. As another example, the second therapeutic agent or regimen and the compound of Formula (I), or a pharmaceutically acceptable salt and/or cocrystal thereof, are provided to the subject concurrently in separate dosage forms.

In still other embodiments, the one or more additional therapeutic agents is administered to the subject after contacting with or administering the compound of Formula (I), or a pharmaceutically acceptable salt and/or cocrystal thereof, (e.g., about one hour after, or about 6 hours after, or about 12 hours after, or about 24 hours after, or about 48 hours after, or about 1 week after, or about 1 month after).

In some embodiments, one or more therapies can be used in combination with the materials and/or methods described herein. In some embodiments, a combination therapy can include one or more of a macrolide antibiotic, an anti-malarial agent, an anti-diabetic agent, an angiotensin receptor inhibitor, an angiotensin-converting enzyme (ACE) inhibitor, a statin, a polymerase inhibitor (e.g., a RNA-dependent RNA polymerase inhibitor), a protease inhibitor, a neuraminidase inhibitor, a fusion inhibitor, a transmembrane protease serine 2 (TMPRSS2) inhibitor, a broad-spectrum antiviral agent, a JAK-STAT pathway inhibitor, a DNA synthesis inhibitor, a phosphodiesterase 5 (PDE5) inhibitor, a monoclonal antibody, passive antibody therapy, recombinant human angiotensin-converting enzyme 2 (rhACE2), traditional Chinese medicine, a pharmaceutically acceptable salt or solvate of any thereof, or two or more of any thereof. In some embodiments, the macrolide antibiotic and/or anti-malarial agent is a lysosomotropic agent. Non-limiting examples of lysosomotropic agents include azithromycin, hydroxychloroquine, chloroquine, and ammonium chloride. Non-limiting examples of an anti-diabetic agent include a biguanide, a sulfonylurea, a glitazar, a thiazolidinedione, a dipeptidyl peptidase 4 (DPP-4) inhibitor, a meglitinide, a sodium-glucose linked transporter 2 (SGLT2) inhibitor, a glitazone, a GRP40 agonist, a glucose-dependent insulinotropic peptide (GIP), an insulin or insulin analogue, an alpha glucosidase inhibitor, a sodium-glucose linked transporter 1 (SGLT1) inhibitor. In some embodiments, the biguanide is metformin. A non-limiting example of an angiotensin receptor inhibitor includes a sartan (e.g., eprosartan, olmesartan, olmesartan medoxomil, valsartan, candesartan, candesartan cilexetil, losartan, telmisartan, irbesartan, BRA-657, and azilsartan medoxomil). Non-limiting examples of an ACE inhibitor include: quinapril, fosinopril perindopril, captopril, enalapril, enalaprilat, ramipril, cilazapril, delapril, fosenopril, zofenopril, indolapril, benazepril, lisinopril, spirapril, trandolapril, perindep, pentopril, moexipril, rescinnamine, and pivopril. Non-limiting examples of a statin include atorvastatin, fluvastatin, lovastatin, pitavastatin, pravastatin, rosuvastatin, and simvastatin, cerivastatin, and mevastatin. Non-limiting examples of polymerase inhibitors include ganciclovir, valganciclovir, and RNA-dependent RNA polymerase (RdRP) inhibitors (e.g., remdesivir, ribavirin, and favipiravir). Non-limiting examples of protease inhibitors include lopinavir, ritonavir, indinavir, atazanavir, nelfinavir, darunavir, tipranavir, amprenavir, and fosamprenavir. A non-limiting example of a neuraminidase inhibitor is oseltamivir. A non-limiting example of a fusion inhibitor is umifenovir. A non-limiting example of a TMPRSS2 inhibitor is camostat. Non-limiting examples of broad-spectrum antiviral agents include nitazoxanide, chloroquine, hydroxychloroquine, and interferon (e.g., interferon alfa). Non-limiting examples of JAK-STAT pathway inhibitors include baricitinib, fedratinib, and ruxolitinib. Non-limiting examples of DNA synthesis inhibitors include tenofovir disoproxil and lamivudine. A non-limiting example of a PDE5 inhibitor is sildenafil. A non-limiting example of traditional Chinese medicine is huaier extract. See, e.g., Liu, Cynthia, et al. “Research and Development on Therapeutic Agents and Vaccines for COVID-19 and Related Human Coronavirus Diseases.” (2020). doi/10.1021/acscentsci.0c00272; Lai, Chih-Cheng, et al. “Severe acute respiratory syndrome coronavirus 2 (SARS-CoV-2) and coronavirus disease-2019 (COVID-19): the epidemic and the challenges.” International journal of antimicrobial agents (2020): 105924; Chang, Yu-Chuan, et al. “Potential therapeutic agents for COVID-19 based on the analysis of protease and RNA polymerase docking.” (2020). doi: 10.20944/preprints202002.0242.v1; NCT04252885; NCT04306497; NCT04287686; NCT04307693; NCT04292899; NCT04304313; NCT04291053; Ko W C, Rolain J M, Lee N Y, et al. Arguments in favor of remdesivir for treating SARS-CoV-2 infections [published online ahead of print, 2020 March 5]. Int J Antimicrob Agents. 2020; 105933. doi:10.1016/j.ijantimicag.2020.105933; Dhama, K., et al. (2020). Coronavirus Disease 2019-COVID-19. doi: 10.20944/preprints202003.0001.v1; Stebbing, Justin, et al. “COVID-19: combining antiviral and anti-inflammatory treatments.” The Lancet Infectious Diseases (2020); Yang, Naidi, and Han-Ming Shen. “Targeting the Endocytic Pathway and Autophagy Process as a Novel Therapeutic Strategy in COVID-19.” Int J Biol Sci 16.10 (2020): 1724-1731; Casadevall, Arturo, and Liise-anne Pirofski. “The convalescent sera option for containing COVID-19.” The Journal of Clinical Investigation 130.4 (2020); Shanmugaraj, Balamurugan, et al. “Perspectives on monoclonal antibody therapy as potential therapeutic intervention for Coronavirus disease-19 (COVID-19).” Asian Pacific journal of allergy and immunology (2020); and Xu, Jimin, et al. “Broad Spectrum Antiviral Agent Niclosamide and Its Therapeutic Potential.” ACS Infectious Diseases (2020), each of which is incorporated by reference herein in its entirety.

Provided herein are methods for treating COVID-19 in a subject in need thereof, the method comprising administering the compound of Formula (I), or a pharmaceutically acceptable salt and/or cocrystal thereof, and a lysosomotropic agent to the subject. In some embodiments, the lysosomotropic agent is selected from azithromycin, hydroxychloroquine, chloroquine, ammonium chloride, and a combination thereof.

In some embodiments, methods provided herein for treating COVID-19 in a subject in need thereof include administering the compound of Formula (I), or a pharmaceutically acceptable salt and/or cocrystal thereof, and azithromycin. In some embodiments, about 500 mg azithromycin is administered to the subject once per day. In some embodiments, about 250 mg azithromycin is administered to the subject once per day. In some embodiments, about 500 mg azithromycin is administered to the subject on Day 1 and about 250 mg azithromycin is administered to the subject once per day on Days 2-5. See, e.g., Gautret et al. Int J Antimicrob Agents. 2020 Mar. 20:105949.

In some embodiments, methods provided herein for treating COVID-19 in a subject in need thereof include administering the compound of Formula (I), or a pharmaceutically acceptable salt and/or cocrystal thereof, and hydroxychloroquine. In some embodiments, about 200 mg hydroxychloroquine is administered to the subject three times per day. In some embodiments, about 200 mg hydroxychloroquine is administered to the subject three times per day for about 10 days. See, e.g., Gautret et al. Int J Antimicrob Agents. 2020 Mar. 20:105949.

In some embodiments, methods provided herein for treating COVID-19 in a subject in need thereof include administering the compound of Formula (I), or a pharmaceutically acceptable salt and/or cocrystal thereof, and chloroquine.

In some embodiments, methods provided herein for treating COVID-19 in a subject in need thereof include administering the compound of Formula (I), or a pharmaceutically acceptable salt and/or cocrystal thereof, azithromycin, and hydroxychloroquine.

In some embodiments, methods provided herein for treating COVID-19 in a subject in need thereof include administering the compound of Formula (I), or a pharmaceutically acceptable salt and/or cocrystal thereof, azithromycin, and chloroquine.

In some embodiments, the chloroquine is chloroquine phosphate (e.g., ARALEN®). In some embodiments, the hydroxychloroquine is hydroxychloroquine sulfate (e.g., PLAQUENIL®).

Also provided herein are methods for treating COVID-19 in a subject in need thereof, the methods comprising administering the compound of Formula (I), or a pharmaceutically acceptable salt and/or cocrystal thereof, and an anti-diabetic agent. In some embodiments, the anti-diabetic agent is metformin.

Also provided herein are methods for treating COVID-19 in a subject in need thereof, the methods comprising administering the compound of Formula (I), or a pharmaceutically acceptable salt and/or cocrystal thereof, and an angiotensin receptor inhibitor. In some embodiments, the angiotensin receptor inhibitor is selected from eprosartan, olmesartan, valsartan, candesartan, losartan, telmisartan, irbesartan, azilsartan medoxomil, and a combination thereof.

Also provided herein are methods for treating COVID-19 in a subject in need thereof, the methods comprising administering the compound of Formula (I), or a pharmaceutically acceptable salt and/or cocrystal thereof, and a statin. In some embodiments, the statin is selected from atorvastatin, fluvastatin, lovastatin, pitavastatin, pravastatin, rosuvastatin, and simvastatin, cerivastatin, mevastatin, and a combination thereof.

In some embodiments, the methods described herein further include the step of identifying a subject (e.g., a patient) in need of such treatment (e.g., by way of biopsy, endoscopy, or other conventional method known in the art).

In some embodiments, the chemical entities, methods, and compositions described herein can be administered to certain treatment-resistant patient populations, e.g., one that is nonresponsive or resistant to treatment with an anti-TNFalpha therapy (e.g., Humira, Enbrel, Remicade, Cimzia, Simponi, Enbrel, xanthine derivatives, e.g., pentoxifylline and Bupropion; (R)-DOI, TCB-2, LSD and LA-SS-Az). In certain embodiments, the patient is undergoing and/or has undergone treatment with an anti-TNFalpha therapy (e.g., Humira, Enbrel, Remicade, Cimzia, Simponi, Enbrel, xanthine derivatives, e.g., pentoxifylline and Bupropion; (R)-DOI, TCB-2, LSD and LA-SS-Az).

In some embodiments, provided herein is a method for inducing cell death of one or more T cells (e.g., in the digestive and/or gastrointestinal tract (GI), skin, eyes, or joints), of a subject, wherein the method comprises contacting the one or more T cells with an effective amount of a compound of Formula (I), or a pharmaceutically acceptable salt and/or cocrystal thereof as defined anywhere herein, wherein the amount of the compound of Formula (I) is less than an amount of niclosamide that is effective in inducing cell death of one or more T cells. In some embodiments, the compound of Formula (I) is selected from a compound of Table 1.

In some embodiments, provided herein is a method for treating a subject having a condition associated with unregulated (abnormal, elevated) recruitment and/or retention of one or more T cells (e.g., at the digestive and/or gastrointestinal tract (GI), e.g., colon, e.g., skin, eyes, or joints) of the subject, wherein the method comprises contacting the one or more T cells with an effective amount of a compound of Formula (I), or a pharmaceutically acceptable salt and/or cocrystal thereof as defined anywhere herein, wherein the amount of the compound of Formula (I) is less than an amount of niclosamide that is effective in treating the subject. In some embodiments, the compound of Formula (I) is selected from a compound of Table 1.

In some embodiments, provided herein is a method for treating a subject having a condition associated with unregulated (abnormal, elevated) activation of one or more T cells (e.g., in the digestive and/or gastrointestinal tract (GI), e.g., colon) of the subject, wherein the method comprises contacting the one or more T cells with an effective amount of a cocrystal comprising (i) a compound of Formula (I), or a pharmaceutically acceptable salt thereof; and (ii) one or more pharmaceutically acceptable coformers as defined anywhere herein, wherein the amount of the compound of Formula (I) is less than an amount of niclosamide that is effective in treating the subject. In some embodiments, the compound of Formula (I) is selected from a compound of Table 1.

In some embodiments, provided herein is a method for treating a condition (or one or more symptoms thereof) characterized by an abnormal inflammatory response in a subject in need thereof (e.g., an autoimmune disorder, e.g., colitis, e.g., autoimmune colitis, e.g., an inflammatory bowel disease; e.g., Crohn's disease or ulcerative colitis), wherein the method comprises administering to the subject an effective amount of a chemical entity (e.g., a compound of Formula (I), or a pharmaceutically acceptable salt and/or cocrystal thereof) as defined anywhere herein, wherein the amount of the compound of Formula (I) is less than an amount of niclosamide that is effective in treating the condition. In some embodiments, the compound of Formula (I) is selected from a compound of Table 1.

In some embodiments, provided herein is a method for treating a condition (or one or more symptoms thereof) characterized by an abnormal inflammatory response in a subject in need thereof (e.g., an autoimmune disorder, e.g., colitis, e.g., autoimmune colitis, e.g., an inflammatory bowel disease; e.g., Crohn's disease or ulcerative colitis), wherein the method comprises topically and locally administering to the subject an effective amount of a chemical entity (e.g., a compound of Formula (I), or a pharmaceutically acceptable salt and/or cocrystal thereof) as defined anywhere herein, wherein the amount of the compound of Formula (I) is less than an amount of niclosamide that is effective in treating the condition. In some embodiments, the compound of Formula (I) is selected from a compound of Table 1.

In some of the foregoing embodiments, the compound of Formula (I), or a pharmaceutically acceptable salt and/or cocrystal thereof is administered by recatal administration (e.g., by enema, rectal foam, suppository, e.g., enemal).

In some embodiments, provided herein is a method for treating a condition (or one or more symptoms thereof) characterized by an abnormal inflammatory response in a subject in need thereof (e.g., an autoimmune disorder, e.g., colitis, e.g., autoimmune colitis, e.g., an inflammatory bowel disease; e.g., Crohn's disease or ulcerative colitis), wherein the method comprises orally administering to the subject an effective amount of a chemical entity (e.g., a compound of Formula (I), or a pharmaceutically acceptable salt and/or cocrystal thereof) as defined anywhere herein, wherein the amount of the compound of Formula (I) is less than an amount of niclosamide that is effective in treating the condition. In some embodiments, the compound of Formula (I) is selected from a compound of Table 1.

In some embodiments, provided herein is a method for treating colitis, e.g., autoimmune colitis, e.g., an inflammatory bowel disease; e.g., Crohn's disease or ulcerative (or one or more symptoms thereof) in a subject, wherein the method comprises administering to the subject an effective amount of a chemical entity (e.g., a compound of Formula (I), or a pharmaceutically acceptable salt and/or cocrystal thereof) as defined anywhere herein, wherein the amount of the compound of Formula (I) is less than an amount of niclosamide that is effective in treating colitis. In some embodiments, the compound of Formula (I) is selected from a compound of Table 1.

In some embodiments, provided herein is a method for treating colitis, e.g., autoimmune colitis, e.g., an inflammatory bowel disease; e.g., Crohn's disease or ulcerative (or one or more symptoms thereof) in a subject, wherein the method comprises administering to the subject an effective amount of a chemical entity (e.g., a compound of Formula (I), or a pharmaceutically acceptable salt and/or cocrystal thereof) as defined anywhere herein, wherein the amount of the compound of Formula (I) present in the subject's colonic tissue and/or small bowel is greater than an amount present after administering niclosamide. In some embodiments, the compound of Formula (I) is selected from a compound of Table 1.

In some embodiments, provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject, wherein the method comprises administering to the subject an effective amount of a chemical entity (e.g., a compound of Formula (I), or a pharmaceutically acceptable salt and/or cocrystal thereof) as defined anywhere herein, wherein the amount of the compound of Formula (I) is less than an amount of niclosamide that is effective in treating the condition. In some embodiments, the compound of Formula (I) is selected from a compound of Table 1.

In some embodiments, provided herein is a method for treating a subject having a condition associated with unregulated (abnormal, elevated) recruitment and/or retention of one or more T cells (e.g., at the digestive and/or gastrointestinal tract (GI), e.g., colon, e.g., skin, eyes, or joints) of the subject, wherein the method comprises contacting the one or more T cells with an effective amount of a compound of Formula (I), or a pharmaceutically acceptable salt and/or cocrystal thereof as defined anywhere herein, wherein the AUC24 of the compound of Formula (I) in the plasma of the subject is greater than the AUC24 of niclosamide in the plasma of a second subject following administration to the second subject of the same amount of niclosamide as the amount of the compound of Formula (I). In some embodiments, the compound of Formula (I) is selected from a compound of Table 1. In some embodiments the two “subjects” are the same. In some embodiments they are different. In some embodiments, the second subject is part of a population of subjects.

In some embodiments, provided herein is a method for treating a subject having a condition associated with unregulated (abnormal, elevated) activation of one or more T cells (e.g., in the digestive and/or gastrointestinal tract (GI), e.g., colon) of the subject, wherein the method comprises contacting the one or more T cells with an effective amount of a cocrystal comprising (i) a compound of Formula (I), or a pharmaceutically acceptable salt thereof; and (ii) one or more pharmaceutically acceptable coformers as defined anywhere herein, wherein the AUC24 of the compound of Formula (I) in the plasma of the subject is greater than the AUC24 of niclosamide in the plasma of a second subject following administration to the second subject of the same amount of niclosamide as the amount of the compound of Formula (I). In some embodiments, the compound of Formula (I) is selected from a compound of Table 1.

In some embodiments, provided herein is a method for treating a condition (or one or more symptoms thereof) characterized by an abnormal inflammatory response in a subject in need thereof (e.g., an autoimmune disorder, e.g., colitis, e.g., autoimmune colitis, e.g., an inflammatory bowel disease; e.g., Crohn's disease or ulcerative colitis), wherein the method comprises administering to the subject an effective amount of a chemical entity (e.g., a compound of Formula (I), or a pharmaceutically acceptable salt and/or cocrystal thereof) as defined anywhere herein, wherein the AUC24 of the compound of Formula (I) in the plasma of the subject is greater than the AUC24 of niclosamide in the plasma of a second subject following administration to the second subject of the same amount of niclosamide as the amount of the compound of Formula (I). In some embodiments, the compound of Formula (I) is selected from a compound of Table 1.

In some embodiments, provided herein is a method for treating a condition (or one or more symptoms thereof) characterized by an abnormal inflammatory response in a subject in need thereof (e.g., an autoimmune disorder, e.g., colitis, e.g., autoimmune colitis, e.g., an inflammatory bowel disease; e.g., Crohn's disease or ulcerative colitis), wherein the method comprises topically and locally administering to the subject an effective amount of a chemical entity (e.g., a compound of Formula (I), or a pharmaceutically acceptable salt and/or cocrystal thereof) as defined anywhere herein, wherein the AUC24 of the compound of Formula (I) in the plasma of the subject is greater than the AUC24 of niclosamide in the plasma of a second subject following administration to the second subject of the same amount of niclosamide as the amount of the compound of Formula (I). In some embodiments, the compound of Formula (I) is selected from a compound of Table 1.

In some embodiments, provided herein is a method for treating a condition (or one or more symptoms thereof) characterized by an abnormal inflammatory response in a subject in need thereof (e.g., an autoimmune disorder, e.g., colitis, e.g., autoimmune colitis, e.g., an inflammatory bowel disease; e.g., Crohn's disease or ulcerative colitis), wherein the method comprises orally administering to the subject an effective amount of a chemical entity (e.g., a compound of Formula (I), or a pharmaceutically acceptable salt and/or cocrystal thereof) as defined anywhere herein, wherein the AUC24 of the compound of Formula (I) in the plasma of the subject is greater than the AUC24 of niclosamide in the plasma of a second subject following administration to the second subject of the same amount of niclosamide as the amount of the compound of Formula (I). In some embodiments, the compound of Formula (I) is selected from a compound of Table 1.

In some embodiments, provided herein is a method for treating colitis, e.g., autoimmune colitis, e.g., an inflammatory bowel disease; e.g., Crohn's disease or ulcerative (or one or more symptoms thereof) in a subject, wherein the method comprises administering to the subject an effective amount of a chemical entity (e.g., a compound of Formula (I), or a pharmaceutically acceptable salt and/or cocrystal thereof) as defined anywhere herein, wherein the AUC24 of the compound of Formula (I) in the plasma of the subject is greater than the AUC24 of niclosamide in the plasma of a second subject following administration to the second subject of the same amount of niclosamide as the amount of the compound of Formula (I). In some embodiments, the compound of Formula (I) is selected from a compound of Table 1.

In some embodiments, provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject, wherein the method comprises administering to the subject an effective amount of a chemical entity (e.g., a compound of Formula (I), or a pharmaceutically acceptable salt and/or cocrystal thereof) as defined anywhere herein, wherein the AUC24 of the compound of Formula (I) in the plasma of the subject is greater than the AUC24 of niclosamide in the plasma of a second subject following administration to the second subject of the same amount of niclosamide as the amount of the compound of Formula (I). In some embodiments, the compound of Formula (I) is selected from a compound of Table 1.

In some embodiments, provided herein is a method for treating a subject having a condition associated with unregulated (abnormal, elevated) recruitment and/or retention of one or more T cells (e.g., at the digestive and/or gastrointestinal tract (GI), e.g., colon, e.g., skin, eyes, or joints) of the subject, wherein the method comprises contacting the one or more T cells with an effective amount of a compound of Formula (I), or a pharmaceutically acceptable salt and/or cocrystal thereof as defined anywhere herein, wherein the t½ of the compound of Formula (I) in the plasma of the subject is longer than the t½ of niclosamide in the plasma of a second subject following administration to the second subject of niclosamide. In some embodiments, the compound of Formula (I) is selected from a compound of Table 1.

In some embodiments, provided herein is a method for treating a subject having a condition associated with unregulated (abnormal, elevated) activation of one or more T cells (e.g., in the digestive and/or gastrointestinal tract (GI), e.g., colon) of the subject, wherein the method comprises contacting the one or more T cells with an effective amount of a cocrystal comprising (i) a compound of Formula (I), or a pharmaceutically acceptable salt thereof; and (ii) one or more pharmaceutically acceptable coformers as defined anywhere herein, wherein the t½ of the compound of Formula (I) in the plasma of the subject is longer than the t½ of niclosamide in the plasma of a second subject following administration to the second subject of niclosamide. In some embodiments, the compound of Formula (I) is selected from a compound of Table 1.

In some embodiments, provided herein is a method for treating a condition (or one or more symptoms thereof) characterized by an abnormal inflammatory response in a subject in need thereof (e.g., an autoimmune disorder, e.g., colitis, e.g., autoimmune colitis, e.g., an inflammatory bowel disease; e.g., Crohn's disease or ulcerative colitis), wherein the method comprises administering to the subject an effective amount of a chemical entity (e.g., a compound of Formula (I), or a pharmaceutically acceptable salt and/or cocrystal thereof) as defined anywhere herein, wherein the t½ of the compound of Formula (I) in the plasma of the subject is longer than the t½ of niclosamide in the plasma of a second subject following administration to the second subject of niclosamide. In some embodiments, the compound of Formula (I) is selected from a compound of Table 1.

In some embodiments, provided herein is a method for treating a condition (or one or more symptoms thereof) characterized by an abnormal inflammatory response in a subject in need thereof (e.g., an autoimmune disorder, e.g., colitis, e.g., autoimmune colitis, e.g., an inflammatory bowel disease; e.g., Crohn's disease or ulcerative colitis), wherein the method comprises topically and locally administering to the subject an effective amount of a chemical entity (e.g., a compound of Formula (I), or a pharmaceutically acceptable salt and/or cocrystal thereof) as defined anywhere herein, wherein the t½ of the compound of Formula (I) in the plasma of the subject is longer than the t½ of niclosamide in the plasma of a second subject following administration to the second subject of niclosamide. In some embodiments, the compound of Formula (I) is selected from a compound of Table 1.

In some embodiments, provided herein is a method for treating a condition (or one or more symptoms thereof) characterized by an abnormal inflammatory response in a subject in need thereof (e.g., an autoimmune disorder, e.g., colitis, e.g., autoimmune colitis, e.g., an inflammatory bowel disease; e.g., Crohn's disease or ulcerative colitis), wherein the method comprises orally administering to the subject an effective amount of a chemical entity (e.g., a compound of Formula (I), or a pharmaceutically acceptable salt and/or cocrystal thereof) as defined anywhere herein, wherein the t½ of the compound of Formula (I) in the plasma of the subject is longer than the t½ of niclosamide in the plasma of a second subject following administration to the second subject of niclosamide. In some embodiments, the compound of Formula (I) is selected from a compound of Table 1.

In some embodiments, provided herein is a method for treating colitis, e.g., autoimmune colitis, e.g., an inflammatory bowel disease; e.g., Crohn's disease or ulcerative (or one or more symptoms thereof) in a subject, wherein the method comprises administering to the subject an effective amount of a chemical entity (e.g., a compound of Formula (I), or a pharmaceutically acceptable salt and/or cocrystal thereof) as defined anywhere herein, wherein the t½ of the compound of Formula (I) in the plasma of the subject is longer than the t½ of niclosamide in the plasma of a second subject following administration to the second subject of niclosamide. In some embodiments, the compound of Formula (I) is selected from a compound of Table 1.

In some embodiments, provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject, wherein the method comprises administering to the subject an effective amount of a chemical entity (e.g., a compound of Formula (I), or a pharmaceutically acceptable salt and/or cocrystal thereof) as defined anywhere herein, wherein the t½ of the compound of Formula (I) in the plasma of the subject is longer than the t½ of niclosamide in the plasma of a second subject following administration to the second subject of niclosamide. In some embodiments, the compound of Formula (I) is selected from a compound of Table. 1.

In some embodiments, provided herein is a method for treating a subject having a condition associated with unregulated (abnormal, elevated) recruitment and/or retention of one or more T cells (e.g., at the digestive and/or gastrointestinal tract (GI), e.g., colon, e.g., skin, eyes, or joints) of the subject, wherein the method comprises contacting the one or more T cells with an effective amount of a compound of Formula (I), or a pharmaceutically acceptable salt and/or cocrystal thereof as defined anywhere herein, wherein the AUC24 of the compound of formula

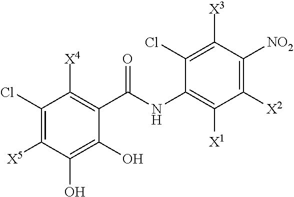

in the plasma of the subject is smaller than the AUC24 of the compound of formula

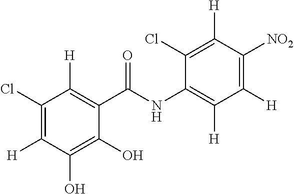 [SMILES: O=C(Nc1ccc([N+](=O)[O-])cc1Cl)c1cc(Cl)cc(O)c1O]

in the plasma of a second subject following administration to the second subject of the same amount of niclosamide as the amount of the compound of Formula (I). In some embodiments, the compound of Formula (I) is selected from a compound of Table 1.

In some embodiments, provided herein is a method for treating a subject having a condition associated with unregulated (abnormal, elevated) activation of one or more T cells (e.g., in the digestive and/or gastrointestinal tract (GI), e.g., colon) of the subject, wherein the method comprises contacting the one or more T cells with an effective amount of a cocrystal comprising (i) a compound of Formula (I), or a pharmaceutically acceptable salt thereof; and (ii) one or more pharmaceutically acceptable coformers as defined anywhere herein, wherein the AUC24 of the compound of formula

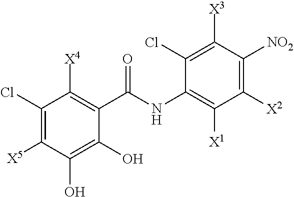

in the plasma of the subject is smaller than the AUC24 of the compound of formula

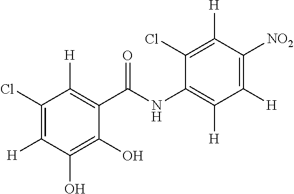 [SMILES: O=C(Nc1ccc([N+](=O)[O-])cc1Cl)c1cc(Cl)cc(O)c1O]

in the plasma of a second subject following administration to the second subject of the same amount of niclosamide as the amount of the compound of Formula (I). In some embodiments, the compound of Formula (I) is selected from a compound of Table 1.

In some embodiments, provided herein is a method for treating a condition (or one or more symptoms thereof) characterized by an abnormal inflammatory response in a subject in need thereof (e.g., an autoimmune disorder, e.g., colitis, e.g., autoimmune colitis, e.g., an inflammatory bowel disease; e.g., Crohn's disease or ulcerative colitis), wherein the method comprises administering to the subject an effective amount of a chemical entity (e.g., a compound of Formula (I), or a pharmaceutically acceptable salt and/or cocrystal thereof) as defined anywhere herein, wherein the AUC24 of the compound of formula

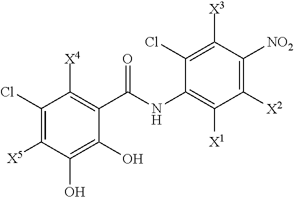

in the plasma of the subject is smaller than the AUC24 of the compound of formula

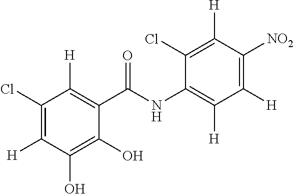 [SMILES: O=C(Nc1ccc([N+](=O)[O-])cc1Cl)c1cc(Cl)cc(O)c1O]

in the plasma of a second subject following administration to the second subject of the same amount of niclosamide as the amount of the compound of Formula (I). In some embodiments, the compound of Formula (I) is selected from a compound of Table 1.

In some embodiments, provided herein is a method for treating a condition (or one or more symptoms thereof) characterized by an abnormal inflammatory response in a subject in need thereof (e.g., an autoimmune disorder, e.g., colitis, e.g., autoimmune colitis, e.g., an inflammatory bowel disease; e.g., Crohn's disease or ulcerative colitis), wherein the method comprises topically and locally administering to the subject an effective amount of a chemical entity (e.g., a compound of Formula (I), or a pharmaceutically acceptable salt and/or cocrystal thereof) as defined anywhere herein, wherein the AUC24 of the compound of formula

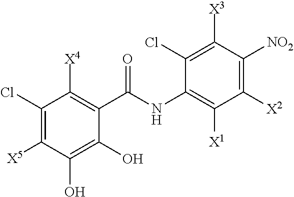

in the plasma of the subject is smaller than the AUC24 of the compound of formula

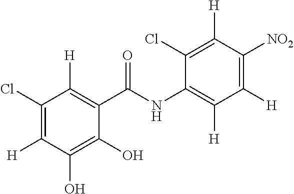 [SMILES: O=C(Nc1ccc([N+](=O)[O-])cc1Cl)c1cc(Cl)cc(O)c1O]

in the plasma of a second subject following administration to the second subject of the same amount of niclosamide as the amount of the compound of Formula (I). In some embodiments, the compound of Formula (I) is selected from a compound of Table 1.

In some embodiments, provided herein is a method for treating a condition (or one or more symptoms thereof) characterized by an abnormal inflammatory response in a subject in need thereof (e.g., an autoimmune disorder, e.g., colitis, e.g., autoimmune colitis, e.g., an inflammatory bowel disease; e.g., Crohn's disease or ulcerative colitis), wherein the method comprises orally administering to the subject an effective amount of a chemical entity (e.g., a compound of Formula (I), or a pharmaceutically acceptable salt and/or cocrystal thereof) as defined anywhere herein, wherein the AUC24 of the compound of formula

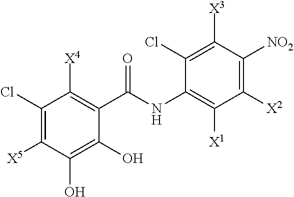

in the plasma of the subject is smaller than the AUC24 of the compound of formula

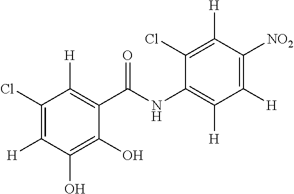 [SMILES: O=C(Nc1ccc([N+](=O)[O-])cc1Cl)c1cc(Cl)cc(O)c1O]

in the plasma of a second subject following administration to the second subject of the same amount of niclosamide as the amount of the compound of Formula (I). In some embodiments, the compound of Formula (I) is selected from a compound of Table 1.

In some embodiments, provided herein is a method for treating colitis, e.g., autoimmune colitis, e.g., an inflammatory bowel disease; e.g., Crohn's disease or ulcerative (or one or more symptoms thereof) in a subject, wherein the method comprises administering to the subject an effective amount of a chemical entity (e.g., a compound of Formula (I), or a pharmaceutically acceptable salt and/or cocrystal thereof) as defined anywhere herein, wherein the AUC24 of the compound of formula

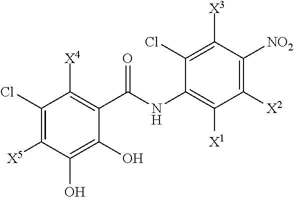

in the plasma of the subject is smaller than the AUC24 of the compound of formula

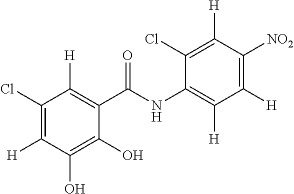 [SMILES: O=C(Nc1ccc([N+](=O)[O-])cc1Cl)c1cc(Cl)cc(O)c1O]

in the plasma of a second subject following administration to the second subject of the same amount of niclosamide as the amount of the compound of Formula (I). In some embodiments, the compound of Formula (I) is selected from a compound of Table 1.

In some embodiments, provided herein is a method for treating a condition (or one or more symptoms thereof) selected from the group consisting of celiac disease, irritable bowel syndrome, mucositis, uveitis, collagenous colitis, lymphocytic colitis, microscopic colitis, radiation enteritis, rheumatoid arthritis, lupus, scleroderma, psoriasis, cutaneous T-cell lymphoma, acute graft vs. host disease and chronic graft vs. host disease in a subject, wherein the method comprises administering to the subject an effective amount of a chemical entity (e.g., a compound of Formula (I), or a pharmaceutically acceptable salt and/or cocrystal thereof) as defined anywhere herein, wherein the AUC24 of the compound of formula

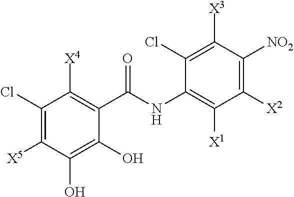

in the plasma of the subject is smaller than the AUC24 of the compound of formula

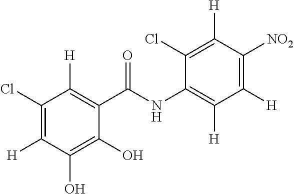 [SMILES: O=C(Nc1ccc([N+](=O)[O-])cc1Cl)c1cc(Cl)cc(O)c1O]

in the plasma of a second subject following administration to the second subject of the same amount of niclosamide as the amount of the compound of Formula (I). In some embodiments, the compound of Formula (I) is selected from a compound of Table 1.

A method of evaluating the metabolic stability of a deuterated niclosamide, comprising:

a. contacting a first amount of the deuterated niclosamide with a metabolizing enzyme source for a period of time sufficient to allow the formation of one or more metabolic products of the deuterated niclosamide and a remaining amount of the deuterated niclosamide;

b. determining at the period of time the remaining amount of the deuterated niclosamide and the amount of at least one of the one or more metabolic products; and

c. comparing the remaining amount of the deuterated niclosamide determined in step b. with the first amount of the deuterated niclosamide and/or comparing the first amount of the deuterated niclosamide with the amount of the at least one of the one or more metabolic products determined in step b. to determine the metabolic stability of the deuterated niclosamide, wherein the metabolic stability is expressed as the % of the amount of the deuterated niclosamide determined in step b. relative to the first amount and/or as the % of the at least one of the one or more metabolic products determined in step b. relative to the first amount of the deuterated niclosamide.

In some embodiments, the method further comprises determining the ratio of the metabolic stability of the deuterated niclosamide determined in step c. to the metabolic stability of niclosamide, wherein the metabolic stability of niclosamide is determined by

d. contacting a first amount of niclosamide with the metabolizing enzyme source of step a. for the period of time of step a. to allow the formation of one or more metabolic products of niclosamide and a remaining amount of niclosamide;

e. determining at the period of time the remaining amount of niclosamide and the amount of at least one of the one or more metabolic products; and

f. comparing the remaining amount of niclosamide determined in step e. with the first amount of niclosamide and/or comparing the first amount of niclosamide with the amount of the at least one of the one or more metabolic products determined in step e. to determine the metabolic stability of niclosamide, wherein the metabolic stability is expressed as the % of the amount of niclosamide determined in step e. relative to the first amount of niclosamide and/or as the % of the at least one of the one or more metabolic products determined in step e. relative to the first amount of niclosamide.

In some embodiments, the amount of deuterated niclosamide in step a. and the amount of niclosamide in step d. are the same.

A method of evaluating the metabolic stability of a deuterated niclosamide in a subject, comprising:

a. administering a first amount of the deuterated niclosamide to a subject;

b. obtaining a serum, blood, tissue, urine, or feces sample from the subject at a period of time following the administration of the compound to the subject, wherein the period of time is sufficient to allow the formation of one or more metabolic products of the deuterated niclosamide and a remaining amount of the deuterated niclosamide in the sample;

c. determining at the period of time the remaining amount of the deuterated niclosamide and the amount of at least one of the one or more metabolic products; and

d. comparing the remaining amount of the deuterated niclosamide determined in step b. in the sample with the first amount of the deuterated niclosamide and/or comparing the first amount of the deuterated niclosamide with the amount of the at least one of the one or more metabolic products determined in step b. in the sample to determine the metabolic stability of the deuterated niclosamide, wherein the metabolic stability is expressed as the % of the remaining amount of the deuterated niclosamide determined in step b. relative to the first amount and/or as the % of the at least one of the one or more metabolic products determined in step b. relative to the first amount of the deuterated niclosamide.

In some embodiments, the method further comprises determining the ratio of the metabolic stability of the deuterated niclosamide determined in step d. to the metabolic stability of niclosamide, wherein the metabolic stability of niclosamide is determined by

e. administering a first amount of the niclosamide to the subject;

f. obtaining a sample from one of serum, blood, tissue, urine, or feces from the subject at the period of time of step b. to allow the formation of one or more metabolic products of the niclosamide and a remaining amount of the niclosamide in the sample, wherein the one of serum, blood, tissue, urine, or feces is the same as in step b.;

g. determining at the period of time the remaining amount of the niclosamide and the amount of at least one of the one or more metabolic products; and

h. comparing the remaining amount of the niclosamide determined in step f. in the sample with the first amount of the niclosamide and/or comparing the first amount of the niclosamide with the amount of the at least one of the one or more metabolic products determined in step f. in the sample to determine the metabolic stability of the niclosamide, wherein the metabolic stability is expressed as the % of the remaining amount of the niclosamide determined in step f. relative to the first amount and/or as the % of the at least one of the one or more metabolic products determined in step f. relative to the first amount of the niclosamide.

In some embodiments, the amount of deuterated niclosamide in step a. and the amount of niclosamide in step e. are the same.

In some embodiments, the compounds of formula (I) have a increased solubility (e.g., in water or aqueous solutions) relative to niclosamide.

In some embodiments, the compounds of formula (I) have a reduced solubility (e.g., in water or aqueous solutions) relative to niclosamide.

In some embodiments, the compounds of formula (I) have a increased flowability relative to niclosamide, and as such, are more amenable, e.g., for forming suspensions in liquid carriers, e.g., carriers suitable for administration by enema.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples.

Compound Preparation

As can be appreciated by the skilled artisan, methods of synthesizing the compounds of the formulae herein will be evident to those of ordinary skill in the art. For example, the compounds described herein can be synthesized. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and R G M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof. The starting materials used in preparing the compounds of the invention are known, made by known methods, or are commercially available. The skilled artisan will also recognize that conditions and reagents described herein that can be interchanged with alternative art-recognized equivalents. For example, in many reactions, triethylamine can be interchanged with other bases, such as non-nucleophilic bases (e.g. diisopropylamine, 1,8-diazabicycloundec-7-ene, 2,6-di-tert-butylpyridine, or tetrabutylphosphazene).

The skilled artisan will recognize a variety of analytical methods that can be used to characterize the compounds described herein, including, for example, ¹H NMR, heteronuclear NMR, mass spectrometry, liquid chromatography, and infrared spectroscopy. The foregoing list is a subset of characterization methods available to a skilled artisan and is not intended to be limiting.

Schemes 1-3 (below) illustrate non-limiting exemplary methods for preparing a compound of Formula (I).

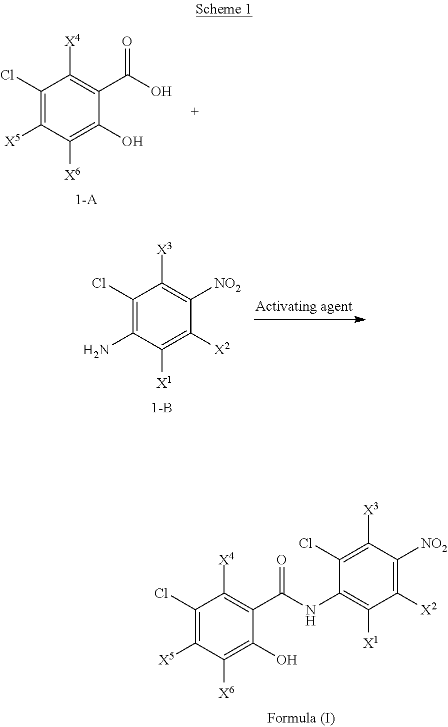

Referring to Scheme 1, a compound of Formula 1-A can be coupled with a compound of Formula 1-B in the presence of an appropriate electrophilic activating agent (e.g., PC13) and an appropriate solvent (e.g., toluene) to provide a compound of Formula (I). X¹, X², X³, X⁴, X⁵, and X⁶ in Formulae 1-A, 1-B, and (I) can be defined as anywhere herein provided that one or more of X¹, X², X³, X⁴, X⁵, and X⁶ is deuterium. As a non-limiting example, the coupling between 1-A and 1-B can be carried out under conditions similar to those described in Antimicrobial Agents and Chemotherapy 2016, 60, 323 and/or Osol, A. (ed.). Remington's Pharmaceutical Sciences. 16th ed. Easton, Pa.: Mack Publishing Co., 1980. (see e.g., page 1182), each of which is incorporated herein by reference in its entirety.

Scheme 2 describes another method for preparing a compound of Formula (I).

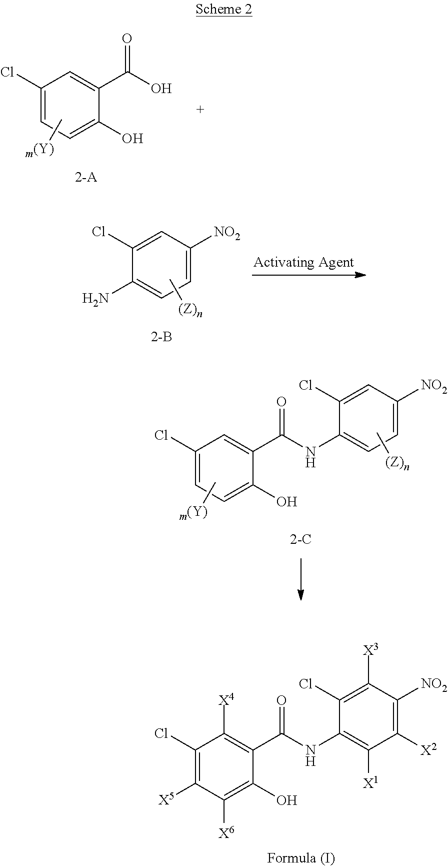

Referring to Scheme 2, a compound of Formula 2-A (wherein m is 0, 1, 2, or 3; and Y is —Br or —I) can be reacted with a compound of Formula 2-B (wherein n is 0, 1, 2, or 3; provided that m+n>0; and Z is —Br or —I) in the presence of an appropriate electrophilic activating agent (e.g., PC3) to provide 2-C. As a non-limiting example, the coupling between 2-A and 2-B can be carried out under conditions similar to those described in Antimicrobial Agents and Chemotherapy 2016, 60, 323 and/or Osol, A. (ed.). Remington's Pharmaceutical Sciences. 16th ed. Easton, Pa.: Mack Publishing Co., 1980. (see e.g., page 1182), each of which is incorporated herein by reference in its entirety. The Y and Z groups of compound 2C can be reduced e.g., in the presence of a metal catalyst (e.g., a palladium catalyst and an appropriate ligand) and a deuterium source (e.g., NaBD₄ or DC(═O)ONa) (see e.g., Org. Chem. Front. 2015, 2, 1071-1075 which is incorporated herein by reference in its entirety) to provide a compound of Formula (I), wherein X¹, X², X³, X⁴, X⁵, and X⁶ can be defined as anywhere herein provided that one or more of X¹, X², X³, X⁴, X⁵, and X⁶ is deuterium.

Scheme 3 describes yet another method for preparing a compound of Formula (I).

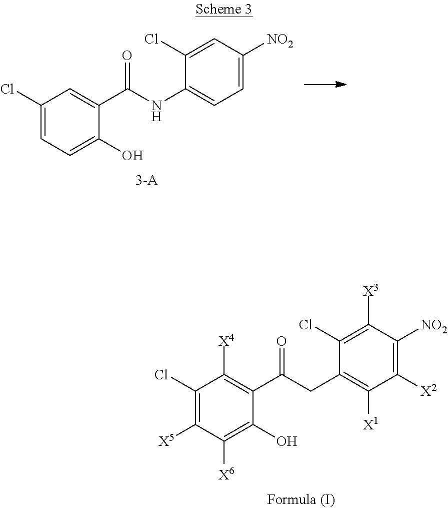

Referring to Scheme 3, niclosamide (3-A) can be reacted with D2 gas in the presence of a metal catalyst (e.g., an iridium catalyst) to provide a compound of Formula (I) (see e.g., J. Label Compd. Radiopharm 2010, 53, 662; Org. Biomol. Chem., 2014, 12, 7927; Org. Biomol. Chem., 2014, 12, 3598; Adv. Synth. Catal. 2014, 356, 3551, each of which is incorporated herein by reference in its entirety).

### OTHER EMBODIMENTS

Exemplary embodiments include the embodiments in the following clauses:

1. A method for treating a digestive symptom in a subject diagnosed as having a COVID-19 viral infection and in need of such treatment, the method comprising orally administering an effective amount of any formula described herein: or a pharmaceutically acceptable salt thereof, to the subject;

wherein the effective amount of niclosamide, or a pharmaceutically acceptable salt thereof, decreases an amount of COVID-19-viral RNA load in a fecal sample, an anal swab sample, or a rectal swab sample obtained from the subject by about 45% to about 99% relative to a baseline amount of COVID-19-viral RNA load,

wherein the baseline amount of COVID-19 viral RNA load is an amount of COVID-19-related viral RNA that is present in a fecal sample, an anal swab sample, or a rectal swab sample obtained from the subject prior to administration of the niclosamide, or a pharmaceutically acceptable salt thereof.

2. The method of clause 1, wherein the method comprises administering deuterated niclosamide.

3. The method of clause 1 or 2, wherein at least some of the COVID-19 viral infection is present in the gastrointestinal tract of the subject.

4. The method of any of clauses 1-3, wherein the effective amount of niclosamide, or a pharmaceutically acceptable salt thereof, decreases a COVID-19 viral load in the gastrointestinal tract of the subject relative to a baseline COVID-19 viral load, wherein the baseline COVID-19 viral load is the COVID-19 viral load in the gastrointestinal tract of the subject prior to administration of the niclosamide, or a pharmaceutically acceptable salt thereof.

5. The method of any of clauses 1-4, wherein the digestive symptom is selected from the group consisting of lack or loss of appetite, diarrhea, vomiting, abdominal pain, and combinations thereof.

6. The method of any of clauses 1-5, wherein the digestive symptom is diarrhea.

7. The method of any of clauses 1-6, wherein the subject does not exhibit an accompanying respiratory symptom.

8. The method of any of clauses 1-7, wherein the subject exhibits an accompanying respiratory symptom.

9. The method of of any of clauses 1-8, wherein the method comprises measuring viral load with rRT-PCR.

10. The method of any of clauses 1-9, wherein the subject suffers from, or is predisposed to suffer from colitis.

11. The method of any of clauses 1-10, wherein the colitis is an autoimmune colitis.

12. The method of any of clauses 1-11, wherein the colitis is an inflammatory bowel disease.

13. The method of any of clauses 1-12, wherein the colitis is ulcerative colitis, Crohn's disease, or iatrogenic autoimmune colitis.

14. The method of any of clauses 1-13, wherein the digestive symptom appears from 2-14 days after the subject's exposure to coronavirus.

15. The method of any of clauses 1-14, wherein the method further comprises administering a second therapeutic agent.

16. The method of any of clauses 1-15, wherein the second therapeutic agent is selected from the group consisting of azithromycin, remdesivir, hydroxychloroquine, colchicine, and chloroquine.

17. The method of any of clauses 1-16, wherein the niclosamide, or a pharmaceutically acceptable salt thereof, is administered by tablet or pill.

18. The method of any of clauses 1-17, wherein the method comprises orally administering the niclosamide, or a pharmaceutically acceptable salt thereof, as a pharmaceutical composition, wherein the pharmaceutical composition is capable of local delivery to the GI tract.

19. The method of any of clauses 1-18, wherein the method comprises orally administering the niclosamide, or a pharmaceutically acceptable salt thereof, as a pharmaceutical composition, wherein the pharmaceutical composition is capable of local delivery to the colon or small intestine.

20. The method of any of clauses 1-19, wherein the decrease occurs within about one week, or about two weeks, or about three weeks, or about four weeks from the start of administration of the niclosamide, or a pharmaceutically acceptable salt thereof.

21. A method for treating a subject having a COVID-19 viral infection and is in need of such treatment, the method comprising orally administering an effective amount of niclosamide: or a pharmaceutically acceptable salt thereof, to the subject so as to treat the COVID-19 viral infection;

wherein the effective amount of niclosamide, or a pharmaceutically acceptable salt thereof, decreases an amount of COVID-19 viral RNA load in a fecal sample, an anal swab sample, or a rectal swab sample obtained from the subject by about 45% to about 99% relative to a baseline amount of COVID-19 viral RNA load,

wherein the baseline amount of COVID-19 viral RNA load is an amount of COVID-19 viral RNA that is present in a fecal sample, an anal swab sample, or a rectal swab sample obtained from the subject prior to administration of the niclosamide, or a pharmaceutically acceptable salt thereof.

22. The method of clause 21, wherein the decrease occurs within about one week from the start of administration of the niclosamide, or a pharmaceutically acceptable salt thereof.

23. The method of clause 21 or 22, wherein the decrease occurs within about two weeks from the start of administration of the niclosamide, or a pharmaceutically acceptable salt thereof.

24. The method of any of clauses 21-23, wherein the decrease occurs within about three weeks from the start of administration of the niclosamide, or a pharmaceutically acceptable salt thereof.

25. The method of any of clauses 21-24, wherein the decrease occurs within about four weeks from the start of administration of the niclosamide, or a pharmaceutically acceptable salt thereof.

### EXAMPLES

### Evaluation of Metabolic Stability

Certain in vitro liver metabolism studies have been described previously in the following references, each of which is incorporated herein in their entirety: Obach, R. S. Drug Metab Disp 1999, 27, p. 1350; Houston, J. B. et al., Drug Metab Rev 1997, 29, p. 891; Houston, J. B. Biochem Pharmacol 1994, 47, p. 1469; Iwatsubo, T et al., Pharmacol Ther 1997, 73, p. 147; and Lave, T. et al., Pharm Res 1997, 14, p. 152.

Microsomal Assay: The metabolic stability of compounds of Formula A or Formula I is tested using pooled liver microsomal incubations. Full scan LC-MS analysis is then performed to detect major metabolites. Samples of the test compounds, exposed to pooled human liver microsomes, are analyzed using HPLC-MS (or MS/MS) detection. For determining metabolic stability, multiple reaction monitoring (MRM) is used to measure the disappearance of the test compounds. For metabolite detection, Q1 full scans are used as survey scans to detect the major metabolites.

Experimental Procedures: Human liver microsomes are obtained from a commercial source (e.g., Absorption Systems L.P. (Exton, Pa.), or XenoTech, LLC (Lenexa, Kans.)). The incubation mixtures are prepared as follows:

Reaction Mixture Composition

| | | |
|---|---|---|
| Liver Microsomes | 0.5-2.0 | mg/mL |
| NADPH | 1 | mM |
| Potassium Phosphate, pH 7.4 | 100 | mM |
| Magnesium Chloride | 10 | mM |
| Test Compound | 0.1-1 | μM. |

Incubation of Test Compounds with Liver Microsomes: The reaction mixture, minus cofactors, is prepared. An aliquot of the reaction mixture (without cofactors) is incubated in a shaking water bath at 37° C. for 3 minutes. Another aliquot of the reaction mixture is prepared as the negative control. The test compound is added into both the reaction mixture and the negative control at a final concentration of 1 μM. An aliquot of the reaction mixture is prepared as a blank control, by the addition of plain organic solvent (no test compound is added). The reaction is initiated by the addition of cofactors (not added to the negative controls), and then incubated in a shaking water bath at 37° C. Aliquots (200 μL) are withdrawn in triplicate at multiple time points (e.g., 0, 15, 30, 60, and 120 minutes) and combined with 800 μL of ice-cold 50/50 acetonitrile/dH₂O to terminate the reaction. The positive controls, testosterone and propranolol, as well as agomelatine, are each run simultaneously with the test compounds in separate reactions.

All samples are analyzed using LC-MS (or MS/MS). An LC-MRM-MS/MS method is used for metabolic stability. Also, Q1 full scan LC-MS methods are performed on the blank matrix and the test compound incubation samples. The Q1 scans serve as survey scans to identify any sample unique peaks that might represent the possible metabolites. The masses of these potential metabolites can be determined from the Q1 scans.

SUPERSOMFS™ Assay. Various human cytochrome P450-specific SUPERSOMES™ are purchased from Gentest (Woburn, Mass., USA). A 1.0 mL reaction mixture containing 25 pmole of SUPERSOMES™, 2.0 mM NADPH, 3.0 mM MgCl, and 1 μM of a compound of Formula A or Formula I in 100 mM potassium phosphate buffer (pH 7.4) is incubated at 37° C. in triplicate. Positive controls contain 1 μM of agomelatine instead of a compound of Formula A or Formula I. Negative controls used Control Insect Cell Cytosol (insect cell microsomes that lacked any human metabolic enzyme) purchased from GenTest (Woburn, Mass., USA). Aliquots (50 μL) are removed from each sample and placed in wells of a multi-well plate at various time points (e.g., 0, 2, 5, 7, 12, 20, and 30 minutes) and to each aliquot is added 50 μL of ice cold acetonitrile with 3 μM haloperidol as an internal standard to stop the reaction.

Plates containing the removed aliquots are placed in −20° C. freezer for 15 minutes to cool. After cooling, 100 μL of deionized water is added to all wells in the plate. Plates are then spun in the centrifuge for 10 minutes at 3000 rpm. A portion of the supernatant (100 μL) is then removed, placed in a new plate and analyzed using Mass Spectrometry.

Methods for evaluating biological activity of the compounds described here, formulating the compounds described here, and forming co-crystals of the compounds described herein can be found, e.g., in WO 2017/040864, which is incorporated by reference in its entirety.

A number of embodiments of the invention have been described. Nevertheless, it will be understood that various modifications may be made without departing from the spirit and scope of the invention. Accordingly, other embodiments are within the scope of the following claims.

1. A compound of Formula (I), 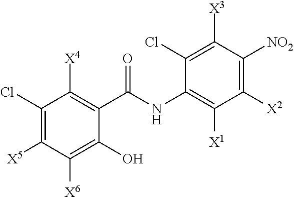
or a pharmaceutically acceptable salt thereof
wherein:
X¹ is hydrogen or deuterium;
X² is hydrogen or deuterium;
X³ is hydrogen or deuterium;
X⁴ is hydrogen or deuterium;
X⁵ is hydrogen or deuterium;
X⁶ is hydrogen or deuterium;
provided that the compound comprises deuterium;
and further provided that if X¹, X², X³ and X⁴ are each deuterium, then at least one of X⁵ or X⁶ is deuterium.

2. The compound of claim 1, wherein X¹ is deuterium.

3. The compound of claim 1, wherein X¹ is hydrogen.

4. The compound of claim 1, wherein X² is deuterium.

5. The compound of claim 1, wherein X² is hydrogen.

6. The compound of claim 1, wherein X³ is deuterium.

7. The compound of claim 1, wherein X³ is hydrogen.

8. The compound of claim 1, wherein X⁴ is deuterium.

9. The compound of claim 1, wherein X⁴ is hydrogen.

10. The compound of claim 1, wherein X⁵ is deuterium.

11. The compound of claim 1, wherein X⁵ is hydrogen.

12. The compound of claim 1, wherein X⁶ is deuterium.

13. The compound of claim 1, wherein X⁶ is hydrogen.

14. The compound of claim 1, wherein the compound is selected from the group consisting of: 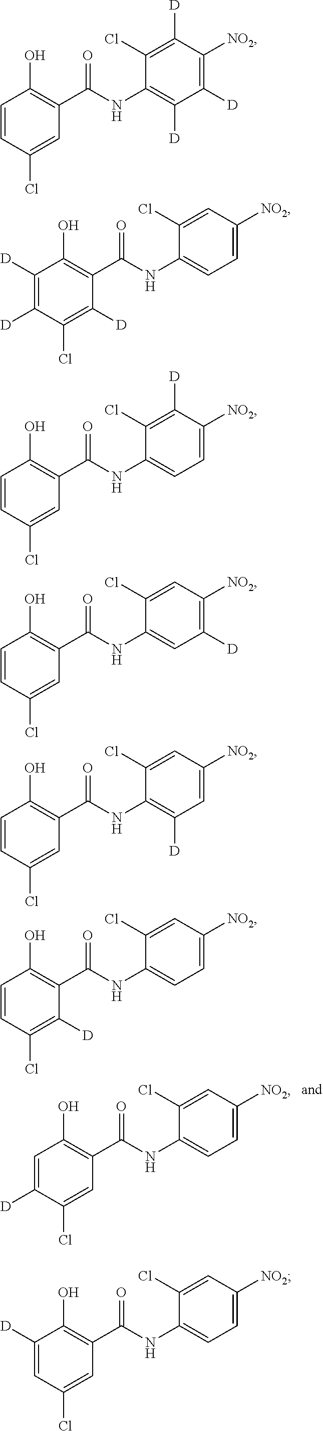
or a pharmaceutically acceptable salt thereof.

15. The compound of claim 1, wherein the compound has a chemical purity of greater than about 99.0%.

16. The compound of claim 1, wherein the compound has reduced particle size.

17. The compound of claim 1, wherein the compound has a particle size distribution D(0.9) of from about 6.0 μm to about 8.0 μm, a particle size distribution D(0.5) of from about 1.0 μm to about 4.0 μm, and a particle size distribution D(0.1) of from about 0.3 μm to about 0.9 μm.

18. The compound of claim 1, wherein the compound has a chemical purity of greater than about 99.0%, a particle size distribution D(0.9) of from about 1.0 μm to about 10.0 μm, a particle size distribution D(0.5) of from about 1.0 μm to about 4.0 μm, and a particle size distribution D(0.1) of from about 0.1 μm to about 1.0 μm.

19. (canceled)

20. A composition comprising: (i) a compound of claim 1, and (ii) one or more pharmaceutically acceptable excipients.

21-26. (canceled)

27. A method for treating a subject having a condition associated with unregulated (such as abnormal or elevated) recruitment and/or retention of one or more T cells at the gastrointestinal tract (GI) of the subject, the method comprising contacting the one or more T cells with an effective amount of a compound of Formula (I) as claimed in claim 1, or a pharmaceutically acceptable salt thereof.

28-60. (canceled)
